SEQUENCE LISTING (4 sequences)

SEQ ID NO: 1 - DNA, 20 bp, Homo sapiens
  source 1..20: mol_type = genomic DNA
gatcatcagcaatgcctcct

SEQ ID NO: 2 - DNA, 20 bp, Homo sapiens
  source 1..20: mol_type = genomic DNA
tgtggtcatgagtccttcca

SEQ ID NO: 3 - DNA, 20 bp, Homo sapiens
  source 1..20: mol_type = genomic DNA
tacggttggtcctttcttcg

SEQ ID NO: 4 - DNA, 20 bp, Homo sapiens
  source 1..20: mol_type = genomic DNA
tttcctttgggtctgaatgc

(19) United States
(12) Patent Application Publication
(10) Pub. No.: US 2026/0279569 A1
(43) Pub. Date: Sep. 17, 2026

(54) Systems and Methods for Sequencing of Cell-Free RNA

(71) Applicant: The Board of Trustees of the Leland Stanford Junior University, Stanford, CA (US)

(72) Inventors: Maximilian Diehn, San Carlos, CA (US); Arash Ash Alizadeh, San Mateo, CA (US); Monica Nesselbush, Stanford, CA (US); Bogdan Luca, Stanford, CA (US)

(73) Assignee: The Board of Trustees of the Leland Stanford Junior University, Stanford, CA (US)

(21) Appl. No.: 19/388,550

(22) Filed: Nov. 13, 2025

Related U.S. Application Data
(63) Continuation of application No. PCT/US2024/029513, filed on May 15, 2024.
(60) Provisional application No. 63/502,368, filed on May 15, 2023.

(51) Int. Cl. G16H 50/20; C12Q 1/6858; C12Q 1/6886; G16B 20/20; G16B 25/20; G16B 30/10; G16H 20/10; G16H 50/70

(52) U.S. Cl. CPC G16H 50/20; C12Q 1/6858; C12Q 1/6886; G16B 20/20; G16B 25/20; G16B 30/10; G16H 20/10; G16H 50/70

(57) ABSTRACT

Systems and methods for sequencing of cell-free RNAs are provided. Targeted sequencing can be performed using a panel of transcripts that are genes of rare abundance within a population of control samples of cell-free nucleic acids.

### CROSS REFERENCE TO RELATED APPLICATIONS

This application is a continuation of PCT Patent Application Ser. No. PCT/US2024/029513, entitled “Systems and Methods for Sequencing of Cell-Free RNA,” filed May 15, 2024, which claims priority to U.S. Provisional Application Ser. No. 63/502,368, entitled “Systems and Methods for Sequencing of Cell-Free RNA,” filed May 15, 2023, the disclosures of which are hereby incorporated by reference in their entireties.

### STATEMENT REGARDING FEDERALLY SPONSORED RESEARCH OR DEVELOPMENT

This invention was made with Government support under contract CA254179 awarded by the National Institutes of Health. The Government has certain rights in the invention.

### SEQUENCE LISTING

The instant application contains a Sequence Listing which has been submitted electronically in XML format and is hereby incorporated by reference in its entirety. Said XML copy, created on May 14, 2024, is named 08574SeqListing.xml and is 4,382 bytes in size.

### TECHNICAL FIELD

The disclosure provides description of an improved method for performing sequencing on cell-free RNA.

### BACKGROUND

Blood-based liquid biopsies enable non-invasive characterization of health, including detection of biological phenomena such as pregnancy and cancer. Liquid biopsies offer many advantages over tissue biopsies because they are minimally invasive, easily repeated over time, and more accurately reflect the geographic heterogeneity among the cellular sources. In patients with advanced cancer disease, analysis of circulating tumor DNA (ctDNA) is used clinically for non-invasive genotyping. However, full clinical evaluation usually requires expression-based analyses, such as for distinguishing between tumor types or subtypes.

### SUMMARY

In some implementations, a method for sequencing of cell-free RNA comprises performing targeted sequencing. The sequencing is targeted at cell-free RNA molecules that are infrequently expressed within liquid biopsies of control individuals.

In some implementations, a panel of nucleic acids is for targeting transcripts that are rarely abundant as cell-free RNA molecules.

In some implementations, a panel of nucleic acids comprises nucleic acid molecules having sequences from or complement to gene transcripts that are rarely abundant as cell-free RNA molecules within control liquid biopsies.

In some implementations, the transcripts that are rarely abundant as cell-free RNA molecules within control liquid biopsies are defined as transcripts that are expressed in less than 50% of a population of control liquid biopsies.

In some implementations, the transcripts that are rarely abundant as cell-free RNA molecules within control liquid biopsies are defined as transcripts that are expressed in less than 5% of a population of control liquid biopsies.

In some implementations, the transcripts that are rarely abundant as cell-free RNA molecules within control liquid biopsies are defined as transcripts that are in the bottom 60% of genes with respect to normalized expression across a population of control liquid biopsies.

In some implementations, the transcripts that are rarely abundant as cell-free RNA molecules within control liquid biopsies are defined as transcripts that are in the bottom 30% of genes with respect to normalized expression across the population of control liquid biopsies.

In some implementations, the transcripts that are rarely abundant as cell-free RNA molecules within control liquid biopsies are defined as transcripts having log transformed and normalized expression values less than zero across a population of control liquid biopsies.

In some implementations, the population of control liquid biopsies comprises at least 5 liquid biopsies.

In some implementations, the population of control liquid biopsies comprises at least 50 liquid biopsies.

In some implementations, the control liquid biopsies are collected from individuals not having one or more the following when the biopsy is collected: an observed pathogenic infection, a diagnosed cancer, a diagnosed metabolic disorder, a diagnosed neurological disorder, a diagnosed immunodeficiency disorder, a diagnosed autoimmune disorder, a diagnosed inflammatory disorder, a diagnosed cardiovascular disorder, a diagnosed renal disorder, a diagnosed hepatic disorder, active pregnancy, a diagnosed pregnancy complication, a diagnosed fetal complication, an organ transplant, active rejection of an organ transplant, obesity, malnourishment, cachexia, and an abnormality on a clinical test.

In some implementations, the transcripts that are rarely abundant as cell-free RNA molecules within control liquid biopsies are defined as transcripts that are expressed in less than 5% of a population of control liquid biopsies.

In some implementations, the transcripts that are rarely abundant as cell-free RNA molecules within control liquid biopsies are defined as transcripts that are in the bottom 60% of genes with respect to normalized expression across a population of control liquid biopsies.

In some implementations, the transcripts that are rarely abundant as cell-free RNA molecules within control liquid biopsies are defined as transcripts that are in the bottom 30% of genes with respect to normalized expression across the population of control liquid biopsies.

In some implementations, the transcripts that are rarely abundant as cell-free RNA molecules within control liquid biopsies are defined as transcripts having log transformed and normalized expression values less than zero across a population of control liquid biopsies.

In some implementations, the population of control liquid biopsies comprises at least 5 liquid biopsies.

In some implementations, the population of control liquid biopsies comprises at least 50 liquid biopsies.

In some implementations, the control liquid biopsies are collected from individuals not having one or more the following when the biopsy is collected: an observed pathogenic infection, a diagnosed cancer, a diagnosed metabolic disorder, a diagnosed neurological disorder, a diagnosed immunodeficiency disorder, a diagnosed autoimmune disorder, a diagnosed inflammatory disorder, a diagnosed cardiovascular disorder, a diagnosed renal disorder, a diagnosed hepatic disorder, active pregnancy, a diagnosed pregnancy complication, a diagnosed fetal complication, an organ transplant, active rejection of an organ transplant, obesity, malnourishment, cachexia, and an abnormality on a clinical test.

In some implementations, the transcripts that are rarely abundant as cell-free RNA molecules within control liquid biopsies comprise 50% of transcripts from Table 3.

In some implementations, the transcripts that are rarely abundant as cell-free RNA molecules within control liquid biopsies comprise 90% of transcripts from Table 3.

In some implementations, the transcripts that are rarely abundant as cell-free RNA molecules within control liquid biopsies comprise 100% of transcripts from Table 3.

In some implementations, the panel of nucleic acid molecules excludes at least 50% of whole-exome gene transcripts that are not transcripts that are rarely abundant as cell-free RNA molecules.

In some implementations, the panel of nucleic acid molecules excludes at least 90% of whole-exome gene transcripts that are not transcripts that are rarely abundant as cell-free RNA molecules.

In some implementations, the panel of nucleic acid molecules consists of 5000 or fewer gene transcripts in addition to transcripts that are rarely abundant as cell-free RNA molecules.

In some implementations, the panel of nucleic acid molecules consists of 500 or fewer gene transcripts in addition to transcripts that are rarely abundant as cell-free RNA molecules.

In some implementations, the panel of nucleic acid molecules further comprises tissue-specific transcripts, cell-type-specific transcripts, clinically relevant transcripts, B-cell receptor and T-cell receptor transcripts, biomarkers, and commonly mutagenized transcripts.

In some implementations, the biomarkers are associated with one of the following biological characteristics: a medical disorder, pregnancy, a fetal complication, a pregnancy complication, a neoplastic growth, cancer, a particular cancer type, a pathogen infection, immune activation, organ transplant rejection, neurodegeneration, tissue of origin, cell type of origin, or activation of a biochemical pathway.

In some implementations, the panel of nucleic acid molecules further comprises a set of control transcripts for normalization between samples.

In some implementations, the panel of nucleic acid molecules are a set of probes for targeted capture hybridization.

In some implementations, the panel of nucleic acid molecules are a set of primers for targeted amplification.

In some implementations, a method for preparing for sequencing of cell-free RNA comprises providing a sample comprising nucleic acids for sequencing.

In some implementations, the nucleic acids for sequencing are cell-free RNA or nucleic acids derived from and representative of cell-free RNA.

In some implementations, a method for preparing for sequencing of cell-free RNA comprises contacting the sample with a panel of nucleic acid molecules that comprises molecules having sequences from or complement to gene transcripts that are rarely abundant as cell-free RNA molecules within control liquid biopsies such that the panel of nucleic acid molecules anneals with a subset of the nucleic acids for sequencing.

In some implementations, the sample of cfRNA is derived from blood, plasma, lymph, cerebrospinal fluid, amniotic fluid, urine, or stool.

In some implementations, a method for preparing for sequencing of cell-free RNA comprises generating a sequencing library derived from the sample.

In some implementations, a method for preparing for sequencing of cell-free RNA comprises performing targeted sequencing of the sequencing library to yield a sequencing result of the cell-free RNA. The sequencing is targeted towards the panel of nucleic acid molecules.

In some implementations, a method for preparing for sequencing of cell-free RNA comprises removing platelet expression from the sequencing result in silico.

In some implementations, a method for preparing for sequencing of cell-free RNA comprises performing differential transcript analysis with the sequencing result and a second sequencing result.

In some implementations, a method for preparing for sequencing of cell-free RNA comprises detecting enrichment of at least one expression signature within the sequencing result.

In some implementations, a method for preparing for sequencing of cell-free RNA comprises detecting sequence mutagenesis within the sequencing result.

In some implementations, a method for preparing for sequencing of cell-free RNA comprises inferring copy number status of one or more genes from the sequencing result.

In some implementations, a method for preparing for sequencing of cell-free RNA comprises utilizing the sequencing result along with a plurality of other sequencing results to train a computational model to predict a categorical status or a likelihood of a biological characteristic. The cell-free RNA sample has a known categorical status of a biological characteristic.

In some implementations, a method for preparing for sequencing of cell-free RNA comprises utilizing the sequencing result as input within a trained computational model to predict a categorical status or a likelihood of a biological characteristic. The computational model has been trained utilizing a cohort of RNA sequencing results having a known categorical status of a biological characteristic.

In some implementations, a method for preparing for sequencing of cell-free RNA comprises deriving one or more features from the sequencing result. The one or features comprises enrichment of one or more gene signatures, enrichment of biochemical pathways, collection of sequence variants, and copy number status.

In some implementations, a method for preparing for sequencing of cell-free RNA comprises utilizing the one or more derived features as input within a trained computational model to predict a categorical status or a likelihood of a biological characteristic. The computational model has been trained utilizing a cohort of RNA sequencing results having a known categorical status of a biological characteristic.

In some implementations, a method for extracting RNA from a cell-free source comprises (a) adding glycogen to a sample comprising cell-free nucleic acids.

In some implementations, a method for extracting RNA from a cell-free source comprises (b) contacting a silica-based column with a sample comprising cell-free nucleic acids.

In some implementations, step (a) is performed before step (b).

In some implementations, a method for extracting RNA from a cell-free source comprises eluting cell-free nucleic acids from the silica-based column to yield a solution of extracted cell-free nucleic acids.

In some implementations, a method for extracting RNA from a cell-free source comprises contacting the solution of extracted cell-free nucleic acids with a DNase.

In some implementations, a method for quantifying cell-free RNA for downstream molecular applications comprises providing a sample comprising cell-free RNA.

In some implementations, a method for quantifying cell-free RNA for downstream molecular applications comprises reverse transcribing the cell-free RNA to yield cDNA.

In some implementations, a method for quantifying cell-free RNA for downstream molecular applications comprises quantifying the concentration of cell-free RNA within the solution using quantitative real-time polymerase chain reaction and the cDNA.

In some implementations, a method for quantifying cell-free RNA for downstream molecular applications comprises performing one or more downstream steps of a molecular protocol using a defined amount of material based on the quantification of cell-free RNA.

In some implementations, the step of quantifying the concentration of cell-free RNA further comprises generating a standard curve based on a set of control standards having known concentration. The control standards are also assessed using quantitative real-time polymerase chain reaction.

In some implementations, the sample further comprises cell-free DNA. A method for quantifying cell-free RNA for downstream molecular applications further comprises quantifying the concentration of cell-free DNA within the sample using quantitative real-time polymerase chain reaction. The cell-free RNA is quantified by using primers that span across an intron of a gene that is relatively stable across cell-free RNA samples and the cell-free DNA is quantified by using primers that anneal to a transcriptionally silent region of a genome that is relatively stable across cell-free DNA samples.

In some implementations, the primers for quantifying cell-free RNA span across an intron of GAPDH and the primers for quantifying cell-free DNA target cover a 78 bp transcriptionally silent region of chromosome 12.

In some implementations, a method for sequencing cell-free RNA comprises providing a library of nucleic acid molecules. The library of nucleic acid molecules was derived from cell-free RNA. The cell-free RNA is derived from a liquid biopsy.

In some implementations, a method for sequencing cell-free RNA comprises sequencing the library of nucleic acid molecules to yield a sequencing result.

In some implementations, a method for sequencing cell-free RNA comprises removing variation due to transcript expression associated with platelets.

In some implementations, the library of nucleic acid molecules was generated by capturing or amplifying nucleic acid molecules.

In some implementations, the library of nucleic acid is a whole exome library.

In some implementations, the library of nucleic acid is a library targeted toward rare abundance genes.

In some implementations, a method for generating a targeted sequencing panel for sequencing of cell-free RNA comprises collecting a population of control liquid biopsies, each comprising cell-free RNA.

In some implementations, a method for generating a targeted sequencing panel for sequencing of cell-free RNA comprises performing sequencing on the cell-free RNA of the control liquid biopsies.

In some implementations, a method for generating a targeted sequencing panel for sequencing of cell-free RNA comprises identifying a set of rare abundance genes within the population of control liquid biopsies as defined by at least one or more of the following: their expression within a percentage of a population of control liquid biopsies or their expression level across the population of control liquid biopsies.

In some implementations, a method for generating a targeted sequencing panel for sequencing of cell-free RNA comprises synthesizing a set of nucleic acid molecules that are for capturing or for amplifying the rare abundance genes to yield the targeted sequencing panel for sequencing of cell-free RNA.

In some implementations, the set of rare abundance genes are defined by at least their expression within a percentage of a population of control liquid biopsies and their expression level across the population of control liquid biopsies.

### BRIEF DESCRIPTION OF THE DRAWINGS

The description and claims will be more fully understood with reference to the following figures and data, which are presented as examples of the disclosure and should not be construed as a complete recitation of the scope of the disclosure.

FIGS. 1A to 1F provides schematics and data charts on optimization of blood collection and cfRNA extraction. FIG. 1A, Representative bioanalyzer trace for cell-free RNA (yellow) and leukocyte cellular RNA (red). FIG. 1B, Concentration of cfRNA per mL of plasma in healthy controls measured via quantitative PCR (see Methods; n=117). Plasma was collected for technical experiments using 2,500G centrifugation for 10 minutes. FIG. 1C, Association between hemolysis and cfRNA plasma concentration. Amount of hemolysis was measured using optical density (OD) at 414 nm. Pearson and Spearman correlations are shown. FIG. 1D, Association between time in −80° C. freezer and cfRNA plasma concentration. Pearson and Spearman correlations are shown. FIG. 1E, Analysis of plasma cfRNA concentrations using different blood collection tubes (BCTs). All samples were spun at 2500G during plasma isolation. Ro, Roche Cell-free DNA BCT. D, Streck Cell-free DNA BCT. R, Streck RNA Complete BCT. E, EDTA. FIG. 1F, Analysis of plasma cfRNA concentrations using different extraction methods. T-R, TRIzol-LS+Qiagen RNeasy Kit. V, QIAamp Viral RNA Kit. Ro, Roche High Pure Vial RNA Kit. M, Qiagen miRNeasy Kit. MPS, Qiagen miRNeasy Serum/Plasma Kit. T-M, TRIzol-LS+Qiagen miRNeasy Kit. P, mirVana PARIS Kit. CCF, QIAamp ccfDNA/RNA Kit. T-V, TRIzol-LS+QIAamp Viral RNA Kit. CNA, QIAamp Circulating Nucleic Acid Kit.

FIGS. 2A to 2K provide data charts on optimization of RARE-Seq library preparation and capture. FIG. 2A, Correlation between cfRNA expression of blood samples collected in EDTA tubes or Streck RNA Complete tubes (average of n=3 pairs). FIG. 2B, Expression correlation from cfRNA extracted using the TRizol LS+QIAamp Viral RNA method (T-V) and the QIAamp Circulating Nucleic Acid Kit (CNA) (average of n=3 pairs). FIG. 2C, Expression correlation between cfRNA libraries generated using stranded and non-stranded methods (average of n=3 pairs). FIG. 2D, Rarefaction analysis representing the relationship between total sequencing depth and unique sequencing depth for cfRNA libraries. The inset plot depicts the percent increase in unique sequencing depth for non-stranded libraries relative to paired stranded libraries. Sequencing depth was downsampled so that pairs had equivalent depth. FIG. 2E, Expression correlation between cfRNA libraries generated with and without S1 nuclease end repair (average of n=3 pairs). FIG. 2F, Rarefaction analysis representing the relationship between total sequencing depth and unique sequencing depth for cfRNA libraries. Log 2NX, log 2 normalized expression. FIG. 2G, Estimated DNA contamination if DNase I digestion performed either on or off Qiagen column. DNA contamination was measured as the percentage of exon-boundary aligning reads that contain adjacent intronic sequence. Conditions were compared using a paired t-test. FIG. 2H Expression correlation between cfRNA samples digested on-column and off-column (average of n=5 pairs). FIG. 2I, Distribution of RNA biotypes in cfRNA using the SMART-Seq whole transcriptome method (n=3). FIG. 2J, Expression correlation from cfRNA libraries generated using SMART-Seq and whole coding transcriptome RARE-Seq (average of n=3 pairs). The histograms depict the log 2nx distribution for each method and the Venn diagram represents the number of coding genes with higher (or equivalent) log 2NX in each method. Sequencing depth was downsampled so that pairs had equivalent depth. Blue=coding genes, gray=non-coding genes. FIG. 2K, Heatmap showing pairwise Pearson correlation between all technical and biological replicates sequenced from a single individual (n=8 replicates).

FIGS. 3A to 3L provide schematics and data showing platelet and non-hematopoietic transcripts are enriched in cfRNA. FIG. 3A, Schematic of the RARE-Seq method. FIG. 3B, Differential expression analysis using whole coding transcriptome RARE-Seq comparing cfRNA and matched leukocyte RNA from healthy donors (n=10 pairs). FIG. 3C, Pre-ranked gene set enrichment analysis using cell type-specific signatures from PanglaoDB (n=170 cell types). The top 15 positively and top 15 negatively enriched signatures are shown, and the dotted line delineates signatures with significant enrichment (P<0.05). FIG. 3D, Relationship between centrifugation speed during plasma isolation and cfRNA concentration per mL plasma (n=10 1200G, n=6 1800G, n=16 2500G). Comparisons were performed using Kruskall-Wallis test. FIG. 3E, Relationship between centrifugation speed and platelet-specific gene expression in cfRNA. Avg log 2NX, mean of normalized expression. Comparisons were performed using Kruskall-Wallis test. FIG. 3F, Principal component analysis of cfRNA expression clusters cfRNA samples according to spin speed (PC1) and sex (PC2). FIG. 3G, Association between average platelet-specific gene expression from FIG. 3E and first principal component from FIG. 3F. Pearson and Spearman correlation are shown. FIG. 3H, Association between average platelet-specific gene expression and first principal component when considering only samples processed using the same centrifugation speed (2500G). FIG. 3I, Schematic of platelet transcript correction approach. See Methods for details. FIG. 3J, Association between platelet-specific gene expression and first principal component, after platelet transcript correction of cfRNA expression. FIG. 3K, Hierarchical clustering of the top 1,000 most variably expressed genes in cfRNA. Clustering was performed using Euclidean distance. FIG. 3L, Hierarchical clustering of the top 1,000 most variably expressed genes after platelet-directed correction.

FIGS. 4A to 4I provide schematics and data showing targeting transcripts absent from healthy cfRNA improves analytical sensitivity for lung cancer detection. FIG. 4A, Schematic depicting the selection of rare abundance genes (RAGs) in plasma. Log 2NX, log 2 normalized expression. Fraction of FIG. 4B, tissue-enriched and FIG. 4C, cancer-enriched genes that overlap RAGs. Tissue-enriched and cancer-enriched genes were selected as defined in Methods. Comparisons were performed using Fisher's exact test. FIG. 4D, Unique depth in libraries captured using either whole coding transcriptome or RAG capture panels (n=5 matched libraries). Libraries were downsampled to have equivalent sequencing depth and filtered to include regions captured by both panels. Comparisons were performed using paired t-test. FIG. 4E, Number of shared genes detected in libraries in FIG. 4D. The threshold for detection was 1 unique read. Comparisons were again performed using paired t-test. FIG. 4F, Histogram depicting the difference in unique depth per gene for libraries in FIG. 4D. FIG. 4G, Schematic of enrichment score (ES) analytical framework (see Methods). FIG. 4H, Relationship between ES detection rate and NCI-H1975 fraction for spikes captured with the RAG panel (n=3 replicates per spike). The presence of cancer RNA was detected in each spike using an NCI-H1975 E S. Logistic regression was used to calculate LOD95 from in silico spikes (shown as grey circles). Results from in vitro spikes are shown as red triangles. FIG. 4I, Empirical LOD of NCI-H1975 spikes captured using the whole coding transcriptome panel (n=1 replicate per spike) evaluated using the same approach as FIG. 4F.

FIGS. 5A to 5I provide data charts on description and validation of RARE-Seq panel targeting rare abundance genes. FIG. 5A, Relationship between average gene expression in healthy cfRNA and overall percentage of healthy cfRNA samples with low or absent gene expression (n=50). Rare abundance gene (RAGs) are shown in red, other genes are shown in grey. Log 2NX, log 2 normalized expression. FIG. 5B, Relationship between average gene expression in healthy cfRNA and expression stability in healthy cfRNA measured by Gini coefficient (n=50). Housekeeping genes added to the RAG-focused capture panel are shown in yellow, other genes are shown in grey. FIG. 5C, Cumulative frequency of non-small cell lung cancer (NSCLC) patients with one or more variants in genes added to the RAG-focused capture panel because they are recurrently altered or frequently aberrantly expressed in NSCLC (n=122). The top 25 genes are shown. Variant data from the TCGA lung adenocarcinoma and lung squamous cell carcinoma project were downloaded from cBioPortal. FIG. 5D, Venn diagram depicting gene sets included in the RAG-focused capture panel (n=5,546 genes total). FIGS. 5E and 5F, Expression correlation between whole coding transcriptome and RAG capture for FIG. 5E, all shared genes and FIG. 5F, housekeeping genes (n=5 pairs). FIG. 5G, Schematic of platelet transcript correction approach using meta-reference controls with variable platelet expression. This approach was developed for samples captured with the RAG-focused panel which excluded platelet genes. FIG. 5H, Association between platelet-specific gene expression and first principal component, after platelet transcript correction of cfRNA expression as depicted in FIG. 5G, The same control cohort from FIGS. 3D to 3K was used (n=32). FIG. 5I, Association between NCI-H1975 spike detection and total sequencing depth. Logistic regression was used to estimate LOD95 of the in silico spikes.

FIGS. 6A to 6L provide data charts on detection of lung adenocarcinoma ctRNA. FIGS. 6A, 6B, 6C, and 6D, Relationship between sample library metrics from healthy controls (n=24), low dose computed tomography controls (LDCT, n=26), and lung adenocarcinoma (LUAD, n=50) patients. Boxplots depict FIG. 6A, cfRNA concentration (ng per mL plasma), FIG. 6B, cfRNA mass used for library preparation, FIG. 6C, total sequencing depth and FIG. 6D, unique sequencing depth. Each comparison was performed using Kruskal-Wallis test. FIG. 6E, Rates of cfRNA detection with respect to the unique sequencing depth per cfRNA sample. FIG. 6F, Sensitivity of cfRNA detection using LUAD Sig ES detection and summarized by driver oncogene sub-type. Detection threshold achieving ≥95% specificity was used. FIGS. 6G and 6H, Representative integrated genomics viewer (IGV) images displaying sequencing reads containing FIG. 6G, EGFR exon 19 deletion and FIG. 6H, alternative splicing of the MET exon 14. FIG. 6I, Count of reads mapped to canonical splice junctions in ROS1 and CD74 gene and to fusion breakpoints for representative sample with tissue-adjudicated ROS1/CD74 gene fusion. Junctions/breakpoints with ≥5 aligned reads are displayed. FIGS. 6J and 6K, LUAD elastic net (EN) model coefficients for top 30 features selected with FIG. 6J, negative coefficients and FIG. 6K, positive coefficients (n=202 features total). FIG. 6L, Relationship between model training cohort size and 10-fold cross validated model performance. The dotted line represents the 10-fold cross validated (10CV) AUC of the final LUAD EN model and the error bars represent the standard deviation of 10CV AUC at each sub-sampling level (n=10 samples per level).

FIGS. 7A to 7F provide data charts showing detection of ctRNA in plasma from NSCLC patients. FIG. 7A, Differential expression analysis of lung adenocarcinoma (LUAD) cfRNA (n=50) compared to non-cancer cfRNA (n=50). FIG. 7B, Average expression of genes enriched in LUAD cfRNA (n=94 genes) in LUAD tumor tissue (n=385) and meta-reference cfRNA (n=15). FIG. 7C, Pre-ranked gene set enrichment analysis using PanglaoDB¹¹ cell-type specific signatures. The top 15 positively and top 15 negatively enriched signatures are shown, and the dotted line delineates signatures with significant enrichment (P<0.05). FIG. 7D, Enrichment scores (ES) for a LUAD-specific gene signature (LUAD Sig; n=72 genes) in cfRNA from LUAD patients (n=50), risk-matched controls (n=26), and healthy controls (n=24). LDCT, low-dose computed tomography. FIG. 7E, Sensitivity of cfRNA detection using LUAD Sig ES detection and summarized by stage. Detection threshold achieving ≥95% specificity was used. FIG. 7F, Relationship between LUAD Sig ES in cfRNA and mean variant allele frequency (VAF) in matched cfDNA (n=15). The comparison was performed using Pearson and Spearman correlation. ND, not detected.

FIGS. 8A to 8G provide schematics and data charts on identifying somatic alterations NSCLC ctRNA. FIG. 8A, Schematic depicting ctRNA variant calling approach (see Methods). FIG. 8B, Oncoprint of single nucleotide variants (SNV), insertions/deletions (Indel), gene fusions, and splice variants found in cfRNA. Stage IV NSCLC samples with ≥1 tissue- or ctDNA-adjudicated variant (n=55) and risk-matched LDCT controls (n=36) were considered. Healthy controls were used to define variant-specific error rates as described in Methods. The bar plot depicts the percentage of samples with each mutation in each gene. LDCT, low-dose computed tomography. FIG. 8C, Percentage of samples with ≥1 variant detected in cfRNA. FIG. 8D, Percentage of tissue- or ctDNA-adjudicated variants that were detected in cfRNA summarized by variant type. FIG. 8E, Relationship between EGFR expression levels and variant detection in cfRNA for samples with tissue- or -ctDNA-adjudicated EGFR variants. Enrichment analysis was performed using fgsea. FIG. 8F, Predicted probability of lung cancer calculated by the weighted elastic net (EN) model for each sample and summarized by stage. Results for the training cohort (LUAD n=50, Control n=50) and the withheld validation cohort (LUAD n=28 timepoints from 14 individuals, control n=10) are shown. FIG. 8G, Receiver operating characteristic (ROC) curves summarizing cfRNA detection using LUAD Sig ES and the EN probabilities. AUC was compared with DeLong's test. Val, validation. AUC, area under ROC curve. ns, not significant.

FIGS. 9A to 9G provide schematics and data charts on detecting mechanisms of resistance to EGFR tyrosine kinase inhibitors in cfRNA. FIG. 9A, Summary of EGFR TKI cohort (n=10 patients, n=24 timepoints). Mechanisms of resistance were determined via tissue biopsy. METamp, MET amplification. C797S, EGFR C797S SNV, tSCLC, histological transformation to small cell lung cancer. FIG. 9B, Enrichment scores (ES) for a small cell lung cancer (SCLC) gene signature (SCLC Sig; n=73 genes) in patients with biopsy-proven tSCLC. The threshold representing ≥95% specificity in controls is shown by the dotted line. FIG. 9C, Vignette of patient with tSCLC after EGFR TKI treatment. The left axis depicts ES or EN scores and the right axis depicts the AF of tumor-adjudicated variants detected in cfRNA. FIG. 9D, Enrichment scores (ES) for a MET amplification-specific gene signature (METamp Sig; n=9 genes) in patients with biopsy-proven MET amplification. FIG. 9E, Vignette of patient with emergence of MET amplification after EGFR TKI treatment. FIG. 9F, EGFR C797S AF in cfRNA from patients with biopsy-proven C797S. FIG. 9G, Vignette of patient with emergence of EGFR C797S mutation after EGFR TKI treatment.

FIGS. 10A to 10H provide data charts on application of RARE-Seq. FIG. 10A, Sensitivity of cfRNA detection of LUAD Sig ES in LUAD cfRNA (n=50), liver hepatocellular carcinoma (LIHC) Sig ES in LIHC cfRNA (n=10), pancreatic adenocarcinoma (PAAD) Sig ES in PAAD in cfRNA (n=10), and prostate adenocarcinoma (PRAD) Sig ES in PRAD cfRNA (n=9). Detection thresholds achieving 90% specificity were used. FIG. 10B, Accuracy of tissue-of-origin (TOO) determination using cancer-TOO gene signatures. The bar plot shading depicts the ES rank for the true cancer type. Samples were considered for TOO analysis if detected by any cancer-specific signature. FIG. 10C, Confusion matrix comparing the predicted cancer type to the clinically diagnosed cancer type. FIG. 10D, Enrichment scores (ES) for normal lung tissue signature (n=5 genes) in cfRNA from patients with benign pulmonary conditions (n=74 timepoints from 70 individuals). COPD, chronic obstructive pulmonary disease. COVID, COVID-19 infection. ARDS, acute respiratory distress syndrome. FIG. 10E, Relationship between normal lung ES and patient smoking history. All samples from FIG. 10D without known pulmonary conditions were considered (n=42). FIG. 10F, Relationship between normal lung ES and ventilator status. All samples from FIG. 10D with known pulmonary conditions were considered (n=32 timepoints from 28 individuals). FIG. 10G, Timecourse depicting the number of unique reads aligned to COVID-19 mRNA vaccine sequence in cfRNA collected pre-vaccination and at various timepoints after two vaccination doses (n=9 timepoints). Log 2NX, log 2 normalized expression. FIG. 10H, Gene ontology (GO) enrichment analysis of genes that are significantly differentially expressed in post-vaccination timepoints. The top 10 gene sets from the MSigDb Hallmark and C5 collections are displayed).

FIG. 11 provides a summary of cancer and non-cancer cfRNA cohorts. LDCT, low-dose computed tomography. ARDS, acute respiratory distress syndrome. COVID, COVID-19 infection. VACC, post-COVID-19 mRNA vaccination. LUAD, lung adenocarcinoma. EGFR TKI, treated with epidermal growth factor receptor tyrosine kinase inhibitor. PAAD, pancreatic adenocarcinoma. PRAD, prostate adenocarcinoma. LIHC, liver hepatocellular carcinoma.

### DETAILED DESCRIPTION

Turning now to the drawings and data, systems and methods for performing sequencing of cell-free RNA (cfRNA) molecules are described. The systems and methods can comprise a means for performing targeted sequencing of the cfRNA. The systems and methods can also comprise various steps and/or components for enhancing the processing and input of cfRNA.

When sequencing cfRNA from a cell-free source, the nucleic acids within that source can be difficult to perform analysis due to the lack of quality nucleic acid molecules. Historically, analysis of cfRNA from a cell-free source focused on micro-RNAs (miRNAs). Expressed nucleic acid molecules of other RNA types (e.g. messenger RNA), however, can greatly enhance detection of biological phenomena and/or medical disorders and thus could have great benefit in the field of diagnostics. Unfortunately, a liquid biopsy from plasma comprises mostly cfRNA molecules that are derived from hematopoietic cells, drowning out signals from other potential sources. It is thus difficult to perform cfRNA analysis for diagnostic purposes, such as the detection of cancer, due to the low signals. Accordingly, new systems and methods are needed to enhance the detection cfRNA from cell-free sources.

Here, systems and methods are directed to sequencing of cfRNA derived from a cell-free source (also referred to as a liquid biopsy or excreta sample). Throughout the disclosure, the term liquid biopsy is utilized and is to refer to sources of cfRNA (inclusive of excreta) and include (but are not limited to) blood, plasma, lymph, cerebrospinal fluid, amniotic fluid, urine, and stool. The systems and methods can perform targeted sequencing to enhance the detection of some cfRNA molecules of the liquid biopsy. In some implementations, targeted sequencing comprises targeting cfRNA molecules that are infrequently found within liquid biopsies of control individuals (e.g., healthy individuals). In some implementations, a panel of capture probes or a panel of sets of primers are utilized to target cfRNA molecules of interest for sequencing.

Various systems and methods are directed to transcript panels and their use in methods for targeted sequencing of cfRNA molecules. A transcript panel can refer to a capture-based panel (e.g., ssDNA molecules for hybridization capture) or amplification-based panel (e.g., a set of primers for amplification). Accordingly, a transcript panel can be utilized during preparation of a sequencing library to perform targeted sequencing.

In some implementations, a transcript panel comprises rare abundance genes (RAGs), which are transcripts (including non-coding transcripts) that infrequently have cfRNA molecules expressed within liquid biopsies of healthy individuals. Targeting of RAGs can have various benefits, including overcoming the drowning signal provided by cfRNA derived from hematopoietic cells and also being able to enhance detection of various biological characteristics (including a medical disorder, pregnancy, a fetal complication, a pregnancy complication, a neoplastic growth, cancer, a particular cancer type, a pathogen infection, immune activation, organ transplant rejection, neurodegeneration, tissue of origin, cell type of origin, activation of a biochemical pathway, etc.).

In some implementations, RAGs are defined by as genes that are expressed below a threshold in control liquid biopsies. Populations of individuals can be utilized to acquire control liquid biopsies for analysis. In some implementations, a control liquid biopsy is a biopsy derived from an individual have generally good health (a control liquid biopsy can also be referred to as a healthy liquid biopsy). Generally good health can mean an individual not having one or more the following when the biopsy is collected: an observed pathogenic infection, a diagnosed cancer, a diagnosed metabolic disorder, a diagnosed neurological disorder, a diagnosed immunodeficiency disorder, a diagnosed autoimmune disorder, a diagnosed inflammatory disorder, a diagnosed cardiovascular disorder, a diagnosed renal disorder, a diagnosed hepatic disorder, active pregnancy, a diagnosed pregnancy complication, a diagnosed fetal complication, having an organ transplant, active rejection of an organ transplant, obesity, malnourishment, cachexia, and having an abnormality on a clinical test. In some implementations, cfRNA are collected to generate diagnostics of a particular disorder. In some the implementations in which a diagnostic of particular disorder (or trait) is to be generated, a control liquid biopsy is a biopsy derived from an individual that is not diagnosed for the particular disorder.

To identify RAGs, any appropriate number of control liquid biopsies can be used to establish which genes are expressed below a threshold of control liquid biopsies. In various instances, the number of control liquid biopsies to identify RAGs is 5 or more control liquid biopsies, 10 or more control liquid biopsies, 15 or more control liquid biopsies, 20 or more control liquid biopsies, 50 or more control liquid biopsies, 100 or more control liquid biopsies, 200 or more control liquid biopsies, 500 or more control liquid biopsies, 1000 or more control liquid biopsies, 2000 or more control liquid biopsies, 5000 or more control liquid biopsies, 10,000 or more control liquid biopsies, 20,000 or more control liquid biopsies, 50,000 or more control liquid biopsies, or 100,000 or more control liquid biopsies.

Various definitions of RAGs can be utilized, based on expression of genes within the control liquid biopsies. In various implementations, RAGs are defined as transcripts that are expressed in less than 50% of control liquid biopsies, RAGs are defined as transcripts that are expressed in less than 40% of control liquid biopsies, RAGs are defined as transcripts that are expressed in less than 30% of control liquid biopsies, RAGs are defined as transcripts that are expressed in less than 20% of control liquid biopsies, RAGs are defined as transcripts that are expressed in less than 10% of control liquid biopsies, RAGs are defined as transcripts that are expressed in less than 5% of control liquid biopsies, or RAGs are defined as transcripts s that are expressed in less than 1% of control liquid biopsies. In some implementations, a clustering technique is utilized to categorize transcripts that are expressed within control liquid biopsies and not expressed within control liquid biopsies.

In some implementations, RAGs are defined by having an expression level below a threshold within control liquid biopsies. In some implementations, expression values are normalized for comparison. In some implementations, expression values are log transformed (e.g., Log 2NX) for comparison. In some instances, RAGs are defined as transcripts that have expression values Log 2NX less than threshold (e.g., Log 2NX<0). In some implementations, RAGs are defined as transcripts that have the lowest expression values with respect to normalized expression in the control liquid biopsies. In various implementations, RAGs are defined as transcripts that are in the bottom 60% of genes with respect to normalized expression, RAGs are defined as transcripts that are in the bottom 50% of genes with respect to normalized expression, RAGs are defined as transcripts that are in the bottom 40% of genes with respect to normalized expression, RAGs are defined as transcripts that are in the bottom 30% of genes with respect to normalized expression, RAGs are defined as transcripts that are in the bottom 20% of genes with respect to normalized expression, RAGs are defined as transcripts that are in the bottom 10% of genes with respect to normalized expression, or RAGs are defined as transcripts that are in the bottom 5% of genes with respect to normalized expression.

In some implementations, RAGs are defined by being detected below a threshold of control liquid biopsies and/or having an expression level below a threshold within control liquid biopsies. Accordingly, any threshold as defined herein for presence within a control biopsy can be combined with any threshold as defined herein for expression level. In one example, within the Examples and Data section below, RAGs were defined as transcripts that are expressed in less than 5% of control liquid biopsies and that are in the bottom 30% of genes with respect to normalized expression. Other definitions can be combined with RAGs for various applications, such as (for example) transcripts having tissue specificity, transcripts having cell-type specificity, transcripts having known clinical relevancy, transcripts having known biological relevancy (e.g., biomarker), and transcripts having known and common mutagenesis profiles such as fusion events and variants.

Provided in Table 3 is an example of a list of RAGs that were defined as being identified in less than 5% of control liquid biopsies and for which average log 2NX was in the bottom 30% of all genes as determined from 50 replicates of 28 healthy controls using the whole exome sequencing (WES) of cell-free RNA (cfRNA) and 307 samples of whole blood gene expression data from the Genotype-Tissue Expression (GTEx) project. In some implementations, a transcript panel comprises nucleic acid molecules for detecting a plurality of RAGs from Table 3. In some implementations, a transcript panel comprises nucleic acid molecules for detecting 1% of the listed RAGs from Table 3. In some implementations, a transcript panel comprises nucleic acid molecules for detecting 5% of the listed RAGs from Table 3. In some implementations, a transcript panel comprises nucleic acid molecules for detecting 10% of the listed RAGs from Table 3. In some implementations, a transcript panel comprises nucleic acid molecules for detecting 20% of the listed RAGs from Table 3. In some implementations, a transcript panel comprises nucleic acid molecules for detecting 30% of the listed RAGs from Table 3. In some implementations, a transcript panel comprises nucleic acid molecules for detecting 40% of the listed RAGs from Table 3. In some implementations, a transcript panel comprises nucleic acid molecules for detecting 50% of the listed RAGs from Table 3. In some implementations, a transcript panel comprises nucleic acid molecules for detecting 60% of the listed RAGs from Table 3. In some implementations, a transcript panel comprises nucleic acid molecules for detecting 70% of the listed RAGs from Table 3. In some implementations, a transcript panel comprises nucleic acid molecules for detecting 80% of the listed RAGs from Table 3. In some implementations, a transcript panel comprises nucleic acid molecules for detecting 90% of the listed RAGs from Table 3. In some implementations, a transcript panel comprises nucleic acid molecules for detecting 100% of the listed RAGs from Table 3.

A transcript panel can target nucleic acids using a capture technique or an amplification technique. To target cfRNA, in some implementations, the RNA is first converted to cDNA before targeting. And in some implementations, the cfRNA is targeted prior to cDNA conversion. A transcript panel can thus comprise nucleic acid molecules that are complementary to the RNA strand, or to either strand of the cDNA, such that they can anneal and/or hybridize to the RNA or cDNA. In some implementations, a transcript panel is utilized to specifically target particular molecules of RNA and/or of the double-stranded cDNA. In some implementations, a capture-based panel comprises a set single-stranded nucleic acid probes for hybridization capture of particular molecules of the RNA and/or of particular molecules of double-stranded cDNA. In some implementations, an amplification-based panel comprises a set of primers for specific reverse-transcription of particular RNA and/or specific amplification of particular molecules of the double-stranded cDNA.

The sequences of the capture probes and/or primers are complementary to the transcripts that are to be targeted. The capture probes and/or primers do not need perfect complementation, but have enough complementation to its target in order to capture via hybridization or anneal for priming of amplification. Design of probes can be based on any appropriate sequence of the target. For example, a reference database such hg19 or hg38 can be utilized to design probes or primers for human transcript targets.

Particular targeting is based on sequence complementation and the genes selected within the panel. In some implementations, the transcript panel particularly targets a set of rare abundance genes (RAGs). In some implementations, the transcript panel excludes genes that are not RAGs (as defined by the criteria or as listed in Table 3). Exclusion of the genes that are not RAGs allows facilitates streamlined sequencing protocols and enhancing sequencing results (e.g., greater sequencing depth in the targeted sequences as compared to the depth afforded by a whole exome panel). The better sequencing results allows for better sensitivity, yielding better results in various applications such as cfRNA-based diagnostics.

In some implementations, a transcript panel excludes at least 50% of whole-exome genes that are not RAGs. In some implementations, a transcript panel excludes at least 60% of whole-exome genes that are not RAGs. In some implementations, a transcript panel excludes at least 70% of whole-exome genes that are not RAGs. In some implementations, a transcript panel excludes at least 80% of whole-exome genes that are not RAGs. In some implementations, a transcript panel excludes at least 90% of whole-exome genes that are not RAGs. In some implementations, a transcript panel excludes at least 95% of whole-exome genes that are not RAGs. In some implementations, a transcript panel excludes at least 99% of whole-exome genes that are not RAGs.

Sequencing protocols and their applications can be enhanced by including a set of control genes, which can provide positive assurance of the sequencing results and facilitate normalization between samples. In some implementations, a transcript panel comprises nucleic acid molecules for detecting a set of control transcripts to provide a normalization between samples. Generally, the set of control transcripts can be any set of transcripts that are commonly detected and having a relatively stable expression level among control liquid biopsies. In some embodiments, a set of control transcripts comprises one or more housekeeping transcripts (e.g., GAPDH, actin, ubiquitin).

In some implementations, a transcript panel consists of 1 control gene. In some implementations, a transcript panel consists of 5 or fewer control genes. In some implementations, a transcript panel consists of 10 or fewer control genes. In some implementations, a transcript panel consists of 20 or fewer control genes. In some implementations, a transcript panel consists of 50 or fewer control genes. In some implementations, a transcript panel consists of 100 or fewer control genes. In some implementations, a transcript panel consists of 500 or fewer control genes. In some implementations, a transcript panel consists of 1000 or fewer control genes.

Sequencing protocols and their applications can be enhanced by assessing genes that provide further insight. For example, if the provision of diagnosing cancer, certain transcripts can provide further diagnostic insight, such as genes that are recurrently mutagenized in cancer. Accordingly, a transcript panel can include additional genes that are not RAGs (as defined by the criteria or as listed in Table 3).

In some implementations, a transcript panel consists of 10 or fewer genes in addition to RAGs. In some implementations, a transcript panel consists of 20 or fewer genes in addition to RAGs. In some implementations, a transcript panel consists of 50 or fewer genes in addition to RAGs. In some implementations, a transcript panel consists of 100 or fewer genes in addition to RAGs. In some implementations, a transcript panel consists of 200 or fewer genes in addition to RAGs. In some implementations, a transcript panel consists of 500 or fewer genes in addition to RAGs. In some implementations, a transcript panel consists of 1000 or fewer genes in addition to RAGs. In some implementations, a transcript panel consists of 2000 or fewer genes in addition to RAGs. In some implementations, a transcript panel consists of 5000 or fewer genes in addition to RAGs. In some implementations, a transcript panel consists of 10,000 or fewer genes in addition to RAGs.

Various types of genes may be included in addition to RAGs, such as (for example) tissue-specific transcripts, cell-type-specific transcripts, clinically relevant transcripts, biomarker transcripts, commonly mutagenized transcripts, and B-cell and T-cell clone transcripts. In some implementations, the transcript panel targets a set of tissue-specific transcripts. In some implementations, the transcript panel targets a set of cell-type-specific transcripts. In some implementations, the transcript panel targets a set of clinically relevant transcripts. In some implementations, the transcript panel targets a set transcripts that are biomarker transcripts. In some implementations, the transcript panel targets a set of mutagenic events. In some implementations, the transcript panel targets a set of B-cell and T-cell clones.

In some implementations, a transcript panel comprises nucleic acid molecules for detecting tissue-specific transcripts. A tissue-specific transcript is a transcript that is uniquely highly expressed within a particular tissue as compared to its expression in all other tissues. In various implementations, a tissue-specific transcript is expressed at least 2-fold within a particular tissue as compared to all other tissues, a tissue-specific transcript is expressed at least 3-fold within a particular tissue as compared to all other tissues, a tissue-specific transcript is expressed at least 4-fold within a particular tissue as compared to all other tissues, a tissue-specific transcript is expressed at least 5-fold within a particular tissue as compared to all other tissues, a tissue-specific transcript is expressed at least 6-fold within a particular tissue as compared to all other tissues, a tissue-specific transcript is expressed at least 7-fold within a particular tissue as compared to all other tissues, a tissue-specific transcript is expressed at least 8-fold within a particular tissue as compared to all other tissues, a tissue-specific transcript is expressed at least 9-fold within a particular tissue as compared to all other tissues, or a tissue-specific transcript is expressed at least 10-fold within a particular tissue as compared to all other tissues. Unique expression can be determined empirically or data derived from a database, such as (for example) data derived from the Genotype-Tissue Expression (GTEx) project or the Human Protein Atlas (HPA).

In some implementations, a transcript panel comprises nucleic acid molecules for detecting cell-type-specific transcripts. A cell-type-specific transcript is a transcript that is uniquely highly expressed within a particular cell type as compared to its expression in other cell types. In various implementations, a cell-type-specific transcript is expressed at least 2-fold within a particular cell type as compared to other cell types, a cell-type-specific transcript is expressed at least 3-fold within a particular cell type as compared to other cell types, a cell-type-specific transcript is expressed at least 4-fold within a particular cell type as compared to other cell types, a cell-type-specific transcript is expressed at least 5-fold within a particular cell type as compared to other cell types, a cell-type-specific transcript is expressed at least 6-fold within a particular cell type as compared to other cell types, a cell-type-specific transcript is expressed at least 7-fold within a particular cell type as compared to other cell types, a cell-type-specific transcript is expressed at least 8-fold within a particular cell type as compared to other cell types, a cell-type-specific transcript is expressed at least 9-fold within a particular cell type as compared to other cell types, or a cell-type-specific transcript is expressed at least 10-fold within a particular cell type as compared to other cell types. Unique expression can be determined empirically or data derived from a database, such as (for example) data derived from PanglaoDb.

In some implementations, a transcript panel comprises nucleic acid molecules for detecting clinically relevant transcripts (e.g., for diagnostic relevance). Clinically relevant transcripts can include transcripts for the diagnosis of medical disorders. For example, transcripts of EGFR, KRAS, MET, ALK, RET, and ROS1 are useful in the diagnosis non-small cell lung cancer (NSCLC).

In some implementations, a transcript panel comprises nucleic acid molecules for detecting transcripts associated with a biological characteristics (e.g., biomarker transcripts). Biological characteristics include (but are not limited to) a medical disorder, pregnancy, a fetal complication, a pregnancy complication, a neoplastic growth, cancer, a particular cancer type, a pathogen infection, immune activation, organ transplant rejection, neurodegeneration, tissue of origin, cell type of origin, and activation of a biochemical pathway.

In some implementations, a transcript panel comprises nucleic acid molecules for detecting transcripts associated with mutagenic events, which may useful for identifying de novo mutagenesis and/or cancer oncogenes. Mutagenic events include (but are not limited to) gene fusions, insertions, deletions, translocations, single nucleotide variants, and splice variants.

In some implementations, a transcript panel comprises nucleic acid molecules for detecting transcripts associated with detection of B-cell and T-cell clones, which can be useful for tracking immunological activity against particular antigens. A transcript panel can target V(D)J recombination of B-cell receptors and T-cell receptors, and thus identify sequences of B-cell and T-cell clones. In some implementations, clones are detected at a single time point to identify current activity of immunogens (e.g., immunogens of a pathogenic infection, a cancer, a vaccine, or an autoimmune disorder). In some implementations, clones are detected at over a plurality of time points to detect changes of activity of immunogens (e.g., detection of minimal residual disease, cancer treatment success, waning immunogenicity from vaccination or pathogenic infection, autoimmune flares).

Several components can be utilized and several steps can be performed to perform sequencing of cfRNA molecules obtained in a cell-free sample. A liquid biopsy can be derived from any appropriate biological source, including (but not limited to) blood, plasma, lymph, cerebrospinal fluid, amniotic fluid, urine, and stool. To extract RNA, any appropriate kit can be utilized (e.g., QIAamp Circulating Nucleic Acid kit). In some implementations, glycogen is added to the cell-free sample comprising nucleic acids prior to adding to a silica-based column. Typically, a sample also comprises cell-free DNA, which may be utilized in other assessments.

In some implementations, cell-free nucleic acids are quantified using quantitative Real-Time Polymerase Chain Reaction (qRT-PCR). In some implementations, both RNA and DNA are simultaneously quantified in the cell-free sample. To quantify RNA and DNA are simultaneously, qRT-PCR can be performed in which primers can detect and quantify RNA using a primer set across an intron and primers can detect and quantify DNA using a primer set targeting an untranscribed region of the genome. For quantifying RNA, an intron of commonly expressed gene (e.g., housekeeping gene) may be utilized. Control standards can be utilized to generate standard curves to quantify the nucleic acids. After quantification, the cell-free samples can be split into aliquots for performing DNA and RNA assessments. In some implementations, DNA is digested from the cell-free sample (e.g., DNase digestion) for performing RNA assessments. In some implementations, DNA digestion is performed after elution from a silica column for performing RNA assessments. In some implementations, one or more downstream steps of the sequencing preparation protocol uses a defined amount of material based on the quantification of cell-free RNA.

In some implementations, cell-free RNA is converted to cDNA. In some implementations, double-stranded cDNA is generated. In some implementations, double-stranded cDNA is treated with a nuclease that removes single-stranded nucleic acid molecules (e.g., S1 endonuclease).

After capture and/or amplification a set of targets, the library can be further processed (e.g., amplified) and sequenced using high-throughput sequencing. In some implementations, sequencing is performed to a depth of less than 10,000 reads, sequencing is performed to a depth of more than 10,000 reads, sequencing is performed to a depth of more than 100,000 reads, sequencing is performed to a depth of more than 1,000,000 reads, sequencing is performed to a depth of more than 10,000,000 reads, or sequencing is performed to a depth of more than 100,000,000 reads.

In some implementations, sequencing is performed to a depth of less than 1× genomes, sequencing is performed to a depth of more than 1× genomes, sequencing is performed to a depth of more than 5× genomes, sequencing is performed to a depth of more than 10× genomes, sequencing is performed to a depth of more than 20× genomes, sequencing is performed to a depth of more than 30× genomes, sequencing is performed to a depth of more than 40× genomes, sequencing is performed to a depth of more than 50× genomes, sequencing is performed to a depth of more than 100×, sequencing is performed to a depth of more than 150× genomes, or sequencing is performed to a depth of more than 200× genomes.

After sequencing, various analyses of the sequencing results can be performed to enhance the detection of the targeted cell-free nucleic acid molecules. In some implementations, only transcripts included within the transcript panel are included for downstream analysis. In some implementations, transcript counts are converted to log-transformed counts per million (CPM) to account for library size and transcriptome complexity. In some implementations, transcript counts are normalized to account for transcript size. In some implementations, counts are normalized to account for sample-to-sample variation. In some implementations, unwanted variation is removed, such as (for example) removal of variation due to transcript expression associated with platelets.

It has been found that platelet derived RNA can confound analysis of cfRNA. Platelets are found in pretty much all types of liquid biopsies, especially when cellular injury is present. Therefore, platelets can confound assessments of liquid biopsies derived from (for example) blood, plasma, lymph, cerebrospinal fluid, amniotic fluid, urine, and stool for assessment of number of biological characteristics (for example) a medical disorder, pregnancy, a fetal complication, a pregnancy complication, a neoplastic growth, cancer, a particular cancer type, a pathogen infection, immune activation, organ transplant rejection, neurodegeneration, tissue of origin, cell type of origin, and activation of a biochemical pathway. In some implementations, a cfRNA sample is centrifuged to remove platelets. In some implementations, platelet expression is removed in silico after sequencing. To remove platelet expression in silico, a program to remove unwanted variation is utilized (e.g., RUVseq R package). In some implementations, a transcript panel for targeted sequencing specifically excludes expression of genes associated with platelets. Even when a transcript panel for targeted sequencing specifically excludes platelet expression, factors of unwanted variation can be estimated using control cfRNA to establish a correction factor correlated with platelet expression. In one example, platelet correction can be performed by ordinary least squares regression of log 2NX on the selected factor of unwanted platelet expression.

In some implementations, the sequencing result is utilized to perform differential transcript expression analysis. In some implementations, differential transcript expression analysis can be utilized for comparison of samples (e.g., medical disorder vs. control; e.g., comparison from one time point to another timepoint). Any appropriate method for performing differential transcript expression analysis can be utilized (e.g., DESeq2 R package). In one example, a generalized linear model can be built using sample type as the covariate. Significantly differentially expressed genes can be identified utilizing an appropriate cut-off and significations (e.g., greater than 1 log fold and adjusted p-value<0.05). Further downstream analysis can be performed on the differentially expressed genes to gain insight on biological phenomena associated with a sample. For instance, gene set enrichment analysis can be performed, which can be utilized to identify molecular signatures associated with various phenomena.

In some implementations, the sequencing result is utilized to detect enrichment of expression signatures related to biological characteristic. Generally, many biological characteristics can be identified within a sequencing result via expression signatures. Examples of biological characteristics include (but are not limited to) a medical disorder, pregnancy, a fetal complication, a pregnancy complication, a neoplastic growth, cancer, a particular cancer type, a pathogen infection, immune activation, organ transplant rejection, neurodegeneration, tissue of origin, cell type of origin, and activation of a biochemical pathway. In some implementations, an enrichment score of an expression signature is computed, providing a scaled assessment on whether a biological characteristic is present in the sample. Diagnoses can be established based on various biological characteristics present within the sequencing result. For example, a liquid biopsy can be utilized to assess the health of a pregnancy or fetus by assessing the cfRNA for expression signatures related to various health statuses and/or complications.

In some implementations, the sequencing result is utilized to detect mutagenesis, including somatic or de novo mutagenic events. Examples of mutagenic events that can be detected include (but are not limited to) gene fusions, insertions, deletions, translocations, single nucleotide variants, and splice variants. In some implementations, the sequencing result is utilized to infer copy number status of one or more genes. In one example, MET gene amplification can be inferred from an expression signature, which is related to targeted therapy resistance in non-small cell lung cancer. As noted, assessment of mutagenesis can be diagnostic and inform treatment options, especially for various cancer types.

In some implementations, a computational model is trained to predict a categorical status or the likelihood of a biological characteristic is present in a cfRNA sample based on the sequencing result. In some implementations, the computational model is trained utilizing the sequencing result directly as input into the model. In some implementations, the computational model utilizes a derived features, such as (for example) normalized expression of individual genes, enrichment of one or more gene signatures, enrichment of biochemical pathways, collection of sequence variants, and copy number status. A trained computational model can then be utilized to assess cfRNA sequencing results of patients, such as for use as a diagnostic in a clinical setting.

For predicting a biological characteristic, any appropriate computational model can be utilized. Example of computational models include (but are not limited to) logistic regression, elastic net, LASSO, random forest, XGBoost, and a neural network. Training can be performed using expression data from samples having a biological characteristic and control samples. A cohort of individuals with a known categorical status of a biological phenomenon has their cfRNA sequenced and processed to train the computational model. Alternatively, because cfRNA samples are based upon expression within particular cell types, samples can be from solid tissue or any other source representative of cfRNA. Upon training, sets of features, weights and hyperparameters providing robust predictability can be selected.

### Sequencing Methods and Diagnostics

Various methods are directed towards performing sequencing of cfRNA. Furthermore, cfRNA sequencing methods can be utilized in a number of diagnostic assessments. Accordingly, an individual can have a liquid biopsy extracted or collected for cfRNA sequencing, which can be prepared utilizing a transcript panel of RAGs. Various biomedical characteristics can be screened for and/or diagnosed via cfRNA sequencing. Based on diagnostic assessments, an individual can be further assessed via clinical evaluations. Diagnostic assessments can also inform treatment options and thus, in some instances, a treatment can be performed by a medical professional, such as a doctor, nurse, dietician, or similar. Sequencing of cfRNA can also be utilized to assess treatment response, treatment outcomes, and/or presence of minimal residual disease.

A number of sequencing methods can be performed to assess cfRNA. Generally, a sequencing method collects a sample comprising cfRNA and prepares it for targeted sequencing utilizing a panel of RAGs.

An example of method for sequencing cfRNA can comprise:
extract or collect a cfRNA sample
enrich cfRNA sample with a targeted RAG panel
sequence enriched cfRNA sample to yield a sequencing result

In some implementations, the cfRNA sample is (or is derived from) a liquid biopsy. In some implementations, the cfRNA sample is (or is derived from) an excreta sample. In some implementations, the cfRNA sample is (or is derived from) blood, plasma, lymph, cerebrospinal fluid, amniotic fluid, urine, or stool.

In some implementations, the RAG panel comprises a set of genes that are expressed in less than a percentage of a population of control liquid biopsies. In some implementations, the RAG panel comprises a set of genes having an expression level below a threshold within a population of control liquid biopsies. In some implementations, the RAG panel comprises a set of genes that are expressed in less than a percentage of a population of control liquid biopsies and having an expression level below a threshold within the population of control liquid biopsies. In some implementations, the RAG panel comprises a set of genes from Table 3. In some implementations, the RAG panel comprises nucleic acid molecules for amplifying RAGs. In some implementations, the RAG panel comprises nucleic acid molecules for capturing RAGs via hybridization.

In some implementations, the method for sequencing cfRNA further comprises:
collect a population of control cfRNA samples
perform sequencing on each cfRNA sample to yield sequencing result
identify RAGs from the population of sequencing results

In some implementations, the control cfRNA samples are extracted or collected from healthy individuals. In some implementations, RAGs are identified by being expressed in less than a percentage of a population of control liquid biopsies, implementations, RAGs are identified by having an expression level below a threshold within a population of control liquid biopsies. In some implementations, RAGs are identified by being expressed in less than a percentage of a population of control liquid biopsies and having an expression level below a threshold within the population of control liquid biopsies.

Sequencing of a cfRNA sample can be inform biological characteristics of the individual from which the cfRNA sample was extracted or collected. Diagnostic methods can be utilized for a variety of purposes, such as (for example) screening individuals for a biological characteristic or for biomedical complications, performing a diagnostic for a particular medical disorder, assessing treatment response, assessing treatment outcome, and screening for minimal residual disease. Diagnostic methods can be utilized for an assessment of number of biomedical conditions, such as (for example) a medical disorder, pregnancy, a fetal complication, a pregnancy complication, a neoplastic growth, cancer, a particular cancer type, a pathogen infection, immune activation, organ transplant rejection, neurodegeneration, tissue of origin, cell type of origin, and activation of a biochemical pathway.

In some implementations, a screening method or a diagnostic comprises:
extract or collect a cfRNA sample
enrich cfRNA sample with a targeted RAG panel
sequence enriched cfRNA sample to yield a sequencing result
identify one or more biomedical conditions within the sequencing result

In some implementations, cfRNA sample is (or is derived from) blood, plasma, lymph, cerebrospinal fluid, amniotic fluid, urine, or stool. In some implementations, the screening method is generalized and assesses a plurality of biomedical condition. In some implementations, the diagnostic method is a particular biomedical condition. In some implementations, a biomedical condition is identified by gene expression signature. In some implementations, a biomedical condition is identified by a computational model trained to predict the biomedical condition utilizing the sequencing result or features derived from the sequencing result.

In some implementations, the biomedical condition is pregnancy. In some implementations, the biomedical condition is a fetal complication. In some implementations, the biomedical condition is a pregnancy complication. When assessing a fetal complication or a pregnancy complication, in some implementations, the cfRNA sample is derived amniotic fluid. When assessing a fetal complication or a pregnancy complication, in some implementations, screening is performed at various timepoints throughout a pregnancy. In some implementations, when a fetal complication or a pregnancy complication is identified, further diagnostic procedures are performed, such as (for example) assessments for gestational diabetes, genetic assessment of the fetus, fetal ultrasound, and maternal blood testing. In some implementations, when a fetal complication or a pregnancy complication is identified, a treatment is performed such as (for example) inducing labor, administering a tocolytic medication, and performing a Caesarian delivery.

In some implementations, the biomedical condition is a pathogenic infection. In some implementations, the biomedical condition is immunological status, such as (for example) a vaccination status or prior pathogenic infection. In some implementations, when assessing a pathogenic infection, the cfRNA sample is also enriched for pathogen sequences. In some implementations, when a pathogenic infection is identified, a treatment is performed (such as) administering an antipathogenic medication (e.g., antibiotic agent, antiviral agent, antiparasitic agent, etc.). In some implementations, the screening method monitors the pathogenic infection, an antipathogenic treatment response, an immunological response, a health condition, or any combination thereof.

In some implementations, the biomedical condition is immune activation, such as (for example) activation in response to a pathogen, activation in response to an immunization, or activation of an autoimmune disorder. In some implementations, the biomedical condition is inflammation. In some implementations, when the biomedical condition is an autoimmune disorder or inflammation, a treatment is performed such as (for example) administering an immune suppressor, and administering an anti-inflammatory agent.

In some implementations, the biomedical condition is an organ transplant rejection. In some implementations, an organ transplant rejection is identified by gene expression signatures related cytotoxicity, gene expression signatures related to tissue of origin, gene expression signatures related to cell type of origin, genetic sequence that differentiate donor from host, or a combination thereof. In some implementations, the screening method is performed periodically after the host receives the transplant. In some implementations, when organ transplant rejection is identified, further diagnostic procedures are performed such as (for example) tissue biopsy of the organ, and medical imaging of the organ. In some implementations, when organ transplant rejection is identified, a treatment is performed such as (for example) administering an increased dose of immunosuppressant agents and administering stronger immunosuppressant agents.

In some implementations, the biomedical condition is neurodegeneration. When assessing for neurodegeneration, in some implementations, the cfRNA sample is (or is derived from) cerebrospinal fluid. In some implementations, neurodegeneration is identified by gene signatures related to the neurodegenerative disorder, gene signatures related to neural tissue of origin, gene signatures related to neural cell types of origin, gene signatures related to inflammation, or a combination thereof. In some implementations, when neurodegeneration is identified, further diagnostic procedures are performed such as (for example) medical screening, assessments of motor activity or speech, and assessments of cognition. In some implementations, when neurodegeneration is identified, a treatment is performed such as (for example) medications for reducing neurodegenerative symptoms.

In some implementations, the biomedical condition is cancer. In some implementations, the screening method is performed as part of a cancer surveillance effort (e.g., before symptoms of cancer are present or are recognized). In some implementations, the screening method is performed during treatment to assess the treatment response. In some implementations, the screening method is performed after treatment to assess whether residual cancer (e.g., MRD) exists after a treatment, which can be performed periodically. In some implementations, the diagnostic method informs cancer subtype, cancer stage, and/or treatment strategy.

The screening can be performed for a number of neoplasm types, including (but not limited to) acute lymphoblastic leukemia (ALL), acute myeloid leukemia (AML), anal cancer, astrocytomas, basal cell carcinoma, bile duct cancer, bladder cancer, breast cancer, Burkitt's lymphoma, cervical cancer, chronic lymphocytic leukemia (CLL) chronic myelogenous leukemia (CML), chronic myeloproliferative neoplasms, colorectal cancer, diffuse large B-cell lymphoma, endometrial cancer, ependymoma, esophageal cancer, esthesioneuroblastoma, Ewing sarcoma, fallopian tube cancer, follicular lymphoma, gallbladder cancer, gastric cancer, gastrointestinal carcinoid tumor, hairy cell leukemia, hepatocellular cancer, Hodgkin lymphoma, hypopharyngeal cancer, Kaposi sarcoma, Kidney cancer, Langerhans cell histiocytosis, laryngeal cancer, leukemia, liver cancer, lung cancer, lymphoma, melanoma, Merkel cell cancer, mesothelioma, mouth cancer, neuroblastoma, non-Hodgkin lymphoma, non-small cell lung cancer, osteosarcoma, ovarian cancer, pancreatic cancer, pancreatic neuroendocrine tumors, pharyngeal cancer, pituitary tumor, prostate cancer, rectal cancer, renal cell cancer, retinoblastoma, skin cancer, small cell lung cancer, small intestine cancer, squamous neck cancer, T-cell lymphoma, testicular cancer, thymoma, thyroid cancer, uterine cancer, upper tract urothelial cancer, vaginal cancer, and vascular tumors. In some implementations, when the cancer to be assessed is colorectal or gastric cancer, the cfRNA sample is (or is derived from) a stool sample. In some implementations, when the cancer to be assessed is bladder, kidney, prostate, or upper tract urothelial cancer, the cfRNA sample is (or is derived from) a urine sample.

In some implementations, when cancer is indicated, a number of follow-up clinical evaluations can be performed, including (but not limited to) physical exam, medical imaging, mammography, endoscopy, stool sampling, pap test, alpha-fetoprotein blood test, CA-125 test, prostate-specific antigen (PSA) test, biopsy extraction, bone marrow aspiration, and tumor marker detection tests. Medical imaging includes (but is not limited to) X-ray, magnetic resonance imaging (MRI), computed tomography (CT), ultrasound, and positron emission tomography (PET). Endoscopy includes (but is not limited to) bronchoscopy, colonoscopy, colposcopy, cystoscopy, esophagoscopy, gastroscopy, laparoscopy, neuroendoscopy, proctoscopy, and sigmoidoscopy.

In some implementations, when cancer is indicated, a number of treatments can be performed, including (but not limited to) surgery, chemotherapy, radiation therapy, immunotherapy, targeted therapy, hormone therapy, stem cell transplant, and blood transfusion. In some implementations, an anti-cancer and/or chemotherapeutic agent is administered, including (but not limited to) alkylating agents, platinum agents, taxanes, vinca agents, anti-estrogen drugs, aromatase inhibitors, ovarian suppression agents, endocrine/hormonal agents, bisphophonate therapy agents and targeted biological therapy agents. Medications include (but are not limited to) cyclophosphamide, fluorouracil (or 5-fluorouracil or 5-FU), methotrexate, thiotepa, carboplatin, cisplatin, taxanes, paclitaxel, protein-bound paclitaxel, docetaxel, vinorelbine, tamoxifen, raloxifene, toremifene, fulvestrant, gemcitabine, irinotecan, ixabepilone, temozolmide, topotecan, vincristine, vinblastine, eribulin, mutamycin, capecitabine, capecitabine, anastrozole, exemestane, letrozole, leuprolide, abarelix, buserlin, goserelin, megestrol acetate, risedronate, pamidronate, ibandronate, alendronate, zoledronate, tykerb, daunorubicin, doxorubicin, epirubicin, idarubicin, valrubicin mitoxantrone, bevacizumab, cetuximab, ipilimumab, ado-trastuzumab emtansine, afatinib, aldesleukin, alectinib, alemtuzumab, atezolizumab, avelumab, axtinib, belimumab, belinostat, bevacizumab, blinatumomab, bortezomib, bosutinib, brentuximab vedoitn, briatinib, cabozantinib, canakinumab, carfilzomib, certinib, cetuximab, cobimetnib, crizotinib, dabrafenib, daratumumab, dasatinib, denosumab, dinutuximab, durvalumab, elotuzumab, enasidenib, erlotinib, everolimus, gefitinib, ibritumomab tiuxetan, ibrutnib, idelalisib, imatinib, ipilimumab, ixazomib, lapatinib, lenvatinib, midostaurin, nectiumumab, neratinib, nilotinib, niraparib, nivolumab, obinutuzumab, ofatumumab, olaparib, loaratumab, osimertinib, palbocicilib, panitumumab, panobinostat, pembrolizumab, pertuzumab, ponatinib, ramucirumab, reorafenib, ribociclib, rituximab, romidepsin, rucaparib, ruxolitinib, siltuximab, sipuleucel-T, sonidebib, sorafenib, temsirolimus, tocilizumab, tofacitinib, tositumomab, trametinib, trastuzumab, vandetanib, vemurafenib, venetoclax, vismodegib, vorinostat, and ziv-aflibercept. An individual may be treated, by a single medication or a combination of medications described herein. A common treatment combination is cyclophosphamide, methotrexate, and 5-fluorouracil (CMF).

### EXAMPLES AND SUPPORTING DATA

The systems and methods of the disclosure will be better understood with the several examples and supporting provided. Validation results show that the use of a targeted sequencing panel that is based on rare abundance genes greatly improves detection of cancer using cfRNA samples. By using the RAG panel, detection of expression signatures was improved 50-fold over whole transcriptome sequencing. This method is also better at detect resistance to targeted treatments as compared to cfDNA methods. The method also allows for assessment for tissue of origin, which can be useful to identify the primary pathologic location. The results can be extrapolated to other cell-free diagnostic techniques, including diagnoses of a medical disorder, pregnancy, a fetal complication, a pregnancy complication, a neoplastic growth, cancer, a particular cancer type, a pathogen infection, immune activation, organ transplant rejection, neurodegeneration, tissue of origin, cell type of origin, and activation of a biochemical pathway.

### Cell-Free RNA Analysis for Non-Invasive Cancer Detection and Characterization

Described herein are system and methods for sequencing analysis of cfRNA, which is termed RARE-Seq (Random priming & Affinity capture of cell-free RNA fragments for Enrichment analysis by Sequencing). This approach improves the limit of detection for circulating tumor RNA (ctRNA) over prior methods while maintaining high specificity for tumor-naïve cancer detection. RARE-Seq has great utility in various clinical applications, including non-invasive ctRNA genotyping, treatment resistance monitoring, tissue-of-origin analysis, and molecular characterization of non-malignant conditions.

### Results

Pre-Analytical Factors Impacting cfRNA Recovery and Analysis

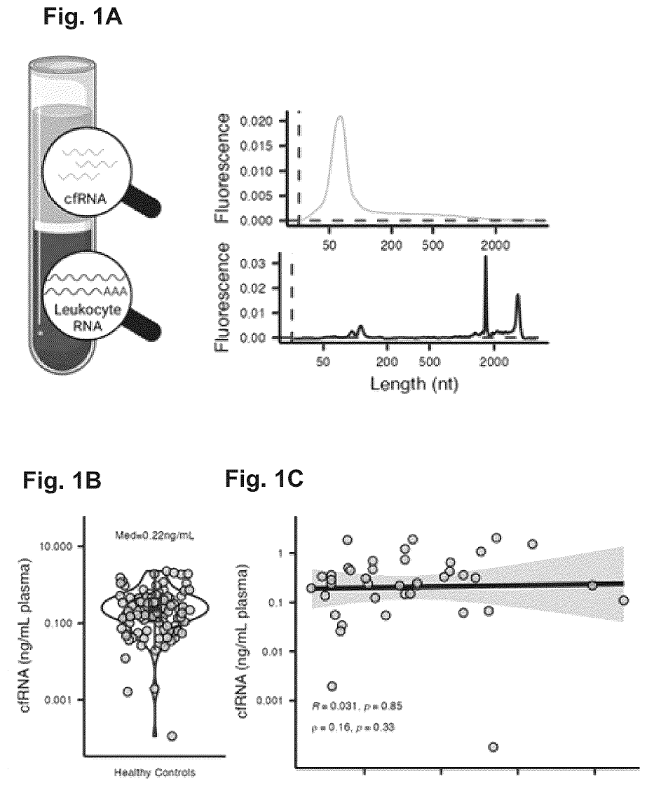

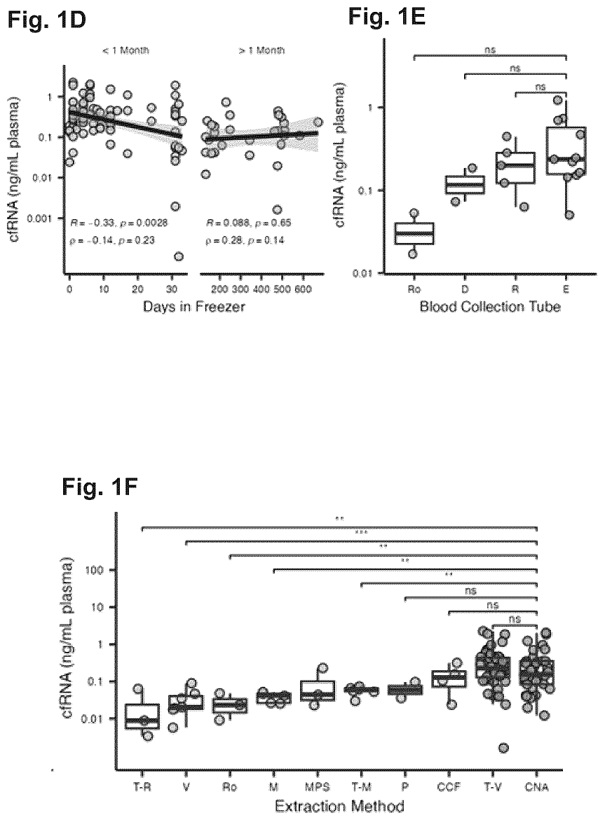

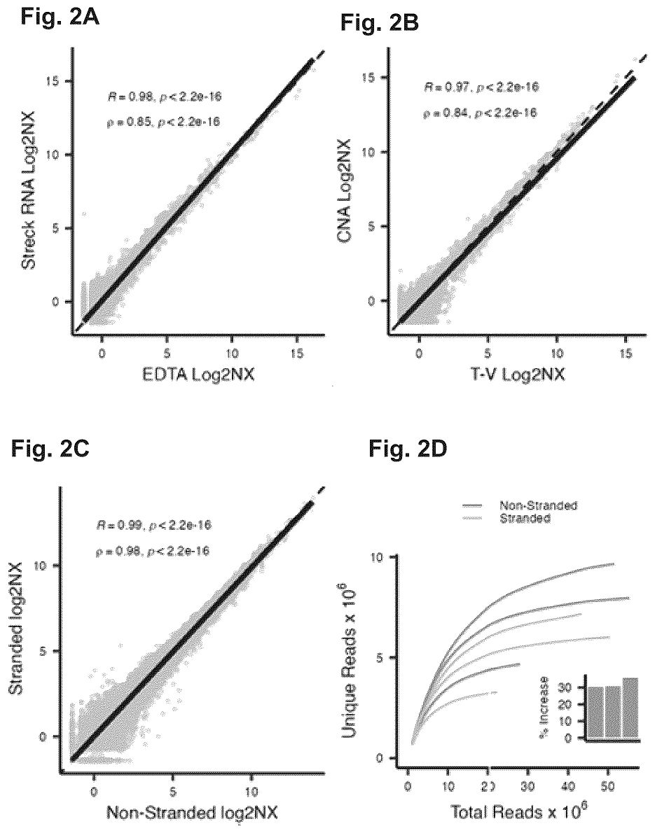

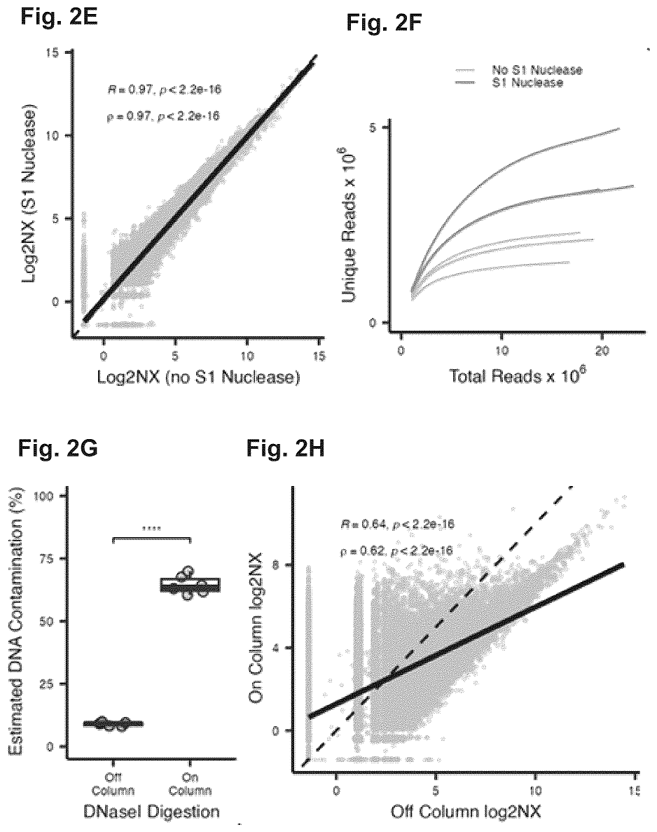

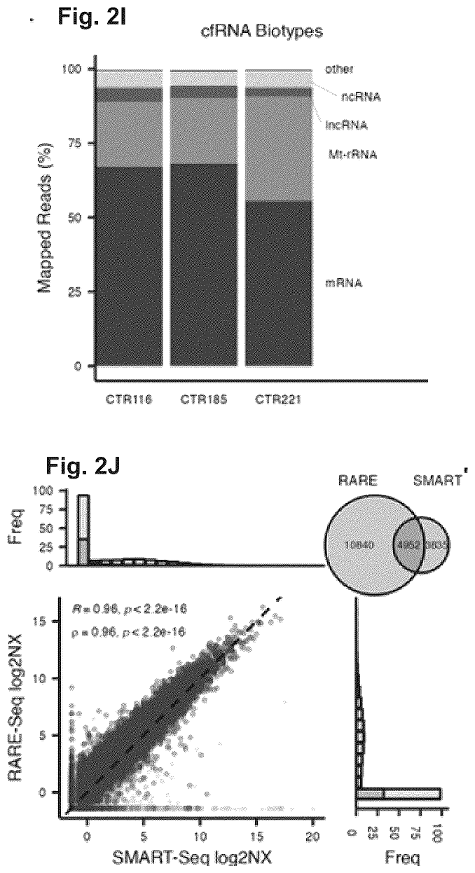

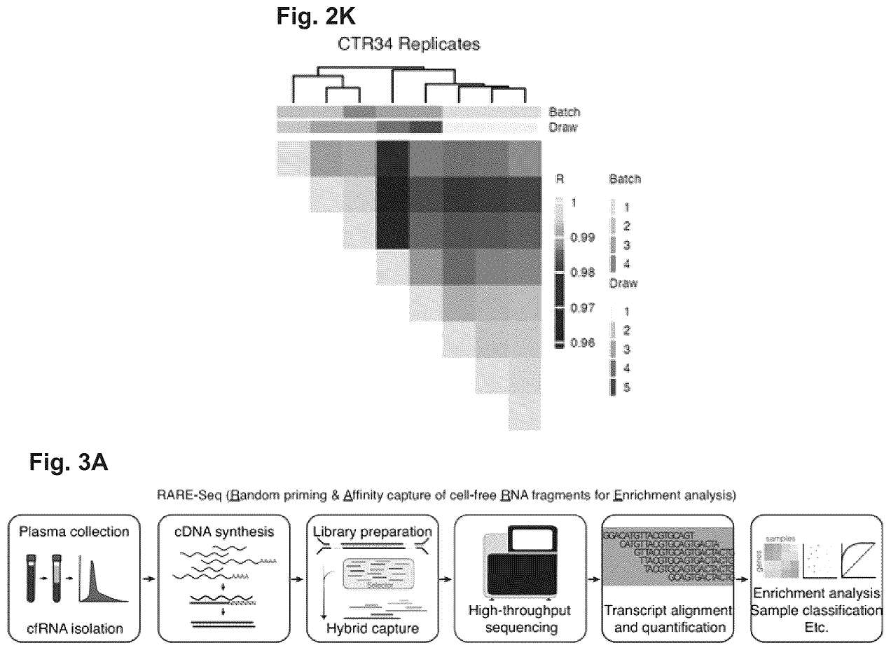

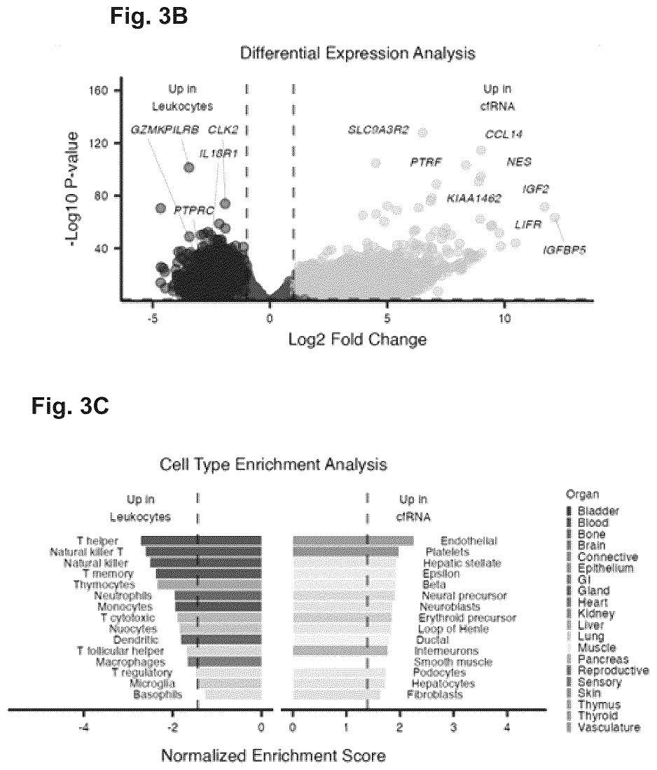

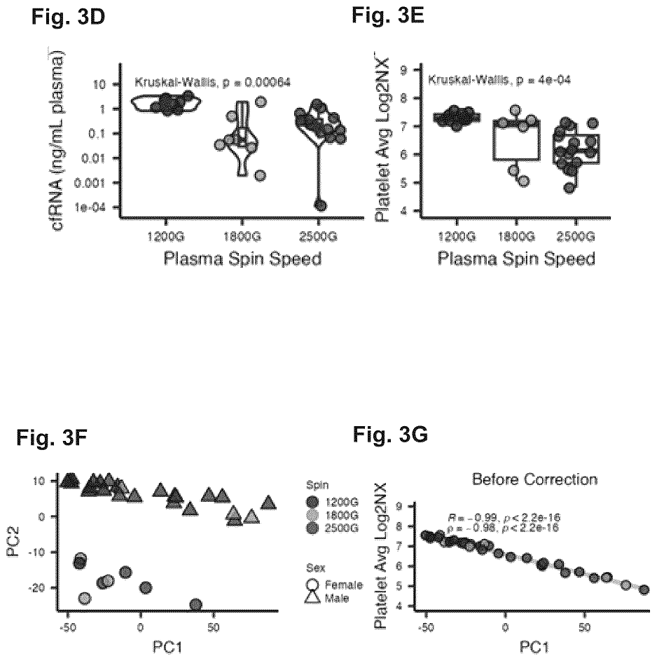

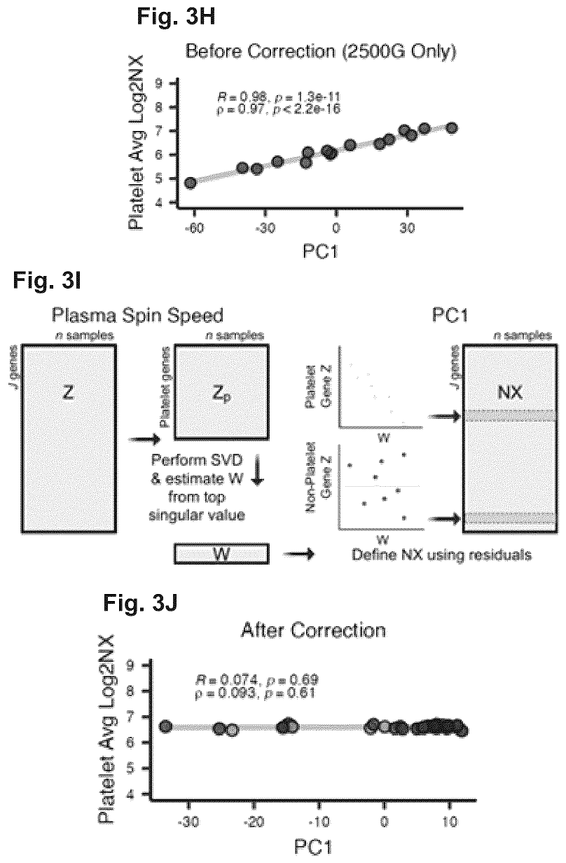

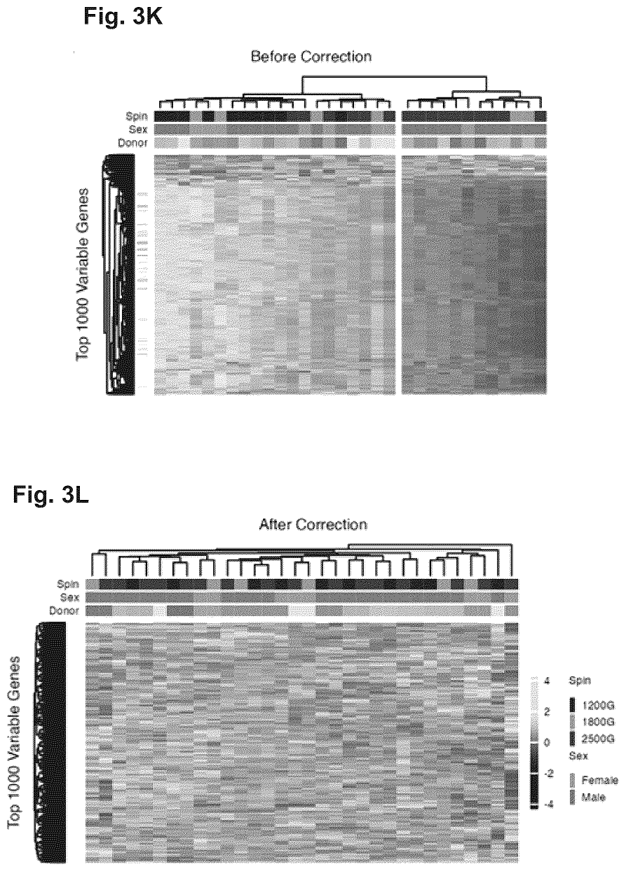

Cell-free RNA is highly fragmented and does not contain detectable 18S and 28S rRNA peaks (FIG. 1A), indicating high levels of degradation. In blood samples from healthy donors with no history of cancer, the median concentration of cfRNA was 220 μg per mL plasma, representing the equivalent of approximately 20 cells (n=117 FIG. 1B). To enhance recovery, factors that impacted cfRNA recovery and downstream expression analysis were evaluated (FIGS. 1C to 1F). Specifically, analysis of multiple blood collection and RNA extraction protocols that allowed optimal RNA recovery from plasma resulted in discovery that factors such as hemolysis and freezer storage time had minimal or no association with cfRNA yields (FIGS. 1C and 1D). Multiple steps in the assay workflow were optimized, including removal of contaminating DNA, complementary DNA (cDNA) synthesis, and end repair. These optimizations enhanced library preparation efficiency from low cfRNA inputs and minimized effects of contaminating cfDNA (FIGS. 2A to 2K). It was further confirmed that the optimizations resulted in reproducible cfRNA expression profiles in healthy controls. The optimizations of the cfRNA method can be utilized in RARE-Seq (Random priming & Affinity capture of cell-free RNA fragments for Enrichment analysis by Sequencing) protocol (FIG. 3A).

Platelet and Non-Hematopoietic Cell Types are Enriched in cfRNA

Expression differences between cellular and cell-free RNA were characterized by applying RARE-Seq to cfRNA and matched leukocyte RNA from 10 healthy adults. Differential expression analysis identified 8,336 significant genes, including 5,271 genes over-represented in cfRNA and 3,065 genes over-represented in leukocyte RNA (FIG. 3B). Using gene set enrichment analysis (GSEA) and cell type-specific gene sets from a single cell RNA-sequencing atlas, it was found that cfRNA was significantly enriched for non-hematopoietic cell types, including endothelial cells, hepatocytes, fibroblasts, and multiple neuronal cell types (n=71 total) (FIG. 3C). In contrast, leukocyte RNA was significantly enriched for 12 major hematopoietic cell types of myeloid and lymphoid lineages, including T cells, NK cells, neutrophils, and monocytes. Only two hematopoietic cell types were enriched in cfRNA: erythroid precursors and platelets, suggesting their significant contributions to the cfRNA pool during erythropoiesis and megakaryocytopoiesis.

Given the significant enrichment of platelet transcripts in cfRNA (FIG. 3C), it was suspected that residual platelets not removed during plasma isolation may contribute RNA to cfRNA samples. To test this idea, cfRNA concentrations were compared between protocols using three different centrifugation speeds to separate acellular from cellular plasma components (n=32). The median concentration was ~7.5-fold higher at 1200×G than 2500×G (1.72 ng/mL vs 0.23 ng/mL; P=0.000052; FIG. 3D). Furthermore, average expression of platelet-specific genes (n=130¹¹) was significantly associated with centrifugation speed (FIG. 3E). Principal component analysis (PCA) revealed that the first principal component (PC1) captured 70% of expression variation and was strongly associated with platelet expression (R=−0.99, P=<2.2e-16) (FIGS. 3F and 3G). Similar results were found even among samples processed with a fixed centrifugation speed, suggesting that differences in platelet counts may also contribute to variation of platelet transcripts in cfRNA samples (R=0.98, P=<1.3e-11) (FIG. 3H). Collectively, these results suggest that platelets represent the largest source of expression variation in cfRNA and that platelet contamination is a critical pre-analytical variable to control for in cfRNA analyses.

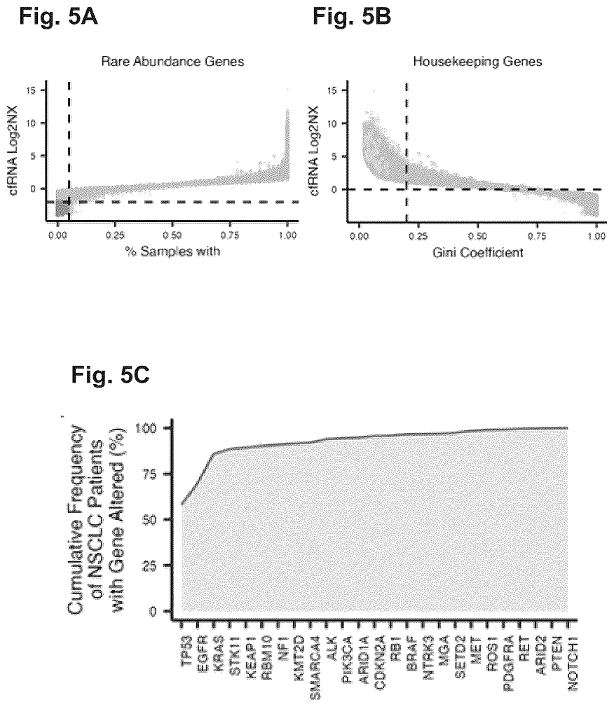

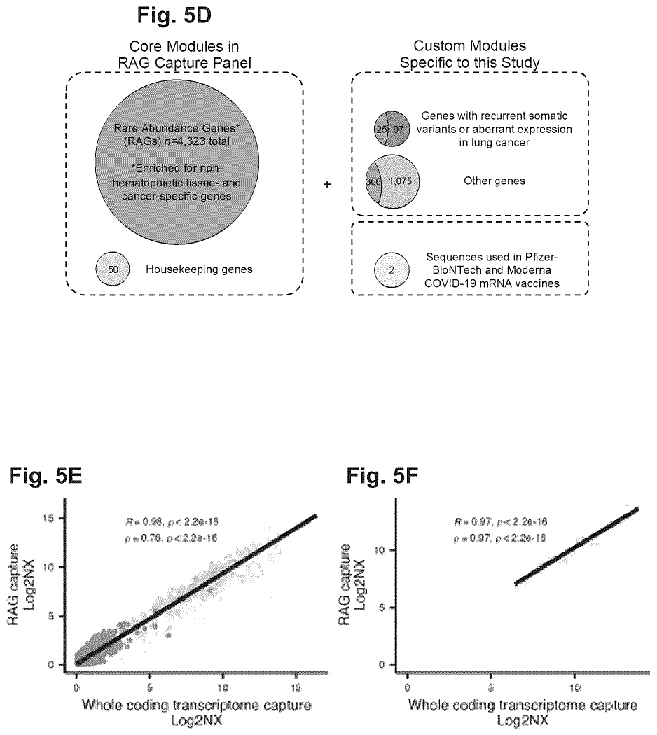

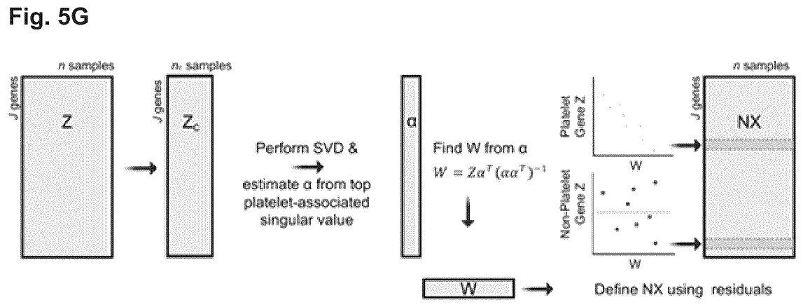

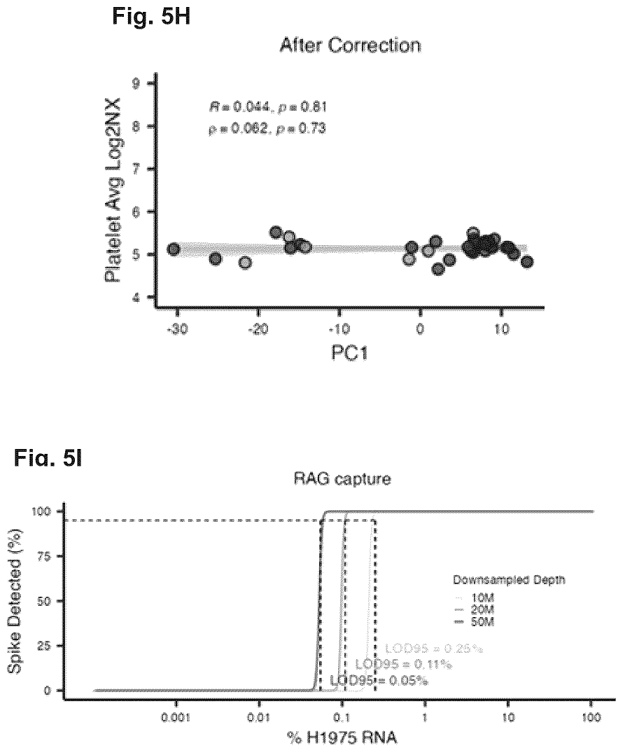

Although individual platelets contain less RNA content than leukocytes, platelets outnumber leukocytes by approximately two orders of magnitude in blood. Additionally, physiological variation in platelet counts span an approximately three-fold range in healthy adults with further variability observed in the context of cancer. Therefore, even efficient methods to minimize plasma contamination by platelets could struggle to effectively reduce levels of platelet-associated RNA. Separately, from a practical standpoint, it is often not feasible to standardize blood banking protocols, especially when profiling previously collected samples. Therefore, it was investigated whether expression contributions from platelet contamination could be algorithmically eliminated. To do so, an in silico approach was developed to remove unwanted gene expression driven by varying platelet contamination levels (FIG. 3I, see also FIG. 5G). This approach successfully removed the unwanted variation contributed by platelets, as shown by the lack of the correlation between platelet expression and PC1 after correction (R=0.07, P=0.69) (FIG. 3J, see also FIG. 5H). Hierarchical clustering further confirmed this effect, as samples clustered according to platelet gene expression before correction (FIG. 3K) but according to inter-individual expression differences after correction (FIG. 3L). Therefore, addressing platelet contamination in cfRNA analyses is critical for reliably uncovering gene expression contributions from non-platelet sources.

Maximizing Sensitivity for ctRNA Detection

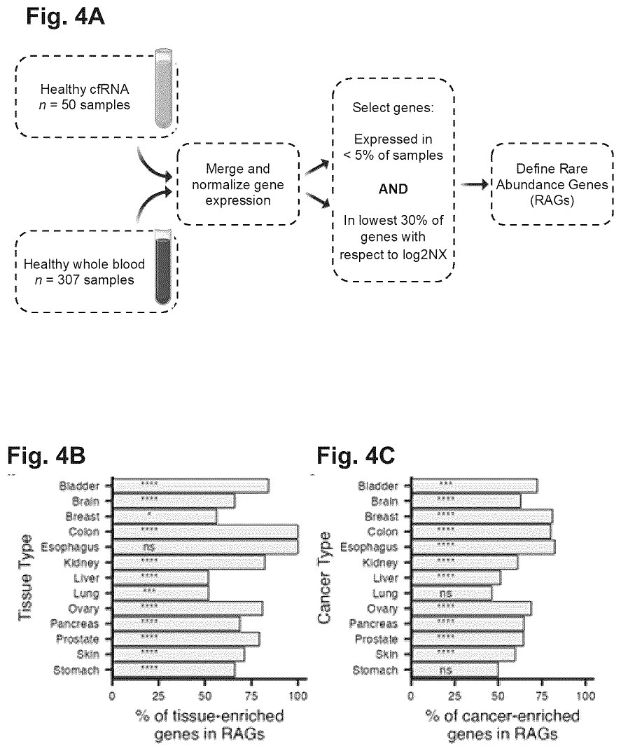

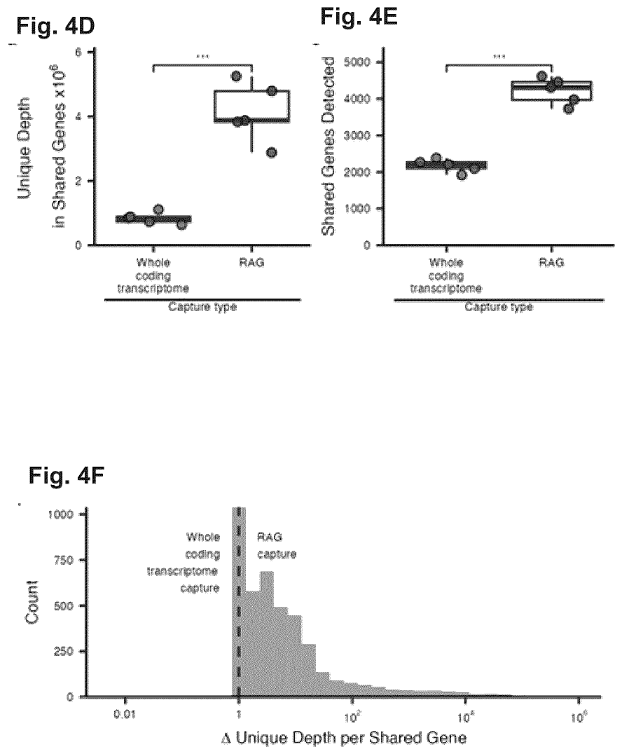

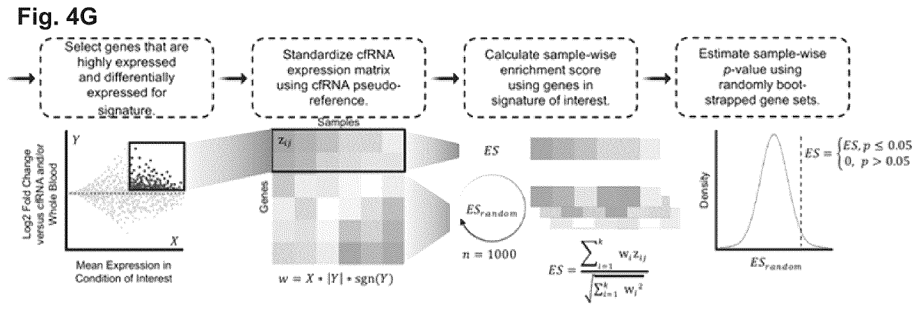

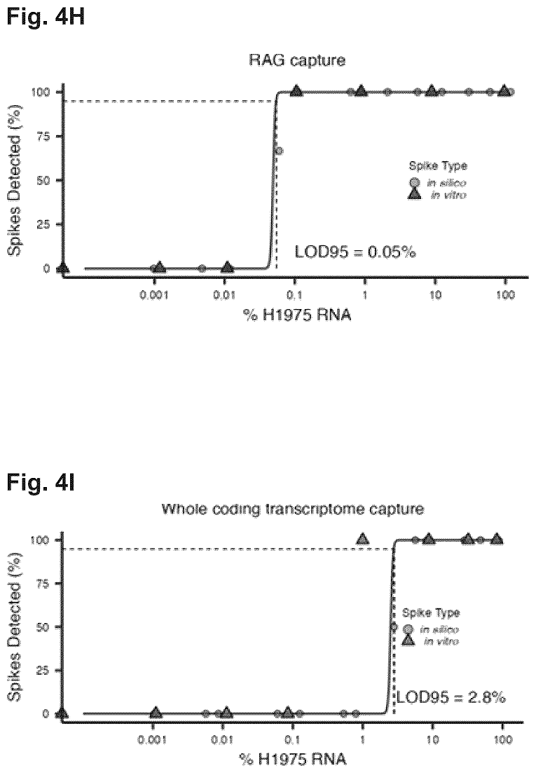

While cfRNA is highly enriched for expression of genes from non-hematopoietic sources, the absolute expression levels of these genes are relatively low compared to hematopoietic-derived transcripts (e.g. globins) and highly expressed housekeeping genes (e.g. beta actin). It was therefore hypothesized that selectively capturing genes absent from or lowly expressed in healthy control cfRNA could enhance detection of non-hematopoietic tissue or disease signatures and expand the utility of RARE-Seq to a wide range of clinical applications. To test this, ‘rare abundance genes’ (RAGs) were identified using whole coding transcriptome RARE-seq data from healthy cfRNA samples (n=50) and RNA-seq data from healthy whole blood samples (n=307) from the GTEx project (FIG. 4A). RAGs were defined based on consistent low or absent expression in both cfRNA and whole blood (FIG. 5A; Table 3). Strikingly, as compared to all protein coding transcripts, RAGs were substantially enriched both for non-hematopoietic tissue-specific genes and tumor-specific genes from diverse cancers (FIGS. 4B and 4C).

Given the enrichment of genes that could reflect tissue injury or the presence of cancer among RAGs, it was examined whether targeted sequencing of RAGs could increase sensitivity for detection of these transcripts. Therefore, a capture panel targeting 4,323 RAGs was therefore designed. This panel was supplemented with 50 housekeeping genes, prioritizing genes with relatively uniform expression across healthy cfRNA and whole blood samples while covering a broad dynamic range of expression (FIG. 5B). Genes known to be recurrently mutated in lung cancers were also added (n=123 genes), including genes such as TP53, EGFR, KRAS, ALK, RET, and ROS1 (FIG. 5C). The full collection of genes targeted by our RAG capture panel is summarized in FIG. 5D.

Five healthy cfRNA samples were profiled using both whole coding transcriptome and the RAG capture panels using RARE-Seq. Gene expression was highly correlated between matched libraries (R=0.98, P=<2.2e-16) (FIG. 5E), including for the housekeeping gene set (R=0.97, P=<2.2e-16) (FIG. 5F). Within the subset of genes shared across both capture panels, RAG capture resulted in significantly higher unique sequencing depth (FIG. 4D), more genes detected (FIG. 4E), and higher unique sequencing depth per gene detected (FIG. 4F). Thus, targeted capture of RAGs effectively increases sensitivity for detection of genes that are lowly expressed in healthy cfRNA.

The limit of 95% detection (LOD95) was determined for transcriptional signatures of interest, using either whole coding transcriptome or RAG-targeted panels. To score the presence of a gene signature of interest in cfRNA samples, an analytical framework was developed to compute a gene signature enrichment score (ES) by comparing the expression of pre-defined signature genes in a sample of interest to a “meta-reference” consisting of cfRNA samples from healthy controls and using bootstrapping to estimate its significance (FIG. 4G). An important advantage of this approach is that unlike machine learning-based methods, it does not require a large training cohort of patient cfRNA samples. This is because the ES strategy relies on existing gene expression datasets to define gene signatures and associated gene-wise weights based on their expression levels in the tissue or condition of interest.

To establish the LOD95 of RARE-Seq, in silico serial dilutions of RNA were generated from the NCI-H1975 NSCLC cell line into healthy control cfRNA at various cancer fractions. To enable using the ES framework, an NCI-H1975-specific gene signature was generated, which included the top differentially expressed genes distinguishing NCI-H1975 from a meta-reference healthy cfRNA profile (H1975 Sig; n=122 genes). Using this signature, the LOD95 for RAG RARE-Seq was 0.05% (FIG. 4H), >50-fold more sensitive than for whole coding transcriptome RARE-Seq (2.8%; FIG. 4I), demonstrating the value of targeting RAGs. To experimentally confirm this result, in vitro dilution experiment was performed by spiking NCI-H1975 RNA into healthy donor cfRNA at defined concentrations. These in vitro spikes demonstrated strong agreement with the in silico results (FIGS. 4H and 4I). The effect of sequencing coverage depth (range 10-50 million reads pairs) on the LOD95 of RAG-targeted RARE-Seq was evaluated and it was found that LOD95 increased with sequencing depth (FIG. 5I). Accordingly, 50 million read pairs were targeted for subsequent experiments using RARE-Seq with the RAG panel.

### Detection of Non-Small Cell Lung Cancer

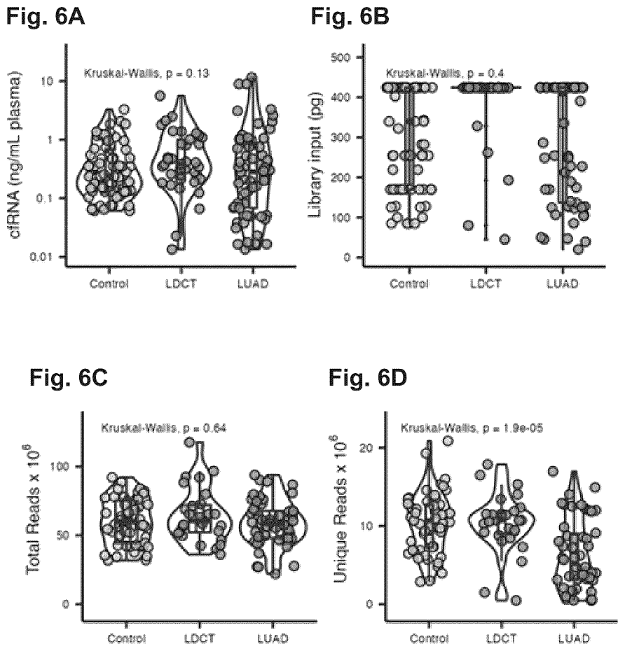

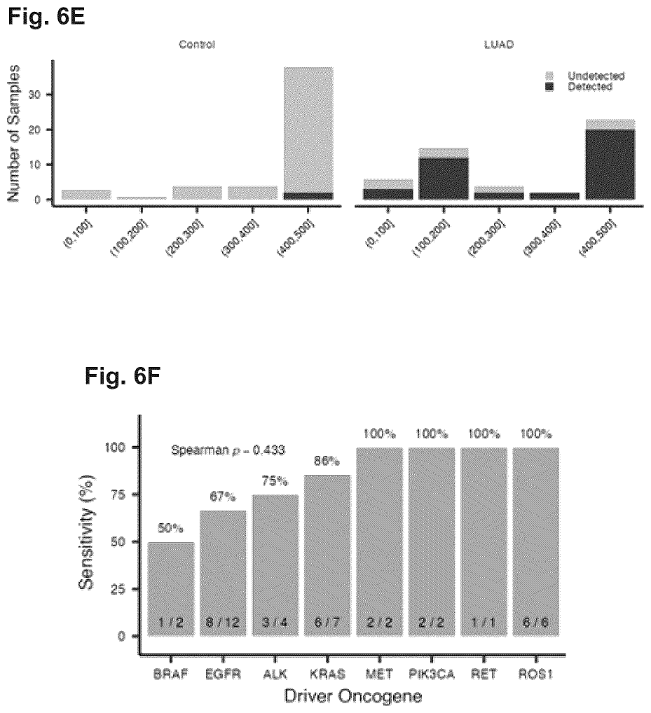

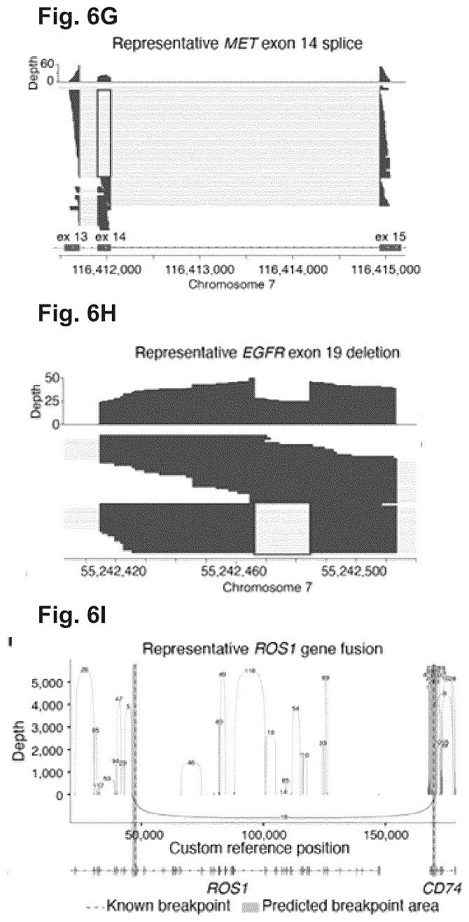

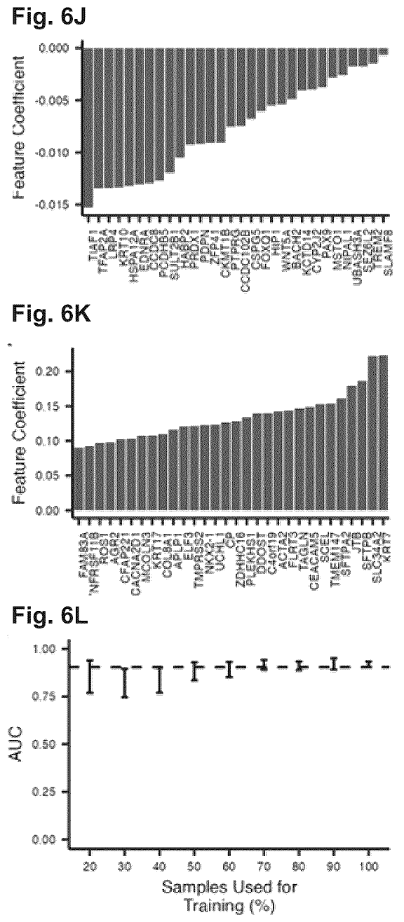

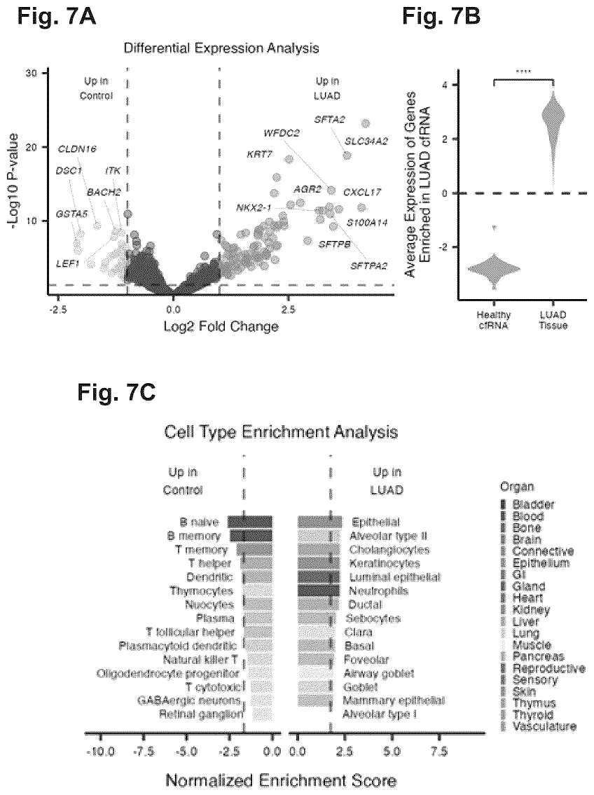

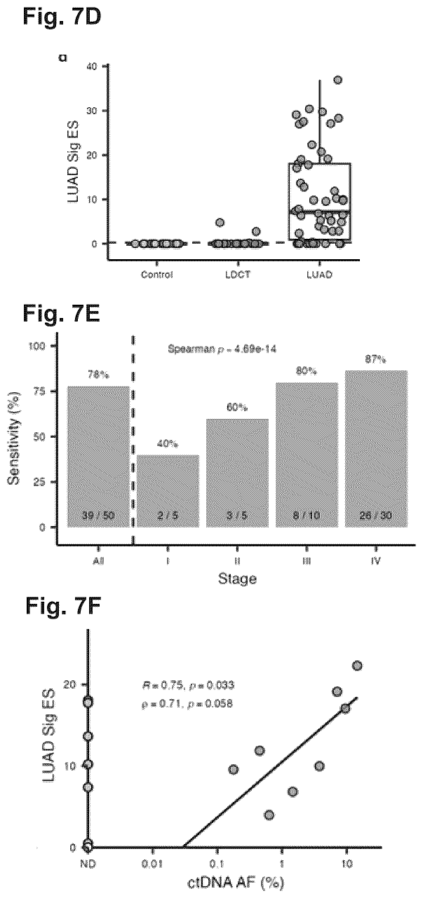

Having developed RARE-Seq, the remainder of the study was focused on generating proof-of-concept data for the potential utility of cfRNA analysis across several clinical applications. Given the higher analytic sensitivity observed by targeting RAGs, the RAG capture panel was employed for these experiments. First, the detection performance was assessed for non-invasive and tumor-naïve lung adenocarcinomas (LUAD), the most common type of NSCLC. cfRNA samples from 50 LUAD patients (stages I-IV) and 50 non-cancer controls were analyzed, including 26 risk-matched controls with history of substantial tobacco exposure collected at the time of low-dose computed tomography (LDCT) screening for lung cancer. Concentrations of cfRNA, input into library preparation, and sequencing depth were similar between groups (FIGS. 6A to 6C). Platelet-adjusted differential expression analysis between LUAD and controls identified 94 genes over-expressed in LUAD, including key genes known to be highly expressed in LUADs such as SFTA2, SLC34A2, NKX2-1 (also known as TTF1; FIG. 7A). LUAD DEGs were confirmed to be highly expressed in LUAD tumors from TCGA while being depleted in meta-reference cfRNA controls (FIG. 7B). Additionally, using cell type-specific gene signatures, GSEA revealed LUAD cfRNA to be most enriched for epithelial cells including pulmonary alveolar type I and II cells, and most depleted for naïve and memory B lymphocytes (FIG. 7C). Thus, cfRNA from LUAD patients is enriched for transcripts that are highly expressed in LUAD tumors.

To further quantify the enrichment of lung adenocarcinoma derived transcripts, the ES framework developed above was applied using an LUAD-specific gene signature (LUAD Sig; n=72 genes) identified using RNA-seq data from LUAD tumors within TCGA, LUAD cell lines from the CCLE, whole blood (GTEx), and meta-reference cfRNA controls (Methods). The LUAD Sig was detected in 39 out of 50 LUAD subjects (78% sensitivity at 95% specificity in non-cancer controls; FIG. 7D) and was significantly associated with stage (sensitivity by stage: I=40%, II=60%, III=80%, IV=87%; P=4.7e-14; FIG. 7E). In addition, in a subset of plasma samples that were also interrogated for circulating tumor DNA, the LUAD ES was significantly correlated with ctDNA variant allele frequency (VAF) (R=0.75, P=0.03) (FIG. 7F), suggesting that RARE-Seq can quantify cancer burden in cfRNA. However, 40% of samples with both ctRNA and ctDNA data were detected by RARE-Seq alone, suggesting that ctRNA and ctDNA analysis may be complimentary for cancer detection. Group-wise differences in unique sequencing depth were not associated with likelihood of detection (FIGS. 6D and 6E), indicating that this variable was not driving detection. In addition, successful detection did not appear to significantly vary as a function of the specific oncogenic driver mutations in LUAD (FIG. 6F).

Genotyping of Somatic Mutations in ctRNA

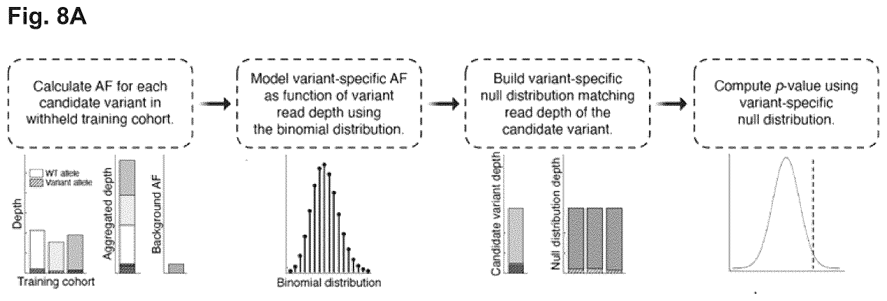

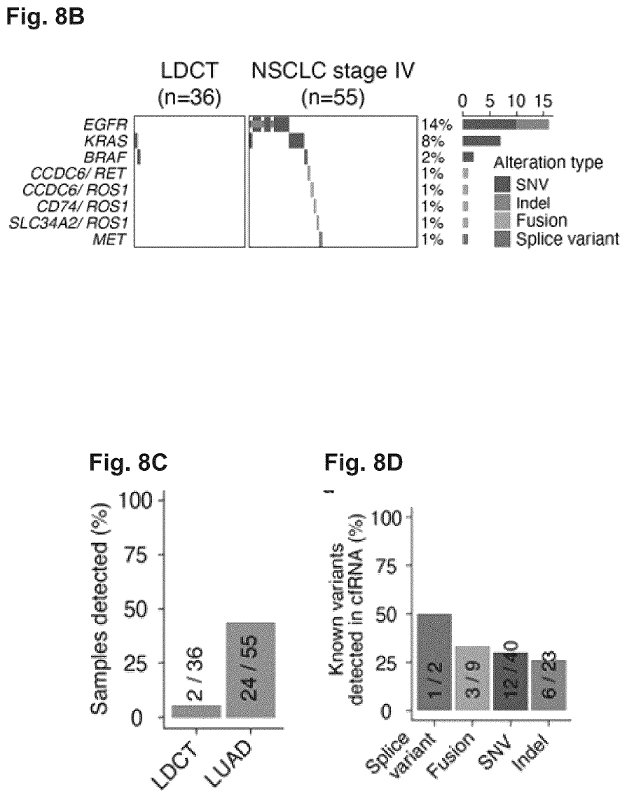

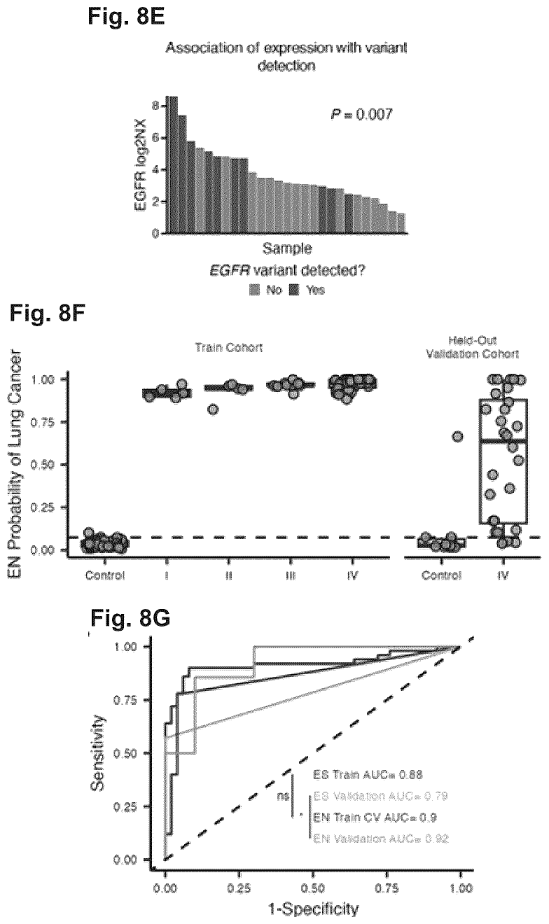

The ability to genotype cancer-derived somatic mutations using RARE-Seq was assessed. While non-invasive tumor genotyping is already routinely performed clinically using ctDNA, ctRNA-based variant detection could be a useful addition to cfRNA analysis. Therefore, a custom ctRNA genotyping approach was developed to detect recurrent, clinically actionable single nucleotide variants (SNVs), insertions/deletions (indels), splice variants, and gene fusions in NSCLC (FIG. 8A). To ensure specificity, putative variants were censored if their VAF did not significantly exceed the background error rate observed in healthy cfRNA controls. Performance of this approach was assessed in 55 stage IV LUAD patients and 36 LDCT controls. One or more somatic driver mutations was detected in 44% of LUAD patients and 5.6% of LDCT controls, including mutations in key NSCLC oncogenic drivers such as EGFR L858R and exon 19 deletions, KRAS G12C, ROS1 and RET fusions, and MET exon skipping events (FIGS. 8B and 8C; FIGS. 6G and 6I). Of the 74 variants identified by tumor tissue genomic DNA or ctDNA genotyping in the same patients, 22 (30%) were also detectable in ctRNA and detection rates were similar across variant types (FIG. 8D). A significant relationship was observed between variant detection rates and corresponding expression levels. For instance, EGFR variants were significantly more frequently detected in cfRNA samples with high EGFR expression (P=0.007; FIG. 8E). Thus, RARE-Seq analysis allows simultaneous identification of actionable somatic mutations in cancer patients.

Given that ctRNA expression signatures and ctRNA somatic variation could individually be used to distinguish between cancer patients and non-cancer controls, it was next investigated whether a machine learning approach could combine these features into a single detection algorithm. A weighted elastic net (EN) classifier was trained to distinguish LUAD and control cfRNA using 10-fold nested cross validation (CV) in a training cohort of 50 cases and 50 controls. To decrease the risk of model overfitting, the gene weights based on LUAD tumor expression data were leveraged during model training. The model demonstrated high classification performance (AUROC=0.9; FIGS. 8F and 8G) and selected 279 features including gene expression features (e.g., key LUAD markers such as SLC34A2, SFTPB, ROS1) and mutation-based features (e.g., detection of at least one ctRNA SNV; FIGS. 6J and 6K). To test whether the training cohort was sufficiently large to estimate classification accuracy, classifier training and cross-validation was repeated using sub-sampling and found stable performance when using 70% of more of the training cohort (FIG. 6L). Importantly, the EN model performed similarly well in a withheld validation cohort collected at an independent institution (AUROC=0.92) (FIGS. 8F and 8G). Performance of the EN classifier was statistically similar to the ES framework in the training cohort (P=0.60), but improved performance in the validation cohort (P=0.04). Therefore, when sufficient samples for training are available, machine learning-based approaches can also be used to train robust classifiers that utilize RARE-Seq data as input.

### Identifying Mechanisms of Resistance to EGFR Targeted Therapy

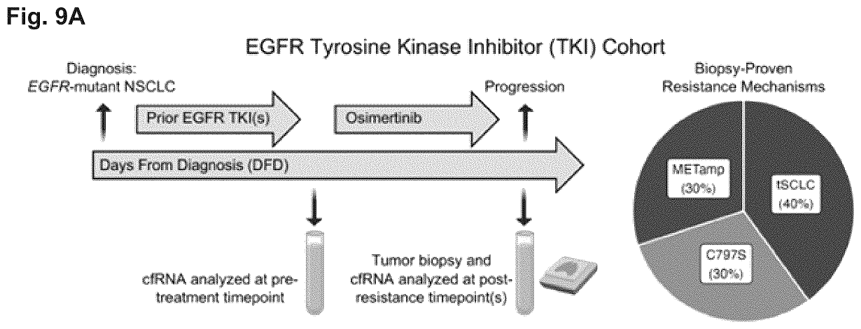

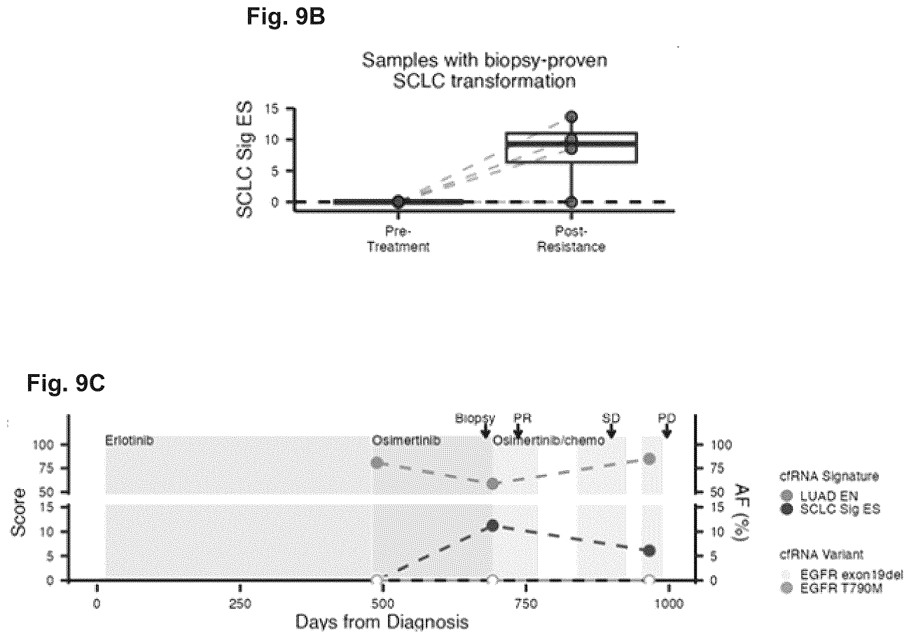

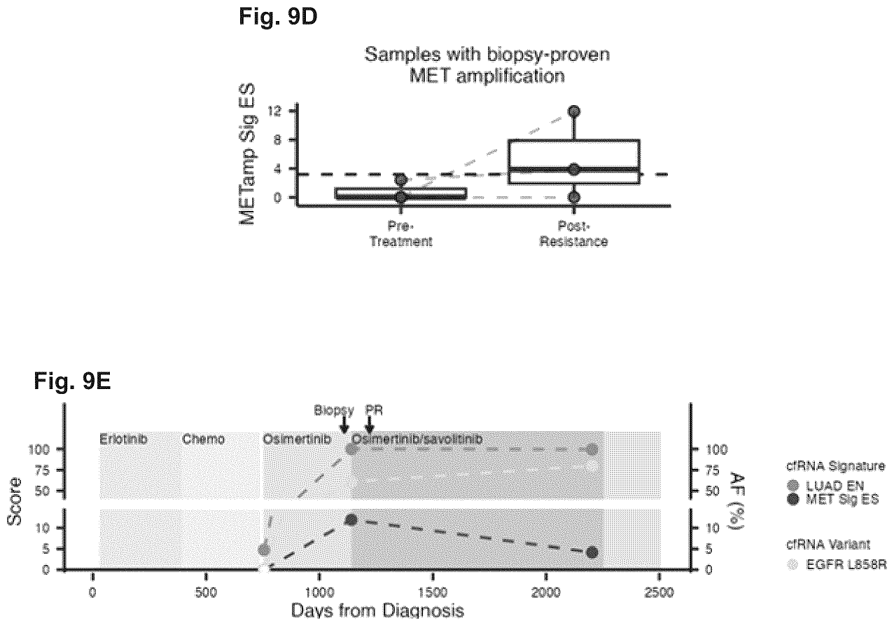

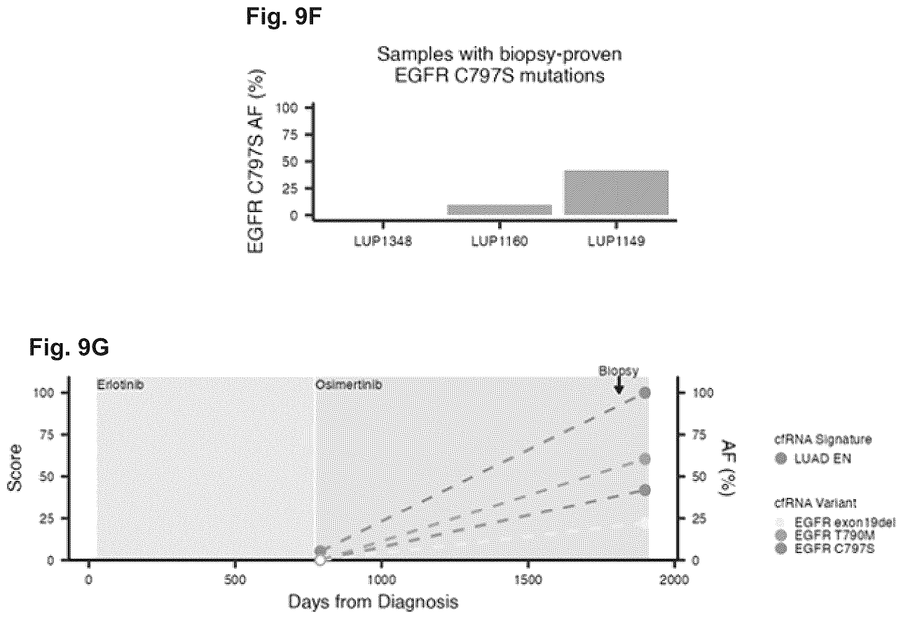

It was hypothesized that ctRNA analysis might allow detection of non-genetic therapeutic resistance mechanisms, such as histologic transformation. To explore this question, experiments were focused on EGFR-mutant LUAD patients treated with EGFR tyrosine kinase inhibitors (TKIs). Acquired resistance to EGFR TKIs is caused by heterogenous mechanisms, including both EGFR-dependent mechanisms (such as secondary point mutations in the EGFR gene) and EGFR-independent mechanisms (such as bypass pathway activation or histologic transformation). Plasma samples were profiled from 10 patients with stage IV EGFR-mutant LUAD who had received osimertinib after progressing on at least one prior EGFR TKI and who then developed resistance to osimertinib. The mechanism of resistance present in each patient was determined by tissue biopsy at the time of progression, revealing small cell histological transformation in 4 patients, MET gene amplifications in 3 patients, and emergent EGFR C797S point mutations in another 3 patients (FIG. 9A). Each patient had blood collections prior to start of osimertinib and one or more collections after progression (n=24 timepoints total).

Histological transformation to small cell lung cancer (SCLC) was evaluated using a SCLC signature (SCLC Sig) defined using public tumor gene expression data. The signature comprised 73 genes, including canonical SCLC markers such as ASCL1, NEUROD1, and INSM1. At time of radiologic progression, the SCLC Sig was detected in three of four (75%) patients with biopsy-proven histological transformation (FIG. 9B). For one of these patients, a plasma sample that was collected 274 days after the initiation of SCLC-targeted chemotherapy (e.g., carboplatin/etoposide) was observed to have decreased SCLC Sig ES, suggesting treatment response of the small cell component (FIG. 9C).

RARE-Seq was assessed for identifying genetic mechanisms of resistance. To detect MET pathway activation caused by MET amplification using ctRNA, a MET amplification signature (METamp Sig) was developed containing nine genes differentially expressed in three EGFR-mutant NSCLC cell lines with acquired resistance due to MET amplification. The METamp Sig was detected in two of three (67%) patients with biopsy-proven MET amplification (FIG. 9D). Notably, the METamp Sig ES decreased in one patient after the MET inhibitor savolitinib was added to osimertinib, coincident with a partial response by imaging (FIG. 9E). EGFR C797S mutant reads were identified in two of three (67%) patients known to have acquired this mutation based on tumor DNA or cfDNA analysis (FIGS. 9F and 9G). Importantly, these resistance mechanisms were not detected in any pre-resistance timepoint, confirming the specificity of the approach. Together, these results demonstrate proof-of-concept that RARE-Seq can identify both non-genetic and genetic mechanisms underlying EGFR TKI resistance, including histologic transformation which is not detectable by mutation-based ctDNA approaches.

Use of ctRNA Analysis for Tissue of Origin Determination

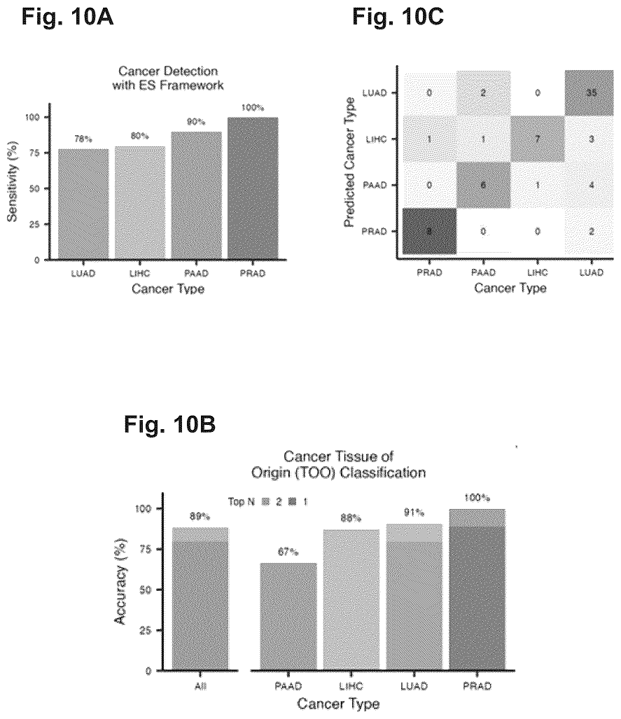

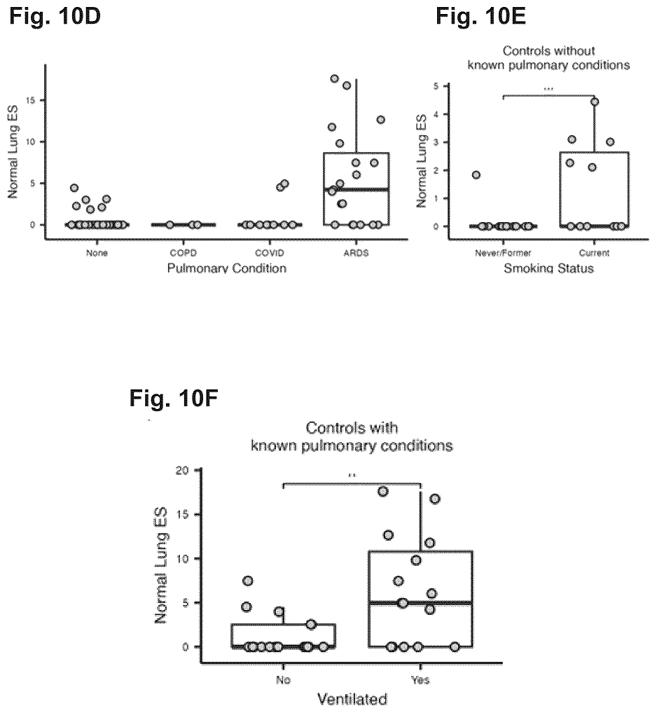

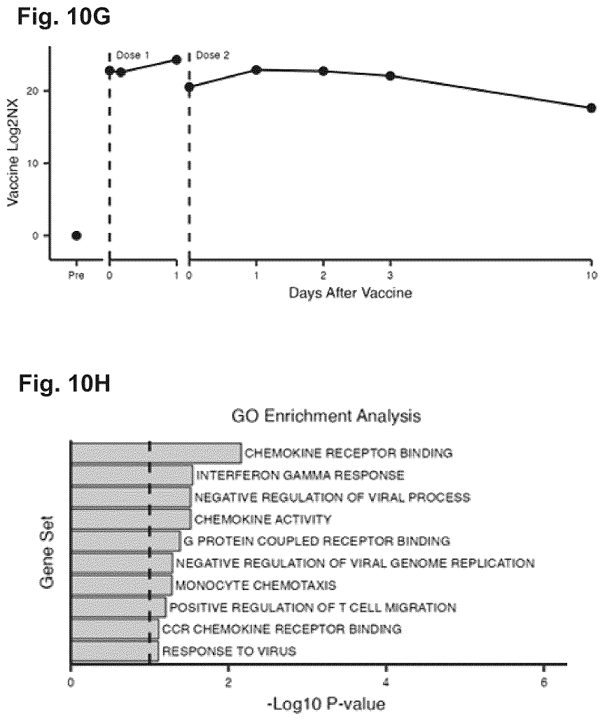

Since transcriptional profiles of cancers can reflect their tissue of origin (TOO), ctRNA analysis can be used to identify the cancer type. The ability to identify a malignancy's tissue of origin non-invasively could be helpful in several clinical settings, including in patients with metastatic cancers of unknown primary. To explore this potential application, plasma samples from patients with four advanced stage carcinoma subtypes were analyzed, including lung adenocarcinomas (LUAD, n=50), pancreatic adenocarcinoma (PAAD, n=10), prostate adenocarcinoma (PRAD, n=9) and hepatocellular carcinoma (LIHC, n=10). Corresponding tumor-specific gene signatures were generated for each cancer type using TCGA data and evaluated using the ES framework. Sensitivity of cancer-specific ES detection was 78%, 80%, 90%, and 100% for LUAD, LIHC, PAAD, and PRAD, respectively (FIG. 10A). To distinguish between cancer types, cancer signatures were generated that included genes uniquely expressed in each cancer compared to all other cancers, as well as to cfRNA from patients without cancer. Using these signatures, the top predicted TOO was correct 84% of the time and the top two predicted TOOs identified the correct tumor type ~89% of the time (FIGS. 10B and 10C). Thus, RARE-Seq could potentially enable non-invasive TOO identification.

### Application of RARE-Seq to Non-Malignant Conditions

RARE-Seq has several application beyond oncology assessment. It was tested whether cfRNA expression can reveal lung injury caused by benign pulmonary conditions. cfRNA was profiled from six patients with active COVID-19 infections (10 timepoints), 19 patients with acute respiratory distress syndrome (ARDS), and three patients with chronic obstructive pulmonary disorder (COPD). Across these diverse pulmonary conditions, lung derived cfRNA was detectable in 47% of patients using a normal lung gene expression signature derived from GTEx (FIG. 10D). In contrast, among adult subjects without known pulmonary conditions (n=42), lung cfRNA was detected in 14%, with detection being significantly more frequent in current smokers than in former- or never-smokers (45.5% vs 3.6%, P=0.004; FIG. 10E). This result suggests that lung cfRNA is induced by ongoing injury to pulmonary epithelium caused by recent exposure to tobacco smoke. Conversely, in patients with known pulmonary conditions, detection was significantly greater in those who were on a ventilator at the time of blood collection (FIG. 10F), likely reflecting the severity of the pulmonary condition as well as insult to pulmonary epithelium from mechanical ventilation. Thus, cfRNA analysis may have utility for assessment of benign conditions involving acute and chronic patterns of tissue injury.

Separately, given the rapidly growing interest in RNA-based vaccines and therapeutics, it was explored whether RARE-Seq has utility in simultaneously tracking RNA-based treatments and their effects on the host. To do so, blood plasma was longitudinally profiled from an individual undergoing their first two mRNA-based COVID-19 vaccinations. RARE-Seq was performed on these samples using the RAG capture panel supplemented with baits targeting the sequence of the mRNA vaccine. Vaccine-aligned RNA was detectable after both vaccinations and persisted at high levels for at least 10 days after injection (FIG. 10G). Intriguingly, genes that were differentially expressed post-vaccination compared to pre-vaccination were enriched for interferon gamma responses, antiviral responses, chemokine activity, and leukocyte migration pathways, suggesting detection of host response to vaccination (FIG. 10H). These results highlight the potential of RARE-Seq for simultaneously measuring pharmacokinetic and pharmacodynamic aspects of RNA-based therapies.

### Summary and Implications of Study

A novel framework for cfRNA profiling provides for a variety of potential clinical applications. It was found that RARE-Seq achieves analytical sensitivity levels between that of tumor-naïve and tumor-informed ctDNA-based methods tracking SNVs, indicating its potential utility for non-invasive profiling of tumor gene expression in patients with low disease burden. A key innovation of the approach is the specific capture of transcripts that are absent or very lowly expressed in the plasma of healthy controls. While analysis of these rare transcripts can be performed after sequencing the whole transcriptome, this approach is significantly less efficient since it predominantly measures leukocyte-derived gene expression and therefore misses rare transcripts. These latter transcripts, which are rare in healthy plasma cfRNA, are highly enriched for cancer- and solid organ-specific genes and therefore important for detection of pathophysiology occurring in tissues outside of the blood.

One important finding was the high proportion of platelet-derived transcripts present in plasma cfRNA. In contrast with cfDNA shed by megakaryocytes, these transcripts appear to at least partially stem from intact platelets that partition with plasma during blood sample processing, and present a potential confounder for cfRNA analyses. Indeed, expression differences in cfRNA of cancer patients versus controls reported in several prior studies appear to largely stem from differences in platelet-derived transcripts. While platelet contamination could be reduced using preanalytical approaches such as increasing centrifugation speed, platelet poor plasma preparations are seldom entirely platelet free. Additionally, such procedures are often not feasible when analyzing historical samples including from completed clinical trials. Therefore, the approach described herein for removing platelet contributions to cfRNA gene expression profiles are broadly useful for future cfRNA liquid biopsy studies.

The exploration of potential applications suggests that ultrasensitive cfRNA analysis may be useful in diverse clinical settings. For example, since the approach enables tumor naïve detection of ctRNA, it could potentially be used for cancer screening, either alone or in combination with other diagnostic modalities. The fact that RARE-Seq detected lung cancer RNA in some samples that were negative by ctDNA analysis suggests the two approaches may be complimentary. A second promising application is non-invasive identification of cancer types. Such an approach could be useful for non-invasive identification of TOO in patients with cancers of unknown primary or allow distinguishing between cancer subtypes.

When considering EGFR-mutant NSCLC patients treated with EGFR TKIs, the results suggest that RARE-Seq provides more comprehensive non-invasive analysis of treatment resistance mechanisms, including those such as histologic transformation that are not driven by recurrent emergent somatic alterations. Since histologic transformation is a common mechanism of resistance that occurs in diverse cancer types and because its diagnosis currently requires invasive tissue biopsies, non-invasive detection using cfRNA can greatly enhance diagnostic methods. The ability of RARE-Seq to simultaneously query for the presence of somatic mutations (e.g., EGFR C797S) and the transcriptional effects of pathway activation via somatic alterations (e.g., MET amplification) or non-genetic mechanism (e.g., histologic transformation) enables broader profiling of diverse resistance mechanisms than currently possible.

Finally, RARE-Seq will be useful in detecting non-cancer derived cfRNA signatures in patients with both malignant and benign conditions. For example, the data demonstrating the presence of normal lung RNA in the plasma of patients with acute lung injury due to conditions such as COVID-19 infection or ARDS suggests cfRNA analysis may also allow blood-based monitoring of tissue injury. Separately, as demonstrated in the analysis of cfRNA in plasma samples after COVID-19 mRNA vaccination, that RARE-Seq enables simultaneous tracking of mRNA vaccines or therapeutics and measurement of host responses.

In summary, a versatile method for cfRNA analysis was developed. The method has utility in diverse clinical settings. The results also include several important biological and technical insights that enable more sensitive cfRNA analysis and that are broadly useful for cfRNA-based liquid biopsy applications. The method enables novel non-invasive diagnostic applications, with the promise of advancing precision medicine and improving patient care.

### Methods

### Human Participants and Cohorts

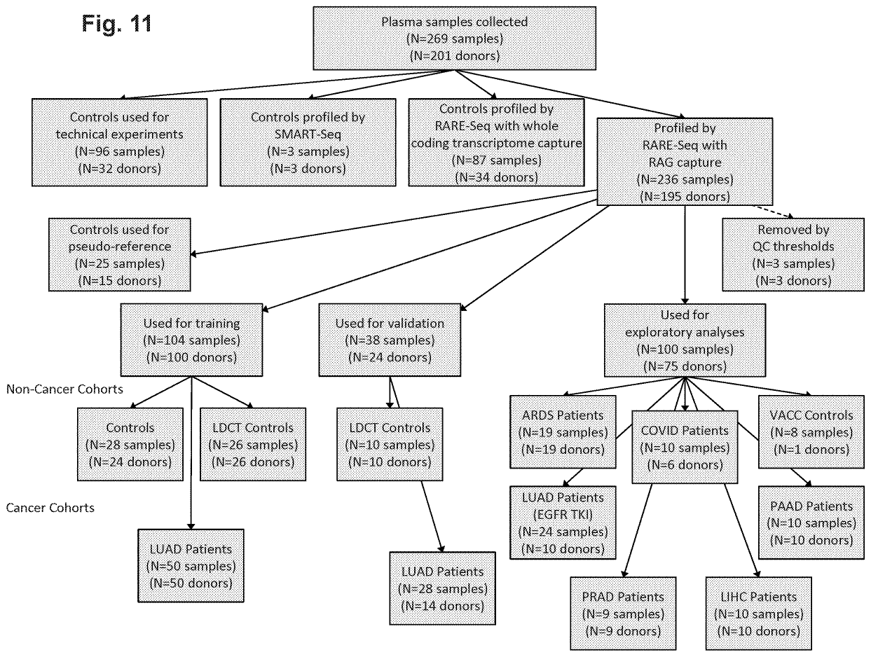

All samples analyzed in this study were collected with informed consent using protocols approved by Institutional Review Boards at their respective centers. Collection centers included Stanford University, Memorial Sloan Kettering Cancer Center (MSKCC), Massachusetts General Hospital (MGH), and University Hospital Zurich, as detailed below. In total, 269 blood samples were collected from 201 individuals (FIG. 11). The clinical and demographic characteristics are presented in Table 1 for non-cancer cancer cohorts and in Table 2 for cancer cohorts.

Cancer cohorts. Blood samples were collected from non-small cell lung cancer (NSCLC) patients enrolled at Stanford University (n=33), MSKCC (n=17), and MGH (n=14). Of these, 64 (98%) were diagnosed with lung adenocarcinoma (LUAD) and 1 patient (2%) had mixed adenosquamous histology. Stage information was determined using the American Joint Committee on Cancer (AJCC) 8^{th} edition. For 10 EGFR-mutant NSCLC patients enrolled at MGH, blood was collected at multiple timepoints prior to treatment with and after progression on the EGFR tyrosine kinase inhibitor (TKI) osimertinib, where available (n=24 plasma samples). Tissue biopsies were collected at the time of progression to identify the mechanism of resistance. For additional exploratory analyses, blood samples were collected at Stanford University from patients diagnosed with advanced stage pancreatic adenocarcinoma (PAAD, n=10) and advanced stage prostate adenocarcinoma (PRAD, n=9) and at University Hospital Zurich from patients with hepatocellular carcinoma (LIHC, n=10).

Non-cancer cohorts. Blood from individuals with no known cancer or benign pulmonary conditions was collected at Stanford University for technical experiments and for method development controls (n=45). A subset of these samples comprised the ‘meta-reference’ set that was used to define expected expression in healthy cfRNA for various downstream analyses (n=15). Blood samples were collected from individuals at the time of low-dose computed tomography (LDCT) screening at Stanford University (n=27) and at MGH (n=10), who qualified for such screening based on smoking history (>=30 pack years) and age (55-80 years). Three individuals at the time of LDCT screening were noted to have chronic obstructive pulmonary disease (COPD). In addition, blood samples were collected from patients treated at Stanford Hospital for acute respiratory distress syndrome (ARDS; n=20) and coronavirus disease 2019 (COVID-19; n=6). Plasma was also was collected from one individual after receiving two doses of the Pfizer-BioNTech COVID-19 mRNA vaccination at Stanford University (n=8 timepoints).

### Blood Collection and Plasma Processing

Peripheral blood samples were collected and processed according to protocols at their respective centers. For technical experiments conducted at Stanford, whole blood was collected in EDTA tubes and plasma isolated using centrifugation at 2,500G for 10 minutes at 4° C. Optical density at 414 nm was measured using a Nanodrop spectrophotometer instrument to quantify levels of hemolysis in plasma. After centrifugation, all plasma was stored at −80° C. until cell-free nucleic acid isolation. cfRNA extraction

Cell-free nucleic acids were extracted from plasma using the miRNA protocol from the QIAamp Circulating Nucleic Acid kit (Qiagen (range 0.2-8.0 mL). Extraction was performed according to manufacturer's instructions with minor modifications. The resulting eluate was incubated with 14 U DNase I (RNase-Free DNase Set, Qiagen) for 30 minutes at room temperature to digest DNA. The digested eluate was purified using the Zymo RNA Clean&Concentrator kit and stored at −80° C.

Plasma processed before February 2021 was extracted using phenol/chloroform phase separation and purified using QIAamp Viral RNA kit (Qiagen). In brief, plasma was first incubated with 3 volumes of TRI Reagent LS (Molecular Research Center), and then incubated with 0.4 volumes of chloroform (relative to plasma input). Phase separation was performed using Maxtract 50 mL conical tubes (Qiagen) spun at 1,500G for 5 minutes at room temperature. The RNA-containing aqueous phase was carefully removed, and RNA extracted according to the QIAamp Viral RNA kit protocol. DNA digestion and clean-up were performed as described above. For the subset of samples digested using the “on column” protocol, 28 U DNase I was added directly to the sample bound to the silica column and incubated for 15 minutes, according to the manufacturer recommendations. Careful analysis was done to compare the cfRNA yield and gene expression distribution between different extraction methods (FIGS. 1E and 2B). As no significant differences were found, cfRNA samples extracted using both methods were combined for the analyses presented in this manuscript.

cfRNA Quantification

Quantitative real-time polymerase chain reaction (qRT-PCR) was used for quantification of cfRNA. An RNA-specific primer was designed to cover a 97-bp amplicon spanning 2 exon boundaries in the housekeeping gene GAPDH (Forward 5′-GATCATCAGCAATGCCTCCT-3′ (SEQ ID NO: 1), Reverse 5′-TGTGGTCATGAGTCCTTCCA-3′ (SEQ ID NO: 2)). A DNA-specific primer was designed to cover a 78 bp transcriptionally silent region of chromosome 12 (Forward 5′-TACGGTTGGTCCTTTCTTCG-3′ (SEQ ID NO: 3), Reverse 5′-TTTCCTTTGGGTCTGAATGC-3′ (SEQ ID NO: 4)). Reverse transcription was first performed using the High-Capacity cDNA Reverse Transcription kit (Applied Biosystems). Quantitative PCR (qPCR) was then run using 2× Power SYBR Green PCR Master Mix (Thermo Fisher Scientific) on an Applied Biosystems 7500 Fast Real-Time PCR or QuantStudio 7 Pro instruments. Universal Human Reference RNA (Thermo Fisher Scientific) was run in parallel to generate a standard curve, and cfRNA concentrations were calculated by comparing the sample's RNA-specific Ct value to the standard curve. An analogous method was used to quantify DNA using Human Genomic DNA (Promega). If DNA was detected using the DNA-specific primer, DNA digestion, clean-up, and quantification was repeated. The cfRNA size distribution was assessed using the Agilent Bioanalyzer RNA 6000 Pico chip.

cfRNA Library Preparation and Sequencing

RARE-Seq. Input mass was 424 pg cfRNA, which represents the 25th percentile of cfRNA yield for 4 mL of plasma in healthy controls (FIG. 1B). For samples with less than 424 pg, all extracted cfRNA was used for library preparation (range 8-424 pg). Double-stranded complementary DNA (cDNA) was synthesized from cfRNA using the NEBNext Ultra™ II RNA First-Strand Synthesis Module and Non-Directional Second Strand Synthesis Module (New England Biolabs). Double-stranded cDNA was treated with 100 U S1 nuclease (Thermo Fisher) for 30 minutes at room temperature to hydrolyze incomplete (i.e., single-stranded) regions. The KAPA Hyper Prep kit (Kapa Biosystems) was used to prepare libraries for sequencing, primarily following manufacturer's instructions. Whole coding transcriptome capture was performed using the Roche Nimblegen SeqCap EZ MedExome Target Enrichment Kit and or the Twist Biosciences Comprehensive Exome Hybridization kit, following respective manufacturer's instructions. Single-plex capture with a custom gene panel targeting rare abundance genes (see ‘RAG capture panel design’ below) was performed using the Twist Biosciences Hybridization kit. Captured libraries were sequenced using 2×150-bp paired-end reads on Illumina HiSeq4000 or NovaSeq6000 instruments.

SMART-Seq. cfRNA from 3 controls was input into library preparation using the SMART-Seq Stranded kit (TaKaRa) per manufacturer's instructions and without additional fragmentation. Libraries were amplified and sequenced using 150-bp paired-end runs on an Illumina NovaSeq6000.

### Leukocyte RNA Processing

After plasma separation, leukocytes were isolated from plasma-depleted whole blood samples using SepMate PBMC isolation tubes (Stem Cell Technologies), according to manufacturer instructions. Leukocyte cell pellets were stored at −80° C. RNA was extracted from leukocyte cell pellets by mixing with 800 ul TRIzol (Invitrogen) and 200 ul chloroform, followed by centrifugation at 13,000G for 15 minutes at 4° C. RNA was then purified from the aqueous supernatant using Qiagen RNeasy kit, per manufacturer's instructions. Total RNA was quantified with the NanoDrop instrument and Agilent Bioanalyzer RNA 6000 Pico chip. 5-10 ng RNA was used for RARE-Seq library preparation and whole coding transcriptome capture as previously described. Fragmentation was performed before first strand synthesis as recommended by manufacturer.

### NCI-H1975 Cell Line RNA Processing

NCI-H1975 lung adenocarcinoma cells were acquired from American Type Culture Collection (ATCC). Cellular RNA was extracted from cell pellets and quantified as described for leukocyte RNA. To create in vitro NCI-H1975 cell line spikes, NCI-H1975 RNA was serially diluted into cfRNA from a single healthy individual to create samples with 10%, 1%, 0.1%, 0.01%, and 0.001% cancer fraction by mass. Triplicate RARE-seq libraries were generated from each mixture as well as NCI-H1975 RNA (100% cancer fraction) and cfRNA alone (0% cancer fraction) and captured with whole coding transcriptome panel. Additional mixtures using cfRNA from a different healthy individual were created for capture with the RAG panel (see ‘RAG capture panel design’ below).

cfDNA Library Preparation and Sequencing

Cell-free DNA (cfDNA) was extracted from plasma using the standard cfDNA protocol from the QIAamp Circulating Nucleic Acid kit (Qiagen). After extraction, cfDNA was quantified with Qubit double-stranded DNA High Sensitivity kit (Thermo Fisher Scientific) and High Sensitivity NGS Fragment Analyzer (Agilent). CAncer Personalized Profiling by deep Sequencing (CAPP-Seq) was used to create sequencing libraries from 32 ng cfDNA. Thereafter, hybridization-based capture (Roche NimbleGen) was used to target genes recurrently mutated in lung cancer. Libraries were sequenced using 2×150 bp paired-end reads on Illumina HiSeq4000 instruments.

### Mapping, Deduplication, and Quality Control for RARE-Seq

FASTQ files were first demultiplexed using a custom pipeline. Fastp (v0.20.0) trimmed the first 10 bases from the 5′ end of Read 1 and the 3′ end of Read 2 as well as removed low quality or too short read pairs from each sample. Remaining high quality reads were aligned to the reference transcriptome (GENCODE v27) and to the human genome (hg19) using STAR 2-pass (v2.7.0). PCR duplicates were removed from both transcriptome-aligned and genome-aligned files using a custom barcoding approach. Deduplicated reads were used for gene-level expression estimation using RSEM (v1.2.28).

Quality control was assessed using metrics calculated by the RNASeQC package (v2.3.5) such as read mapping quality and mapping rates, including exonic, intronic, and intergenic rates and ribosomal RNA rates. In addition, DNA contamination was estimated by calculating the percent of reads that map to intronic sequences out of the total number of reads that span an exon boundary. Samples were removed from downstream analysis if they had fewer than 20 million reads or if they had greater than 20% estimated DNA contamination. Together, these QC thresholds removed three samples.

### Gene Expression Normalization and Platelet Correction

RSEM expected counts for captured genes were used for expression analyses (tximport R package v1.22). Counts were first normalized using the Trimmed Mean of M-values (TMM) method, which accounts for sample-to-sample variation in library size and transcriptome complexity (edgeR R package v3.36). Log-transformed and normalized expression values are referred to as ‘log 2NX’ herein. A modified version of the Remove Unwanted Variation (RUV) approach was then used to correct for expression variation caused by platelets (RUVseq R package v1.28; D. Risso, et al., Nat. Biotechnol. 32, 896-902 (2014), the disclosure of which is hereby incorporated by reference). For whole coding transcriptome libraries, a factor of unwanted platelet variation was estimated by RUVg using 130 platelet cell-type marker genes from PanglaoDB as the set of negative control genes (FIG. 3I) (O Franzen, et al., Database (Oxford). 2019 Jan. 1; 2019:baz046, the disclosure of which is hereby incorporated by reference). For RAG captured libraries, since most platelet genes were intentionally excluded from panel (see ‘RAG capture panel design’ below), factors of unwanted variation were estimated using the healthy control meta-reference cfRNA (n=15) as negative control samples within RUVs. The factor of unwanted platelet variation was selected to be the factor that most correlated with platelet expression, defined as average log 2NX across 21 captured platelet cell-type marker genes (e.g., PPBP) (FIG. 8G). For both RUVg and RUVs, platelet correction was done by ordinary least squares regression of log 2NX on the selected factor of unwanted platelet expression. RUVg and RUVs performed similarly in whole coding transcriptome samples (FIGS. 3J and 6H). After normalization and correction, quality control was performed by calculating the Pearson correlation between each sample's gene expression profile and the average of all other samples from the same sample group (i.e., ‘within-group correlation’). For controls with more than one biological replicate, the replicate with the highest within-group correlation was selected for downstream analyses.

### Differential Gene Expression Analysis

Differential gene expression analysis was performed using DESeq2 (DESeq2 R package v1.34; M. Love, et al., Genome Biol. 15, 550 (2014), the disclosure of which is hereby incorporated by reference). For analysis of cfRNA and cellular RNA differential expression, a generalized linear model (GLM) was built using sample type as the covariate. For analysis of cancer versus control differential expression, the GLM used condition (i.e., cancer or control) and the factor of unwanted platelet variation determined by RUV as covariates. Wald test was used to determine significance of log fold change estimates followed by Benjamini-Hochberg multiple hypothesis correction. For the COVID-19 vaccination time series analysis, significance of differential expression was evaluated using a likelihood ratio test comparing the full GLM including vaccine timepoint and factor of unwanted variation compared to a reduced model with vaccine terms removed. For all analyses, significantly differentially expressed genes were defined by absolute log fold change greater than one and adjusted p-value<0.05. Functional analysis of differentially expressed genes used gene set enrichment analysis (GSEA) of pre-ranked log fold change estimates (fgsea R package v1.20) or gene ontology (GO) analysis (goseq R package v1.46). GSEA and GO analysis were performed using either cell marker genes from the PanglaoDB single-cell RNA sequencing database and/or the molecular signatures database (MSigDb) (O Franzen, et al., Database (Oxford). 2019 Jan. 1; 2019:baz046; A. Subramanian, et al., Proc. Natl. Acad. Sci. U.S.A. 102, 15545-15550 (2005); A. Liberzon, et al., Cell Syst 1, 417-425 (2015); and A. LIberzon, et al., Bioinformatics 27, 1739-1740 (2011); the disclosures of which are each hereby incorporated by reference).

### RAG-Focused Capture Panel Design

To identify rare-abundance genes (RAGs) in cfRNA, gene expression data were analyzed from controls generated with the whole coding transcriptome RARE-Seq method (n=50 biological replicates from n=28 individuals). In addition, gene expression data from whole blood samples generated by The Genotype-Tissue Expression (GTEx) project were accessed through the UCSC Xena repository (n=307). Expression was TMM-normalized, and k-means clustering (k=2) was used to categorize genes as expressed or unexpressed in each sample. RAGs were defined as genes that were expressed in less than 5 percent of all samples and for which average log 2NX was in the bottom 30 percent of all genes for both cfRNA and whole blood. RAGs are listed in Table 3. Next, expression uniformity was calculated in cfRNA and whole blood using Gini coefficient, and endogenous control genes were selected from housekeeping genes with a Gini<0.2 in circulation. In addition, lung cancer-associated genes were manually curated for inclusion in the panel, including genes that are recurrently mutated or rearranged in lung cancer or that are aberrantly expressed in lung cancer histological types. In total, the RAG-focused capture panel included 80,866 probes targeting 7,766,820 bp and 5,546 unique genes. Probes were also designed to target the sequences of the Pfizer-BioNTech and Moderna COVID-19 mRNA vaccines and were used to supplement the RAG capture panel for samples collected post-vaccination.

### Cell Type, Tissue, and Cancer Gene Signatures

For cell type-specific signatures, 7,481 marker genes from 170 human cell types were downloaded from the PanglaoDB single-cell RNA sequencing database (O Franzen, et al., Database (Oxford). 2019 Jan. 1; 2019:baz046, the disclosure of which is hereby incorporated by reference).

Tissue- and cancer-enriched signatures were identified using gene expression data from the Genotype-Tissue Expression (GTEx) and The Cancer Genome Atlas (TCGA) projects was analyzed. Gene-level read counts generated by the UCSC Toil RNA sequencing bioinformatic pipeline were accessed in the UCSC Xena repository. Gene expression was TMM-normalized and outlier samples were removed using a within-group correlation metric. The TCGA cohort was further filtered to exclude tumors with less than 60 percent tumor purity using consensus purity estimates (CPE). The normal tissue types that were evaluated from GTEx included bladder (n=9), brain (n=1,107), breast (n=168), colon (n=261), esophagus (n=598), kidney (n=24), liver (n=97), lung (n=241), ovary (n=79), pancreas (n=145), prostate (n=86), skin (n=497), stomach (n=167), and whole blood (n=290). Genes were defined as tissue-enriched if expression was 5-fold higher in each tissue compared to all other tissues evaluated, and if average tissue log 2NX was greater than zero. Similarly, the cancer tissue types that were evaluated from TCGA included bladder (BLCA, n=344), brain (GBM, n=144; LGG, n=183), breast (BRCA, n=914), colon (COAD, n=270), esophagus (ESCA, n=181), kidney (KICH, n=65; KIRC, n=350; KIRP, n=262), liver (LIHC, n=164), lung (LUAD, n=309; LUSC, n=365), ovary (OV, n=412), pancreas (PAAD, n=177), prostate (PRAD, n=473), melanoma (SKCM, n=94), and stomach (STAD, n=413). Genes were defined as cancer-enriched if expression was 5-fold higher in each cancer tissue type compared to all other cancer types evaluated, and if average cancer tissue log 2NX was greater than zero.

Cancer-specific gene signatures were created to include the genes most differentially expressed in each cancer tissue compared to whole blood and meta-reference cfRNA, and therefore most likely to be detected in cfRNA mixtures with high hematopoietic background. For this purpose, the TCGA cancer cohort was supplemented with gene expression data from the Cancer Cell Line Encyclopedia (CCLE) accessed through Xena (LIHC, n=25; LUAD, n=76; PAAD, n=40, PRAD, n=7, SCLC, n=50) and an additional SCLC study (n=79). DESeq2 differential gene expression analysis quantified the gene-wise fold change between cancer tissue expression and whole blood and meta-reference cfRNA expression. Each gene was also categorized as expressed or unexpressed in cancer tissue, whole blood, and meta-reference cfRNA using K-means clustering (k=2) of average gene expression in each group. Genes were selected for cancer-specific signatures if they were:
1. Found in top 1% of genes over-expressed in the cancer tissue of interest OR in whole blood and meta-reference cfRNA, and
2. Categorized as expressed in the cancer tissue of interest OR in whole blood and meta-reference cfRNA.

An analogous method was used to generate cancer TOO gene signatures except that gene expression was compared between LIHC, LUAD, PAAD, PRAD, whole blood, and meta-reference cfRNA, rather than analyzing each cancer type individually. Genes were selected for cancer TOO signatures if they were:
1. Found in the top 1% of genes over-expressed in cancer tissue compared to whole blood and meta-reference cfRNA,
2. Found in the top 5% of genes over-expressed in the cancer tissue of interest compared to each other cancer type, and
3. Categorized as expressed in the cancer tissue of interest, and unexpressed in whole blood and meta-reference cfRNA.

For all signatures, a gene weight was defined as the sum of cancer tissue log 2NX and cancer tissue versus meta-reference cfRNA log fold change, so that the weight preserved the sign of the differential expression analysis (i.e., positive for genes over-expressed in cancer tissue).

### Gene Signature Detection Using Enrichment Scores

The enrichment score (ES) analytical framework was developed to be broadly applicable, as any gene signature of interest can be used. Gene expression of a sample was first standardized into Z-scores using average and standard deviation of meta-reference cfRNA expression. Next, Z-scores for a gene signature of interest were aggregated using the Stouffer equation and the gene weights, resulting in a single ES for each sample. If applicable, Z-scores were aggregated separately for genes with positive and negative weights and summated. Bootstrapping was used to create random gene signatures (n=1,000) of the same size (but excluding genes within the gene signature), and the bootstrapped ES distribution was used to estimate an empirical p-value for each signature in each sample. The ES was set to zero if the empirical p-value was less than 0.05.

### Limit of Detection Estimation Using H1975 Mixtures

The 95% limit of detection (LOD95) of RARE-Seq was determined using in silico RNA mixtures. Sequencing data for NCI-H1975 RNA and healthy control cfRNA was generated and analyzed as described for the respective sample type. Reads were randomly subsampled from each replicate and merged in pre-specified cancer fractions ranging from 0% to 100%, and the enrichment score analytical framework was used to detect cancer in each sample. Here, an NCI-H1975 gene signature (H1975 Sig) was created by comparing NCI-H1975 samples processed by RARE-Seq but not used for spike creation (n=4) and meta-reference cfRNA (n=15). Genes were selected if they were: 1. Found in top 1% of genes over-expressed in NCI-H1975 compared to meta-reference cfRNA, and

### 2. Categorized as Expressed in NCI-H1975.

The limit of blank (LOB) was calculated from the average and standard deviation of H1975 Sig ES found in cfRNA controls not used to create the gene signature (n=12) and was set as the detection threshold for remaining spikes. Logistic regression was employed to model the relationship between cancer fraction and H1975 Sig detection, and LOD95 was defined as the cancer fraction at which sensitivity exceeded 95%. This process was repeated for a range of RNA inputs and sequencing depths. The in vitro NCI-H1975 mixtures described above were processed as other cfRNA samples and detected using H1975 Sig to confirm the in silico results.

### Cancer Detection Using Elastic Net Classification

Statistical learning models, including elastic net (EN) logistic regression, random forest, and XGBoost, were trained to classify LUAD patient cfRNA (n=50) from controls (n=50). Nested 10-fold cross-validation (10CV) was used to tune hyperparameters and estimate model performance (caret R package v6.0). Features considered for model training included expression from all captured genes, AF from all recurrent, somatic variants queried in ctRNA, and binarized values representing presence of SNVs, fusions, indels, splice variants, or variants of any type detected in a given sample. Pre-processing removed features with near-zero variance in the training cohort as well as genes that were not significantly differentially expressed between LUAD tumor tissue and meta-reference cfRNA, as described for the enrichment score framework. The gene weights used for the enrichment score framework were also evaluated as penalty weights within a weighted elastic net model. To evaluate the impact of cohort size on model performance, 10CV was repeated after sub-sampling 20-100% of the cohort and the standard deviation of AUC was determined at each sub-sampling level across n=10 random samples. All trained models worked reasonably well for classification, but the best performance was achieved with weighted EN logistic regression. The final weighted EN model built using the full training cohort selected 279 genes out of 1,006 features considered, including features related to variant detection in ctRNA. The final model was validated using a withheld cfRNA cohort collected at an independent institution (n=28 timepoints from 14 LUAD patients, n=10 LDCT controls). ROC curves and metrics such as sensitivity, specificity, and AUC were generated using the pROC R package (v1.18).

### Cancer Tissue-of-Origin Classification

Samples that were detected by any cancer-specific gene signature were considered for tissue-of-origin (TOO) analysis. TOO was classified using a modified enrichment score framework. Here, Z-scores were aggregated by calculating the 90^{th} percentile of all Z-scores in a gene signature of interest. For each sample, scores for all cancer TOO gene signatures were ranked. Accuracy was determined based on if the diagnosed cancer type had the highest cancer TOO score detected, or the second-highest score.

ctRNA Variant Calling

Variant calling was performed for a targeted list of actionable NSCLC alterations compiled based on National Cancer Comprehensive Network (NCCN) guidelines. For each candidate variant, the null hypothesis that variant allele frequency (AF) was consistent with a position- and depth-specific background error distribution was tested. The background error rate, b, was calculated as the fraction of reads supporting the substitution across a training cohort consisting of control cfRNA (n=53). The background error distribution was obtained by sampling 10,000 positive values from a binomial distribution with the candidate variant depth as the “number of trials” and b as “success rate”, from which a p-value was estimated. The resulting p-values were adjusted for multiple hypothesis testing using the Benjamini-Hochberg method and substitutions with a q-value<0.05 were considered significant. Candidate fusions identified by STAR-Fusion (v1.6.0; A. Dobin, et al., Bioinformatics 29, 15-21 (2013), the disclosure of which is hereby incorporated by reference) were called if present in the list of known fusion partners in lung cancer as annotated on cBioPortal. MET exon 14 skipping alterations were called if the mapping contained splices linking exons 13 to 15. Indels in EGFR exons 19 and 20 were called if greater than 6 base-pairs.

ctDNA Variant Calling

Patient-specific variant lists were collected from prior clinical tumor genotyping if available or otherwise pre-treatment plasma, including only those variants which were also captured by the lung cancer CAPP-Seq panel. CAPP-Seq sequencing data was analyzed as follows: In brief, reads were demultiplexed, trimmed to remove low quality bases from the 3′ end using fastp, and aligned to the hg19 human genome using BWA ALN. PCR duplicates were removed using a custom barcoding approach. Single nucleotide variants (SNVs) and insertion/deletion events (indels) were called using the previously reported integrated digital error suppression (iDES) pipeline (A. M. Newman, et al., Nat. Biotechnol. 34, 547-555 (2016), the disclosure of which is hereby incorporated by reference). Gene fusions were identified using FACTERA (A. M. Newman, et al., Bioinformatics 30, 3390-3393 (2014), the disclosure of which is hereby incorporated by reference). The ctDNA sample allele frequency (AF) was calculated by averaging AF of detected patient-specific variants.

### MET Amplification Gene Signature

EGFR-mutant NSCLC cell line pairs with acquired resistance to EGFR TKI due to MET amplification (PC9/PC9-PERC17, MGH1157-1/MGH1157-3, MGH170-1C #7/MGH170-1D #2) were generated. The PC9-PERC17 line was generated by in vitro culture of PC9 cells with erlotinib. MGH1157-1 and MGH1157-3 cell lines were developed from sequential biopsies of a patient before and after treatment with the combination of gefitinib and nazartanib, respectively. Clinical profiling of the MGH1157-3 tumor using MET FISH demonstrated MET amplification. MGH170 cell lines were developed from a patient with acquired MET amplification (confirmed by FISH) after erlotinib treatment. Single cell clones were isolated from two separate metastatic lesions at the time of rapid autopsy following disease progression, the MGH170-1C #7 clonal cell line was sensitive to EGFR TKI, while the MGH170-1 D #2 was found to be resistant. MET amplification was confirmed in all resistant cell lines, and MET dependency was confirmed by sensitivity to combination EGFR+MET TKIs. All tissue samples were obtained after patients signed informed consent to participate in a Dana-Farber-Harvard Cancer Center Institutional Review Board-approved protocol giving permission for research to be performed on their samples. mRNA-seq was performed on total RNA from pre-treatment and resistant cell lines (n=3 replicates at each timepoint for n=3 cell lines, or n=18 samples total) and differential expression analysis was performed using DESeq2 with MET amplification status as covariate. Genes were selected for a MET amplification gene signature if they were:
1. Found in the top 1% of genes over-expressed in MET-amplified cell lines compared to meta-reference cfRNA,
2. Significantly differentially expressed in MET-amplified cell lines compared to non-amplified cell lines, and
3. Categorized as expressed in MET-amplified cell lines.

Here, the gene weight was defined as the sum of MET-amplified cell line log 2NX and MET-amplified versus meta-reference cfRNA log fold change, so that the weight preserved the sign of the differential expression analysis (i.e., positive for genes over-expressed in MET-amplified cell lines).

### Statistical Analysis

All statistical analyses were performed in R (v4.1.3). Statistical tests used throughout the manuscript include the Wilcoxon rank-sum test, paired t-test, Fisher's exact test, Kruskal-Wallis analysis of variance, Pearson correlation, Spearman correlation, and DeLong's test for comparing AUC. Unless otherwise specified, all statistical tests comparing two groups used the two-sided Wilcoxon rank-sum test and all statistical tests comparing three or more groups used Kruskall-Wallis analysis of variance. Unless p-values are stated, significance labels are used in figure panels as follows: *P<0.05; **P<0.01; ***P<0.001, ****P<0.0001. Multiple hypothesis correction was performed using the Benjamini-Hochberg (BH) method. Unless otherwise specified, the box plots depict the interquartile range (box), median (center line), and minimum/maximum (whiskers). Unless otherwise specified, the sample size (n) provided in text, figure panels, and figure legends refers to biologically independent individuals. Details of the statistical methods (including R packages) used in the enrichment score analytical framework, elastic net model training, and variant calling approaches are described in their respective section of Methods.

### Optimizing Blood Collection and RNA Extraction

It was determined that a median concentration per mL plasma is 220 pg cfRNA, as determined using RNA-specific quantitative RT-PCR (FIG. 1B). Given these low concentrations, it was first sought to optimize blood collection and RNA extraction. Since hemolysis has previously been shown to be an important pre-analytical factor affecting other blood-based biomarkers, its impact on cfRNA concentrations was examined. Levels of hemoglobin did not correlate with cfRNA amount in the cohort, suggesting that hemolysis is not a significant pre-analytical variable in this setting (FIG. 1C). In contrast, the length of time that plasma was stored at −80° C. did negatively correlate with cfRNA levels. However, this effect was only observed during the first month of storage, after which there was no association between storage time and cfRNA yields (FIG. 1D). Next, the effect of different blood collection tubes on cfRNA concentration was examined. All types of blood collection tubes (BCTs) tested, including with and without cell-free nucleic acid preservatives, enabled isolation of cfRNA, with minimal concentration differences (FIG. 1E). Ten cfRNA isolation protocols were compared. Two silica column-based purification methods protocols resulted in significantly higher concentrations (QIAamp Circulating Nucleic Acid kit and a customized version of the QIAamp Viral RNA kit; FIG. 1F) and were used for subsequent experiments.

### Optimizing RNA Sequencing Library Preparation

Next, a library preparation protocol was developed and optimized that coupled random primer-based cDNA synthesis with ligation of sequencing adapters containing unique molecular identifying (UMI) barcodes, enabling precise enumeration of unique cfRNA molecules recovered from plasma. After generation of such cfRNA-derived sequencing libraries, affinity hybridization was used to biotinylated oligonucleotides to capture the coding transcriptome. Sequencing data was processed using a custom bioinformatics pipeline as described herein. It was confirmed that the measured cfRNA expression was highly correlated regardless of the tube type (FIG. 2A) and extraction method (FIG. 2B) employed. It was postulated that a protocol that preserved the orientation of the originating RNA molecule (i.e., strandedness) might be useful for cfRNA analysis since this approach has been reported to be useful for analyzing differential expression in whole blood. However, while gene-specific expression levels were nearly identical between stranded and non-stranded libraries, non-stranded libraries contained ~30% more unique molecules (FIGS. 2C and 2D), suggesting that the stranded library preparation protocol resulted in loss of transcripts. It was also examined if RNA transcript recovery could be improved using methods previously shown to overcome the highly damaged nature of FFPE nucleic acids. It was found that additional end repair with single-strand specific S1 nuclease improved transcript recovery by >2-fold while faithfully preserving expression levels (FIGS. 2E and 2F). The protocol therefore incorporated non-stranded library preparation and S1 nuclease end repair, but other protocols could be utilized to achieve results.

Digesting Contaminating cfDNA from Extracted Nucleic Acids

Since cfRNA analyses are known to be confounded by the presence of contaminating cfDNA, the optimal approach for digestion of DNA using DNase I was assessed. While most silica column-based RNA isolation protocols recommend that enzymatic DNA digestion be performed while RNA is bound to the column, it was found that this can result in incomplete DNA removal. Performing the enzymatic digestion step on the eluted nucleic acids decreased cfDNA contamination levels, from 63.6% to 9.1% (FIG. 2G). Expression correlation substantially decreased between samples with and without DNA contamination, underscoring the importance of removing DNA prior to sequencing library preparation (FIG. 2H). To further protect from DNA contamination in cfRNA analysis, cfRNA samples with high levels of estimated DNA contamination were excluded from subsequent experiments.

### Validating RARE-Seq Optimizations

Before applying to clinical samples, the RARE-Seq method was benchmarked against the widely used SMART-Seq whole transcriptome method, which includes nuclear and mitochondrial ribosomal RNA depletion but no other enrichment steps. Consistent with previous reports, RNAs encoding protein coding genes were the most abundant biotype in cfRNA analyzed using SMART-Seq, representing 67.1% of mapped reads (range 55.6-68.2%) (FIG. 2I). Protein coding gene expression was highly concordant between SMART-Seq and RARE-Seq libraries from matched cfRNA (R=0.96, P=<2.2e-16) (FIG. 2J). However, 80.4% of coding genes had equal or higher sequencing depth in RARE-Seq, corresponding to a 44% increase in total sequencing depth (FIG. 2J). RARE-Seq reproducibility was assessed using eight replicates from the same individual, which were collected on four separate days and sequenced in five batches. Three libraries were made from the same cfRNA pool, serving as technical replicates. High average correlations of 0.988 and 0.997 were observed for biological and technical replicates, respectively (FIG. 2K). Together, these results suggest that RARE-Seq is a robust and reproducible method for measuring plasma cfRNA expression.

TABLE 1
Non-cancer cohort demographics.

| | Control | | | | LDCT | | | | ARDS | | COVID | | VACC | |
|---|---|---|---|---|---|---|---|---|---|---|---|---|---|---|
| | Meta-Reference | | Training | | Training | | Validation | | Other | | Other | | Other | |
| No. of samples | 25 | | 28 | | 26 | | 10 | | 19 | | 10 | | 8 | |
| No. of donors | 15 | | 24 | | 26 | | 10 | | 19 | | 6 | | 1 | |
| Age (years) | 37 | (23-52) | 31 | (24-48) | 62 | (56-76) | 62 | (54-77) | 65 | (32-80) | 58 | (38-69) | 29 | (29-29) |

Sex

| | | | | | | | | | | | | | | |
|---|---|---|---|---|---|---|---|---|---|---|---|---|---|---|
| Male | 12 | (46.0%) | 24 | (85.7%) | 23 | (88.5%) | 4 | (40.0%) | 9 | (47.4%) | 7 | (70.0%) | 0 | (0.0%) |
| Female | 13 | (52.0%) | 4 | (14.3%) | 3 | (11.5%) | 6 | (60.0%) | 10 | (52.6%) | 3 | (30.0%) | 8 | (100.0%) |

Smoking Status

| | | | | | | | | | | | | | | |
|---|---|---|---|---|---|---|---|---|---|---|---|---|---|---|
| Current | 0 | (0.0%) | 0 | (0.0%) | 18 | (69.2%) | 3 | (30.0%) | 3 | (15.8%) | 0 | (0.0%) | 0 | (0.0%) |
| Former | 3 | (12.0%) | 3 | (10.7%) | 8 | (30.8%) | 7 | (70.0%) | 7 | (36.8%) | 3 | (30.0%) | 0 | (0.0%) |
| Never | 22 | (88.0%) | 22 | (78.6%) | 0 | (0.0%) | 0 | (0.0%) | 9 | (47.4%) | 4 | (40.0%) | 8 | (100.0%) |
| Unknown | 0 | (0.0%) | 3 | (10.7%) | 0 | (0.0%) | 0 | (0.0%) | 0 | (0.0%) | 3 | (30.0%) | 0 | (0.0%) |

Institution

| Stanford University | 25 | 28 | 26 | 0 | 19 | 10 | 8 |
|---|---|---|---|---|---|---|---|
| Massachusetts | | | 0 | 10 | | | |
| General Hospital | | | | | | | |

LDCT, low-dose computed tomography. ARDS, acute respiratory distress syndrome. COVID, COVID-19 infection. VACC, post-COVID-19 mRNA vaccination.

TABLE 2
Cancer cohort demographics.

| | LUAD | | | | LUAD (TKI) | | PAAD | | PRAD | | LIHC | |
|---|---|---|---|---|---|---|---|---|---|---|---|---|
| | Training | | Validation | | Other | | Other | | Other | | Other | |
| No. of samples | 50 | | 28 | | 24 | | 10 | | 9 | | 10 | |
| No. of donors | 50 | | 14 | | 10 | | 10 | | 9 | | 10 | |
| Age (years) | 66 | (38-87) | 61 | (30-77) | 59 | (30-77) | 64 | (42-82) | 74 | (54-86) | 62 | (41-82) |

Sex

| | | | | | | | | | | | | |
|---|---|---|---|---|---|---|---|---|---|---|---|---|
| Male | 21 | (42.0%) | 8 | (28.6%) | 5 | (20.8%) | 4 | (40.0%) | 9 | (100.0%) | 6 | (60.0%) |
| Female | 29 | (58.0%) | 20 | (71.4%) | 19 | (79.2%) | 6 | (60.0%) | 0 | (0.0%) | 4 | (40.0%) |

Smoking Status

| | | | | | | | | | | | | |
|---|---|---|---|---|---|---|---|---|---|---|---|---|
| Current | 2 | (4.0%) | 0 | (0.0%) | 0 | (0.0%) | 0 | (0.0%) | 0 | (0.0%) | 6 | (60.0%) |
| Former | 29 | (58.0%) | 9 | (32.1%) | 7 | (29.2%) | 3 | (30.0%) | 3 | (33.3%) | 1 | (10.0%) |
| Never | 19 | (38.0%) | 19 | (67.9%) | 17 | (70.8%) | 7 | (70.0%) | 4 | (44.4%) | 3 | (30.0%) |
| Unknown | 0 | (0.0%) | 0 | (0.0%) | 0 | (0.0%) | 0 | (0.0%) | 2 | (22.2%) | 0 | (0.0%) |
| Pack Years | 20.6 | (27.9) | 1.1 | (2.0) | 1.0 | (1.9) | 0.8 | (2.5) | 7.3 | (14.9) | 26.0 | (25.9) |

Stage

| | | | | | | | | | | | | |
|---|---|---|---|---|---|---|---|---|---|---|---|---|
| I | 5 | (10.0%) | 0 | (0.0%) | 0 | (0.0%) | 0 | (0.0%) | 0 | (0.0%) | 3 | (30.0%) |
| II | 5 | (10.0%) | 0 | (0.0%) | 0 | (0.0%) | 0 | (0.0%) | 0 | (0.0%) | 5 | (50.0%) |
| III | 10 | (20.0%) | 0 | (0.0%) | 0 | (0.0%) | 0 | (0.0%) | 1 | (11.1%) | 0 | (0.0%) |
| IV | 30 | (60.0%) | 28 | (100.0%) | 24 | (100.0%) | 10 | (100.0%) | 8 | (88.9%) | 2 | (20.0%) |

Institution

| Massachusetts General | 0 | 28 | 24 |
|---|---|---|---|
| Hospital | | | |
| Memorial Sloan Kettering | 17 | 0 | |
| Cancer Center | | | |

| | | | | |
|---|---|---|---|---|
| Stanford University | 33 | 0 | 10 | 9 |

| | |
|---|---|
| University Hospital | 10 |
| Zurich | |

LUAD, lung adenocarcinoma. TKI, treated with epidermal growth factor receptor (EGFR) tyrosine kinase inhibitor. PAAD, pancreatic adenocarcinoma. PRAD, prostate adenocarcinoma. LIHC, liver hepatocellular carcinoma.

TABLE 3
Rare Abundance Genes (RAGs)
RAGs defined as expressed in less than 5% of healthy samples and
for which average log2NX was in the bottom 30 percent of all genes.

| | HGNC |
|---|---|
| Ensembl Gene | Gene |
| ID | Symbol |
| ENSG00000000003 | TSPAN6 |
| ENSG00000000005 | TNMD |
| ENSG00000001626 | CFTR |
| ENSG00000002746 | HECW1 |
| ENSG00000003137 | CYP26B1 |
| ENSG00000004846 | ABCB5 |
| ENSG00000004848 | ARX |
| ENSG00000004948 | CALCR |
| ENSG00000005001 | PRSS22 |
| ENSG00000005073 | HOXA11 |
| ENSG00000005108 | THSD7A |
| ENSG00000005421 | PON1 |
| ENSG00000005981 | ASB4 |
| ENSG00000006047 | YBX2 |
| ENSG00000006059 | KRT33A |
| ENSG00000006071 | ABCC8 |
| ENSG00000006116 | CACNG3 |
| ENSG00000006128 | TAC1 |
| ENSG00000006283 | CACNA1G |
| ENSG00000006377 | DLX6 |
| ENSG00000006468 | ETV1 |
| ENSG00000006606 | CCL26 |
| ENSG00000006611 | USH1C |
| ENSG00000006659 | LGALS14 |
| ENSG00000006747 | SCIN |
| ENSG00000006788 | MYH13 |
| ENSG00000006837 | CDKL3 |
| ENSG00000007001 | UPP2 |
| ENSG00000007062 | PROM1 |
| ENSG00000007171 | NOS2 |
| ENSG00000007174 | DNAH9 |
| ENSG00000007216 | SLC13A2 |
| ENSG00000007306 | CEACAM7 |
| ENSG00000007908 | SELE |
| ENSG00000007952 | NOX1 |
| ENSG00000008118 | CAMK1G |
| ENSG00000008196 | TFAP2B |
| ENSG00000008197 | TFAP2D |
| ENSG00000009709 | PAX7 |
| ENSG00000009765 | IYD |
| ENSG00000010379 | SLC6A13 |
| ENSG00000010438 | PRSS3 |
| ENSG00000010932 | FMO1 |
| ENSG00000011083 | SLC6A7 |
| ENSG00000011332 | DPF1 |
| ENSG00000011347 | SYT7 |
| ENSG00000011677 | GABRA3 |
| ENSG00000012504 | NR1H4 |
| ENSG00000013293 | SLC7A14 |
| ENSG00000015413 | DPEP1 |
| ENSG00000015520 | NPC1L1 |
| ENSG00000015592 | STMN4 |
| ENSG00000016082 | ISL1 |
| ENSG00000016402 | IL20RA |
| ENSG00000016490 | CLCA1 |
| ENSG00000016602 | CLCA4 |
| ENSG00000018236 | CNTN1 |
| ENSG00000019186 | CYP24A1 |
| ENSG00000019505 | SYT13 |
| ENSG00000019549 | SNAI2 |
| ENSG00000021461 | CYP3A43 |
| ENSG00000021488 | SLC7A9 |
| ENSG00000021645 | NRXN3 |
| ENSG00000021826 | CPS1 |
| ENSG00000021852 | C8B |
| ENSG00000022355 | GABRA1 |
| ENSG00000025423 | HSD17B6 |
| ENSG00000027644 | INSRR |
| ENSG00000029559 | IBSP |
| ENSG00000030304 | MUSK |
| ENSG00000033122 | LRRC7 |
| ENSG00000034239 | EFCAB1 |
| ENSG00000034971 | MYOC |
| ENSG00000036473 | OTC |
| ENSG00000036530 | CYP46A1 |
| ENSG00000036565 | SLC18A1 |
| ENSG00000036672 | USP2 |
| ENSG00000036828 | CASR |
| ENSG00000037965 | HOXC8 |
| ENSG00000038295 | TLL1 |
| ENSG00000039139 | DNAH5 |
| ENSG00000039537 | C6 |
| ENSG00000039600 | SOX30 |
| ENSG00000039987 | BEST2 |
| ENSG00000040731 | CDH10 |
| ENSG00000041515 | MYO16 |
| ENSG00000042304 | C2orf83 |
| ENSG00000042781 | USH2A |
| ENSG00000042813 | ZPBP |
| ENSG00000043039 | BARX2 |
| ENSG00000043355 | ZIC2 |
| ENSG00000044012 | GUCA2B |
| ENSG00000044524 | EPHA3 |
| ENSG00000046604 | DSG2 |
| ENSG00000046774 | MAGEC2 |
| ENSG00000047457 | CP |
| ENSG00000047662 | FAM184B |
| ENSG00000047936 | ROS1 |
| ENSG00000048540 | LMO3 |
| ENSG00000048545 | GUCA1A |
| ENSG00000049283 | EPN3 |
| ENSG00000050030 | KIAA2022 |
| ENSG00000052850 | ALX4 |
| ENSG00000053328 | METTL24 |
| ENSG00000053747 | LAMA3 |
| ENSG00000054796 | SPO11 |
| ENSG00000054803 | CBLN4 |
| ENSG00000054938 | CHRDL2 |
| ENSG00000055732 | MCOLN3 |
| ENSG00000055813 | CCDC85A |
| ENSG00000056291 | NPFFR2 |
| ENSG00000056487 | PHF21B |
| ENSG00000056998 | GYG2 |
| ENSG00000057149 | SERPINB3 |
| ENSG00000057468 | MSH4 |
| ENSG00000057593 | F7 |
| ENSG00000058085 | LAMC2 |
| ENSG00000058335 | RASGRF1 |
| ENSG00000058404 | CAMK2B |
| ENSG00000060566 | CREB3L3 |
| ENSG00000060709 | RIMBP2 |
| ENSG00000060718 | COL11A1 |
| ENSG00000061455 | PRDM6 |
| ENSG00000061492 | WNT8A |
| ENSG00000062038 | CDH3 |
| ENSG00000062096 | ARSF |
| ENSG00000062370 | ZNF112 |
| ENSG00000063515 | GSC2 |
| ENSG00000064195 | DLX3 |
| ENSG00000064218 | DMRT3 |
| ENSG00000064309 | CDON |
| ENSG00000064655 | EYA2 |
| ENSG00000064692 | SNCAIP |
| ENSG00000064835 | POU1F1 |
| ENSG00000065325 | GLP2R |
| ENSG00000065371 | ROPN1 |
| ENSG00000065609 | SNAP91 |
| ENSG00000066032 | CTNNA2 |
| ENSG00000066230 | SLC9A3 |
| ENSG00000066248 | NGEF |
| ENSG00000066382 | MPPED2 |
| ENSG00000066405 | CLDN18 |
| ENSG00000066468 | FGFR2 |
| ENSG00000066813 | ACSM2B |
| ENSG00000067842 | ATP2B3 |
| ENSG00000068615 | REEP1 |
| ENSG00000068781 | STON1-GTF2A1L |
| ENSG00000068985 | PAGE1 |
| ENSG00000069011 | PITX1 |
| ENSG00000069018 | TRPC7 |
| ENSG00000069206 | ADAM7 |
| ENSG00000069431 | ABCC9 |
| ENSG00000069482 | GAL |
| ENSG00000069696 | DRD4 |
| ENSG00000069764 | PLA2G10 |
| ENSG00000069812 | HES2 |
| ENSG00000070019 | GUCY2C |
| ENSG00000070031 | SCT |
| ENSG00000070159 | PTPN3 |
| ENSG00000070193 | FGF10 |
| ENSG00000070388 | FGF22 |
| ENSG00000070601 | FRMPD1 |
| ENSG00000070729 | CNGB1 |
| ENSG00000070748 | CHAT |
| ENSG00000070808 | CAMK2A |
| ENSG00000070886 | EPHA8 |
| ENSG00000070915 | SLC12A3 |
| ENSG00000070985 | TRPM5 |
| ENSG00000071203 | MS4A12 |
| ENSG00000071677 | PRLH |
| ENSG00000071909 | MYO3B |
| ENSG00000071991 | CDH19 |
| ENSG00000072041 | SLC6A15 |
| ENSG00000072080 | SPP2 |
| ENSG00000072133 | RPS6KA6 |
| ENSG00000072182 | ASIC4 |
| ENSG00000072315 | TRPC5 |
| ENSG00000072657 | TRHDE |
| ENSG00000073067 | CYP2W1 |
| ENSG00000073146 | MOV10L1 |
| ENSG00000073598 | FNDC8 |
| ENSG00000073734 | ABCB11 |
| ENSG00000074047 | GLI2 |
| ENSG00000074211 | PPP2R2C |
| ENSG00000074317 | SNCB |
| ENSG00000074771 | NOX3 |
| ENSG00000075035 | WSCD2 |
| ENSG00000075043 | KCNQ2 |
| ENSG00000075073 | TACR2 |
| ENSG00000075213 | SEMA3A |
| ENSG00000075290 | WNT8B |
| ENSG00000075388 | FGF4 |
| ENSG00000075429 | CACNG5 |
| ENSG00000075461 | CACNG4 |
| ENSG00000075643 | MOCOS |
| ENSG00000075673 | ATP12A |
| ENSG00000075886 | TUBA3D |
| ENSG00000075891 | PAX2 |
| ENSG00000076716 | GPC4 |
| ENSG00000077009 | NMRK2 |
| ENSG00000077063 | CTTNBP2 |
| ENSG00000077080 | ACTL6B |
| ENSG00000077092 | RARB |
| ENSG00000077264 | PAK3 |
| ENSG00000077274 | CAPN6 |
| ENSG00000077279 | DCX |
| ENSG00000077327 | SPAG6 |
| ENSG00000077498 | TYR |
| ENSG00000077800 | FKBP6 |
| ENSG00000077935 | SMC1B |
| ENSG00000078098 | FAP |
| ENSG00000078295 | ADCY2 |
| ENSG00000078328 | RBFOX1 |
| ENSG00000078549 | ADCYAP1R1 |
| ENSG00000078579 | FGF20 |
| ENSG00000078725 | BRINP1 |
| ENSG00000078795 | PKD2L2 |
| ENSG00000078898 | BPIFB2 |
| ENSG00000079101 | CLUL1 |
| ENSG00000079112 | CDH17 |
| ENSG00000079150 | FKBP7 |
| ENSG00000079557 | AFM |
| ENSG00000079689 | SCGN |
| ENSG00000079841 | RIMS1 |
| ENSG00000080031 | PTPRH |
| ENSG00000080166 | DCT |
| ENSG00000080224 | EPHA6 |
| ENSG00000080293 | SCTR |
| ENSG00000080511 | RDH8 |
| ENSG00000080572 | PIH1D3 |
| ENSG00000080644 | CHRNA3 |
| ENSG00000080709 | KCNN2 |
| ENSG00000080910 | CFHR2 |
| ENSG00000081051 | AFP |
| ENSG00000081138 | CDH7 |
| ENSG00000081248 | CACNA1S |
| ENSG00000081479 | LRP2 |
| ENSG00000081800 | SLC13A1 |
| ENSG00000081818 | PCDHB4 |
| ENSG00000081842 | PCDHA6 |
| ENSG00000081853 | PCDHGA2 |
| ENSG00000082126 | MPP4 |
| ENSG00000082175 | PGR |
| ENSG00000082482 | KCNK2 |
| ENSG00000082497 | SERTAD4 |
| ENSG00000082556 | OPRK1 |
| ENSG00000082684 | SEMA5B |
| ENSG00000083067 | TRPM3 |
| ENSG00000083307 | GRHL2 |
| ENSG00000083782 | EPYC |
| ENSG00000084453 | SLCO1A2 |
| ENSG00000084628 | NKAIN1 |
| ENSG00000084734 | GCKR |
| ENSG00000085552 | IGSF9 |
| ENSG00000086159 | AQP6 |
| ENSG00000086205 | FOLH1 |
| ENSG00000086696 | HSD17B2 |
| ENSG00000086991 | NOX4 |
| ENSG00000087128 | TMPRSS11E |
| ENSG00000087250 | MT3 |
| ENSG00000087494 | PTHLH |
| ENSG00000087510 | TFAP2C |
| ENSG00000088002 | SULT2B1 |
| ENSG00000088320 | REM1 |
| ENSG00000088386 | SLC15A1 |
| ENSG00000088756 | ARHGAP28 |
| ENSG00000088782 | DEFB127 |
| ENSG00000088926 | F11 |
| ENSG00000089101 | CFAP61 |
| ENSG00000089116 | LHX5 |
| ENSG00000089225 | TBX5 |
| ENSG00000089250 | NOS1 |
| ENSG00000089356 | FXYD3 |
| ENSG00000089558 | KCNH4 |
| ENSG00000090402 | SI |
| ENSG00000090512 | FETUB |
| ENSG00000090534 | THPO |
| ENSG00000090539 | CHRD |
| ENSG00000090932 | DLL3 |
| ENSG00000091010 | POU4F3 |
| ENSG00000091128 | LAMB4 |
| ENSG00000091137 | SLC26A4 |
| ENSG00000091138 | SLC26A3 |
| ENSG00000091482 | SMPX |
| ENSG00000091656 | ZFHX4 |
| ENSG00000091664 | SLC17A6 |
| ENSG00000091879 | ANGPT2 |
| ENSG00000092009 | CMA1 |
| ENSG00000092345 | DAZL |
| ENSG00000092377 | TBL1Y |
| ENSG00000092607 | TBX15 |
| ENSG00000092850 | TEKT2 |
| ENSG00000092969 | TGFB2 |
| ENSG00000094661 | OR1I1 |
| ENSG00000094755 | GABRP |
| ENSG00000094796 | KRT31 |
| ENSG00000095110 | NXPE1 |
| ENSG00000095464 | PDE6C |
| ENSG00000095587 | TLL2 |
| ENSG00000095596 | CYP26A1 |
| ENSG00000095627 | TDRD1 |
| ENSG00000095752 | IL11 |
| ENSG00000095777 | MYO3A |
| ENSG00000095917 | TPSD1 |
| ENSG00000095981 | KCNK16 |
| ENSG00000096264 | NCR2 |
| ENSG00000096395 | MLN |
| ENSG00000097096 | SYDE2 |
| ENSG00000099399 | MAGEB2 |
| ENSG00000099617 | EFNA2 |
| ENSG00000099625 | CBARP |
| ENSG00000099715 | PCDH11Y |
| ENSG00000099721 | AMELY |
| ENSG00000099769 | IGFALS |
| ENSG00000099812 | MISP |
| ENSG00000099834 | CDHR5 |
| ENSG00000099957 | P2RX6 |
| ENSG00000099960 | SLC7A4 |
| ENSG00000100012 | SEC14L3 |
| ENSG00000100033 | PRODH |
| ENSG00000100053 | CRYBB3 |
| ENSG00000100078 | PLA2G3 |
| ENSG00000100101 | RP1-37E16.12 |
| ENSG00000100121 | GGTLC2 |
| ENSG00000100122 | CRYBB1 |
| ENSG00000100146 | SOX10 |
| ENSG00000100156 | SLC16A8 |
| ENSG00000100170 | SLC5A1 |
| ENSG00000100191 | SLC5A4 |
| ENSG00000100196 | KDELR3 |
| ENSG00000100249 | C22orf31 |
| ENSG00000100253 | MIOX |
| ENSG00000100302 | RASD2 |
| ENSG00000100312 | ACR |
| ENSG00000100341 | PNPLA5 |
| ENSG00000100344 | PNPLA3 |
| ENSG00000100433 | KCNK10 |
| ENSG00000100557 | C14orf105 |
| ENSG00000100565 | LRRC74A |
| ENSG00000100593 | ISM2 |
| ENSG00000100604 | CHGA |
| ENSG00000100625 | SIX4 |
| ENSG00000100626 | GALNT16 |
| ENSG00000100652 | SLC10A1 |
| ENSG00000100665 | SERPINA4 |
| ENSG00000100739 | BDKRB1 |
| ENSG00000100842 | EFS |
| ENSG00000100867 | DHRS2 |
| ENSG00000100884 | CPNE6 |
| ENSG00000100987 | VSX1 |
| ENSG00000101049 | SGK2 |
| ENSG00000101074 | R3HDML |
| ENSG00000101076 | HNF4A |
| ENSG00000101098 | RIMS4 |
| ENSG00000101115 | SALL4 |
| ENSG00000101134 | DOK5 |
| ENSG00000101144 | BMP7 |
| ENSG00000101180 | HRH3 |
| ENSG00000101197 | BIRC7 |
| ENSG00000101198 | NKAIN4 |
| ENSG00000101200 | AVP |
| ENSG00000101203 | COL20A1 |
| ENSG00000101204 | CHRNA4 |
| ENSG00000101222 | SPEF1 |
| ENSG00000101251 | SEL1L2 |
| ENSG00000101276 | SLC52A3 |
| ENSG00000101280 | ANGPT4 |
| ENSG00000101282 | RSPO4 |
| ENSG00000101292 | PROKR2 |
| ENSG00000101311 | FERMT1 |
| ENSG00000101323 | HAO1 |
| ENSG00000101327 | PDYN |
| ENSG00000101349 | PAK7 |
| ENSG00000101405 | OXT |
| ENSG00000101435 | CST9L |
| ENSG00000101438 | SLC32A1 |
| ENSG00000101440 | ASIP |
| ENSG00000101441 | CST4 |
| ENSG00000101443 | WFDC2 |
| ENSG00000101446 | SPINT3 |
| ENSG00000101448 | EPPIN |
| ENSG00000101463 | SYNDIG1 |
| ENSG00000101489 | CELF4 |
| ENSG00000101542 | CDH20 |
| ENSG00000101670 | LIPG |
| ENSG00000101680 | LAMA1 |
| ENSG00000101746 | NOL4 |
| ENSG00000101812 | H2BFM |
| ENSG00000101825 | MXRA5 |
| ENSG00000101850 | GPR143 |
| ENSG00000101871 | MID1 |
| ENSG00000101883 | RHOXF1 |
| ENSG00000101890 | GUCY2F |
| ENSG00000101892 | ATP1B4 |
| ENSG00000101951 | PAGE4 |
| ENSG00000101958 | GLRA2 |
| ENSG00000101981 | F9 |
| ENSG00000102021 | LUZP4 |
| ENSG00000102048 | ASB9 |
| ENSG00000102076 | OPN1LW |
| ENSG00000102104 | RS1 |
| ENSG00000102128 | RAB40AL |
| ENSG00000102195 | GPR50 |
| ENSG00000102239 | BRS3 |
| ENSG00000102243 | VGLL1 |
| ENSG00000102271 | KLHL4 |
| ENSG00000102290 | PCDH11X |
| ENSG00000102313 | ITIH6 |
| ENSG00000102359 | SRPX2 |
| ENSG00000102383 | ZDHHC15 |
| ENSG00000102385 | DRP2 |
| ENSG00000102387 | TAF7L |
| ENSG00000102452 | NALCN |
| ENSG00000102466 | FGF14 |
| ENSG00000102468 | HTR2A |
| ENSG00000102539 | MLNR |
| ENSG00000102678 | FGF9 |
| ENSG00000102683 | SGCG |
| ENSG00000102794 | IRG1 |
| ENSG00000102891 | MT4 |
| ENSG00000102924 | CBLN1 |
| ENSG00000102962 | CCL22 |
| ENSG00000102970 | CCL17 |
| ENSG00000103021 | CCDC113 |
| ENSG00000103023 | PRSS54 |
| ENSG00000103067 | ESRP2 |
| ENSG00000103089 | FA2H |
| ENSG00000103241 | FOXF1 |
| ENSG00000103310 | ZP2 |
| ENSG00000103375 | AQP8 |
| ENSG00000103449 | SALL1 |
| ENSG00000103460 | TOX3 |
| ENSG00000103546 | SLC6A2 |
| ENSG00000103599 | IQCH |
| ENSG00000103710 | RASL12 |
| ENSG00000103742 | IGDCC4 |
| ENSG00000104044 | OCA2 |
| ENSG00000104055 | TGM5 |
| ENSG00000104059 | FAM189A1 |
| ENSG00000104112 | SCG3 |
| ENSG00000104140 | RHOV |
| ENSG00000104213 | PDGFRL |
| ENSG00000104237 | RP1 |
| ENSG00000104313 | EYA1 |
| ENSG00000104321 | TRPA1 |
| ENSG00000104327 | CALB1 |
| ENSG00000104369 | JPH1 |
| ENSG00000104371 | DKK4 |
| ENSG00000104413 | ESRP1 |
| ENSG00000104415 | WISP1 |
| ENSG00000104435 | STMN2 |
| ENSG00000104499 | GML |
| ENSG00000104537 | ANXA13 |
| ENSG00000104723 | TUSC3 |
| ENSG00000104755 | ADAM2 |
| ENSG00000104804 | TULP2 |
| ENSG00000104808 | DHDH |
| ENSG00000104818 | CGB2 |
| ENSG00000104826 | LHB |
| ENSG00000104827 | CGB |
| ENSG00000104848 | KCNA7 |
| ENSG00000104888 | SLC17A7 |
| ENSG00000104901 | DKKL1 |
| ENSG00000104938 | CLEC4M |
| ENSG00000104941 | RSPH6A |
| ENSG00000104953 | TLE6 |
| ENSG00000105131 | EPHX3 |
| ENSG00000105141 | CASP14 |
| ENSG00000105143 | SLC1A6 |
| ENSG00000105198 | LGALS13 |
| ENSG00000105219 | CNTD2 |
| ENSG00000105251 | SHD |
| ENSG00000105261 | OVOL3 |
| ENSG00000105388 | CEACAM5 |
| ENSG00000105392 | CRX |
| ENSG00000105419 | MEIS3 |
| ENSG00000105428 | ZNRF4 |
| ENSG00000105467 | SYNGR4 |
| ENSG00000105523 | FAM83E |
| ENSG00000105549 | THEG |
| ENSG00000105550 | FGF21 |
| ENSG00000105605 | CACNG7 |
| ENSG00000105642 | KCNN1 |
| ENSG00000105650 | PDE4C |
| ENSG00000105664 | COMP |
| ENSG00000105668 | UPK1A |
| ENSG00000105675 | ATP4A |
| ENSG00000105679 | GAPDHS |
| ENSG00000105695 | MAG |
| ENSG00000105696 | TMEM59L |
| ENSG00000105784 | RUNDC3B |
| ENSG00000105792 | CFAP69 |
| ENSG00000105825 | TFPI2 |
| ENSG00000105852 | PON3 |
| ENSG00000105877 | DNAH11 |
| ENSG00000105880 | DLX5 |
| ENSG00000105889 | STEAP1B |
| ENSG00000105894 | PTN |
| ENSG00000105929 | ATP6V0A4 |
| ENSG00000105954 | NPVF |
| ENSG00000105976 | MET |
| ENSG00000105989 | WNT2 |
| ENSG00000105996 | HOXA2 |
| ENSG00000105997 | HOXA3 |
| ENSG00000106004 | HOXA5 |
| ENSG00000106006 | HOXA6 |
| ENSG00000106013 | ANKRD7 |
| ENSG00000106025 | TSPAN12 |
| ENSG00000106031 | HOXA13 |
| ENSG00000106038 | EVX1 |
| ENSG00000106113 | CRHR2 |
| ENSG00000106128 | GHRHR |
| ENSG00000106178 | CCL24 |
| ENSG00000106236 | NPTX2 |
| ENSG00000106258 | CYP3A5 |
| ENSG00000106278 | PTPRZ1 |
| ENSG00000106302 | HYAL4 |
| ENSG00000106304 | SPAM1 |
| ENSG00000106328 | FSCN3 |
| ENSG00000106331 | PAX4 |
| ENSG00000106384 | MOGAT3 |
| ENSG00000106410 | NOBOX |
| ENSG00000106436 | MYL10 |
| ENSG00000106483 | SFRP4 |
| ENSG00000106511 | MEOX2 |
| ENSG00000106536 | POU6F2 |
| ENSG00000106541 | AGR2 |
| ENSG00000106631 | MYL7 |
| ENSG00000106633 | GCK |
| ENSG00000106648 | GALNTL5 |
| ENSG00000106686 | SPATA6L |
| ENSG00000106688 | SLC1A1 |
| ENSG00000106689 | LHX2 |
| ENSG00000106819 | ASPN |
| ENSG00000106823 | ECM2 |
| ENSG00000106852 | LHX6 |
| ENSG00000107014 | RLN2 |
| ENSG00000107018 | RLN1 |
| ENSG00000107105 | ELAVL2 |
| ENSG00000107159 | CA9 |
| ENSG00000107165 | TYRP1 |
| ENSG00000107187 | LHX3 |
| ENSG00000107295 | SH3GL2 |
| ENSG00000107447 | DNTT |
| ENSG00000107518 | ATRNL1 |
| ENSG00000107593 | PKD2L1 |
| ENSG00000107807 | TLX1 |
| ENSG00000107821 | KAZALD1 |
| ENSG00000107831 | FGF8 |
| ENSG00000107859 | PITX3 |
| ENSG00000107984 | DKK1 |
| ENSG00000108001 | EBF3 |
| ENSG00000108018 | SORCS1 |
| ENSG00000108176 | DNAJC12 |
| ENSG00000108231 | LGI1 |
| ENSG00000108242 | CYP2C18 |
| ENSG00000108255 | CRYBA1 |
| ENSG00000108342 | CSF3 |
| ENSG00000108379 | WNT3 |
| ENSG00000108381 | ASPA |
| ENSG00000108417 | KRT37 |
| ENSG00000108511 | HOXB6 |
| ENSG00000108602 | ALDH3A1 |
| ENSG00000108684 | ASIC2 |
| ENSG00000108688 | CCL7 |
| ENSG00000108700 | CCL8 |
| ENSG00000108702 | CCL1 |
| ENSG00000108759 | KRT32 |
| ENSG00000108813 | DLX4 |
| ENSG00000108825 | PTGES3L- |
| | AARSD1 |
| ENSG00000108830 | RND2 |
| ENSG00000108849 | PPY |
| ENSG00000108878 | CACNG1 |
| ENSG00000108947 | EFNB3 |
| ENSG00000109101 | FOXN1 |
| ENSG00000109132 | PHOX2B |
| ENSG00000109158 | GABRA4 |
| ENSG00000109163 | GNRHR |
| ENSG00000109181 | UGT2B10 |
| ENSG00000109182 | CWH43 |
| ENSG00000109193 | SULT1E1 |
| ENSG00000109205 | ODAM |
| ENSG00000109208 | SMR3A |
| ENSG00000109255 | NMU |
| ENSG00000109424 | UCP1 |
| ENSG00000109471 | IL2 |
| ENSG00000109511 | ANXA10 |
| ENSG00000109576 | AADAT |
| ENSG00000109625 | CPZ |
| ENSG00000109684 | CLNK |
| ENSG00000109705 | NKX3-2 |
| ENSG00000109738 | GLRB |
| ENSG00000109758 | HGFAC |
| ENSG00000109794 | FAM149A |
| ENSG00000109819 | PPARGC1A |
| ENSG00000109832 | DDX25 |
| ENSG00000109851 | DBX1 |
| ENSG00000109991 | P2RX3 |
| ENSG00000110148 | CCKBR |
| ENSG00000110195 | FOLR1 |
| ENSG00000110243 | APOA5 |
| ENSG00000110244 | APOA4 |
| ENSG00000110328 | GALNT18 |
| ENSG00000110375 | UPK2 |
| ENSG00000110427 | KIAA1549L |
| ENSG00000110484 | SCGB2A2 |
| ENSG00000110675 | ELMOD1 |
| ENSG00000110680 | CALCA |
| ENSG00000110786 | PTPN5 |
| ENSG00000110881 | ASIC1 |
| ENSG00000110887 | DAO |
| ENSG00000110900 | TSPAN11 |
| ENSG00000110975 | SYT10 |
| ENSG00000111012 | CYP27B1 |
| ENSG00000111046 | MYF6 |
| ENSG00000111049 | MYF5 |
| ENSG00000111218 | PRMT8 |
| ENSG00000111241 | FGF6 |
| ENSG00000111254 | AKAP3 |
| ENSG00000111262 | KCNA1 |
| ENSG00000111291 | GPRC5D |
| ENSG00000111305 | GSG1 |
| ENSG00000111344 | RASAL1 |
| ENSG00000111404 | RERGL |
| ENSG00000111405 | ENDOU |
| ENSG00000111432 | FZD10 |
| ENSG00000111536 | IL26 |
| ENSG00000111664 | GNB3 |
| ENSG00000111700 | SLCO1B3 |
| ENSG00000111701 | APOBEC1 |
| ENSG00000111704 | NANOG |
| ENSG00000111713 | GYS2 |
| ENSG00000111732 | AICDA |
| ENSG00000111780 | AL021546.6 |
| ENSG00000111783 | RFX4 |
| ENSG00000111816 | FRK |
| ENSG00000111834 | RSPH4A |
| ENSG00000111981 | ULBP1 |
| ENSG00000112038 | OPRM1 |
| ENSG00000112041 | TULP1 |
| ENSG00000112115 | IL17A |
| ENSG00000112116 | IL17F |
| ENSG00000112164 | GLP1R |
| ENSG00000112175 | BMP5 |
| ENSG00000112183 | RBM24 |
| ENSG00000112186 | CAP2 |
| ENSG00000112214 | FHL5 |
| ENSG00000112218 | GPR63 |
| ENSG00000112238 | PRDM13 |
| ENSG00000112246 | SIM1 |
| ENSG00000112273 | HDGFL1 |
| ENSG00000112276 | BVES |
| ENSG00000112280 | COL9A1 |
| ENSG00000112319 | EYA4 |
| ENSG00000112333 | NR2E1 |
| ENSG00000112337 | SLC17A2 |
| ENSG00000112414 | ADGRG6 |
| ENSG00000112462 | OR12D3 |
| ENSG00000112494 | UNC93A |
| ENSG00000112499 | SLC22A2 |
| ENSG00000112530 | PACRG |
| ENSG00000112539 | C6orf118 |
| ENSG00000112541 | PDE10A |
| ENSG00000112559 | MDFI |
| ENSG00000112619 | PRPH2 |
| ENSG00000112706 | IMPG1 |
| ENSG00000112761 | WISP3 |
| ENSG00000112812 | PRSS16 |
| ENSG00000112818 | MEP1A |
| ENSG00000112837 | TBX18 |
| ENSG00000112852 | PCDHB2 |
| ENSG00000112902 | SEMA5A |
| ENSG00000112964 | GHR |
| ENSG00000112981 | NME5 |
| ENSG00000113073 | SLC4A9 |
| ENSG00000113100 | CDH9 |
| ENSG00000113196 | HAND1 |
| ENSG00000113205 | PCDHB3 |
| ENSG00000113209 | PCDHB5 |
| ENSG00000113211 | PCDHB6 |
| ENSG00000113212 | PCDHB7 |
| ENSG00000113248 | PCDHB15 |
| ENSG00000113249 | HAVCR1 |
| ENSG00000113262 | GRM6 |
| ENSG00000113302 | IL12B |
| ENSG00000113327 | GABRG2 |
| ENSG00000113361 | CDH6 |
| ENSG00000113396 | SLC27A6 |
| ENSG00000113430 | IRX4 |
| ENSG00000113492 | AGXT2 |
| ENSG00000113494 | PRLR |
| ENSG00000113520 | IL4 |
| ENSG00000113525 | IL5 |
| ENSG00000113578 | FGF1 |
| ENSG00000113600 | C9 |
| ENSG00000113722 | CDX1 |
| ENSG00000113739 | STC2 |
| ENSG00000113805 | CNTN3 |
| ENSG00000113946 | CLDN16 |
| ENSG00000114113 | RBP2 |
| ENSG00000114124 | GRK7 |
| ENSG00000114200 | BCHE |
| ENSG00000114204 | SERPINI2 |
| ENSG00000114248 | LRRC31 |
| ENSG00000114251 | WNT5A |
| ENSG00000114279 | FGF12 |
| ENSG00000114349 | GNAT1 |
| ENSG00000114455 | HHLA2 |
| ENSG00000114547 | ROPN1B |
| ENSG00000114638 | UPK1B |
| ENSG00000114646 | CSPG5 |
| ENSG00000114757 | PEX5L |
| ENSG00000114771 | AADAC |
| ENSG00000114786 | ABHD14A- |
| | ACY1 |
| ENSG00000115008 | IL1A |
| ENSG00000115009 | CCL20 |
| ENSG00000115041 | KCNIP3 |
| ENSG00000115194 | SLC30A3 |
| ENSG00000115221 | ITGB6 |
| ENSG00000115226 | FNDC4 |
| ENSG00000115252 | PDE1A |
| ENSG00000115263 | GCG |
| ENSG00000115297 | TLX2 |
| ENSG00000115353 | TACR1 |
| ENSG00000115361 | ACADL |
| ENSG00000115363 | EVA1A |
| ENSG00000115423 | DNAH6 |
| ENSG00000115474 | KCNJ13 |
| ENSG00000115488 | NEU2 |
| ENSG00000115592 | PRKAG3 |
| ENSG00000115593 | SMYD1 |
| ENSG00000115596 | WNT6 |
| ENSG00000115598 | IL1RL2 |
| ENSG00000115616 | SLC9A2 |
| ENSG00000115648 | MLPH |
| ENSG00000115665 | SLC5A7 |
| ENSG00000115844 | DLX2 |
| ENSG00000115850 | LCT |
| ENSG00000115963 | RND3 |
| ENSG00000116031 | CD207 |
| ENSG00000116035 | VAX2 |
| ENSG00000116039 | ATP6V1B1 |
| ENSG00000116141 | MARK1 |
| ENSG00000116147 | TNR |
| ENSG00000116176 | TPSG1 |
| ENSG00000116183 | PAPPA2 |
| ENSG00000116194 | ANGPTL1 |
| ENSG00000116218 | NPHS2 |
| ENSG00000116254 | CHD5 |
| ENSG00000116703 | PDC |
| ENSG00000116721 | PRAMEF1 |
| ENSG00000116726 | PRAMEF12 |
| ENSG00000116745 | RPE65 |
| ENSG00000116748 | AMPD1 |
| ENSG00000116783 | TNNI3K |
| ENSG00000116785 | CFHR3 |
| ENSG00000116833 | NR5A2 |
| ENSG00000116882 | HAO2 |
| ENSG00000116885 | OSCP1 |
| ENSG00000116981 | NT5C1A |
| ENSG00000116996 | ZP4 |
| ENSG00000117069 | ST6GALNAC5 |
| ENSG00000117122 | MFAP2 |
| ENSG00000117148 | ACTL8 |
| ENSG00000117152 | RGS4 |
| ENSG00000117154 | IGSF21 |
| ENSG00000117215 | PLA2G2D |
| ENSG00000117407 | ARTN |
| ENSG00000117472 | TSPAN1 |
| ENSG00000117477 | CCDC181 |
| ENSG00000117501 | MROH9 |
| ENSG00000117594 | HSD11B1 |
| ENSG00000117598 | PLPPR5 |
| ENSG00000117600 | PLPPR4 |
| ENSG00000117707 | PROX1 |
| ENSG00000117971 | CHRNB4 |
| ENSG00000118004 | COLEC11 |
| ENSG00000118017 | A4GNT |
| ENSG00000118094 | TREH |
| ENSG00000118156 | ZNF541 |
| ENSG00000118160 | SLC8A2 |
| ENSG00000118231 | CRYGD |
| ENSG00000118245 | TNP1 |
| ENSG00000118298 | CA14 |
| ENSG00000118307 | CASC1 |
| ENSG00000118322 | ATP10B |
| ENSG00000118402 | ELOVL4 |
| ENSG00000118434 | SPACA1 |
| ENSG00000118491 | ZC2HC1B |
| ENSG00000118492 | ADGB |
| ENSG00000118526 | TCF21 |
| ENSG00000118702 | GHRH |
| ENSG00000118729 | CASQ2 |
| ENSG00000118733 | OLFM3 |
| ENSG00000118946 | PCDH17 |
| ENSG00000118972 | FGF23 |
| ENSG00000118997 | DNAH7 |
| ENSG00000119125 | GDA |
| ENSG00000119147 | C2orf40 |
| ENSG00000119283 | TRIM67 |
| ENSG00000119547 | ONECUT2 |
| ENSG00000119614 | VSX2 |
| ENSG00000119698 | PPP4R4 |
| ENSG00000119703 | ZC2HC1C |
| ENSG00000119715 | ESRRB |
| ENSG00000119737 | GPR75 |
| ENSG00000119913 | TECTB |
| ENSG00000119919 | NKX2-3 |
| ENSG00000119946 | CNNM1 |
| ENSG00000119973 | PRLHR |
| ENSG00000120054 | CPN1 |
| ENSG00000120057 | SFRP5 |
| ENSG00000120068 | HOXB8 |
| ENSG00000120075 | HOXB5 |
| ENSG00000120088 | CRHR1 |
| ENSG00000120094 | HOXB1 |
| ENSG00000120149 | MSX2 |
| ENSG00000120160 | EQTN |
| ENSG00000120210 | INSL6 |
| ENSG00000120211 | INSL4 |
| ENSG00000120215 | MLANA |
| ENSG00000120235 | IFNA6 |
| ENSG00000120242 | IFNA8 |
| ENSG00000120251 | GRIA2 |
| ENSG00000120289 | MAGEB4 |
| ENSG00000120322 | PCDHB8 |
| ENSG00000120324 | PCDHB10 |
| ENSG00000120327 | PCDHB14 |
| ENSG00000120328 | PCDHB12 |
| ENSG00000120329 | SLC25A2 |
| ENSG00000120332 | TNN |
| ENSG00000120337 | TNFSF18 |
| ENSG00000120341 | SEC16B |
| ENSG00000120436 | GPR31 |
| ENSG00000120440 | TTLL2 |
| ENSG00000120457 | KCNJ5 |
| ENSG00000120471 | TP53AIP1 |
| ENSG00000120498 | TEX11 |
| ENSG00000120500 | ARR3 |
| ENSG00000120563 | LYZL1 |
| ENSG00000120658 | ENOX1 |
| ENSG00000120659 | TNFSF11 |
| ENSG00000120669 | SOHLH2 |
| ENSG00000120729 | MYOT |
| ENSG00000120820 | GLT8D2 |
| ENSG00000120907 | ADRA1A |
| ENSG00000120937 | NPPB |
| ENSG00000120952 | PRAMEF2 |
| ENSG00000121005 | CRISPLD1 |
| ENSG00000121075 | TBX4 |
| ENSG00000121101 | TEX14 |
| ENSG00000121207 | LRAT |
| ENSG00000121211 | MND1 |
| ENSG00000121270 | ABCC11 |
| ENSG00000121314 | TAS2R8 |
| ENSG00000121318 | TAS2R10 |
| ENSG00000121335 | PRB2 |
| ENSG00000121351 | IAPP |
| ENSG00000121361 | KCNJ8 |
| ENSG00000121377 | TAS2R7 |
| ENSG00000121380 | BCL2L14 |
| ENSG00000121381 | TAS2R9 |
| ENSG00000121440 | PDZRN3 |
| ENSG00000121446 | RGSL1 |
| ENSG00000121634 | GJA8 |
| ENSG00000121690 | DEPDC7 |
| ENSG00000121743 | GJA3 |
| ENSG00000121764 | HCRTR1 |
| ENSG00000121853 | GHSR |
| ENSG00000121871 | SLITRK3 |
| ENSG00000121904 | CSMD2 |
| ENSG00000121905 | HPCA |
| ENSG00000122012 | SV2C |
| ENSG00000122133 | PAEP |
| ENSG00000122136 | OBP2A |
| ENSG00000122145 | TBX22 |
| ENSG00000122180 | MYOG |
| ENSG00000122194 | PLG |
| ENSG00000122254 | HS3ST2 |
| ENSG00000122304 | PRM2 |
| ENSG00000122375 | OPN4 |
| ENSG00000122574 | WIPF3 |
| ENSG00000122584 | NXPH1 |
| ENSG00000122585 | NPY |
| ENSG00000122592 | HOXA7 |
| ENSG00000122691 | TWIST1 |
| ENSG00000122711 | SPINK4 |
| ENSG00000122728 | TAF1L |
| ENSG00000122735 | DNAI1 |
| ENSG00000122756 | CNTFR |
| ENSG00000122787 | AKR1D1 |
| ENSG00000122824 | NUDT10 |
| ENSG00000122859 | NEUROG3 |
| ENSG00000122863 | CHST3 |
| ENSG00000122870 | BICC1 |
| ENSG00000123165 | ACTRT1 |
| ENSG00000123171 | CCDC70 |
| ENSG00000123307 | NEUROD4 |
| ENSG00000123364 | HOXC13 |
| ENSG00000123388 | HOXC11 |
| ENSG00000123407 | HOXC12 |
| ENSG00000123454 | DBH |
| ENSG00000123496 | IL13RA2 |
| ENSG00000123500 | COL10A1 |
| ENSG00000123561 | SERPINA7 |
| ENSG00000123569 | H2BFWT |
| ENSG00000123570 | RAB9B |
| ENSG00000123572 | NRK |
| ENSG00000123576 | ESX1 |
| ENSG00000123584 | MAGEA9B |
| ENSG00000123594 | ATXN3L |
| ENSG00000123843 | C4BPB |
| ENSG00000123901 | GPR83 |
| ENSG00000123977 | DAW1 |
| ENSG00000123999 | INHA |
| ENSG00000124003 | MOGAT1 |
| ENSG00000124089 | MC3R |
| ENSG00000124091 | GCNT7 |
| ENSG00000124092 | CTCFL |
| ENSG00000124116 | WFDC3 |
| ENSG00000124134 | KCNS1 |
| ENSG00000124140 | SLC12A5 |
| ENSG00000124143 | ARHGAP40 |
| ENSG00000124157 | SEMG2 |
| ENSG00000124159 | MATN4 |
| ENSG00000124194 | GDAP1L1 |
| ENSG00000124196 | GTSF1L |
| ENSG00000124205 | EDN3 |
| ENSG00000124227 | ANKRD60 |
| ENSG00000124232 | RBPJL |
| ENSG00000124233 | SEMG1 |
| ENSG00000124237 | C20orf85 |
| ENSG00000124249 | KCNK15 |
| ENSG00000124251 | TP53TG5 |
| ENSG00000124260 | MAGEA10 |
| ENSG00000124343 | XG |
| ENSG00000124391 | IL17C |
| ENSG00000124429 | POF1B |
| ENSG00000124449 | IRGC |
| ENSG00000124467 | PSG8 |
| ENSG00000124479 | NDP |
| ENSG00000124493 | GRM4 |
| ENSG00000124557 | BTN1A1 |
| ENSG00000124564 | SLC17A3 |
| ENSG00000124568 | SLC17A1 |
| ENSG00000124610 | HIST1H1A |
| ENSG00000124657 | OR2B6 |
| ENSG00000124664 | SPDEF |
| ENSG00000124678 | TCP11 |
| ENSG00000124721 | DNAH8 |
| ENSG00000124743 | KLHL31 |
| ENSG00000124749 | COL21A1 |
| ENSG00000124780 | KCNK17 |
| ENSG00000124812 | CRISP1 |
| ENSG00000124818 | OPN5 |
| ENSG00000124827 | GCM2 |
| ENSG00000124875 | CXCL6 |
| ENSG00000124900 | TRIM51 |
| ENSG00000124935 | SCGB1D2 |
| ENSG00000124939 | SCGB2A1 |
| ENSG00000125046 | SSUH2 |
| ENSG00000125084 | WNT |
| ENSG00000125207 | PIWVIL1 |
| ENSG00000125255 | SLC10A2 |
| ENSG00000125285 | SOX21 |
| ENSG00000125337 | KIF25 |
| ENSG00000125355 | TMEM255A |
| ENSG00000125363 | AMELX |
| ENSG00000125378 | BMP4 |
| ENSG00000125398 | SOX9 |
| ENSG00000125409 | TEKT3 |
| ENSG00000125492 | BARHL1 |
| ENSG00000125508 | SRMS |
| ENSG00000125522 | NPBWR2 |
| ENSG00000125533 | BHLHE23 |
| ENSG00000125571 | IL37 |
| ENSG00000125675 | GRIA3 |
| ENSG00000125695 | RP11- |
| | 51F16.8 |
| ENSG00000125787 | GNRH2 |
| ENSG00000125788 | DEFB126 |
| ENSG00000125798 | FOXA2 |
| ENSG00000125813 | PAX1 |
| ENSG00000125815 | CST8 |
| ENSG00000125816 | NKX2-4 |
| ENSG00000125820 | NKX2-2 |
| ENSG00000125823 | CSTL1 |
| ENSG00000125831 | CST11 |
| ENSG00000125845 | BMP2 |
| ENSG00000125848 | FLRT3 |
| ENSG00000125850 | OVOL2 |
| ENSG00000125851 | PCSK2 |
| ENSG00000125861 | GFRA4 |
| ENSG00000125872 | LRRN4 |
| ENSG00000125878 | TCF15 |
| ENSG00000125879 | OTOR |
| ENSG00000125888 | BANF2 |
| ENSG00000125895 | TMEM74B |
| ENSG00000125903 | DEFB129 |
| ENSG00000125931 | CITED1 |
| ENSG00000125965 | GDF5 |
| ENSG00000125975 | C20orf173 |
| ENSG00000125998 | FAM83C |
| ENSG00000125999 | BPIFB1 |
| ENSG00000126010 | GRPR |
| ENSG00000126218 | F10 |
| ENSG00000126231 | PROZ |
| ENSG00000126233 | SLURP1 |
| ENSG00000126259 | KIRREL2 |
| ENSG00000126266 | FFAR1 |
| ENSG00000126337 | KRT36 |
| ENSG00000126500 | FLRT1 |
| ENSG00000126545 | CSN1S1 |
| ENSG00000126549 | STATH |
| ENSG00000126550 | HTN1 |
| ENSG00000126562 | WNK4 |
| ENSG00000126583 | PRKCG |
| ENSG00000126733 | DACH2 |
| ENSG00000126752 | SSX1 |
| ENSG00000126778 | SIX1 |
| ENSG00000126838 | PZP |
| ENSG00000126856 | PRDM7 |
| ENSG00000126890 | CTAG2 |
| ENSG00000126950 | TMEM35 |
| ENSG00000126952 | NXF5 |
| ENSG00000127074 | RGS13 |
| ENSG00000127083 | OMD |
| ENSG00000127129 | EDN2 |
| ENSG00000127241 | MASP1 |
| ENSG00000127318 | IL22 |
| ENSG00000127324 | TSPAN8 |
| ENSG00000127325 | BEST3 |
| ENSG00000127362 | TAS2R3 |
| ENSG00000127364 | TAS2R4 |
| ENSG00000127366 | TAS2R5 |
| ENSG00000127377 | CRYGN |
| ENSG00000127412 | TRPV5 |
| ENSG00000127472 | PLA2G5 |
| ENSG00000127515 | OR7A10 |
| ENSG00000127529 | OR7C2 |
| ENSG00000127530 | OR7C1 |
| ENSG00000127588 | GNG13 |
| ENSG00000127743 | IL17B |
| ENSG00000127780 | OR1E2 |
| ENSG00000127863 | TNFRSF19 |
| ENSG00000127928 | GNGT1 |
| ENSG00000128040 | SPINK2 |
| ENSG00000128045 | RASL11B |
| ENSG00000128242 | GAL3ST1 |
| ENSG00000128250 | RFPL1 |
| ENSG00000128253 | RFPL2 |
| ENSG00000128276 | RFPL3 |
| ENSG00000128285 | MCHR1 |
| ENSG00000128310 | GALR3 |
| ENSG00000128313 | APOL5 |
| ENSG00000128346 | C22orf23 |
| ENSG00000128408 | RIBC2 |
| ENSG00000128422 | KRT17 |
| ENSG00000128510 | CPA4 |
| ENSG00000128519 | TAS2R16 |
| ENSG00000128564 | VGF |
| ENSG00000128573 | FOXP2 |
| ENSG00000128606 | LRRC17 |
| ENSG00000128610 | FEZF1 |
| ENSG00000128617 | OPN1SW |
| ENSG00000128645 | HOXD1 |
| ENSG00000128652 | HOXD3 |
| ENSG00000128655 | PDE11A |
| ENSG00000128683 | GAD1 |
| ENSG00000128709 | HOXD9 |
| ENSG00000128710 | HOXD10 |
| ENSG00000128713 | HOXD11 |
| ENSG00000128714 | HOXD13 |
| ENSG00000129028 | THAP10 |
| ENSG00000129048 | ACKR4 |
| ENSG00000129151 | BBOX1 |
| ENSG00000129152 | MYOD1 |
| ENSG00000129159 | KCNC1 |
| ENSG00000129167 | TPH1 |
| ENSG00000129170 | CSRP3 |
| ENSG00000129214 | SHBG |
| ENSG00000129221 | AIPL1 |
| ENSG00000129437 | KLK14 |
| ENSG00000129451 | KLK10 |
| ENSG00000129455 | KLK8 |
| ENSG00000129514 | FOXA1 |
| ENSG00000129654 | FOXJ1 |
| ENSG00000129673 | AANAT |
| ENSG00000129744 | ART1 |
| ENSG00000129862 | VCY1B |
| ENSG00000129864 | VCY |
| ENSG00000129873 | CDY2B |
| ENSG00000129910 | CDH15 |
| ENSG00000129965 | INS-IGF2 |
| ENSG00000129988 | LBP |
| ENSG00000129990 | SYT5 |
| ENSG00000130032 | PRRG3 |
| ENSG00000130037 | KCNA5 |
| ENSG00000130045 | NXNL2 |
| ENSG00000130054 | FAM155B |
| ENSG00000130055 | GDPD2 |
| ENSG00000130167 | TSPAN16 |
| ENSG00000130173 | C19orf80 |
| ENSG00000130182 | ZSCAN10 |
| ENSG00000130224 | LRCH2 |
| ENSG00000130226 | DPP6 |
| ENSG00000130234 | ACE2 |
| ENSG00000130283 | GDF1 |
| ENSG00000130287 | NCAN |
| ENSG00000130368 | MAS1 |
| ENSG00000130377 | ACSBG2 |
| ENSG00000130383 | FUT5 |
| ENSG00000130385 | BMP15 |
| ENSG00000130427 | EPO |
| ENSG00000130433 | CACNG6 |
| ENSG00000130477 | UNC13A |
| ENSG00000130528 | HRC |
| ENSG00000130540 | SULT4A1 |
| ENSG00000130545 | CRB3 |
| ENSG00000130561 | SAG |
| ENSG00000130643 | CALY |
| ENSG00000130675 | MNX1 |
| ENSG00000130700 | GATA5 |
| ENSG00000130701 | RBBP8NL |
| ENSG00000130711 | PRDM12 |
| ENSG00000130720 | FIBCD1 |
| ENSG00000130751 | NPAS1 |
| ENSG00000130762 | ARHGEF16 |
| ENSG00000130822 | PNCK |
| ENSG00000130829 | DUSP9 |
| ENSG00000130876 | SLC7A10 |
| ENSG00000130943 | PKDREJ |
| ENSG00000130950 | NUTM2F |
| ENSG00000130957 | FBP2 |
| ENSG00000130988 | RGN |
| ENSG00000131015 | ULBP2 |
| ENSG00000131019 | ULBP3 |
| ENSG00000131044 | TTLL9 |
| ENSG00000131050 | BPIFA2 |
| ENSG00000131055 | COX4I2 |
| ENSG00000131059 | BPIFA3 |
| ENSG00000131068 | DEFB118 |
| ENSG00000131080 | EDA2R |
| ENSG00000131094 | C1QL1 |
| ENSG00000131096 | PYY |
| ENSG00000131097 | HIGD1B |
| ENSG00000131126 | TEX101 |
| ENSG00000131142 | CCL25 |
| ENSG00000131152 | RP11- |
| | 178L8.4 |
| ENSG00000131183 | SLC34A1 |
| ENSG00000131233 | GJA9 |
| ENSG00000131264 | CDX4 |
| ENSG00000131379 | C3orf20 |
| ENSG00000131482 | G6PC |
| ENSG00000131620 | ANO1 |
| ENSG00000131650 | KREMEN2 |
| ENSG00000131668 | BARX1 |
| ENSG00000131686 | CA6 |
| ENSG00000131721 | RHOXF2 |
| ENSG00000131737 | KRT34 |
| ENSG00000131738 | KRT33B |
| ENSG00000131808 | FSHB |
| ENSG00000131864 | USP29 |
| ENSG00000131910 | NR0B2 |
| ENSG00000131914 | LIN28A |
| ENSG00000131941 | RHPN2 |
| ENSG00000131951 | LRRC9 |
| ENSG00000132026 | RTBDN |
| ENSG00000132031 | MATN3 |
| ENSG00000132164 | SLC6A11 |
| ENSG00000132297 | HHLA1 |
| ENSG00000132321 | IQCA1 |
| ENSG00000132429 | POPDC3 |
| ENSG00000132437 | DDC |
| ENSG00000132446 | FTHL17 |
| ENSG00000132464 | ENAM |
| ENSG00000132517 | SLC52A1 |
| ENSG00000132518 | GUCY2D |
| ENSG00000132554 | RGS22 |
| ENSG00000132631 | SCP2D1 |
| ENSG00000132671 | SSTR4 |
| ENSG00000132677 | RHBG |
| ENSG00000132681 | ATP1A4 |
| ENSG00000132692 | BCAN |
| ENSG00000132698 | RAB25 |
| ENSG00000132702 | HAPLN2 |
| ENSG00000132703 | APCS |
| ENSG00000132746 | ALDH3B2 |
| ENSG00000132821 | VSTM2L |
| ENSG00000132837 | DMGDH |
| ENSG00000132840 | BHMT2 |
| ENSG00000132854 | KANK4 |
| ENSG00000132872 | SYT4 |
| ENSG00000132874 | SLC14A2 |
| ENSG00000132911 | NMUR2 |
| ENSG00000132915 | PDE6A |
| ENSG00000132932 | ATP8A2 |
| ENSG00000132938 | MTUS2 |
| ENSG00000132958 | TPTE2 |
| ENSG00000132975 | GPR12 |
| ENSG00000133019 | CHRM3 |
| ENSG00000133020 | MYH8 |
| ENSG00000133083 | DCLK1 |
| ENSG00000133105 | RXFP2 |
| ENSG00000133107 | TRPC4 |
| ENSG00000133115 | STOML3 |
| ENSG00000133124 | IRS4 |
| ENSG00000133135 | RNF128 |
| ENSG00000133328 | HRASLS2 |
| ENSG00000133433 | GSTT2B |
| ENSG00000133475 | GGT2 |
| ENSG00000133636 | NTS |
| ENSG00000133640 | LRRIQ1 |
| ENSG00000133665 | DYDC2 |
| ENSG00000133863 | TEX15 |
| ENSG00000133878 | DUSP26 |
| ENSG00000133937 | GSC |
| ENSG00000133958 | UNC79 |
| ENSG00000133980 | VRTN |
| ENSG00000134007 | ADAM20 |
| ENSG00000134020 | PEBP4 |
| ENSG00000134042 | MRO |
| ENSG00000134115 | CNTN6 |
| ENSG00000134138 | MEIS2 |
| ENSG00000134160 | TRPM1 |
| ENSG00000134183 | GNAT2 |
| ENSG00000134193 | REG4 |
| ENSG00000134200 | TSHB |
| ENSG00000134207 | SYT6 |
| ENSG00000134216 | CHIA |
| ENSG00000134249 | ADAM30 |
| ENSG00000134253 | TRIM45 |
| ENSG00000134258 | VTCN1 |
| ENSG00000134259 | NGF |
| ENSG00000134323 | MYCN |
| ENSG00000134343 | ANO3 |
| ENSG00000134363 | FST |
| ENSG00000134365 | CFHR4 |
| ENSG00000134376 | CRB1 |
| ENSG00000134389 | CFHR5 |
| ENSG00000134398 | ERN2 |
| ENSG00000134438 | RAX |
| ENSG00000134443 | GRP |
| ENSG00000134532 | SOX5 |
| ENSG00000134533 | RERG |
| ENSG00000134538 | SLCO1B1 |
| ENSG00000134551 | PRH2 |
| ENSG00000134569 | LRP4 |
| ENSG00000134588 | USP26 |
| ENSG00000134595 | SOX3 |
| ENSG00000134640 | MTNR1B |
| ENSG00000134716 | CYP2J2 |
| ENSG00000134757 | DSG3 |
| ENSG00000134760 | DSG1 |
| ENSG00000134762 | DSC3 |
| ENSG00000134765 | DSC1 |
| ENSG00000134775 | FHOD3 |
| ENSG00000134812 | GIF |
| ENSG00000134873 | CLDN10 |
| ENSG00000134874 | DZIP1 |
| ENSG00000134901 | KDELC1 |
| ENSG00000134917 | ADAMTS8 |
| ENSG00000134940 | ACRV1 |
| ENSG00000134962 | KLB |
| ENSG00000135063 | FAM189A2 |
| ENSG00000135097 | MSI1 |
| ENSG00000135100 | HNF1A |
| ENSG00000135116 | HRK |
| ENSG00000135175 | OCM2 |
| ENSG00000135220 | UGT2A3 |
| ENSG00000135222 | CSN2 |
| ENSG00000135226 | UGT2B28 |
| ENSG00000135248 | FAM71F1 |
| ENSG00000135253 | KCP |
| ENSG00000135298 | ADGRB3 |
| ENSG00000135312 | HTR1B |
| ENSG00000135324 | MRAP2 |
| ENSG00000135333 | EPHA7 |
| ENSG00000135346 | CGA |
| ENSG00000135355 | GJA10 |
| ENSG00000135373 | EHF |
| ENSG00000135374 | ELF5 |
| ENSG00000135406 | PRPH |
| ENSG00000135409 | AMHR2 |
| ENSG00000135413 | LACRT |
| ENSG00000135436 | FAM186B |
| ENSG00000135443 | KRT85 |
| ENSG00000135454 | B4GALNT1 |
| ENSG00000135502 | SLC26A10 |
| ENSG00000135517 | MIP |
| ENSG00000135549 | PKIB |
| ENSG00000135569 | TAAR5 |
| ENSG00000135577 | NMBR |
| ENSG00000135625 | EGR4 |
| ENSG00000135638 | EMX1 |
| ENSG00000135697 | BCO1 |
| ENSG00000135702 | CHST5 |
| ENSG00000135747 | ZNF670- |
| | ZNF695 |
| ENSG00000135750 | KCNK1 |
| ENSG00000135773 | CAPN9 |
| ENSG00000135824 | RGS8 |
| ENSG00000135902 | CHRND |
| ENSG00000135903 | PAX3 |
| ENSG00000135914 | HTR2B |
| ENSG00000135917 | SLC19A3 |
| ENSG00000135931 | ARMC9 |
| ENSG00000135973 | GPR45 |
| ENSG00000136110 | LECT1 |
| ENSG00000136155 | SCEL |
| ENSG00000136267 | DGKB |
| ENSG00000136297 | MMD2 |
| ENSG00000136327 | NKX2-8 |
| ENSG00000136352 | NKX2-1 |
| ENSG00000136449 | MYCBPAP |
| ENSG00000136487 | GH2 |
| ENSG00000136488 | CSH1 |
| ENSG00000136531 | SCN2A |
| ENSG00000136535 | TBR1 |
| ENSG00000136546 | SCN7A |
| ENSG00000136574 | GATA4 |
| ENSG00000136688 | IL36G |
| ENSG00000136694 | IL36A |
| ENSG00000136695 | IL36RN |
| ENSG00000136696 | IL36B |
| ENSG00000136697 | IL1F10 |
| ENSG00000136698 | CFC1 |
| ENSG00000136750 | GAD2 |
| ENSG00000136834 | OR1J1 |
| ENSG00000136839 | OR13C9 |
| ENSG00000136883 | KIF12 |
| ENSG00000136918 | WDR38 |
| ENSG00000136928 | GABBR2 |
| ENSG00000136931 | NR5A1 |
| ENSG00000136939 | OR1L4 |
| ENSG00000136943 | CTSV |
| ENSG00000136944 | LMX1B |
| ENSG00000137077 | CCL21 |
| ENSG00000137080 | IFNA21 |
| ENSG00000137090 | DMRT1 |
| ENSG00000137142 | IGFBPL1 |
| ENSG00000137203 | TFAP2A |
| ENSG00000137204 | SLC22A7 |
| ENSG00000137251 | TINAG |
| ENSG00000137252 | HCRTR2 |
| ENSG00000137273 | FOXF2 |
| ENSG00000137434 | C6orf52 |
| ENSG00000137440 | FGFBP1 |
| ENSG00000137463 | MGARP |
| ENSG00000137473 | TTC29 |
| ENSG00000137558 | PI15 |
| ENSG00000137561 | TTPA |
| ENSG00000137571 | SLCO5A1 |
| ENSG00000137634 | NXPE4 |
| ENSG00000137648 | TMPRSS4 |
| ENSG00000137673 | MMP7 |
| ENSG00000137674 | MMP20 |
| ENSG00000137675 | MMP27 |
| ENSG00000137691 | C11orf70 |
| ENSG00000137707 | BTG4 |
| ENSG00000137709 | POU2F3 |
| ENSG00000137727 | ARHGAP20 |
| ENSG00000137745 | MMP13 |
| ENSG00000137766 | UNC13C |
| ENSG00000137809 | ITGA11 |
| ENSG00000137821 | LRRC49 |
| ENSG00000137825 | ITPKA |
| ENSG00000137843 | PAK6 |
| ENSG00000137860 | SLC28A2 |
| ENSG00000137868 | STRA6 |
| ENSG00000137872 | SEMA6D |
| ENSG00000137875 | BCL2L10 |
| ENSG00000137948 | BRDT |
| ENSG00000137960 | GIPC2 |
| ENSG00000137968 | SLC44A5 |
| ENSG00000137975 | CLCA2 |
| ENSG00000137976 | DNASE2B |
| ENSG00000138028 | CGREF1 |
| ENSG00000138039 | LHCGR |
| ENSG00000138068 | SULT6B1 |
| ENSG00000138075 | ABCG5 |
| ENSG00000138083 | SIX3 |
| ENSG00000138100 | TRIM54 |
| ENSG00000138109 | CYP2C9 |
| ENSG00000138135 | CH25H |
| ENSG00000138136 | LBX1 |
| ENSG00000138152 | BTBD16 |
| ENSG00000138271 | GPR87 |
| ENSG00000138308 | PLA2G12B |
| ENSG00000138347 | MYPN |
| ENSG00000138379 | MSTN |
| ENSG00000138395 | CDK15 |
| ENSG00000138400 | MDH1B |
| ENSG00000138435 | CHRNA1 |
| ENSG00000138472 | GUCA1C |
| ENSG00000138483 | CCDC54 |
| ENSG00000138587 | MNS1 |
| ENSG00000138622 | HCN4 |
| ENSG00000138650 | PCDH10 |
| ENSG00000138653 | NDST4 |
| ENSG00000138669 | PRKG2 |
| ENSG00000138675 | FGF5 |
| ENSG00000138684 | IL21 |
| ENSG00000138696 | BMPR1B |
| ENSG00000138741 | TRPC3 |
| ENSG00000138759 | FRAS1 |
| ENSG00000138769 | CDKL2 |
| ENSG00000138771 | SHROOM3 |
| ENSG00000138792 | ENPEP |
| ENSG00000138813 | C4orf17 |
| ENSG00000138823 | MTTP |
| ENSG00000138892 | TTLL8 |
| ENSG00000138944 | KIAA1644 |
| ENSG00000139053 | PDE6H |
| ENSG00000139144 | PIK3C2G |
| ENSG00000139151 | PLCZ1 |
| ENSG00000139155 | SLCO1C1 |
| ENSG00000139209 | SLC38A4 |
| ENSG00000139219 | COL2A1 |
| ENSG00000139220 | PPFIA2 |
| ENSG00000139223 | ANP32D |
| ENSG00000139263 | LRIG3 |
| ENSG00000139269 | INHBE |
| ENSG00000139287 | TPH2 |
| ENSG00000139292 | LGR5 |
| ENSG00000139304 | PTPRQ |
| ENSG00000139330 | KERA |
| ENSG00000139352 | ASCL1 |
| ENSG00000139364 | TMEM132B |
| ENSG00000139445 | FOXN4 |
| ENSG00000139515 | PDX1 |
| ENSG00000139540 | SLC39A5 |
| ENSG00000139549 | DHH |
| ENSG00000139574 | NPFF |
| ENSG00000139610 | CELA1 |
| ENSG00000139648 | KRT71 |
| ENSG00000139656 | SMIM2 |
| ENSG00000139767 | SRRM4 |
| ENSG00000139780 | METTL21C |
| ENSG00000139797 | RNF113B |
| ENSG00000139800 | ZIC5 |
| ENSG00000139865 | TTC6 |
| ENSG00000139874 | SSTR1 |
| ENSG00000139910 | NOVA1 |
| ENSG00000139915 | MDGA2 |
| ENSG00000139971 | C14orf37 |
| ENSG00000139973 | SYT16 |
| ENSG00000139985 | ADAM21 |
| ENSG00000139988 | RDH12 |
| ENSG00000140015 | KCNH5 |
| ENSG00000140057 | AK7 |
| ENSG00000140067 | FAM181A |
| ENSG00000140093 | SERPINA10 |
| ENSG00000140107 | SLC25A47 |
| ENSG00000140254 | DUOXA1 |
| ENSG00000140274 | DUOXA2 |
| ENSG00000140279 | DUOX2 |
| ENSG00000140285 | FGF7 |
| ENSG00000140297 | GCNT3 |
| ENSG00000140459 | CYP11A1 |
| ENSG00000140465 | CYP1A1 |
| ENSG00000140478 | GOLGA6D |
| ENSG00000140481 | CCDC33 |
| ENSG00000140505 | CYP1A2 |
| ENSG00000140506 | LMAN1L |
| ENSG00000140522 | RLBP1 |
| ENSG00000140527 | WDR93 |
| ENSG00000140538 | NTRK3 |
| ENSG00000140557 | ST8SIA2 |
| ENSG00000140600 | SH3GL3 |
| ENSG00000140623 | SEPT12 |
| ENSG00000140798 | ABCC12 |
| ENSG00000140832 | MARVELD3 |
| ENSG00000140835 | CHST4 |
| ENSG00000140873 | ADAMTS18 |
| ENSG00000140937 | CDH11 |
| ENSG00000140955 | ADAD2 |
| ENSG00000141052 | MYOCD |
| ENSG00000141194 | OR4D1 |
| ENSG00000141200 | KIF2B |
| ENSG00000141255 | SPATA22 |
| ENSG00000141314 | RHBDL3 |
| ENSG00000141316 | SPACA3 |
| ENSG00000141371 | C17orf64 |
| ENSG00000141391 | PRELID3A |
| ENSG00000141431 | ASXL3 |
| ENSG00000141433 | ADCYAP1 |
| ENSG00000141434 | MEP1B |
| ENSG00000141437 | SLC25A52 |
| ENSG00000141441 | GAREM |
| ENSG00000141449 | GREB1L |
| ENSG00000141485 | SLC13A5 |
| ENSG00000141579 | ZNF750 |
| ENSG00000141639 | MAPK4 |
| ENSG00000141665 | FBXO15 |
| ENSG00000141668 | CBLN2 |
| ENSG00000141738 | GRB7 |
| ENSG00000141748 | ARL5C |
| ENSG00000141750 | STAC2 |
| ENSG00000141934 | PLPP2 |
| ENSG00000141946 | ZIM3 |
| ENSG00000141977 | CIB3 |
| ENSG00000141979 | CTD- |
| | 3222D19.2 |
| ENSG00000142149 | HUNK |
| ENSG00000142182 | DNMT3L |
| ENSG00000142224 | IL19 |
| ENSG00000142273 | CBLC |
| ENSG00000142319 | SLC6A3 |
| ENSG00000142449 | FBN3 |
| ENSG00000142484 | TM4SF5 |
| ENSG00000142511 | GPR32 |
| ENSG00000142513 | ACPT |
| ENSG00000142515 | KLK3 |
| ENSG00000142530 | FAM71E1 |
| ENSG00000142538 | PTH2 |
| ENSG00000142549 | IGLON5 |
| ENSG00000142609 | CFAP74 |
| ENSG00000142611 | PRDM16 |
| ENSG00000142619 | PADI3 |
| ENSG00000142623 | PADI1 |
| ENSG00000142661 | MYOM3 |
| ENSG00000142677 | IL22RA1 |
| ENSG00000142698 | C1orf94 |
| ENSG00000142700 | DMRTA2 |
| ENSG00000143001 | TMEM61 |
| ENSG00000143006 | DMRTB1 |
| ENSG00000143028 | SYPL2 |
| ENSG00000143032 | BARHL2 |
| ENSG00000143036 | SLC44A3 |
| ENSG00000143061 | IGSF3 |
| ENSG00000143105 | KCNA10 |
| ENSG00000143107 | FNDC7 |
| ENSG00000143125 | PROK1 |
| ENSG00000143147 | GPR161 |
| ENSG00000143171 | RXRG |
| ENSG00000143194 | MAEL |
| ENSG00000143199 | ADCY10 |
| ENSG00000143217 | PVRL4 |
| ENSG00000143278 | F13B |
| ENSG00000143340 | FAM163A |
| ENSG00000143355 | LHX9 |
| ENSG00000143375 | CGN |
| ENSG00000143469 | SYT14 |
| ENSG00000143473 | KCNH1 |
| ENSG00000143494 | VASH2 |
| ENSG00000143512 | HHIPL2 |
| ENSG00000143552 | NUP210L |
| ENSG00000143556 | S100A7 |
| ENSG00000143768 | LEFTY2 |
| ENSG00000143816 | WNT9A |
| ENSG00000143839 | REN |
| ENSG00000143858 | SYT2 |
| ENSG00000143867 | OSR1 |
| ENSG00000143921 | ABCG8 |
| ENSG00000143954 | REG3G |
| ENSG00000143994 | ABHD1 |
| ENSG00000144015 | TRIM43 |
| ENSG00000144031 | ANKRD53 |
| ENSG00000144035 | NAT8 |
| ENSG00000144045 | DQX1 |
| ENSG00000144061 | NPHP1 |
| ENSG00000144119 | C1QL2 |
| ENSG00000144191 | CNGA3 |
| ENSG00000144227 | NXPH2 |
| ENSG00000144229 | THSD7B |
| ENSG00000144278 | GALNT13 |
| ENSG00000144285 | SCN1A |
| ENSG00000144331 | ZNF385B |
| ENSG00000144339 | TMEFF2 |
| ENSG00000144355 | DLX1 |
| ENSG00000144362 | PHOSPHO2 |
| ENSG00000144366 | GULP1 |
| ENSG00000144369 | FAM171B |
| ENSG00000144395 | CCDC150 |
| ENSG00000144406 | UNC80 |
| ENSG00000144410 | CPO |
| ENSG00000144452 | ABCA12 |
| ENSG00000144460 | NYAP2 |
| ENSG00000144481 | TRPM8 |
| ENSG00000144488 | ESPNL |
| ENSG00000144550 | CPNE9 |
| ENSG00000144583 | MARCH4 |
| ENSG00000144619 | CNTN4 |
| ENSG00000144644 | GADL1 |
| ENSG00000144649 | FAM198A |
| ENSG00000144681 | STAC |
| ENSG00000144730 | IL17RD |
| ENSG00000144771 | LRTM1 |
| ENSG00000144785 | RP11- |
| | 977G19.10 |
| ENSG00000144820 | ADGRG7 |
| ENSG00000144821 | MYH15 |
| ENSG00000144834 | TAGLN3 |
| ENSG00000144837 | PLA1A |
| ENSG00000144847 | IGSF11 |
| ENSG00000144852 | NR1I2 |
| ENSG00000144857 | BOC |
| ENSG00000144868 | TMEM108 |
| ENSG00000144891 | AGTR1 |
| ENSG00000144962 | SPATA16 |
| ENSG00000145002 | FAM86B2 |
| ENSG00000145040 | UCN2 |
| ENSG00000145075 | CCDC39 |
| ENSG00000145087 | STXBP5L |
| ENSG00000145103 | ILDR1 |
| ENSG00000145147 | SLIT2 |
| ENSG00000145198 | VWA5B2 |
| ENSG00000145242 | EPHA5 |
| ENSG00000145248 | SLC10A4 |
| ENSG00000145283 | SLC10A6 |
| ENSG00000145309 | CABS1 |
| ENSG00000145358 | DDIT4L |
| ENSG00000145384 | FABP2 |
| ENSG00000145451 | GLRA3 |
| ENSG00000145506 | NKD2 |
| ENSG00000145526 | CDH18 |
| ENSG00000145536 | ADAMTS16 |
| ENSG00000145626 | UGT3A1 |
| ENSG00000145642 | FAM159B |
| ENSG00000145681 | HAPLN1 |
| ENSG00000145700 | ANKRD31 |
| ENSG00000145721 | LIX1 |
| ENSG00000145757 | SPATA9 |
| ENSG00000145777 | TSLP |
| ENSG00000145794 | MEGF10 |
| ENSG00000145808 | ADAMTS19 |
| ENSG00000145826 | LECT2 |
| ENSG00000145832 | SLC25A48 |
| ENSG00000145839 | IL9 |
| ENSG00000145861 | C1QTNF2 |
| ENSG00000145863 | GABRA6 |
| ENSG00000145864 | GABRB2 |
| ENSG00000145879 | SPINK7 |
| ENSG00000145888 | GLRA1 |
| ENSG00000145934 | TENM2 |
| ENSG00000145975 | FAM217A |
| ENSG00000146005 | PSD2 |
| ENSG00000146006 | LRRTM2 |
| ENSG00000146013 | GFRA3 |
| ENSG00000146038 | DCDC2 |
| ENSG00000146039 | SLC17A4 |
| ENSG00000146047 | HIST1H2BA |
| ENSG00000146049 | KAAG1 |
| ENSG00000146090 | RASGEF1C |
| ENSG00000146147 | MLIP |
| ENSG00000146151 | HMGCLL1 |
| ENSG00000146166 | LGSN |
| ENSG00000146197 | SCUBE3 |
| ENSG00000146205 | ANO7 |
| ENSG00000146216 | TTBK1 |
| ENSG00000146221 | TCTE1 |
| ENSG00000146233 | CYP39A1 |
| ENSG00000146250 | PRSS35 |
| ENSG00000146267 | FAXC |
| ENSG00000146276 | GABRR1 |
| ENSG00000146352 | CLVS2 |
| ENSG00000146360 | GPR6 |
| ENSG00000146374 | RSPO3 |
| ENSG00000146378 | TAAR2 |
| ENSG00000146383 | TAAR6 |
| ENSG00000146385 | TAAR8 |
| ENSG00000146399 | TAAR1 |
| ENSG00000146411 | SLC2A12 |
| ENSG00000146453 | PNLDC1 |
| ENSG00000146469 | VIP |
| ENSG00000146477 | SLC22A3 |
| ENSG00000146530 | VWDE |
| ENSG00000146618 | FERD3L |
| ENSG00000146678 | IGFBP1 |
| ENSG00000146755 | TRIM50 |
| ENSG00000146809 | ASB15 |
| ENSG00000146839 | ZAN |
| ENSG00000146856 | AGBL3 |
| ENSG00000146857 | STRA8 |
| ENSG00000146910 | CNPY1 |
| ENSG00000146926 | ASB10 |
| ENSG00000146938 | NLGN4X |
| ENSG00000146950 | SHROOM2 |
| ENSG00000147003 | TMEM27 |
| ENSG00000147027 | TMEM47 |
| ENSG00000147041 | SYTL5 |
| ENSG00000147059 | SPIN2A |
| ENSG00000147081 | AKAP4 |
| ENSG00000147082 | CCNB3 |
| ENSG00000147100 | SLC16A2 |
| ENSG00000147117 | ZNF157 |
| ENSG00000147127 | RAB41 |
| ENSG00000147145 | LPAR4 |
| ENSG00000147160 | AWAT2 |
| ENSG00000147183 | CPXCR1 |
| ENSG00000147223 | RIPPLY1 |
| ENSG00000147246 | HTR2C |
| ENSG00000147255 | IGSF1 |
| ENSG00000147256 | ARHGAP36 |
| ENSG00000147257 | GPC3 |
| ENSG00000147262 | GPR119 |
| ENSG00000147378 | FATE1 |
| ENSG00000147381 | MAGEA4 |
| ENSG00000147432 | CHRNB3 |
| ENSG00000147434 | CHRNA6 |
| ENSG00000147481 | SNTG1 |
| ENSG00000147485 | PXDNL |
| ENSG00000147488 | ST18 |
| ENSG00000147509 | RGS20 |
| ENSG00000147570 | DNAJC5B |
| ENSG00000147571 | CRH |
| ENSG00000147573 | TRIM55 |
| ENSG00000147588 | PMP2 |
| ENSG00000147596 | PRDM14 |
| ENSG00000147606 | SLC26A7 |
| ENSG00000147613 | PSKH2 |
| ENSG00000147614 | ATP6V0D2 |
| ENSG00000147647 | DPYS |
| ENSG00000147655 | RSPO2 |
| ENSG00000147676 | MAL2 |
| ENSG00000147697 | GSDMC |
| ENSG00000147724 | FAM135B |
| ENSG00000147799 | ARHGAP39 |
| ENSG00000147869 | CER1 |
| ENSG00000147873 | IFNA5 |
| ENSG00000147885 | IFNA16 |
| ENSG00000147896 | IFNK |
| ENSG00000148082 | SHC3 |
| ENSG00000148123 | PLPPR1 |
| ENSG00000148136 | OR13C4 |
| ENSG00000148156 | ACTL7B |
| ENSG00000148215 | OR5C1 |
| ENSG00000148357 | HMCN2 |
| ENSG00000148377 | IDI2 |
| ENSG00000148386 | LCN9 |
| ENSG00000148482 | SLC39A12 |
| ENSG00000148483 | TMEM236 |
| ENSG00000148513 | ANKRD30A |
| ENSG00000148541 | FAM13C |
| ENSG00000148602 | LRIT1 |
| ENSG00000148604 | RGR |
| ENSG00000148680 | HTR7 |
| ENSG00000148702 | HABP2 |
| ENSG00000148704 | VAX1 |
| ENSG00000148734 | NPFFR1 |
| ENSG00000148735 | PLEKHS1 |
| ENSG00000148798 | INA |
| ENSG00000148826 | NKX6-2 |
| ENSG00000148935 | GAS2 |
| ENSG00000148942 | SLC5A12 |
| ENSG00000148948 | LRRC4C |
| ENSG00000149021 | SCGB1A1 |
| ENSG00000149043 | SYT8 |
| ENSG00000149050 | ZNF214 |
| ENSG00000149054 | ZNF215 |
| ENSG00000149090 | PAMR1 |
| ENSG00000149133 | OR5F1 |
| ENSG00000149201 | CCDC81 |
| ENSG00000149256 | TENM4 |
| ENSG00000149295 | DRD2 |
| ENSG00000149300 | C11orf52 |
| ENSG00000149305 | HTR3B |
| ENSG00000149380 | P4HA3 |
| ENSG00000149435 | GGTLC1 |
| ENSG00000149452 | SLC22A8 |
| ENSG00000149488 | TMC2 |
| ENSG00000149506 | ZP1 |
| ENSG00000149507 | OOSP2 |
| ENSG00000149571 | KIRREL3 |
| ENSG00000149575 | SCN2B |
| ENSG00000149596 | JPH2 |
| ENSG00000149599 | DUSP15 |
| ENSG00000149609 | C20orf144 |
| ENSG00000149633 | KIAA1755 |
| ENSG00000149634 | SPATA25 |
| ENSG00000149635 | OCSTAMP |
| ENSG00000149651 | SPINT4 |
| ENSG00000149654 | CDH22 |
| ENSG00000149735 | GPHA2 |
| ENSG00000149742 | SLC22A9 |
| ENSG00000149926 | FAM57B |
| ENSG00000149927 | DOC2A |
| ENSG00000149948 | HMGA2 |
| ENSG00000149968 | MMP3 |
| ENSG00000149972 | CNTN5 |
| ENSG00000150051 | MKX |
| ENSG00000150201 | FXYD4 |
| ENSG00000150244 | TRIM48 |
| ENSG00000150261 | OR8K1 |
| ENSG00000150269 | OR5M9 |
| ENSG00000150275 | PCDH15 |
| ENSG00000150361 | KLHL1 |
| ENSG00000150394 | CDH8 |
| ENSG00000150471 | ADGRL3 |
| ENSG00000150526 | MIA2 |
| ENSG00000150551 | LYPD1 |
| ENSG00000150556 | LYPD6B |
| ENSG00000150627 | WDR17 |
| ENSG00000150628 | SPATA4 |
| ENSG00000150656 | CNDP1 |
| ENSG00000150667 | FSIP1 |
| ENSG00000150676 | CCDC83 |
| ENSG00000150722 | PPP1R1C |
| ENSG00000150750 | C11orf53 |
| ENSG00000150773 | PIH1D2 |
| ENSG00000150783 | TEX12 |
| ENSG00000150873 | C2orf50 |
| ENSG00000150893 | FREM2 |
| ENSG00000151005 | TKTL2 |
| ENSG00000151025 | GPR158 |
| ENSG00000151033 | LYZL2 |
| ENSG00000151079 | KCNA6 |
| ENSG00000151322 | NPAS3 |
| ENSG00000151338 | MIPOL1 |
| ENSG00000151360 | ALLC |
| ENSG00000151364 | KCTD14 |
| ENSG00000151365 | THRSP |
| ENSG00000151379 | MSGN1 |
| ENSG00000151388 | ADAMTS12 |
| ENSG00000151418 | ATP6V1G3 |
| ENSG00000151475 | SLC25A31 |
| ENSG00000151572 | ANO4 |
| ENSG00000151577 | DRD3 |
| ENSG00000151615 | POU4F2 |
| ENSG00000151617 | EDNRA |
| ENSG00000151704 | KCNJ1 |
| ENSG00000151773 | CCDC122 |
| ENSG00000151778 | SERP2 |
| ENSG00000151790 | TDO2 |
| ENSG00000151812 | SLC35F4 |
| ENSG00000151834 | GABRA2 |
| ENSG00000151838 | CCDC175 |
| ENSG00000151892 | GFRA1 |
| ENSG00000151917 | BEND6 |
| ENSG00000151962 | RBM46 |
| ENSG00000151967 | SCHIP1 |
| ENSG00000152034 | MCHR2 |
| ENSG00000152049 | KCNE4 |
| ENSG00000152076 | CCDC74B |
| ENSG00000152086 | TUBA3E |
| ENSG00000152092 | ASTN1 |
| ENSG00000152093 | CFC1B |
| ENSG00000152154 | TMEM178A |
| ENSG00000152192 | POU4F1 |
| ENSG00000152208 | GRID2 |
| ENSG00000152214 | RIT2 |
| ENSG00000152254 | G6PC2 |
| ENSG00000152266 | PTH |
| ENSG00000152292 | SH2D6 |
| ENSG00000152402 | GUCY1A2 |
| ENSG00000152430 | BOLL |
| ENSG00000152467 | ZSCAN1 |
| ENSG00000152527 | PLEKHH2 |
| ENSG00000152578 | GRIA4 |
| ENSG00000152580 | IGSF10 |
| ENSG00000152591 | DSPP |
| ENSG00000152592 | DMP1 |
| ENSG00000152595 | MEPE |
| ENSG00000152611 | CAPSL |
| ENSG00000152669 | CCNO |
| ENSG00000152670 | DDX4 |
| ENSG00000152705 | CATSPER3 |
| ENSG00000152779 | SLC16A12 |
| ENSG00000152785 | BMP3 |
| ENSG00000152822 | GRM1 |
| ENSG00000152910 | CNTNAP4 |
| ENSG00000152936 | LMNTD1 |
| ENSG00000152939 | MARVELD2 |
| ENSG00000152954 | NRSN1 |
| ENSG00000152977 | ZIC1 |
| ENSG00000153012 | LGI2 |
| ENSG00000153060 | TEKT5 |
| ENSG00000153086 | ACMSD |
| ENSG00000153093 | ACOXL |
| ENSG00000153132 | CLGN |
| ENSG00000153157 | SYCP2L |
| ENSG00000153165 | RGPD3 |
| ENSG00000153230 | OR14K1 |
| ENSG00000153233 | PTPRR |
| ENSG00000153237 | CCDC148 |
| ENSG00000153246 | PLA2R1 |
| ENSG00000153266 | FEZF2 |
| ENSG00000153291 | SLC25A27 |
| ENSG00000153292 | ADGRF1 |
| ENSG00000153294 | ADGRF4 |
| ENSG00000153303 | FRMD1 |
| ENSG00000153347 | FAM81B |
| ENSG00000153404 | PLEKHG4B |
| ENSG00000153446 | C16orf89 |
| ENSG00000153495 | TEX29 |
| ENSG00000153498 | SPACA7 |
| ENSG00000153684 | GOLGA8F |
| ENSG00000153707 | PTPRD |
| ENSG00000153779 | TGIF2LX |
| ENSG00000153789 | FAM92B |
| ENSG00000153802 | TMPRSS11D |
| ENSG00000153820 | SPHKAP |
| ENSG00000153822 | KCNJ16 |
| ENSG00000153930 | ANKFN1 |
| ENSG00000153956 | CACNA2D1 |
| ENSG00000153976 | HS3ST3A1 |
| ENSG00000153993 | SEMA3D |
| ENSG00000154007 | ASB17 |
| ENSG00000154040 | CABYR |
| ENSG00000154065 | ANKRD29 |
| ENSG00000154080 | CHST9 |
| ENSG00000154118 | JPH3 |
| ENSG00000154143 | PANX3 |
| ENSG00000154162 | CDH12 |
| ENSG00000154227 | CERS3 |
| ENSG00000154252 | GAL3ST2 |
| ENSG00000154269 | ENPP3 |
| ENSG00000154342 | WNT3A |
| ENSG00000154415 | PPP1R3A |
| ENSG00000154438 | ASZ1 |
| ENSG00000154478 | GPR26 |
| ENSG00000154479 | CCDC173 |
| ENSG00000154485 | MMP21 |
| ENSG00000154493 | C10orf90 |
| ENSG00000154548 | SRSF12 |
| ENSG00000154611 | PSMA8 |
| ENSG00000154639 | CXADR |
| ENSG00000154645 | CHODL |
| ENSG00000154646 | TMPRSS15 |
| ENSG00000154654 | NCAM2 |
| ENSG00000154678 | PDE1C |
| ENSG00000154864 | PIEZO2 |
| ENSG00000154975 | CA10 |
| ENSG00000154997 | SEPT14 |
| ENSG00000155011 | DKK2 |
| ENSG00000155026 | RSPH10B |
| ENSG00000155052 | CNTNAP5 |
| ENSG00000155066 | PROM2 |
| ENSG00000155087 | ODF1 |
| ENSG00000155249 | OR4K1 |
| ENSG00000155269 | GPR78 |
| ENSG00000155428 | TRIM74 |
| ENSG00000155495 | MAGEC1 |
| ENSG00000155511 | GRIA1 |
| ENSG00000155622 | XAGE2 |
| ENSG00000155714 | PDZD9 |
| ENSG00000155719 | OTOA |
| ENSG00000155754 | ALS2CR11 |
| ENSG00000155760 | FZD7 |
| ENSG00000155761 | SPAG17 |
| ENSG00000155816 | FMN2 |
| ENSG00000155833 | CYLC2 |
| ENSG00000155875 | SAXO1 |
| ENSG00000155886 | SLC24A2 |
| ENSG00000155890 | TRIM42 |
| ENSG00000155897 | ADCY8 |
| ENSG00000155918 | RAET1L |
| ENSG00000156006 | NAT2 |
| ENSG00000156009 | MAGEA8 |
| ENSG00000156049 | GNA14 |
| ENSG00000156076 | WIF1 |
| ENSG00000156096 | UGT2B4 |
| ENSG00000156103 | MMP16 |
| ENSG00000156150 | ALX3 |
| ENSG00000156194 | PPEF2 |
| ENSG00000156218 | ADAMTSL3 |
| ENSG00000156219 | ART3 |
| ENSG00000156222 | SLC28A1 |
| ENSG00000156234 | CXCL13 |
| ENSG00000156269 | NAA11 |
| ENSG00000156282 | CLDN17 |
| ENSG00000156284 | CLDN8 |
| ENSG00000156395 | SORCS3 |
| ENSG00000156413 | FUT6 |
| ENSG00000156427 | FGF18 |
| ENSG00000156466 | GDF6 |
| ENSG00000156486 | KCNS2 |
| ENSG00000156509 | FBXO43 |
| ENSG00000156564 | LRFN2 |
| ENSG00000156574 | NODAL |
| ENSG00000156687 | UNC5D |
| ENSG00000156689 | GLYATL2 |
| ENSG00000156920 | ADGRG4 |
| ENSG00000156925 | ZIC3 |
| ENSG00000156959 | LHFPL4 |
| ENSG00000156968 | MPV17L |
| ENSG00000157005 | SST |
| ENSG00000157060 | SHCBP1L |
| ENSG00000157087 | ATP2B2 |
| ENSG00000157093 | LYZL4 |
| ENSG00000157103 | SLC6A1 |
| ENSG00000157111 | TMEM171 |
| ENSG00000157119 | KLHL40 |
| ENSG00000157131 | C8A |
| ENSG00000157211 | CDCP2 |
| ENSG00000157214 | STEAP2 |
| ENSG00000157219 | HTR5A |
| ENSG00000157315 | TMED6 |
| ENSG00000157330 | C1orf158 |
| ENSG00000157368 | IL34 |
| ENSG00000157399 | ARSE |
| ENSG00000157423 | HYDIN |
| ENSG00000157470 | FAM81A |
| ENSG00000157502 | MUM1L1 |
| ENSG00000157542 | KCNJ6 |
| ENSG00000157578 | LCA5L |
| ENSG00000157653 | C9orf43 |
| ENSG00000157654 | PALM2-AKAP2 |
| ENSG00000157680 | DGKI |
| ENSG00000157703 | SVOPL |
| ENSG00000157765 | SLC34A2 |
| ENSG00000157766 | ACAN |
| ENSG00000157782 | CABP1 |
| ENSG00000157851 | DPYSL5 |
| ENSG00000157856 | DRC1 |
| ENSG00000157884 | CIB4 |
| ENSG00000157890 | MEGF11 |
| ENSG00000157999 | ANKRD61 |
| ENSG00000158008 | EXTL1 |
| ENSG00000158014 | SLC30A2 |
| ENSG00000158055 | GRHL3 |
| ENSG00000158077 | NLRP14 |
| ENSG00000158125 | XDH |
| ENSG00000158164 | TMSB15A |
| ENSG00000158220 | ESYT3 |
| ENSG00000158246 | FAM46B |
| ENSG00000158258 | CLSTN2 |
| ENSG00000158296 | SLC13A3 |
| ENSG00000158315 | RHBDL2 |
| ENSG00000158423 | RIBC1 |
| ENSG00000158458 | NRG2 |
| ENSG00000158485 | CD1B |
| ENSG00000158486 | DNAH3 |
| ENSG00000158488 | CD1E |
| ENSG00000158497 | HMHB1 |
| ENSG00000158525 | CPA5 |
| ENSG00000158553 | POM121L2 |
| ENSG00000158571 | PFKFB1 |
| ENSG00000158639 | PAGE5 |
| ENSG00000158748 | HTR6 |
| ENSG00000158764 | ITLN2 |
| ENSG00000158786 | PLA2G2F |
| ENSG00000158806 | NPM2 |
| ENSG00000158815 | FGF17 |
| ENSG00000158816 | VWA5B1 |
| ENSG00000158865 | SLC5A11 |
| ENSG00000158901 | WFDC8 |
| ENSG00000158955 | WNT9B |
| ENSG00000159166 | LAD1 |
| ENSG00000159167 | STC1 |
| ENSG00000159182 | PRAC1 |
| ENSG00000159184 | HOXB13 |
| ENSG00000159197 | KCNE2 |
| ENSG00000159208 | CIART |
| ENSG00000159212 | CLIC6 |
| ENSG00000159217 | IGF2BP1 |
| ENSG00000159224 | GIP |
| ENSG00000159248 | GJD2 |
| ENSG00000159261 | CLDN14 |
| ENSG00000159263 | SIM2 |
| ENSG00000159289 | GOLGA6A |
| ENSG00000159337 | PLA2G4D |
| ENSG00000159387 | IRX6 |
| ENSG00000159398 | CES5A |
| ENSG00000159409 | CELF3 |
| ENSG00000159450 | TCHH |
| ENSG00000159455 | LCE2B |
| ENSG00000159495 | TGM7 |
| ENSG00000159516 | SPRR2G |
| ENSG00000159527 | PGLYRP3 |
| ENSG00000159648 | TEPP |
| ENSG00000159650 | UROC1 |
| ENSG00000159708 | LRRC36 |
| ENSG00000159723 | AGRP |
| ENSG00000159763 | PIP |
| ENSG00000159871 | LYPD5 |
| ENSG00000159905 | ZNF221 |
| ENSG00000159961 | OR3A3 |
| ENSG00000160161 | CILP2 |
| ENSG00000160181 | TFF2 |
| ENSG00000160182 | TFF1 |
| ENSG00000160188 | RSPH1 |
| ENSG00000160202 | CRYAA |
| ENSG00000160207 | HSF2BP |
| ENSG00000160224 | AIRE |
| ENSG00000160282 | FTCD |
| ENSG00000160321 | ZNF208 |
| ENSG00000160339 | FCN2 |
| ENSG00000160345 | C9orf116 |
| ENSG00000160349 | LCN1 |
| ENSG00000160396 | HIPK4 |
| ENSG00000160472 | TMEM190 |
| ENSG00000160505 | NLRP4 |
| ENSG00000160606 | TLCD1 |
| ENSG00000160716 | CHRNB2 |
| ENSG00000160801 | PTH1R |
| ENSG00000160838 | LRRC71 |
| ENSG00000160870 | CYP3A7 |
| ENSG00000160886 | LY6K |
| ENSG00000160951 | PTGER1 |
| ENSG00000160963 | COL26A1 |
| ENSG00000160973 | FOXH1 |
| ENSG00000160994 | CCDC105 |
| ENSG00000161055 | SCGB3A1 |
| ENSG00000161082 | CELF5 |
| ENSG00000161103 | AC008132.13 |
| ENSG00000161133 | USP41 |
| ENSG00000161180 | CCDC116 |
| ENSG00000161243 | FBXO27 |
| ENSG00000161270 | NPHS1 |
| ENSG00000161594 | KLHL10 |
| ENSG00000161609 | CCDC155 |
| ENSG00000161610 | HCRT |
| ENSG00000161634 | DCD |
| ENSG00000161649 | CD300LG |
| ENSG00000161652 | IZUMO2 |
| ENSG00000161682 | FAM171A2 |
| ENSG00000161798 | AQP5 |
| ENSG00000161807 | OR7G1 |
| ENSG00000161849 | KRT84 |
| ENSG00000161850 | KRT82 |
| ENSG00000161860 | SYCE2 |
| ENSG00000161896 | IP6K3 |
| ENSG00000161973 | CCDC42 |
| ENSG00000161992 | PRR35 |
| ENSG00000162006 | MSLNL |
| ENSG00000162009 | SSTR5 |
| ENSG00000162039 | MEIOB |
| ENSG00000162040 | HS3ST6 |
| ENSG00000162068 | NTN3 |
| ENSG00000162105 | SHANK2 |
| ENSG00000162188 | GNG3 |
| ENSG00000162344 | FGF19 |
| ENSG00000162365 | CYP4A22 |
| ENSG00000162374 | ELAVL4 |
| ENSG00000162391 | FAM151A |
| ENSG00000162399 | BSND |
| ENSG00000162409 | PRKAA2 |
| ENSG00000162456 | KNCN |
| ENSG00000162460 | TMEM82 |
| ENSG00000162493 | PDPN |
| ENSG00000162494 | LRRC38 |
| ENSG00000162510 | MATN1 |
| ENSG00000162520 | SYNC |
| ENSG00000162571 | TTLL10 |
| ENSG00000162592 | CCDC27 |
| ENSG00000162594 | IL23R |
| ENSG00000162595 | DIRAS3 |
| ENSG00000162598 | C1orf87 |
| ENSG00000162620 | LRRIQ3 |
| ENSG00000162621 | LRRC53 |
| ENSG00000162624 | LHX8 |
| ENSG00000162627 | SNX7 |
| ENSG00000162630 | B3GALT2 |
| ENSG00000162631 | NTNG1 |
| ENSG00000162641 | AKNAD1 |
| ENSG00000162643 | WDR63 |
| ENSG00000162669 | HFM1 |
| ENSG00000162670 | BRINP3 |
| ENSG00000162687 | KCNT2 |
| ENSG00000162723 | SLAMF9 |
| ENSG00000162727 | OR2M5 |
| ENSG00000162728 | KCNJ9 |
| ENSG00000162738 | VANGL2 |
| ENSG00000162753 | SLC9C2 |
| ENSG00000162755 | KLHDC9 |
| ENSG00000162761 | LMX1A |
| ENSG00000162763 | LRRC52 |
| ENSG00000162771 | FAM71A |
| ENSG00000162779 | AXDND1 |
| ENSG00000162782 | TDRD5 |
| ENSG00000162814 | SPATA17 |
| ENSG00000162843 | WDR64 |
| ENSG00000162877 | PM20D1 |
| ENSG00000162891 | IL20 |
| ENSG00000162897 | FCAMR |
| ENSG00000162913 | C1orf145 |
| ENSG00000162951 | LRRTM1 |
| ENSG00000162975 | KCNF1 |
| ENSG00000162981 | FAM84A |
| ENSG00000162989 | KCNJ3 |
| ENSG00000162992 | NEUROD1 |
| ENSG00000162998 | FRZB |
| ENSG00000163012 | ZSWIM2 |
| ENSG00000163032 | VSNL1 |
| ENSG00000163040 | CCDC74A |
| ENSG00000163046 | ANKRD30BL |
| ENSG00000163060 | TEKT4 |
| ENSG00000163064 | EN1 |
| ENSG00000163071 | SPATA18 |
| ENSG00000163075 | CFAP221 |
| ENSG00000163081 | CCDC140 |
| ENSG00000163098 | BIRC8 |
| ENSG00000163106 | HPGDS |
| ENSG00000163114 | PDHA2 |
| ENSG00000163116 | STPG2 |
| ENSG00000163121 | NEURL3 |
| ENSG00000163132 | MSX1 |
| ENSG00000163145 | C1QTNF7 |
| ENSG00000163157 | TMOD4 |
| ENSG00000163202 | LCE3D |
| ENSG00000163206 | SMCP |
| ENSG00000163207 | IVL |
| ENSG00000163216 | SPRR2D |
| ENSG00000163217 | BMP10 |
| ENSG00000163218 | PGLYRP4 |
| ENSG00000163239 | TDRD10 |
| ENSG00000163254 | CRYGC |
| ENSG00000163263 | C1orf189 |
| ENSG00000163273 | NPPC |
| ENSG00000163283 | ALPP |
| ENSG00000163285 | GABRG1 |
| ENSG00000163286 | ALPPL2 |
| ENSG00000163288 | GABRB1 |
| ENSG00000163293 | NIPAL1 |
| ENSG00000163295 | ALPI |
| ENSG00000163331 | DAPL1 |
| ENSG00000163347 | CLDN1 |
| ENSG00000163352 | LENEP |
| ENSG00000163357 | DCST1 |
| ENSG00000163377 | FAM19A4 |
| ENSG00000163380 | LMOD3 |
| ENSG00000163394 | CCKAR |
| ENSG00000163395 | IGFN1 |
| ENSG00000163424 | C3orf30 |
| ENSG00000163440 | PDCL2 |
| ENSG00000163467 | TSACC |
| ENSG00000163485 | ADORA1 |
| ENSG00000163491 | NEK10 |
| ENSG00000163497 | FEV |
| ENSG00000163499 | CRYBA2 |
| ENSG00000163501 | IHH |
| ENSG00000163515 | RETNLB |
| ENSG00000163518 | FCRL4 |
| ENSG00000163530 | DPPA2 |
| ENSG00000163576 | EFHB |
| ENSG00000163581 | SLC2A2 |
| ENSG00000163618 | CADPS |
| ENSG00000163623 | NKX6-1 |
| ENSG00000163624 | CDS1 |
| ENSG00000163630 | SYNPR |
| ENSG00000163632 | C3orf49 |
| ENSG00000163645 | ERICH6 |
| ENSG00000163646 | CLRN1 |
| ENSG00000163666 | HESX1 |
| ENSG00000163673 | DCLK3 |
| ENSG00000163689 | C3orf67 |
| ENSG00000163705 | FANCD2OS |
| ENSG00000163746 | PLSCR2 |
| ENSG00000163749 | CCDC158 |
| ENSG00000163762 | TM4SF18 |
| ENSG00000163792 | TCF23 |
| ENSG00000163793 | DNAJC5G |
| ENSG00000163810 | TGM4 |
| ENSG00000163817 | SLC6A20 |
| ENSG00000163825 | RTP3 |
| ENSG00000163873 | GRIK3 |
| ENSG00000163879 | DNALI1 |
| ENSG00000163884 | KLF15 |
| ENSG00000163885 | CFAP100 |
| ENSG00000163888 | CAMK2N2 |
| ENSG00000163909 | HEYL |
| ENSG00000163914 | RHO |
| ENSG00000163982 | OTOP1 |
| ENSG00000164007 | CLDN19 |
| ENSG00000164049 | FBXW12 |
| ENSG00000164076 | CAMKV |
| ENSG00000164078 | MST1R |
| ENSG00000164089 | ETNPPL |
| ENSG00000164093 | PITX2 |
| ENSG00000164099 | PRSS12 |
| ENSG00000164107 | HAND2 |
| ENSG00000164112 | TMEM155 |
| ENSG00000164113 | ADAD1 |
| ENSG00000164122 | ASB5 |
| ENSG00000164123 | C4orf45 |
| ENSG00000164128 | NPY1R |
| ENSG00000164129 | NPY5R |
| ENSG00000164161 | HHIP |
| ENSG00000164175 | SLC45A2 |
| ENSG00000164176 | EDIL3 |
| ENSG00000164185 | ZNF474 |
| ENSG00000164188 | RANBP3L |
| ENSG00000164197 | RNF180 |
| ENSG00000164199 | ADGRV1 |
| ENSG00000164220 | F2RL2 |
| ENSG00000164256 | PRDM9 |
| ENSG00000164265 | SCGB3A2 |
| ENSG00000164266 | SPINK1 |
| ENSG00000164270 | HTR4 |
| ENSG00000164283 | ESM1 |
| ENSG00000164287 | CDC20B |
| ENSG00000164294 | GPX8 |
| ENSG00000164299 | SPZ1 |
| ENSG00000164303 | ENPP6 |
| ENSG00000164304 | CAGE1 |
| ENSG00000164318 | EGFLAM |
| ENSG00000164325 | TMEM174 |
| ENSG00000164326 | CARTPT |
| ENSG00000164334 | FAM170A |
| ENSG00000164362 | TERT |
| ENSG00000164363 | SLC6A18 |
| ENSG00000164393 | ADGRF2 |
| ENSG00000164399 | IL3 |
| ENSG00000164400 | CSF2 |
| ENSG00000164404 | GDF9 |
| ENSG00000164411 | GJB7 |
| ENSG00000164418 | GRIK2 |
| ENSG00000164434 | FABP7 |
| ENSG00000164438 | TLX3 |
| ENSG00000164451 | FAM26D |
| ENSG00000164458 | T |
| ENSG00000164484 | TMEM200A |
| ENSG00000164485 | IL22RA2 |
| ENSG00000164488 | DACT2 |
| ENSG00000164500 | C7orf72 |
| ENSG00000164508 | HIST1H2AA |
| ENSG00000164509 | IL31RA |
| ENSG00000164520 | RAET1E |
| ENSG00000164532 | TBX20 |
| ENSG00000164542 | KIAA0895 |
| ENSG00000164588 | HCN1 |
| ENSG00000164591 | MYOZ3 |
| ENSG00000164600 | NEUROD6 |
| ENSG00000164604 | GPR85 |
| ENSG00000164619 | BMPER |
| ENSG00000164627 | KIF6 |
| ENSG00000164645 | C7orf62 |
| ENSG00000164647 | STEAP1 |
| ENSG00000164651 | SP8 |
| ENSG00000164675 | IQUB |
| ENSG00000164690 | SHH |
| ENSG00000164694 | FNDC1 |
| ENSG00000164695 | CHMP4C |
| ENSG00000164729 | SLC35G3 |
| ENSG00000164742 | ADCY1 |
| ENSG00000164743 | C8orf48 |
| ENSG00000164744 | SUN3 |
| ENSG00000164746 | C7orf57 |
| ENSG00000164749 | HNF4G |
| ENSG00000164756 | SLC30A8 |
| ENSG00000164761 | TNFRSF11B |
| ENSG00000164778 | EN2 |
| ENSG00000164794 | KCNV1 |
| ENSG00000164796 | CSMD3 |
| ENSG00000164816 | DEFA5 |
| ENSG00000164822 | DEFA6 |
| ENSG00000164825 | DEFB1 |
| ENSG00000164841 | TMEM74 |
| ENSG00000164871 | SPAG11B |
| ENSG00000164893 | SLC7A13 |
| ENSG00000164900 | GBX1 |
| ENSG00000164920 | OSR2 |
| ENSG00000164932 | CTHRC1 |
| ENSG00000164935 | DCSTAMP |
| ENSG00000164946 | FREM1 |
| ENSG00000164972 | C9orf24 |
| ENSG00000165023 | DIRAS2 |
| ENSG00000165059 | PRKACG |
| ENSG00000165061 | ZMAT4 |
| ENSG00000165066 | NKX6-3 |
| ENSG00000165072 | MAMDC2 |
| ENSG00000165076 | PRSS37 |
| ENSG00000165078 | CPA6 |
| ENSG00000165084 | C8orf34 |
| ENSG00000165091 | TMC1 |
| ENSG00000165105 | RASEF |
| ENSG00000165120 | SSMEM1 |
| ENSG00000165152 | TMEM246 |
| ENSG00000165164 | CFAP47 |
| ENSG00000165171 | WBSCR27 |
| ENSG00000165182 | CXorf58 |
| ENSG00000165186 | PTCHD1 |
| ENSG00000165188 | RNF183 |
| ENSG00000165192 | ASB11 |
| ENSG00000165194 | PCDH19 |
| ENSG00000165197 | FIGF |
| ENSG00000165202 | OR1Q1 |
| ENSG00000165204 | OR1K1 |
| ENSG00000165215 | CLDN3 |
| ENSG00000165246 | NLGN4Y |
| ENSG00000165269 | AQP7 |
| ENSG00000165300 | SLITRK5 |
| ENSG00000165323 | FAT3 |
| ENSG00000165325 | CCDC67 |
| ENSG00000165349 | SLC7A3 |
| ENSG00000165370 | GPR101 |
| ENSG00000165376 | CLDN2 |
| ENSG00000165379 | LRFN5 |
| ENSG00000165383 | LRRC18 |
| ENSG00000165409 | TSHR |
| ENSG00000165443 | PHYHIPL |
| ENSG00000165449 | SLC16A9 |
| ENSG00000165462 | PHOX2A |
| ENSG00000165471 | MBL2 |
| ENSG00000165478 | HEPACAM |
| ENSG00000165495 | PKNOX2 |
| ENSG00000165496 | RPL10L |
| ENSG00000165509 | MAGEC3 |
| ENSG00000165553 | NGB |
| ENSG00000165556 | CDX2 |
| ENSG00000165566 | AMER2 |
| ENSG00000165583 | SSX5 |
| ENSG00000165584 | SSX3 |
| ENSG00000165588 | OTX2 |
| ENSG00000165606 | DRGX |
| ENSG00000165621 | OXGR1 |
| ENSG00000165623 | UCMA |
| ENSG00000165633 | VSTM4 |
| ENSG00000165643 | SOHLH1 |
| ENSG00000165694 | FRMD7 |
| ENSG00000165695 | AK8 |
| ENSG00000165730 | STOX1 |
| ENSG00000165762 | OR4K2 |
| ENSG00000165794 | SLC39A2 |
| ENSG00000165799 | RNASE7 |
| ENSG00000165805 | C12orf50 |
| ENSG00000165807 | PPP1R36 |
| ENSG00000165816 | VWA2 |
| ENSG00000165828 | PRAP1 |
| ENSG00000165837 | ERICH6B |
| ENSG00000165841 | CYP2C19 |
| ENSG00000165863 | C10orf82 |
| ENSG00000165868 | HSPA12A |
| ENSG00000165899 | OTOGL |
| ENSG00000165935 | SMCO2 |
| ENSG00000165953 | SERPINA12 |
| ENSG00000165966 | PDZRN4 |
| ENSG00000165970 | SLC6A5 |
| ENSG00000165972 | CCDC38 |
| ENSG00000165973 | NELL1 |
| ENSG00000165985 | C1QL3 |
| ENSG00000165995 | CACNB2 |
| ENSG00000166006 | KCNC2 |
| ENSG00000166049 | PASD1 |
| ENSG00000166069 | TMCO5A |
| ENSG00000166073 | GPR176 |
| ENSG00000166090 | IL25 |
| ENSG00000166106 | ADAMTS15 |
| ENSG00000166111 | SVOP |
| ENSG00000166118 | SPATA19 |
| ENSG00000166143 | PPP1R14D |
| ENSG00000166152 | C16orf78 |
| ENSG00000166153 | DEPDC4 |
| ENSG00000166159 | LRTM2 |
| ENSG00000166160 | OPN1MW2 |
| ENSG00000166173 | LARP6 |
| ENSG00000166183 | ASPG |
| ENSG00000166206 | GABRB3 |
| ENSG00000166220 | TBATA |
| ENSG00000166246 | C16orf71 |
| ENSG00000166257 | SCN3B |
| ENSG00000166268 | MYRFL |
| ENSG00000166292 | TMEM100 |
| ENSG00000166317 | SYNPO2L |
| ENSG00000166329 | CCDC182 |
| ENSG00000166349 | RAG1 |
| ENSG00000166351 | POTED |
| ENSG00000166359 | WDR88 |
| ENSG00000166363 | OR10A5 |
| ENSG00000166368 | OR2D2 |
| ENSG00000166391 | MOGAT2 |
| ENSG00000166396 | SERPINB7 |
| ENSG00000166407 | LMO1 |
| ENSG00000166415 | WDR72 |
| ENSG00000166426 | CRABP1 |
| ENSG00000166448 | TMEM130 |
| ENSG00000166450 | PRTG |
| ENSG00000166455 | C16orf46 |
| ENSG00000166509 | CLEC3A |
| ENSG00000166510 | CCDC68 |
| ENSG00000166535 | A2ML1 |
| ENSG00000166558 | SLC38A8 |
| ENSG00000166569 | CPLX4 |
| ENSG00000166573 | GALR1 |
| ENSG00000166578 | IQCD |
| ENSG00000166589 | CDH16 |
| ENSG00000166596 | CFAP52 |
| ENSG00000166603 | MC4R |
| ENSG00000166634 | SERPINB12 |
| ENSG00000166670 | MMP10 |
| ENSG00000166682 | TMPRSS5 |
| ENSG00000166736 | HTR3A |
| ENSG00000166743 | ACSM1 |
| ENSG00000166748 | AGBL1 |
| ENSG00000166796 | LDHC |
| ENSG00000166800 | LDHAL6A |
| ENSG00000166823 | MESP1 |
| ENSG00000166828 | SCNN1G |
| ENSG00000166856 | GPR182 |
| ENSG00000166862 | CACNG2 |
| ENSG00000166863 | TAC3 |
| ENSG00000166869 | CHP2 |
| ENSG00000166884 | OR4D6 |
| ENSG00000166922 | SCG5 |
| ENSG00000166923 | GREM1 |
| ENSG00000166924 | NYAP1 |
| ENSG00000166926 | MS4A6E |
| ENSG00000166930 | MS4A5 |
| ENSG00000166948 | TGM6 |
| ENSG00000166959 | MS4A8 |
| ENSG00000166960 | CCDC178 |
| ENSG00000166961 | MS4A15 |
| ENSG00000166984 | TCP10L2 |
| ENSG00000167011 | NAT16 |
| ENSG00000167014 | C15orf43 |
| ENSG00000167080 | B4GALNT2 |
| ENSG00000167098 | SUN5 |
| ENSG00000167104 | BPIFB6 |
| ENSG00000167131 | CCDC103 |
| ENSG00000167139 | TBC1D21 |
| ENSG00000167157 | PRRX2 |
| ENSG00000167165 | UGT1A6 |
| ENSG00000167178 | ISLR2 |
| ENSG00000167183 | PRR15L |
| ENSG00000167194 | C16orf92 |
| ENSG00000167195 | GOLGA6C |
| ENSG00000167311 | ART5 |
| ENSG00000167332 | OR51E2 |
| ENSG00000167346 | MMP26 |
| ENSG00000167359 | OR51I1 |
| ENSG00000167360 | OR51Q1 |
| ENSG00000167414 | GNG8 |
| ENSG00000167419 | LPO |
| ENSG00000167531 | LALBA |
| ENSG00000167554 | ZNF610 |
| ENSG00000167580 | AQP2 |
| ENSG00000167612 | ANKRD33 |
| ENSG00000167617 | CDC42EP5 |
| ENSG00000167619 | TMEM145 |
| ENSG00000167646 | DNAAF3 |
| ENSG00000167653 | PSCA |
| ENSG00000167654 | ATCAY |
| ENSG00000167656 | LY6D |
| ENSG00000167741 | GGT6 |
| ENSG00000167749 | KLK4 |
| ENSG00000167751 | KLK2 |
| ENSG00000167754 | KLK5 |
| ENSG00000167755 | KLK6 |
| ENSG00000167757 | KLK11 |
| ENSG00000167759 | KLK13 |
| ENSG00000167769 | ACER1 |
| ENSG00000167771 | RCOR2 |
| ENSG00000167774 | CTD-2550O8.5 |
| ENSG00000167780 | SOAT2 |
| ENSG00000167791 | CABP2 |
| ENSG00000167800 | TBX10 |
| ENSG00000167822 | OR8J3 |
| ENSG00000167825 | OR5I1 |
| ENSG00000167858 | TEKT1 |
| ENSG00000167889 | MGAT5B |
| ENSG00000167910 | CYP7A1 |
| ENSG00000167916 | KRT24 |
| ENSG00000167941 | SOST |
| ENSG00000167945 | PRR25 |
| ENSG00000167964 | RAB26 |
| ENSG00000168032 | ENTPD3 |
| ENSG00000168065 | SLC22A11 |
| ENSG00000168070 | C11orf85 |
| ENSG00000168124 | OR1F1 |
| ENSG00000168131 | OR2B2 |
| ENSG00000168135 | KCNJ4 |
| ENSG00000168143 | FAM83B |
| ENSG00000168148 | HIST3H3 |
| ENSG00000168158 | OR2C1 |
| ENSG00000168243 | GNG4 |
| ENSG00000168263 | KCNV2 |
| ENSG00000168267 | PTF1A |
| ENSG00000168269 | FOXI1 |
| ENSG00000168314 | MOBP |
| ENSG00000168333 | C8orf22 |
| ENSG00000168348 | INSM2 |
| ENSG00000168356 | SCN11A |
| ENSG00000168398 | BDKRB2 |
| ENSG00000168412 | MTNR1A |
| ENSG00000168418 | KCNG4 |
| ENSG00000168427 | KLHL30 |
| ENSG00000168447 | SCNN1B |
| ENSG00000168453 | HR |
| ENSG00000168481 | LGI3 |
| ENSG00000168491 | CCDC110 |
| ENSG00000168505 | GBX2 |
| ENSG00000168509 | HFE2 |
| ENSG00000168515 | SCGB1D1 |
| ENSG00000168539 | CHRM1 |
| ENSG00000168582 | CRYGA |
| ENSG00000168589 | DYNLRB2 |
| ENSG00000168594 | ADAM29 |
| ENSG00000168619 | ADAM18 |
| ENSG00000168621 | GDNF |
| ENSG00000168631 | DPCR1 |
| ENSG00000168634 | WFDC13 |
| ENSG00000168658 | VWA3B |
| ENSG00000168671 | UGT3A2 |
| ENSG00000168676 | KCTD19 |
| ENSG00000168702 | LRP1B |
| ENSG00000168703 | WFDC12 |
| ENSG00000168746 | LINC01620 |
| ENSG00000168748 | CA7 |
| ENSG00000168757 | TSPY2 |
| ENSG00000168772 | CXXC4 |
| ENSG00000168779 | SHOX2 |
| ENSG00000168811 | IL12A |
| ENSG00000168828 | OR13J1 |
| ENSG00000168830 | HTR1E |
| ENSG00000168843 | FSTL5 |
| ENSG00000168875 | SOX14 |
| ENSG00000168907 | PLA2G4F |
| ENSG00000168917 | SLC35G2 |
| ENSG00000168930 | TRIM49 |
| ENSG00000168938 | PPIC |
| ENSG00000168955 | TM4SF20 |
| ENSG00000168959 | GRM5 |
| ENSG00000169006 | NTSR2 |
| ENSG00000169035 | KLK7 |
| ENSG00000169059 | VCX3A |
| ENSG00000169064 | ZBBX |
| ENSG00000169067 | ACTBL2 |
| ENSG00000169071 | ROR2 |
| ENSG00000169126 | ARMC4 |
| ENSG00000169154 | GOT1L1 |
| ENSG00000169169 | CPT1C |
| ENSG00000169181 | GSG1L |
| ENSG00000169194 | IL13 |
| ENSG00000169208 | OR10G3 |
| ENSG00000169213 | RAB3B |
| ENSG00000169214 | OR6F1 |
| ENSG00000169218 | RSPO1 |
| ENSG00000169248 | CXCL11 |
| ENSG00000169271 | HSPB3 |
| ENSG00000169282 | KCNAB1 |
| ENSG00000169297 | NR0B1 |
| ENSG00000169302 | STK32A |
| ENSG00000169306 | IL1RAPL1 |
| ENSG00000169314 | C22orf15 |
| ENSG00000169327 | OR5AU1 |
| ENSG00000169340 | PDILT |
| ENSG00000169344 | UMOD |
| ENSG00000169393 | ELSPBP1 |
| ENSG00000169402 | RSPH10B2 |
| ENSG00000169427 | KCNK9 |
| ENSG00000169435 | RASSF6 |
| ENSG00000169436 | COL22A1 |
| ENSG00000169469 | SPRR1B |
| ENSG00000169474 | SPRR1A |
| ENSG00000169484 | OR4K14 |
| ENSG00000169488 | OR4K15 |
| ENSG00000169495 | HTRA4 |
| ENSG00000169509 | CRCT1 |
| ENSG00000169515 | CCDC8 |
| ENSG00000169548 | ZNF280A |
| ENSG00000169550 | MUC15 |
| ENSG00000169551 | CT55 |
| ENSG00000169562 | GJB1 |
| ENSG00000169575 | VPREB1 |
| ENSG00000169594 | BNC1 |
| ENSG00000169605 | GKN1 |
| ENSG00000169618 | PROKR1 |
| ENSG00000169676 | DRD5 |
| ENSG00000169684 | CHRNA5 |
| ENSG00000169688 | MT1B |
| ENSG00000169717 | ACTRT2 |
| ENSG00000169758 | TMEM266 |
| ENSG00000169760 | NLGN1 |
| ENSG00000169777 | TAS2R1 |
| ENSG00000169783 | LINGO1 |
| ENSG00000169789 | PRY |
| ENSG00000169800 | RBMY1F |
| ENSG00000169807 | PRY2 |
| ENSG00000169836 | TACR3 |
| ENSG00000169840 | GSX1 |
| ENSG00000169851 | PCDH7 |
| ENSG00000169856 | ONECUT1 |
| ENSG00000169876 | MUC17 |
| ENSG00000169885 | CALML6 |
| ENSG00000169894 | MUC3A |
| ENSG00000169900 | PYDC1 |
| ENSG00000169903 | TM4SF4 |
| ENSG00000169906 | S100G |
| ENSG00000169933 | FRMPD4 |
| ENSG00000169953 | HSFY2 |
| ENSG00000169989 | TIGD4 |
| ENSG00000170044 | ZPLD1 |
| ENSG00000170054 | SERPINA9 |
| ENSG00000170099 | SERPINA6 |
| ENSG00000170122 | FOXD4 |
| ENSG00000170153 | RNF150 |
| ENSG00000170162 | VGLL2 |
| ENSG00000170166 | HOXD4 |
| ENSG00000170178 | HOXD12 |
| ENSG00000170214 | ADRA1B |
| ENSG00000170231 | FABP6 |
| ENSG00000170236 | USP50 |
| ENSG00000170255 | MRGPRX1 |
| ENSG00000170262 | MRAP |
| ENSG00000170264 | FAM161A |
| ENSG00000170276 | HSPB2 |
| ENSG00000170279 | C7orf33 |
| ENSG00000170289 | CNGB3 |
| ENSG00000170324 | FRMPD2 |
| ENSG00000170367 | CST5 |
| ENSG00000170369 | CST2 |
| ENSG00000170370 | EMX2 |
| ENSG00000170373 | CST1 |
| ENSG00000170374 | SP7 |
| ENSG00000170381 | SEMA3E |
| ENSG00000170419 | VSTM2A |
| ENSG00000170423 | KRT78 |
| ENSG00000170426 | SDR9C7 |
| ENSG00000170454 | KRT75 |
| ENSG00000170465 | KRT6C |
| ENSG00000170482 | SLC23A1 |
| ENSG00000170484 | KRT74 |
| ENSG00000170498 | KISS1 |
| ENSG00000170500 | LONRF2 |
| ENSG00000170516 | COX7B2 |
| ENSG00000170523 | KRT83 |
| ENSG00000170537 | TMC7 |
| ENSG00000170549 | IRX1 |
| ENSG00000170558 | CDH2 |
| ENSG00000170561 | IRX2 |
| ENSG00000170577 | SIX2 |
| ENSG00000170579 | DLGAP1 |
| ENSG00000170605 | OR9K2 |
| ENSG00000170608 | FOXA3 |
| ENSG00000170613 | FAM71B |
| ENSG00000170615 | SLC26A5 |
| ENSG00000170624 | SGCD |
| ENSG00000170647 | TMEM133 |
| ENSG00000170681 | MURC |
| ENSG00000170683 | OR10A3 |
| ENSG00000170689 | HOXB9 |
| ENSG00000170703 | TTLL6 |
| ENSG00000170743 | SYT9 |
| ENSG00000170745 | KCNS3 |
| ENSG00000170748 | RBMXL2 |
| ENSG00000170775 | GPR37 |
| ENSG00000170777 | TPD52L3 |
| ENSG00000170782 | OR10A4 |
| ENSG00000170786 | SDR16C5 |
| ENSG00000170788 | DYDC1 |
| ENSG00000170790 | OR10A2 |
| ENSG00000170807 | LMOD2 |
| ENSG00000170819 | BFSP2 |
| ENSG00000170820 | FSHR |
| ENSG00000170848 | PSG6 |
| ENSG00000170890 | PLA2G1B |
| ENSG00000170893 | TRH |
| ENSG00000170920 | OR7G3 |
| ENSG00000170923 | OR7G2 |
| ENSG00000170925 | TEX13B |
| ENSG00000170927 | PKHD1 |
| ENSG00000170929 | OR1M1 |
| ENSG00000170935 | NCBP2L |
| ENSG00000170948 | MBD3L1 |
| ENSG00000170950 | PGK2 |
| ENSG00000170953 | OR8B12 |
| ENSG00000170959 | DCDC1 |
| ENSG00000170961 | HAS2 |
| ENSG00000170965 | PLAC1 |
| ENSG00000170967 | DDI1 |
| ENSG00000171004 | HS6ST2 |
| ENSG00000171014 | OR4D5 |
| ENSG00000171016 | PYGO1 |
| ENSG00000171017 | LRRC8E |
| ENSG00000171053 | PATE1 |
| ENSG00000171054 | OR13H1 |
| ENSG00000171060 | C8orf74 |
| ENSG00000171094 | ALK |
| ENSG00000171102 | OBP2B |
| ENSG00000171116 | HSFX1 |
| ENSG00000171119 | NRTN |
| ENSG00000171124 | FUT3 |
| ENSG00000171126 | KCNG3 |
| ENSG00000171133 | OR2K2 |
| ENSG00000171136 | RLN3 |
| ENSG00000171160 | MORN4 |
| ENSG00000171180 | OR2M4 |
| ENSG00000171189 | GRIK1 |
| ENSG00000171199 | PROL1 |
| ENSG00000171201 | SMR3B |
| ENSG00000171209 | CSN3 |
| ENSG00000171217 | CLDN20 |
| ENSG00000171234 | UGT2B7 |
| ENSG00000171243 | SOSTDC1 |
| ENSG00000171246 | NPTX1 |
| ENSG00000171303 | KCNK3 |
| ENSG00000171357 | LURAP1 |
| ENSG00000171360 | KRT38 |
| ENSG00000171388 | APLN |
| ENSG00000171396 | KRTAP4-4 |
| ENSG00000171402 | XAGE3 |
| ENSG00000171403 | KRT9 |
| ENSG00000171405 | XAGE5 |
| ENSG00000171431 | KRT20 |
| ENSG00000171433 | GLOD5 |
| ENSG00000171435 | KSR2 |
| ENSG00000171446 | KRT27 |
| ENSG00000171450 | CDK5R2 |
| ENSG00000171459 | OR1L6 |
| ENSG00000171478 | SPACA5B |
| ENSG00000171481 | OR1L3 |
| ENSG00000171487 | NLRP5 |
| ENSG00000171489 | SPACA5 |
| ENSG00000171495 | MROH2B |
| ENSG00000171496 | OR1L8 |
| ENSG00000171501 | OR1N2 |
| ENSG00000171505 | OR1N1 |
| ENSG00000171509 | RXFP1 |
| ENSG00000171517 | LPAR3 |
| ENSG00000171532 | NEUROD2 |
| ENSG00000171533 | MAP6 |
| ENSG00000171540 | OTP |
| ENSG00000171551 | ECEL1 |
| ENSG00000171561 | OR2AT4 |
| ENSG00000171587 | DSCAM |
| ENSG00000171595 | DNAI2 |
| ENSG00000171695 | LKAAEAR1 |
| ENSG00000171711 | DEFB4A |
| ENSG00000171722 | C1orf111 |
| ENSG00000171724 | VAT1L |
| ENSG00000171759 | PAH |
| ENSG00000171772 | SYCE1 |
| ENSG00000171773 | NXNL1 |
| ENSG00000171790 | SLFNL1 |
| ENSG00000171794 | UTF1 |
| ENSG00000171804 | WDR87 |
| ENSG00000171811 | CFAP46 |
| ENSG00000171815 | PCDHB1 |
| ENSG00000171819 | ANGPTL7 |
| ENSG00000171855 | IFNB1 |
| ENSG00000171864 | PRND |
| ENSG00000171872 | KLF17 |
| ENSG00000171873 | ADRA1D |
| ENSG00000171877 | FRMD5 |
| ENSG00000171885 | AQP4 |
| ENSG00000171903 | CYP4F11 |
| ENSG00000171931 | FBXW10 |
| ENSG00000171936 | OR10H3 |
| ENSG00000171942 | OR10H2 |
| ENSG00000171944 | OR52A5 |
| ENSG00000171951 | SCG2 |
| ENSG00000171956 | FOXB1 |
| ENSG00000171987 | C11orf40 |
| ENSG00000171989 | LDHAL6B |
| ENSG00000172000 | ZNF556 |
| ENSG00000172014 | ANKRD20A4 |
| ENSG00000172016 | REG3A |
| ENSG00000172020 | GAP43 |
| ENSG00000172031 | EPHX4 |
| ENSG00000172058 | SERF1A |
| ENSG00000172061 | LRRC15 |
| ENSG00000172073 | TEX37 |
| ENSG00000172137 | CALB2 |
| ENSG00000172139 | SLC9C1 |
| ENSG00000172146 | OR1A1 |
| ENSG00000172150 | OR1A2 |
| ENSG00000172154 | OR8I2 |
| ENSG00000172155 | LCE1D |
| ENSG00000172156 | CCL11 |
| ENSG00000172188 | OR4C11 |
| ENSG00000172199 | OR8U1 |
| ENSG00000172201 | ID4 |
| ENSG00000172209 | GPR22 |
| ENSG00000172238 | ATOH1 |
| ENSG00000172288 | CDY1 |
| ENSG00000172289 | OR10V1 |
| ENSG00000172296 | SPTLC3 |
| ENSG00000172318 | B3GALT1 |
| ENSG00000172320 | OR5A1 |
| ENSG00000172324 | OR5A2 |
| ENSG00000172346 | CSDC2 |
| ENSG00000172350 | ABCG4 |
| ENSG00000172352 | CDY1B |
| ENSG00000172361 | CFAP53 |
| ENSG00000172362 | OR5B12 |
| ENSG00000172365 | OR5B2 |
| ENSG00000172377 | OR9I1 |
| ENSG00000172399 | MYOZ2 |
| ENSG00000172404 | DNAJB7 |
| ENSG00000172410 | INSL5 |
| ENSG00000172421 | EFCAB3 |
| ENSG00000172425 | TTC36 |
| ENSG00000172457 | OR9G4 |
| ENSG00000172458 | IL17D |
| ENSG00000172461 | FUT9 |
| ENSG00000172464 | OR5AP2 |
| ENSG00000172468 | HSFY1 |
| ENSG00000172476 | RAB40A |
| ENSG00000172478 | C2orf54 |
| ENSG00000172487 | OR8J1 |
| ENSG00000172489 | OR5T3 |
| ENSG00000172497 | ACOT12 |
| ENSG00000172519 | OR10H5 |
| ENSG00000172538 | FAM170B |
| ENSG00000172551 | MUCL1 |
| ENSG00000172554 | SNTG2 |
| ENSG00000172568 | FNDC9 |
| ENSG00000172640 | OR10AD1 |
| ENSG00000172680 | MOS |
| ENSG00000172689 | MS4A10 |
| ENSG00000172717 | FAM71D |
| ENSG00000172724 | CCL19 |
| ENSG00000172733 | PURG |
| ENSG00000172738 | TMEM217 |
| ENSG00000172742 | OR4D9 |
| ENSG00000172752 | COL6A5 |
| ENSG00000172769 | OR5B3 |
| ENSG00000172772 | OR10W1 |
| ENSG00000172782 | FADS6 |
| ENSG00000172789 | HOXC5 |
| ENSG00000172817 | CYP7B1 |
| ENSG00000172818 | OVOL1 |
| ENSG00000172828 | CES3 |
| ENSG00000172901 | LVRN |
| ENSG00000172935 | MRGPRF |
| ENSG00000172938 | MRGPRD |
| ENSG00000172940 | SLC22A13 |
| ENSG00000172969 | FRG2C |
| ENSG00000172986 | GXYLT2 |
| ENSG00000172987 | HPSE2 |
| ENSG00000172995 | ARPP21 |
| ENSG00000173077 | DEC1 |
| ENSG00000173080 | RXFP4 |
| ENSG00000173088 | C10orf131 |
| ENSG00000173093 | CCDC63 |
| ENSG00000173124 | ACSM6 |
| ENSG00000173156 | RHOD |
| ENSG00000173157 | ADAMTS20 |
| ENSG00000173227 | SYT12 |
| ENSG00000173237 | C11orf86 |
| ENSG00000173239 | LIPM |
| ENSG00000173250 | GPR151 |
| ENSG00000173253 | DMRT2 |
| ENSG00000173261 | PLAC8L1 |
| ENSG00000173285 | OR10K1 |
| ENSG00000173302 | GPR148 |
| ENSG00000173335 | CST9 |
| ENSG00000173349 | SFT2D3 |
| ENSG00000173376 | NDNF |
| ENSG00000173389 | IQCF1 |
| ENSG00000173401 | GLIPR1L1 |
| ENSG00000173404 | INSM1 |
| ENSG00000173406 | DAB1 |
| ENSG00000173431 | RNASE8 |
| ENSG00000173452 | TMEM196 |
| ENSG00000173464 | RNASE11 |
| ENSG00000173467 | AGR3 |
| ENSG00000173557 | C2orf70 |
| ENSG00000173572 | NLRP13 |
| ENSG00000173610 | UGT2A1 |
| ENSG00000173612 | GPRC6A |
| ENSG00000173627 | APOBEC4 |
| ENSG00000173662 | TAS1R1 |
| ENSG00000173673 | HES3 |
| ENSG00000173679 | OR1L1 |
| ENSG00000173698 | ADGRG2 |
| ENSG00000173699 | SPATA3 |
| ENSG00000173702 | MUC13 |
| ENSG00000173705 | SUSD5 |
| ENSG00000173714 | WFIKKN2 |
| ENSG00000173728 | C1orf100 |
| ENSG00000173769 | TOPAZ1 |
| ENSG00000173805 | HAP1 |
| ENSG00000173809 | TDRD12 |
| ENSG00000173826 | KCNH6 |
| ENSG00000173838 | MARCH10 |
| ENSG00000173908 | KRT28 |
| ENSG00000173947 | PIFO |
| ENSG00000173976 | RAX2 |
| ENSG00000173988 | LRRC63 |
| ENSG00000174015 | SPERT |
| ENSG00000174016 | FAM46D |
| ENSG00000174038 | C9orf131 |
| ENSG00000174145 | NWD2 |
| ENSG00000174156 | GSTA3 |
| ENSG00000174225 | ARL13A |
| ENSG00000174226 | SNX31 |
| ENSG00000174236 | REP15 |
| ENSG00000174279 | EVX2 |
| ENSG00000174325 | DIRC1 |
| ENSG00000174332 | GLIS1 |
| ENSG00000174339 | OR2Y1 |
| ENSG00000174343 | CHRNA9 |
| ENSG00000174370 | C11orf45 |
| ENSG00000174417 | TRHR |
| ENSG00000174429 | ABRA |
| ENSG00000174448 | STARD6 |
| ENSG00000174450 | GOLGA6L2 |
| ENSG00000174453 | VWC2L |
| ENSG00000174460 | ZCCHC12 |
| ENSG00000174473 | GALNTL6 |
| ENSG00000174498 | IGDCC3 |
| ENSG00000174502 | SLC26A9 |
| ENSG00000174521 | TTC9B |
| ENSG00000174527 | MYO1H |
| ENSG00000174562 | KLK15 |
| ENSG00000174567 | GOLT1A |
| ENSG00000174576 | NPAS4 |
| ENSG00000174599 | TRAM1L1 |
| ENSG00000174667 | OR7D4 |
| ENSG00000174672 | BRSK2 |
| ENSG00000174740 | PABPC5 |
| ENSG00000174792 | C4orf26 |
| ENSG00000174808 | BTC |
| ENSG00000174827 | PDZK1 |
| ENSG00000174844 | DNAH12 |
| ENSG00000174876 | AMY1B |
| ENSG00000174898 | CATSPERD |
| ENSG00000174899 | PQLC2L |
| ENSG00000174914 | OR9G1 |
| ENSG00000174937 | OR5M3 |
| ENSG00000174938 | SEZ6L2 |
| ENSG00000174939 | ASPHD1 |
| ENSG00000174945 | AMZ1 |
| ENSG00000174948 | GPR149 |
| ENSG00000174950 | CD164L2 |
| ENSG00000174957 | OR5J2 |
| ENSG00000174963 | ZIC4 |
| ENSG00000174970 | OR10AG1 |
| ENSG00000174982 | OR4S2 |
| ENSG00000174990 | CA5A |
| ENSG00000174992 | ZG16 |
| ENSG00000175018 | TEX36 |
| ENSG00000175065 | DSG4 |
| ENSG00000175077 | RTP1 |
| ENSG00000175093 | SPSB4 |
| ENSG00000175097 | RAG2 |
| ENSG00000175121 | WFDC5 |
| ENSG00000175143 | OR2T1 |
| ENSG00000175170 | FAM182B |
| ENSG00000175175 | PPM1E |
| ENSG00000175189 | INHBC |
| ENSG00000175202 | HIGD2B |
| ENSG00000175229 | GAL3ST3 |
| ENSG00000175262 | C1orf127 |
| ENSG00000175264 | CHST1 |
| ENSG00000175267 | VWA3A |
| ENSG00000175311 | ANKS4B |
| ENSG00000175315 | CST6 |
| ENSG00000175318 | GRAMD2 |
| ENSG00000175325 | PROP1 |
| ENSG00000175329 | ISX |
| ENSG00000175336 | APOF |
| ENSG00000175356 | SCUBE2 |
| ENSG00000175398 | OR10P1 |
| ENSG00000175426 | PCSK1 |
| ENSG00000175485 | OR52W1 |
| ENSG00000175497 | DPP10 |
| ENSG00000175513 | TSGA10IP |
| ENSG00000175514 | GPR152 |
| ENSG00000175520 | UBQLN3 |
| ENSG00000175600 | SUGCT |
| ENSG00000175619 | OR4B1 |
| ENSG00000175646 | PRM1 |
| ENSG00000175664 | TEX26 |
| ENSG00000175697 | GPR156 |
| ENSG00000175718 | RBMXL3 |
| ENSG00000175766 | EIF4E1B |
| ENSG00000175779 | C15orf53 |
| ENSG00000175785 | PRIMA1 |
| ENSG00000175809 | ZNF645 |
| ENSG00000175820 | CCDC168 |
| ENSG00000175832 | ETV4 |
| ENSG00000175868 | CALCB |
| ENSG00000175874 | CREG2 |
| ENSG00000175877 | WBSCR28 |
| ENSG00000175879 | HOXD8 |
| ENSG00000175894 | TSPEAR |
| ENSG00000175946 | KLHL38 |
| ENSG00000176009 | ASCL3 |
| ENSG00000176029 | C11orf16 |
| ENSG00000176040 | TMPRSS7 |
| ENSG00000176134 | AL445665.1 |
| ENSG00000176136 | MC5R |
| ENSG00000176165 | FOXG1 |
| ENSG00000176177 | ENTHD1 |
| ENSG00000176194 | CIDEA |
| ENSG00000176198 | OR11H4 |
| ENSG00000176200 | OR4D11 |
| ENSG00000176204 | LRRTM4 |
| ENSG00000176219 | OR11H6 |
| ENSG00000176230 | OR4K17 |
| ENSG00000176231 | OR10H4 |
| ENSG00000176239 | OR51B6 |
| ENSG00000176244 | ACBD7 |
| ENSG00000176246 | OR4L1 |
| ENSG00000176253 | OR4K13 |
| ENSG00000176256 | HMGB4 |
| ENSG00000176269 | OR4F21 |
| ENSG00000176281 | OR4K5 |
| ENSG00000176294 | OR4N2 |
| ENSG00000176299 | OR4M1 |
| ENSG00000176302 | FOXR1 |
| ENSG00000176358 | TAC4 |
| ENSG00000176381 | PRR18 |
| ENSG00000176387 | HSD11B2 |
| ENSG00000176399 | DMRTA1 |
| ENSG00000176402 | GJC3 |
| ENSG00000176406 | RIMS2 |
| ENSG00000176428 | VPS37D |
| ENSG00000176495 | OR5AN1 |
| ENSG00000176532 | PRR15 |
| ENSG00000176540 | OR4C5 |
| ENSG00000176547 | OR4C3 |
| ENSG00000176555 | OR4S1 |
| ENSG00000176566 | DCAF4L2 |
| ENSG00000176571 | CNBD1 |
| ENSG00000176601 | MAP3K19 |
| ENSG00000176605 | C14orf177 |
| ENSG00000176635 | HORMAD2 |
| ENSG00000176678 | FOXL1 |
| ENSG00000176679 | TGIF2LY |
| ENSG00000176692 | FOXC2 |
| ENSG00000176695 | OR4F17 |
| ENSG00000176697 | BDNF |
| ENSG00000176723 | ZNF843 |
| ENSG00000176732 | PFN4 |
| ENSG00000176742 | OR51V1 |
| ENSG00000176746 | MAGEB6 |
| ENSG00000176769 | TCERG1L |
| ENSG00000176771 | NCKAP5 |
| ENSG00000176774 | MAGEB18 |
| ENSG00000176782 | DEFB104A |
| ENSG00000176787 | OR52E2 |
| ENSG00000176797 | DEFB103A |
| ENSG00000176798 | OR51L1 |
| ENSG00000176842 | IRX5 |
| ENSG00000176884 | GRIN1 |
| ENSG00000176887 | SOX11 |
| ENSG00000176893 | OR51G2 |
| ENSG00000176895 | OR51A7 |
| ENSG00000176900 | OR51T1 |
| ENSG00000176909 | MAMSTR |
| ENSG00000176919 | C8G |
| ENSG00000176920 | FUT2 |
| ENSG00000176922 | OR51S1 |
| ENSG00000176925 | OR51F2 |
| ENSG00000176927 | EFCAB5 |
| ENSG00000176956 | LY6H |
| ENSG00000176971 | FIBIN |
| ENSG00000176979 | TRIM60 |
| ENSG00000176988 | FMR1NB |
| ENSG00000177023 | DEFB104B |
| ENSG00000177025 | C19orf18 |
| ENSG00000177047 | IFNW1 |
| ENSG00000177098 | SCN4B |
| ENSG00000177103 | DSCAML1 |
| ENSG00000177108 | ZDHHC22 |
| ENSG00000177138 | FAM9B |
| ENSG00000177143 | CETN1 |
| ENSG00000177151 | OR2T35 |
| ENSG00000177174 | OR14C36 |
| ENSG00000177181 | RIMKLA |
| ENSG00000177182 | CLVS1 |
| ENSG00000177186 | OR2M7 |
| ENSG00000177201 | OR2T12 |
| ENSG00000177202 | SPACA4 |
| ENSG00000177212 | OR2T33 |
| ENSG00000177238 | TRIM72 |
| ENSG00000177243 | DEFB103B |
| ENSG00000177257 | DEFB4B |
| ENSG00000177275 | OR2AJ1 |
| ENSG00000177283 | FZD8 |
| ENSG00000177291 | GJD4 |
| ENSG00000177294 | FBXO39 |
| ENSG00000177300 | CLDN22 |
| ENSG00000177354 | C10orf71 |
| ENSG00000177363 | LRRN4CL |
| ENSG00000177398 | UMODL1 |
| ENSG00000177414 | UBE2U |
| ENSG00000177453 | NIM1K |
| ENSG00000177459 | ERICH5 |
| ENSG00000177462 | OR2T8 |
| ENSG00000177468 | OLIG3 |
| ENSG00000177476 | OR2G3 |
| ENSG00000177489 | OR2G2 |
| ENSG00000177494 | ZBED2 |
| ENSG00000177504 | VCX2 |
| ENSG00000177511 | ST8SIA3 |
| ENSG00000177519 | RPRM |
| ENSG00000177535 | OR2B11 |
| ENSG00000177551 | NHLH2 |
| ENSG00000177558 | FAM187B |
| ENSG00000177614 | PGBD5 |
| ENSG00000177627 | C12orf54 |
| ENSG00000177673 | C2orf57 |
| ENSG00000177679 | SRRM3 |
| ENSG00000177684 | DEFB114 |
| ENSG00000177689 | MAGEB10 |
| ENSG00000177692 | DNAJC28 |
| ENSG00000177693 | OR4F4 |
| ENSG00000177694 | NAALADL2 |
| ENSG00000177710 | SLC35G5 |
| ENSG00000177752 | YIPF7 |
| ENSG00000177800 | TMEM78 |
| ENSG00000177807 | KCNJ10 |
| ENSG00000177839 | PCDHB9 |
| ENSG00000177875 | CCDC184 |
| ENSG00000177938 | CAPZA3 |
| ENSG00000177947 | ODF3 |
| ENSG00000177984 | LCN15 |
| ENSG00000177992 | SPATA31E1 |
| ENSG00000177994 | C2orf73 |
| ENSG00000178015 | GPR150 |
| ENSG00000178021 | TSPYL6 |
| ENSG00000178031 | ADAMTSL1 |
| ENSG00000178055 | PRSS42 |
| ENSG00000178084 | HTR3C |
| ENSG00000178115 | GOLGA8Q |
| ENSG00000178125 | PPP1R42 |
| ENSG00000178150 | ZNF114 |
| ENSG00000178171 | AMER3 |
| ENSG00000178172 | SPINK6 |
| ENSG00000178187 | ZNF454 |
| ENSG00000178201 | VN1R1 |
| ENSG00000178222 | RNF212 |
| ENSG00000178233 | TMEM151B |
| ENSG00000178235 | SLITRK1 |
| ENSG00000178257 | PRM3 |
| ENSG00000178279 | TNP2 |
| ENSG00000178287 | SPAG11A |
| ENSG00000178301 | AQP11 |
| ENSG00000178343 | SHISA3 |
| ENSG00000178358 | OR2D3 |
| ENSG00000178363 | CALML3 |
| ENSG00000178372 | CALML5 |
| ENSG00000178394 | HTR1A |
| ENSG00000178395 | CCDC185 |
| ENSG00000178401 | DNAJC22 |
| ENSG00000178403 | NEUROG2 |
| ENSG00000178460 | MCMDC2 |
| ENSG00000178462 | TUBAL3 |
| ENSG00000178473 | UCN3 |
| ENSG00000178522 | AMBN |
| ENSG00000178531 | CTXN1 |
| ENSG00000178568 | ERBB4 |
| ENSG00000178586 | OR6B3 |
| ENSG00000178591 | DEFB125 |
| ENSG00000178597 | PSAPL1 |
| ENSG00000178602 | OTOS |
| ENSG00000178645 | C10orf53 |
| ENSG00000178662 | CSRNP3 |
| ENSG00000178690 | DYNAP |
| ENSG00000178734 | LMO7DN |
| ENSG00000178750 | STX19 |
| ENSG00000178772 | CPN2 |
| ENSG00000178773 | CPNE7 |
| ENSG00000178776 | C5orf46 |
| ENSG00000178795 | GDPD4 |
| ENSG00000178796 | RIIAD1 |
| ENSG00000178804 | H1FOO |
| ENSG00000178821 | TMEM52 |
| ENSG00000178826 | TMEM139 |
| ENSG00000178828 | RNF186 |
| ENSG00000178882 | FAM101A |
| ENSG00000178919 | FOXE1 |
| ENSG00000178928 | TPRX1 |
| ENSG00000178934 | LGALS7B |
| ENSG00000178965 | ERICH3 |
| ENSG00000178997 | EXD1 |
| ENSG00000179002 | TAS1R2 |
| ENSG00000179008 | C14orf39 |
| ENSG00000179023 | KLHDC7A |
| ENSG00000179046 | TRIML2 |
| ENSG00000179055 | OR13D1 |
| ENSG00000179058 | C9orf50 |
| ENSG00000179059 | ZFP42 |
| ENSG00000179071 | CCDC89 |
| ENSG00000179083 | FAM133A |
| ENSG00000179097 | HTR1F |
| ENSG00000179111 | HES7 |
| ENSG00000179133 | C10orf67 |
| ENSG00000179142 | CYP11B2 |
| ENSG00000179148 | ALOXE3 |
| ENSG00000179172 | HNRNPCL1 |
| ENSG00000179178 | TMEM125 |
| ENSG00000179213 | SIGLECL1 |
| ENSG00000179256 | SMCO3 |
| ENSG00000179270 | C2orf71 |
| ENSG00000179284 | DAND5 |
| ENSG00000179292 | TMEM151A |
| ENSG00000179300 | ZCCHC5 |
| ENSG00000179363 | TMEM31 |
| ENSG00000179397 | C1orf101 |
| ENSG00000179399 | GPC5 |
| ENSG00000179407 | DNAJB8 |
| ENSG00000179412 | HNRNPCL4 |
| ENSG00000179431 | FJX1 |
| ENSG00000179455 | MKRN3 |
| ENSG00000179468 | OR9A2 |
| ENSG00000179477 | ALOX12B |
| ENSG00000179520 | SLC17A8 |
| ENSG00000179528 | LBX2 |
| ENSG00000179546 | HTR1D |
| ENSG00000179564 | LSMEM2 |
| ENSG00000179580 | RNF151 |
| ENSG00000179600 | GPHB5 |
| ENSG00000179603 | GRM8 |
| ENSG00000179615 | OR2AP1 |
| ENSG00000179626 | OR6C4 |
| ENSG00000179636 | TPPP2 |
| ENSG00000179673 | RPRML |
| ENSG00000179674 | ARL14 |
| ENSG00000179695 | OR6C2 |
| ENSG00000179709 | NLRP8 |
| ENSG00000179751 | SYCN |
| ENSG00000179772 | FOXS1 |
| ENSG00000179774 | ATOH7 |
| ENSG00000179796 | LRRC3B |
| ENSG00000179813 | FAM216B |
| ENSG00000179817 | MRGPRX4 |
| ENSG00000179826 | MRGPRX3 |
| ENSG00000179846 | NKPD1 |
| ENSG00000179873 | NLRP11 |
| ENSG00000179902 | C1orf194 |
| ENSG00000179913 | B3GNT3 |
| ENSG00000179915 | NRXN1 |
| ENSG00000179919 | OR10A7 |
| ENSG00000179930 | ZNF648 |
| ENSG00000179934 | CCR8 |
| ENSG00000179938 | GOLGA8J |
| ENSG00000180016 | OR1E1 |
| ENSG00000180043 | FAM71E2 |
| ENSG00000180044 | C3orf80 |
| ENSG00000180053 | NKX2-6 |
| ENSG00000180071 | ANKRD18A |
| ENSG00000180083 | WFDC11 |
| ENSG00000180090 | OR3A1 |
| ENSG00000180113 | TDRD6 |
| ENSG00000180116 | C12orf40 |
| ENSG00000180176 | TH |
| ENSG00000180205 | WFDC9 |
| ENSG00000180210 | F2 |
| ENSG00000180219 | FAM71C |
| ENSG00000180245 | RRH |
| ENSG00000180251 | SLC9A4 |
| ENSG00000180264 | ADGRD2 |
| ENSG00000180269 | GPR139 |
| ENSG00000180287 | PLD5 |
| ENSG00000180305 | WFDC10A |
| ENSG00000180318 | ALX1 |
| ENSG00000180332 | KCTD4 |
| ENSG00000180336 | C17orf104 |
| ENSG00000180347 | CCDC129 |
| ENSG00000180383 | DEFB124 |
| ENSG00000180386 | KRTAP9-7 |
| ENSG00000180424 | DEFB123 |
| ENSG00000180432 | CYP8B1 |
| ENSG00000180433 | OR6K6 |
| ENSG00000180440 | SERTM1 |
| ENSG00000180475 | OR10Q1 |
| ENSG00000180481 | GLIPR1L2 |
| ENSG00000180483 | DEFB119 |
| ENSG00000180525 | PRR26 |
| ENSG00000180532 | ZSCAN4 |
| ENSG00000180535 | BHLHA15 |
| ENSG00000180592 | SKIDA1 |
| ENSG00000180613 | GSX2 |
| ENSG00000180616 | SSTR2 |
| ENSG00000180638 | SLC47A2 |
| ENSG00000180658 | OR2A4 |
| ENSG00000180660 | MAB21L1 |
| ENSG00000180697 | C3orf22 |
| ENSG00000180708 | OR10K2 |
| ENSG00000180720 | CHRM4 |
| ENSG00000180730 | SHISA2 |
| ENSG00000180745 | CLRN3 |
| ENSG00000180772 | AGTR2 |
| ENSG00000180777 | ANKRD30B |
| ENSG00000180785 | OR51E1 |
| ENSG00000180801 | ARSJ |
| ENSG00000180806 | HOXC9 |
| ENSG00000180818 | HOXC10 |
| ENSG00000180828 | BHLHE22 |
| ENSG00000180872 | DEFB112 |
| ENSG00000180875 | GREM2 |
| ENSG00000180878 | C11orf42 |
| ENSG00000180919 | OR56B4 |
| ENSG00000180929 | GPR62 |
| ENSG00000180934 | OR56A1 |
| ENSG00000180974 | OR52E4 |
| ENSG00000180988 | OR52N2 |
| ENSG00000180998 | GPR137C |
| ENSG00000180999 | C1orf105 |
| ENSG00000181001 | OR52N1 |
| ENSG00000181009 | OR52N5 |
| ENSG00000181013 | C17orf47 |
| ENSG00000181023 | OR56B1 |
| ENSG00000181072 | CHRM2 |
| ENSG00000181074 | OR52N4 |
| ENSG00000181085 | MAPK15 |
| ENSG00000181092 | ADIPOQ |
| ENSG00000181195 | PENK |
| ENSG00000181215 | C4orf50 |
| ENSG00000181234 | TMEM132C |
| ENSG00000181240 | SLC25A41 |
| ENSG00000181264 | TMEM136 |
| ENSG00000181273 | OR5AK2 |
| ENSG00000181291 | TMEM132E |
| ENSG00000181322 | NME9 |
| ENSG00000181323 | SPEM1 |
| ENSG00000181333 | HEPHL1 |
| ENSG00000181355 | OFCC1 |
| ENSG00000181371 | OR5M8 |
| ENSG00000181374 | CCL13 |
| ENSG00000181378 | CCDC108 |
| ENSG00000181408 | UTS2R |
| ENSG00000181433 | SAGE1 |
| ENSG00000181449 | SOX2 |
| ENSG00000181499 | OR6T1 |
| ENSG00000181518 | OR8D4 |
| ENSG00000181541 | MAB21L2 |
| ENSG00000181552 | EDDM3B |
| ENSG00000181562 | EDDM3A |
| ENSG00000181577 | C6orf223 |
| ENSG00000181585 | TMIE |
| ENSG00000181609 | OR52D1 |
| ENSG00000181616 | OR52H1 |
| ENSG00000181617 | FDCSP |
| ENSG00000181626 | ANKRD62 |
| ENSG00000181656 | GPR88 |
| ENSG00000181693 | OR8H1 |
| ENSG00000181698 | OR5T1 |
| ENSG00000181718 | OR5T2 |
| ENSG00000181733 | OR2Z1 |
| ENSG00000181752 | OR8K5 |
| ENSG00000181761 | OR8H3 |
| ENSG00000181767 | OR8H2 |
| ENSG00000181773 | GPR3 |
| ENSG00000181778 | TMEM252 |
| ENSG00000181781 | ODF3L2 |
| ENSG00000181785 | OR5AS1 |
| ENSG00000181786 | ACTL9 |
| ENSG00000181803 | OR6S1 |
| ENSG00000181867 | FTMT |
| ENSG00000181903 | OR4C6 |
| ENSG00000181927 | OR4P4 |
| ENSG00000181939 | OR4C15 |
| ENSG00000181958 | OR4A15 |
| ENSG00000181961 | OR4A16 |
| ENSG00000181963 | OR52K2 |
| ENSG00000181965 | NEUROG1 |
| ENSG00000182013 | PNMAL1 |
| ENSG00000182035 | ADIG |
| ENSG00000182040 | USH1G |
| ENSG00000182050 | MGAT4C |
| ENSG00000182053 | TRIM49B |
| ENSG00000182070 | OR52A1 |
| ENSG00000182077 | PTCHD3 |
| ENSG00000182083 | OR6B2 |
| ENSG00000182103 | FAM181B |
| ENSG00000182111 | ZNF716 |
| ENSG00000182132 | KCNIP1 |
| ENSG00000182156 | ENPP7 |
| ENSG00000182168 | UNC5C |
| ENSG00000182170 | MRGPRG |
| ENSG00000182177 | ASB18 |
| ENSG00000182187 | CRYGB |
| ENSG00000182218 | HHIPL1 |
| ENSG00000182223 | ZAR1 |
| ENSG00000182255 | KCNA4 |
| ENSG00000182256 | GABRG3 |
| ENSG00000182261 | NLRP10 |
| ENSG00000182263 | FIGN |
| ENSG00000182264 | IZUMO1 |
| ENSG00000182271 | TMIGD1 |
| ENSG00000182315 | MBD3L3 |
| ENSG00000182327 | GLTPD2 |
| ENSG00000182329 | KIAA2012 |
| ENSG00000182330 | PRAMEF8 |
| ENSG00000182334 | OR5P3 |
| ENSG00000182346 | DAOA |
| ENSG00000182348 | ZNF804B |
| ENSG00000182393 | IFNL1 |
| ENSG00000182415 | CDY2A |
| ENSG00000182447 | OTOL1 |
| ENSG00000182450 | KCNK4 |
| ENSG00000182459 | TEX19 |
| ENSG00000182489 | XKRX |
| ENSG00000182508 | LHFPL1 |
| ENSG00000182521 | TBPL2 |
| ENSG00000182533 | CAV3 |
| ENSG00000182545 | RNASE10 |
| ENSG00000182575 | NXPH3 |
| ENSG00000182583 | VCX |
| ENSG00000182585 | EPGN |
| ENSG00000182591 | KRTAP11-1 |
| ENSG00000182601 | HS3ST4 |
| ENSG00000182612 | TSPAN10 |
| ENSG00000182613 | OR2V2 |
| ENSG00000182631 | RXFP3 |
| ENSG00000182634 | OR10G7 |
| ENSG00000182645 | CCDC172 |
| ENSG00000182652 | OR4Q3 |
| ENSG00000182667 | NTM |
| ENSG00000182674 | KCNB2 |
| ENSG00000182676 | PPP1R27 |
| ENSG00000182687 | GALR2 |
| ENSG00000182698 | RESP18 |
| ENSG00000182747 | SLC35D3 |
| ENSG00000182752 | PAPPA |
| ENSG00000182759 | MAFA |
| ENSG00000182771 | GRID1 |
| ENSG00000182783 | OR2T29 |
| ENSG00000182791 | CCDC87 |
| ENSG00000182793 | GSTA5 |
| ENSG00000182798 | MAGEB17 |
| ENSG00000182816 | KRTAP13-2 |
| ENSG00000182836 | PLCXD3 |
| ENSG00000182854 | OR4F15 |
| ENSG00000182870 | GALNT9 |
| ENSG00000182896 | TMEM95 |
| ENSG00000182898 | TCHHL1 |
| ENSG00000182901 | RGS7 |
| ENSG00000182916 | TCEAL7 |
| ENSG00000182931 | WFDC10B |
| ENSG00000182938 | OTOP3 |
| ENSG00000182950 | ODF3L1 |
| ENSG00000182963 | GJC1 |
| ENSG00000182968 | SOX1 |
| ENSG00000182974 | RP11-294C11.1 |
| ENSG00000183024 | OR1G1 |
| ENSG00000183032 | SLC25A21 |
| ENSG00000183034 | OTOP2 |
| ENSG00000183035 | CYLC1 |
| ENSG00000183036 | PCP4 |
| ENSG00000183066 | WBP2NL |
| ENSG00000183067 | IGSF5 |
| ENSG00000183072 | NKX2-5 |
| ENSG00000183090 | FREM3 |
| ENSG00000183098 | GPC6 |
| ENSG00000183114 | FAM43B |
| ENSG00000183117 | CSMD1 |
| ENSG00000183128 | CALHM3 |
| ENSG00000183145 | RIPPLY3 |
| ENSG00000183146 | PRORY |
| ENSG00000183148 | ANKRD20A2 |
| ENSG00000183153 | GJD3 |
| ENSG00000183166 | CALN1 |
| ENSG00000183185 | GABRR3 |
| ENSG00000183186 | C2CD4C |
| ENSG00000183196 | CHST6 |
| ENSG00000183206 | POTEC |
| ENSG00000183230 | CTNNA3 |
| ENSG00000183246 | RIMBP3C |
| ENSG00000183248 | PRR36 |
| ENSG00000183251 | OR51B4 |
| ENSG00000183269 | OR52E8 |
| ENSG00000183273 | CCDC60 |
| ENSG00000183287 | CCBE1 |
| ENSG00000183292 | TISP43 |
| ENSG00000183303 | OR5P2 |
| ENSG00000183304 | FAM9A |
| ENSG00000183305 | MAGEA2B |
| ENSG00000183310 | OR2T34 |
| ENSG00000183313 | OR52L1 |
| ENSG00000183317 | EPHA10 |
| ENSG00000183318 | SPDYE4 |
| ENSG00000183324 | REC114 |
| ENSG00000183346 | C10orf107 |
| ENSG00000183378 | OVCH2 |
| ENSG00000183379 | SYNDIG1L |
| ENSG00000183389 | OR56A4 |
| ENSG00000183395 | PMCH |
| ENSG00000183396 | TMEM89 |
| ENSG00000183423 | LRIT3 |
| ENSG00000183434 | TFDP3 |
| ENSG00000183439 | TRIM61 |
| ENSG00000183454 | GRIN2A |
| ENSG00000183463 | URAD |
| ENSG00000183476 | SH2D7 |
| ENSG00000183549 | ACSM5 |
| ENSG00000183559 | C10orf120 |
| ENSG00000183560 | IZUMO1R |
| ENSG00000183571 | PGPEP1L |
| ENSG00000183607 | GKN2 |
| ENSG00000183629 | GOLGA8G |
| ENSG00000183631 | PRR32 |
| ENSG00000183632 | TP53TG3 |
| ENSG00000183638 | RP1L1 |
| ENSG00000183640 | KRTAP8-1 |
| ENSG00000183644 | C11orf88 |
| ENSG00000183654 | MARCH11 |
| ENSG00000183662 | FAM19A1 |
| ENSG00000183668 | PSG9 |
| ENSG00000183671 | GPR1 |
| ENSG00000183682 | BMP8A |
| ENSG00000183695 | MRGPRX2 |
| ENSG00000183706 | OR4N4 |
| ENSG00000183709 | IFNL2 |
| ENSG00000183715 | OPCML |
| ENSG00000183729 | NPBWR1 |
| ENSG00000183733 | FIGLA |
| ENSG00000183747 | ACSM2A |
| ENSG00000183753 | BPY2 |
| ENSG00000183760 | PAPL |
| ENSG00000183770 | FOXL2 |
| ENSG00000183775 | KCTD16 |
| ENSG00000183778 | B3GALT5 |
| ENSG00000183780 | SLC35F3 |
| ENSG00000183783 | KCTD8 |
| ENSG00000183791 | TCEB3C |
| ENSG00000183795 | BPY2B |
| ENSG00000183798 | EMILIN3 |
| ENSG00000183801 | OLFML1 |
| ENSG00000183807 | FAM162B |
| ENSG00000183831 | ANKRD45 |
| ENSG00000183833 | MAATS1 |
| ENSG00000183840 | GPR39 |
| ENSG00000183850 | ZNF730 |
| ENSG00000183853 | KIRREL |
| ENSG00000183862 | CNGA2 |
| ENSG00000183876 | ARSI |
| ENSG00000183888 | C1orf64 |
| ENSG00000183908 | LRRC55 |
| ENSG00000183914 | DNAH2 |
| ENSG00000183921 | SDR42E2 |
| ENSG00000183971 | NPW |
| ENSG00000183977 | PP2D1 |
| ENSG00000183979 | NPB |
| ENSG00000184012 | TMPRSS2 |
| ENSG00000184022 | OR2T10 |
| ENSG00000184029 | DSCR4 |
| ENSG00000184032 | KRTAP20-2 |
| ENSG00000184033 | CTAG1B |
| ENSG00000184058 | TBX1 |
| ENSG00000184108 | TRIML1 |
| ENSG00000184140 | OR4F6 |
| ENSG00000184144 | CNTN2 |
| ENSG00000184148 | SPRR4 |
| ENSG00000184155 | OR10J5 |
| ENSG00000184156 | KCNQ3 |
| ENSG00000184160 | ADRA2C |
| ENSG00000184166 | OR1D2 |
| ENSG00000184185 | KCNJ12 |
| ENSG00000184194 | GPR173 |
| ENSG00000184210 | DGAT2L6 |
| ENSG00000184254 | ALDH1A3 |
| ENSG00000184258 | CDR1 |
| ENSG00000184261 | KCNK12 |
| ENSG00000184276 | DEFB108B |
| ENSG00000184302 | SIX6 |
| ENSG00000184304 | PRKD1 |
| ENSG00000184330 | S100A7A |
| ENSG00000184344 | GDF3 |
| ENSG00000184345 | IQCF2 |
| ENSG00000184350 | MRGPRE |
| ENSG00000184351 | KRTAP19-1 |
| ENSG00000184361 | SPATA32 |
| ENSG00000184363 | PKP3 |
| ENSG00000184368 | MAP7D2 |
| ENSG00000184374 | COLEC10 |
| ENSG00000184388 | PABPC1L2B |
| ENSG00000184389 | A3GALT2 |
| ENSG00000184394 | OR4N5 |
| ENSG00000184408 | KCND2 |
| ENSG00000184434 | LRRC19 |
| ENSG00000184454 | NCMAP |
| ENSG00000184459 | BPIFC |
| ENSG00000184471 | C1QTNF8 |
| ENSG00000184478 | OR56A3 |
| ENSG00000184486 | POU3F2 |
| ENSG00000184492 | FOXD4L1 |
| ENSG00000184502 | GAST |
| ENSG00000184507 | NUTM1 |
| ENSG00000184524 | CEND1 |
| ENSG00000184530 | C6orf58 |
| ENSG00000184544 | DHRS7C |
| ENSG00000184560 | C17orf74 |
| ENSG00000184564 | SLITRK6 |
| ENSG00000184571 | PIWIL3 |
| ENSG00000184599 | FAM19A3 |
| ENSG00000184601 | C14orf180 |
| ENSG00000184647 | PRSS55 |
| ENSG00000184650 | ODF4 |
| ENSG00000184672 | RALYL |
| ENSG00000184697 | CLDN6 |
| ENSG00000184698 | OR51M1 |
| ENSG00000184709 | LRRC26 |
| ENSG00000184716 | SERINC4 |
| ENSG00000184724 | KRTAP6-1 |
| ENSG00000184735 | DDX53 |
| ENSG00000184761 | AC013269.5 |
| ENSG00000184788 | SATL1 |
| ENSG00000184811 | TUSC5 |
| ENSG00000184814 | PRR23B |
| ENSG00000184828 | ZBTB7C |
| ENSG00000184845 | DRD1 |
| ENSG00000184895 | SRY |
| ENSG00000184908 | CLCNKB |
| ENSG00000184911 | DMRTC1B |
| ENSG00000184933 | OR6A2 |
| ENSG00000184937 | WT1 |
| ENSG00000184945 | AQP12A |
| ENSG00000184949 | FAM227A |
| ENSG00000184954 | OR6C70 |
| ENSG00000184984 | CHRM5 |
| ENSG00000184995 | IFNE |
| ENSG00000184999 | SLC22A10 |
| ENSG00000185002 | RFX6 |
| ENSG00000185008 | ROBO2 |
| ENSG00000185013 | NT5C1B |
| ENSG00000185028 | LRRC14B |
| ENSG00000185038 | MROH2A |
| ENSG00000185046 | ANKS1B |
| ENSG00000185053 | SGCZ |
| ENSG00000185055 | EFCAB10 |
| ENSG00000185056 | C5orf47 |
| ENSG00000185069 | KRT76 |
| ENSG00000185070 | FLRT2 |
| ENSG00000185087 | FAM169B |
| ENSG00000185105 | MYADML2 |
| ENSG00000185133 | INPP5J |
| ENSG00000185149 | NPY2R |
| ENSG00000185155 | MIXL1 |
| ENSG00000185176 | AQP12B |
| ENSG00000185177 | ZNF479 |
| ENSG00000185231 | MC2R |
| ENSG00000185247 | MAGEA11 |
| ENSG00000185264 | TEX33 |
| ENSG00000185267 | CDNF |
| ENSG00000185269 | NOTUM |
| ENSG00000185271 | KLHL33 |
| ENSG00000185274 | WBSCR17 |
| ENSG00000185290 | NUPR1L |
| ENSG00000185294 | SPPL2C |
| ENSG00000185306 | C12orf56 |
| ENSG00000185313 | SCN10A |
| ENSG00000185332 | TMEM105 |
| ENSG00000185345 | PARK2 |
| ENSG00000185352 | HS6ST3 |
| ENSG00000185372 | OR2V1 |
| ENSG00000185385 | OR7A17 |
| ENSG00000185448 | FAM47A |
| ENSG00000185467 | KPNA7 |
| ENSG00000185479 | KRT6B |
| ENSG00000185483 | ROR1 |
| ENSG00000185518 | SV2B |
| ENSG00000185519 | FAM131C |
| ENSG00000185523 | SPATA45 |
| ENSG00000185565 | LSAMP |
| ENSG00000185594 | SPATA8 |
| ENSG00000185610 | DBX2 |
| ENSG00000185615 | PDIA2 |
| ENSG00000185634 | SHC4 |
| ENSG00000185640 | KRT79 |
| ENSG00000185652 | NTF3 |
| ENSG00000185662 | SMIM23 |
| ENSG00000185666 | SYN3 |
| ENSG00000185668 | POU3F1 |
| ENSG00000185674 | LYG2 |
| ENSG00000185681 | MORN5 |
| ENSG00000185686 | PRAME |
| ENSG00000185689 | C6orf201 |
| ENSG00000185737 | NRG3 |
| ENSG00000185742 | C11orf87 |
| ENSG00000185758 | CLDN24 |
| ENSG00000185774 | KCNIP4 |
| ENSG00000185775 | SPATA31A6 |
| ENSG00000185792 | NLRP9 |
| ENSG00000185818 | NAT8L |
| ENSG00000185821 | OR6C76 |
| ENSG00000185823 | NPAP1 |
| ENSG00000185860 | CCDC190 |
| ENSG00000185863 | TMEM210 |
| ENSG00000185873 | TMPRSS11B |
| ENSG00000185888 | PRSS38 |
| ENSG00000185894 | BPY2C |
| ENSG00000185899 | TAS2R60 |
| ENSG00000185915 | KLHL34 |
| ENSG00000185924 | RTN4RL1 |
| ENSG00000185926 | OR4C46 |
| ENSG00000185933 | CALHM1 |
| ENSG00000185940 | KRTAP5-5 |
| ENSG00000185942 | NKAIN3 |
| ENSG00000185958 | FAM186A |
| ENSG00000185960 | SHOX |
| ENSG00000185962 | LCE3A |
| ENSG00000185966 | LCE3E |
| ENSG00000185972 | CCIN |
| ENSG00000185974 | GRK1 |
| ENSG00000185982 | DEFB128 |
| ENSG00000185985 | SLITRK2 |
| ENSG00000185988 | PLK5 |
| ENSG00000186007 | LEMD1 |
| ENSG00000186009 | ATP4B |
| ENSG00000186038 | HTR3E |
| ENSG00000186051 | TAL2 |
| ENSG00000186075 | ZPBP2 |
| ENSG00000186086 | NBPF6 |
| ENSG00000186090 | HTR3D |
| ENSG00000186092 | OR4F5 |
| ENSG00000186094 | AGBL4 |
| ENSG00000186103 | ARGFX |
| ENSG00000186113 | OR5D14 |
| ENSG00000186115 | CYP4F2 |
| ENSG00000186118 | TEX38 |
| ENSG00000186119 | OR5D18 |
| ENSG00000186136 | TAS2R42 |
| ENSG00000186143 | PRR30 |
| ENSG00000186146 | DEFB131 |
| ENSG00000186150 | UBL4B |
| ENSG00000186160 | CYP4Z1 |
| ENSG00000186188 | FFAR4 |
| ENSG00000186190 | BPIFB3 |
| ENSG00000186191 | BPIFB4 |
| ENSG00000186198 | SLC51B |
| ENSG00000186207 | LCE5A |
| ENSG00000186212 | SOWAHB |
| ENSG00000186226 | LCE1E |
| ENSG00000186231 | KLHL32 |
| ENSG00000186280 | KDM4D |
| ENSG00000186288 | PABPC1L2A |
| ENSG00000186297 | GABRA5 |
| ENSG00000186306 | OR10T2 |
| ENSG00000186326 | RGS9BP |
| ENSG00000186329 | TMEM212 |
| ENSG00000186334 | SLC36A3 |
| ENSG00000186335 | SLC36A2 |
| ENSG00000186367 | KIAA1024L |
| ENSG00000186377 | CYP4X1 |
| ENSG00000186393 | KRT26 |
| ENSG00000186439 | TRDN |
| ENSG00000186440 | OR6P1 |
| ENSG00000186442 | KRT3 |
| ENSG00000186451 | SPATA12 |
| ENSG00000186452 | TMPRSS12 |
| ENSG00000186453 | FAM228A |
| ENSG00000186458 | DEFB132 |
| ENSG00000186471 | AKAP14 |
| ENSG00000186472 | PCLO |
| ENSG00000186474 | KLK12 |
| ENSG00000186479 | RGS7BP |
| ENSG00000186487 | MYT1L |
| ENSG00000186493 | C5orf38 |
| ENSG00000186509 | OR9Q1 |
| ENSG00000186510 | CLCNKA |
| ENSG00000186513 | OR9Q2 |
| ENSG00000186526 | CYP4F8 |
| ENSG00000186562 | DEFB105A |
| ENSG00000186572 | DEFB107A |
| ENSG00000186579 | DEFB106A |
| ENSG00000186599 | DEFB105B |
| ENSG00000186603 | HPDL |
| ENSG00000186628 | FSD2 |
| ENSG00000186675 | MAGEE2 |
| ENSG00000186684 | CYP27C1 |
| ENSG00000186710 | CFAP73 |
| ENSG00000186714 | CCDC73 |
| ENSG00000186723 | OR10H1 |
| ENSG00000186732 | MPPED1 |
| ENSG00000186765 | FSCN2 |
| ENSG00000186766 | FOXI2 |
| ENSG00000186777 | ZNF732 |
| ENSG00000186788 | SPATA31D3 |
| ENSG00000186790 | FOXE3 |
| ENSG00000186795 | KCNK18 |
| ENSG00000186803 | IFNA10 |
| ENSG00000186832 | KRT16 |
| ENSG00000186838 | SELV |
| ENSG00000186844 | LCE1A |
| ENSG00000186860 | KRTAP17-1 |
| ENSG00000186867 | QRFPR |
| ENSG00000186881 | OR13F1 |
| ENSG00000186889 | TMEM17 |
| ENSG00000186895 | FGF3 |
| ENSG00000186897 | C1QL4 |
| ENSG00000186910 | SERPINA11 |
| ENSG00000186912 | P2RY4 |
| ENSG00000186924 | KRTAP22-1 |
| ENSG00000186925 | KRTAP19-6 |
| ENSG00000186930 | KRTAP6-2 |
| ENSG00000186943 | OR13C8 |
| ENSG00000186952 | TMEM232 |
| ENSG00000186965 | KRTAP19-2 |
| ENSG00000186967 | KRTAP19-4 |
| ENSG00000186970 | KRTAP15-1 |
| ENSG00000186971 | KRTAP13-4 |
| ENSG00000186973 | FAM183A |
| ENSG00000186976 | EFCAB6 |
| ENSG00000186977 | KRTAP19-5 |
| ENSG00000186980 | KRTAP23-1 |
| ENSG00000187003 | ACTL7A |
| ENSG00000187005 | KRTAP21-1 |
| ENSG00000187021 | PNLIPRP1 |
| ENSG00000187026 | KRTAP21-2 |
| ENSG00000187033 | SAMD7 |
| ENSG00000187048 | CYP4A11 |
| ENSG00000187054 | TMPRSS11A |
| ENSG00000187068 | C3orf70 |
| ENSG00000187080 | OR2AK2 |
| ENSG00000187082 | DEFB106B |
| ENSG00000187094 | CCK |
| ENSG00000187105 | HEATR4 |
| ENSG00000187123 | LYPD6 |
| ENSG00000187135 | VSTM2B |
| ENSG00000187140 | FOXD3 |
| ENSG00000187144 | SPATA21 |
| ENSG00000187151 | ANGPTL5 |
| ENSG00000187166 | H1FNT |
| ENSG00000187170 | LCE4A |
| ENSG00000187173 | LCE2A |
| ENSG00000187175 | KRTAP12-1 |
| ENSG00000187180 | LCE2C |
| ENSG00000187186 | RP11-195F19.5 |
| ENSG00000187191 | DAZ3 |
| ENSG00000187223 | LCE2D |
| ENSG00000187238 | LCE3B |
| ENSG00000187242 | KRT12 |
| ENSG00000187258 | NPSR1 |
| ENSG00000187268 | FAM9C |
| ENSG00000187272 | KRTAP9-8 |
| ENSG00000187288 | CIDEC |
| ENSG00000187323 | DCC |
| ENSG00000187372 | PCDHB13 |
| ENSG00000187398 | LUZP2 |
| ENSG00000187416 | LHFPL3 |
| ENSG00000187475 | HIST1H1T |
| ENSG00000187486 | KCNJ11 |
| ENSG00000187492 | CDHR4 |
| ENSG00000187510 | PLEKHG7 |
| ENSG00000187516 | HYPM |
| ENSG00000187527 | ATP13A5 |
| ENSG00000187533 | PRR27 |
| ENSG00000187537 | POTEM |
| ENSG00000187545 | PRAMEF10 |
| ENSG00000187546 | AGMO |
| ENSG00000187550 | SBK2 |
| ENSG00000187553 | CYP26C1 |
| ENSG00000187556 | NANOS3 |
| ENSG00000187559 | FOXD4L3 |
| ENSG00000187566 | NHLRC1 |
| ENSG00000187569 | DPPA3 |
| ENSG00000187581 | COX8C |
| ENSG00000187600 | TMEM247 |
| ENSG00000187612 | OR5W2 |
| ENSG00000187616 | TMEM8C |
| ENSG00000187634 | SAMD11 |
| ENSG00000187658 | C5orf52 |
| ENSG00000187664 | HAPLN4 |
| ENSG00000187672 | ERC2 |
| ENSG00000187682 | ERAS |
| ENSG00000187689 | AMTN |
| ENSG00000187690 | CXorf67 |
| ENSG00000187701 | OR2T27 |
| ENSG00000187714 | SLC18A3 |
| ENSG00000187715 | KBTBD12 |
| ENSG00000187720 | THSD4 |
| ENSG00000187726 | DNAJB13 |
| ENSG00000187730 | GABRD |
| ENSG00000187733 | AMY1C |
| ENSG00000187747 | OR52B6 |
| ENSG00000187753 | C9orf153 |
| ENSG00000187754 | SSX7 |
| ENSG00000187766 | KRTAP10-8 |
| ENSG00000187772 | LIN28B |
| ENSG00000187773 | FAM69C |
| ENSG00000187783 | TMEM72 |
| ENSG00000187791 | FAM205C |
| ENSG00000187806 | TMEM202 |
| ENSG00000187821 | HELT |
| ENSG00000187823 | ZCCHC16 |
| ENSG00000187833 | C2orf78 |
| ENSG00000187848 | P2RX2 |
| ENSG00000187855 | ASCL4 |
| ENSG00000187857 | OR6C75 |
| ENSG00000187867 | PALM3 |
| ENSG00000187871 | GFRAL |
| ENSG00000187889 | C1orf168 |
| ENSG00000187905 | LRRC74B |
| ENSG00000187908 | DMBT1 |
| ENSG00000187918 | OR51I2 |
| ENSG00000187944 | C2orf66 |
| ENSG00000187950 | OVCH1 |
| ENSG00000187957 | DNER |
| ENSG00000187959 | CPSF4L |
| ENSG00000187969 | ZCCHC13 |
| ENSG00000187980 | PLA2G2C |
| ENSG00000187987 | ZSCAN23 |
| ENSG00000188000 | OR7D2 |
| ENSG00000188015 | S100A3 |
| ENSG00000188032 | C19orf67 |
| ENSG00000188039 | NWD1 |
| ENSG00000188050 | RNF133 |
| ENSG00000188051 | TMEM221 |
| ENSG00000188060 | RAB42 |
| ENSG00000188064 | WNT7B |
| ENSG00000188076 | SCGB1C1 |
| ENSG00000188086 | PRSS45 |
| ENSG00000188089 | PLA2G4E |
| ENSG00000188095 | MESP2 |
| ENSG00000188100 | FAM25A |
| ENSG00000188112 | C6orf132 |
| ENSG00000188120 | DAZ1 |
| ENSG00000188124 | OR2AG2 |
| ENSG00000188133 | TMEM215 |
| ENSG00000188153 | COL4A5 |
| ENSG00000188155 | KRTAP10-6 |
| ENSG00000188162 | OTOG |
| ENSG00000188163 | FAM166A |
| ENSG00000188176 | SMTNL2 |
| ENSG00000188219 | POTEE |
| ENSG00000188263 | IL17REL |
| ENSG00000188266 | HYKK |
| ENSG00000188269 | OR7A5 |
| ENSG00000188293 | IGFL1 |
| ENSG00000188306 | LRRIQ4 |
| ENSG00000188316 | ENO4 |
| ENSG00000188324 | OR6C6 |
| ENSG00000188334 | BSPH1 |
| ENSG00000188338 | SLC38A3 |
| ENSG00000188340 | OR6N2 |
| ENSG00000188368 | PRR19 |
| ENSG00000188373 | C10orf99 |
| ENSG00000188379 | IFNA2 |
| ENSG00000188386 | PPP3R2 |
| ENSG00000188393 | CLEC2A |
| ENSG00000188394 | GPR21 |
| ENSG00000188408 | MAGEB5 |
| ENSG00000188425 | NANOS2 |
| ENSG00000188467 | SLC24A5 |
| ENSG00000188488 | SERPINA5 |
| ENSG00000188501 | LCTL |
| ENSG00000188505 | NCCRP1 |
| ENSG00000188508 | KRTDAP |
| ENSG00000188517 | COL25A1 |
| ENSG00000188558 | OR2G6 |
| ENSG00000188573 | FBLL1 |
| ENSG00000188580 | NKAIN2 |
| ENSG00000188581 | KRTAP1-1 |
| ENSG00000188611 | ASAH2 |
| ENSG00000188613 | NANOS1 |
| ENSG00000188620 | HMX3 |
| ENSG00000188624 | IGFL3 |
| ENSG00000188626 | GOLGA8M |
| ENSG00000188655 | RNASE9 |
| ENSG00000188659 | SAXO2 |
| ENSG00000188674 | C2orf80 |
| ENSG00000188676 | IDO2 |
| ENSG00000188691 | OR56A5 |
| ENSG00000188694 | KRTAP24-1 |
| ENSG00000188710 | QRFP |
| ENSG00000188716 | DUPD1 |
| ENSG00000188729 | OSTN |
| ENSG00000188730 | WVC2 |
| ENSG00000188738 | FSIP2 |
| ENSG00000188763 | FZD9 |
| ENSG00000188766 | SPRED3 |
| ENSG00000188770 | OPTC |
| ENSG00000188771 | PLET1 |
| ENSG00000188778 | ADRB3 |
| ENSG00000188782 | CATSPER4 |
| ENSG00000188784 | PLA2G2E |
| ENSG00000188800 | TMCO2 |
| ENSG00000188803 | SHISA6 |
| ENSG00000188816 | HMX2 |
| ENSG00000188817 | SNTN |
| ENSG00000188828 | GLRA4 |
| ENSG00000188833 | ENTPD8 |
| ENSG00000188859 | FAM78B |
| ENSG00000188869 | TMC3 |
| ENSG00000188883 | KLRG2 |
| ENSG00000188909 | BSX |
| ENSG00000188910 | GJB3 |
| ENSG00000188916 | FAM196A |
| ENSG00000188931 | CFAP126 |
| ENSG00000188937 | NYX |
| ENSG00000188958 | UTS2B |
| ENSG00000188959 | C9orf152 |
| ENSG00000188984 | AADACL3 |
| ENSG00000188991 | SLC15A5 |
| ENSG00000188992 | LIPI |
| ENSG00000188993 | LRRC66 |
| ENSG00000188996 | HUS1B |
| ENSG00000189023 | MAGEB16 |
| ENSG00000189030 | VHLL |
| ENSG00000189037 | DUSP21 |
| ENSG00000189045 | ANKDD1B |
| ENSG00000189051 | RNF222 |
| ENSG00000189052 | CGB5 |
| ENSG00000189056 | RELN |
| ENSG00000189064 | GAGE2A |
| ENSG00000189099 | PRSS48 |
| ENSG00000189108 | IL1RAPL2 |
| ENSG00000189120 | SP6 |
| ENSG00000189132 | FAM47B |
| ENSG00000189134 | NKAPL |
| ENSG00000189139 | FSCB |
| ENSG00000189143 | CLDN4 |
| ENSG00000189157 | FAM47E |
| ENSG00000189167 | ZAR1L |
| ENSG00000189169 | KRTAP10-12 |
| ENSG00000189181 | OR14I1 |
| ENSG00000189182 | KRT77 |
| ENSG00000189184 | PCDH18 |
| ENSG00000189186 | DCAF8L2 |
| ENSG00000189195 | BTBD8 |
| ENSG00000189252 | SPANXN3 |
| ENSG00000189253 | TRIM64B |
| ENSG00000189280 | GJB5 |
| ENSG00000189292 | FAM150B |
| ENSG00000189299 | FOXR2 |
| ENSG00000189320 | FAM180A |
| ENSG00000189325 | C6orf222 |
| ENSG00000189326 | SPANXN4 |
| ENSG00000189357 | SPATA31D4 |
| ENSG00000189366 | ALG1L |
| ENSG00000189375 | TBC1D28 |
| ENSG00000189377 | CXCL17 |
| ENSG00000189401 | OTUD6A |
| ENSG00000189410 | SH2D5 |
| ENSG00000189431 | RASSF10 |
| ENSG00000189433 | GJB4 |
| ENSG00000196071 | OR2L13 |
| ENSG00000196081 | ZNF724P |
| ENSG00000196090 | PTPRT |
| ENSG00000196098 | OR5K4 |
| ENSG00000196099 | OR6M1 |
| ENSG00000196104 | SPOCK3 |
| ENSG00000196109 | ZNF676 |
| ENSG00000196119 | OR8A1 |
| ENSG00000196131 | VN1R2 |
| ENSG00000196132 | MYT1 |
| ENSG00000196156 | KRTAP4-3 |
| ENSG00000196166 | C8orf86 |
| ENSG00000196171 | OR6K2 |
| ENSG00000196184 | OR10J1 |
| ENSG00000196196 | HRCT1 |
| ENSG00000196208 | GREB1 |
| ENSG00000196224 | KRTAP5-3 |
| ENSG00000196228 | SULT1C3 |
| ENSG00000196240 | OR2T2 |
| ENSG00000196242 | OR2C3 |
| ENSG00000196248 | OR10S1 |
| ENSG00000196260 | SFTA2 |
| ENSG00000196266 | OR10J3 |
| ENSG00000196277 | GRM7 |
| ENSG00000196289 | BECN2 |
| ENSG00000196335 | STK31 |
| ENSG00000196337 | CGB7 |
| ENSG00000196341 | OR8D1 |
| ENSG00000196344 | ADH7 |
| ENSG00000196350 | ZNF729 |
| ENSG00000196361 | ELAVL3 |
| ENSG00000196368 | NUDT11 |
| ENSG00000196376 | SLC35F1 |
| ENSG00000196381 | ZNF781 |
| ENSG00000196391 | ZNF774 |
| ENSG00000196406 | SPANXD |
| ENSG00000196408 | NOXO1 |
| ENSG00000196420 | S100A5 |
| ENSG00000196427 | NBPF4 |
| ENSG00000196431 | CRYBA4 |
| ENSG00000196433 | ASMT |
| ENSG00000196460 | RFX8 |
| ENSG00000196468 | FGF16 |
| ENSG00000196475 | GK2 |
| ENSG00000196482 | ESRRG |
| ENSG00000196503 | ARL9 |
| ENSG00000196539 | OR2T3 |
| ENSG00000196542 | SPTSSB |
| ENSG00000196570 | PFN3 |
| ENSG00000196578 | OR5AC2 |
| ENSG00000196581 | AJAP1 |
| ENSG00000196600 | SLC22A25 |
| ENSG00000196604 | POTEF |
| ENSG00000196620 | UGT2B15 |
| ENSG00000196632 | WNK3 |
| ENSG00000196639 | HRH1 |
| ENSG00000196660 | SLC30A10 |
| ENSG00000196661 | OR8B3 |
| ENSG00000196666 | FAM180B |
| ENSG00000196711 | FAM150A |
| ENSG00000196734 | LCE1B |
| ENSG00000196748 | CLPSL2 |
| ENSG00000196754 | S100A2 |
| ENSG00000196767 | POU3F4 |
| ENSG00000196772 | OR14A16 |
| ENSG00000196778 | OR52K1 |
| ENSG00000196800 | SPINK14 |
| ENSG00000196805 | SPRR2B |
| ENSG00000196811 | CHRNG |
| ENSG00000196826 | CTD-2192J16.17 |
| ENSG00000196832 | OR11G2 |
| ENSG00000196834 | POTEI |
| ENSG00000196844 | PATE2 |
| ENSG00000196859 | KRT39 |
| ENSG00000196860 | TOMM20L |
| ENSG00000196862 | RGPD4 |
| ENSG00000196900 | TEX43 |
| ENSG00000196917 | HCAR1 |
| ENSG00000196932 | TMEM26 |
| ENSG00000196944 | OR2T4 |
| ENSG00000196946 | ZNF705A |
| ENSG00000196972 | SMIM10L2B |
| ENSG00000196990 | FAM163B |
| ENSG00000197079 | KRT35 |
| ENSG00000197084 | LCE1C |
| ENSG00000197106 | SLC6A17 |
| ENSG00000197110 | IFNL3 |
| ENSG00000197123 | ZNF679 |
| ENSG00000197125 | OR8B8 |
| ENSG00000197140 | ADAM32 |
| ENSG00000197168 | NEK5 |
| ENSG00000197172 | MAGEA6 |
| ENSG00000197177 | ADGRA1 |
| ENSG00000197181 | PIWIL2 |
| ENSG00000197191 | CYSRT1 |
| ENSG00000197213 | ZSCAN5B |
| ENSG00000197233 | OR1J2 |
| ENSG00000197241 | SLC2A7 |
| ENSG00000197245 | FAM110D |
| ENSG00000197261 | C6orf141 |
| ENSG00000197273 | GUCA2A |
| ENSG00000197279 | ZNF165 |
| ENSG00000197309 | OR10D3 |
| ENSG00000197360 | ZNF98 |
| ENSG00000197361 | FBXL22 |
| ENSG00000197364 | S100A7L2 |
| ENSG00000197376 | OR8S1 |
| ENSG00000197403 | OR6N1 |
| ENSG00000197406 | DIO3 |
| ENSG00000197408 | CYP2B6 |
| ENSG00000197410 | DCHS2 |
| ENSG00000197416 | FABP12 |
| ENSG00000197428 | OR51D1 |
| ENSG00000197430 | OPALIN |
| ENSG00000197437 | OR13G1 |
| ENSG00000197444 | OGDHL |
| ENSG00000197446 | CYP2F1 |
| ENSG00000197454 | OR2L5 |
| ENSG00000197472 | ZNF695 |
| ENSG00000197479 | PCDHB11 |
| ENSG00000197487 | GALP |
| ENSG00000197496 | SLC2A10 |
| ENSG00000197532 | OR6Y1 |
| ENSG00000197565 | COL4A6 |
| ENSG00000197584 | KCNMB2 |
| ENSG00000197587 | DMBX1 |
| ENSG00000197591 | OR11L1 |
| ENSG00000197594 | ENPP1 |
| ENSG00000197641 | SERPINB13 |
| ENSG00000197651 | CCER1 |
| ENSG00000197658 | SLC22A24 |
| ENSG00000197683 | KRTAP26-1 |
| ENSG00000197706 | OR6C74 |
| ENSG00000197734 | C14orf178 |
| ENSG00000197745 | SCGB1D4 |
| ENSG00000197748 | CFAP43 |
| ENSG00000197753 | LHFPL5 |
| ENSG00000197757 | HOXC6 |
| ENSG00000197768 | C9orf173 |
| ENSG00000197769 | MAP1LC3C |
| ENSG00000197786 | OR5B17 |
| ENSG00000197790 | OR52M1 |
| ENSG00000197826 | C4orf22 |
| ENSG00000197838 | CYP2A13 |
| ENSG00000197849 | OR8G1 |
| ENSG00000197859 | ADAMTSL2 |
| ENSG00000197870 | PRB3 |
| ENSG00000197887 | OR1S2 |
| ENSG00000197888 | UGT2B17 |
| ENSG00000197889 | MEIG1 |
| ENSG00000197891 | SLC22A12 |
| ENSG00000197901 | SLC22A6 |
| ENSG00000197919 | IFNA1 |
| ENSG00000197921 | HES5 |
| ENSG00000197935 | ZNF311 |
| ENSG00000197938 | OR5H2 |
| ENSG00000197953 | AADACL2 |
| ENSG00000197977 | ELOVL2 |
| ENSG00000197980 | LEKR1 |
| ENSG00000197991 | RP11-310K10.1 |
| ENSG00000198010 | DLGAP2 |
| ENSG00000198021 | SPANXA1 |
| ENSG00000198028 | ZNF560 |
| ENSG00000198033 | TUBA3C |
| ENSG00000198049 | AVPR1B |
| ENSG00000198062 | POTEH |
| ENSG00000198074 | AKR1B10 |
| ENSG00000198075 | SULT1C4 |
| ENSG00000198077 | CYP2A7 |
| ENSG00000198083 | KRTAP9-9 |
| ENSG00000198088 | NUP62CL |
| ENSG00000198090 | KRTAP4-6 |
| ENSG00000198092 | TMPRSS11F |
| ENSG00000198104 | OR2T6 |
| ENSG00000198108 | CHSY3 |
| ENSG00000198128 | OR2L3 |
| ENSG00000198129 | DEFB107B |
| ENSG00000198173 | FAM47C |
| ENSG00000198183 | BPIFA1 |
| ENSG00000198185 | ZNF334 |
| ENSG00000198203 | SULT1C2 |
| ENSG00000198211 | RP11-566K11.2 |
| ENSG00000198271 | KRTAP4-5 |
| ENSG00000198283 | OR5B21 |
| ENSG00000198300 | PEG3 |
| ENSG00000198326 | TMEM239 |
| ENSG00000198354 | DCAF12L2 |
| ENSG00000198390 | KRTAP13-1 |
| ENSG00000198398 | TMEM207 |
| ENSG00000198443 | KRTAP4-1 |
| ENSG00000198445 | CCT8L2 |
| ENSG00000198471 | RTP2 |
| ENSG00000198488 | B3GNT6 |
| ENSG00000198515 | CNGA1 |
| ENSG00000198535 | C2CD4A |
| ENSG00000198542 | ITGBL1 |
| ENSG00000198569 | SLC34A3 |
| ENSG00000198570 | RD3 |
| ENSG00000198573 | SPANXC |
| ENSG00000198597 | ZNF536 |
| ENSG00000198601 | OR2M2 |
| ENSG00000198633 | ZNF534 |
| ENSG00000198643 | FAM3D |
| ENSG00000198670 | LPA |
| ENSG00000198674 | OR10G6 |
| ENSG00000198681 | MAGEA1 |
| ENSG00000198691 | ABCA4 |
| ENSG00000198704 | GPX6 |
| ENSG00000198723 | C19orf45 |
| ENSG00000198729 | PPP1R14C |
| ENSG00000198732 | SMOC1 |
| ENSG00000198739 | LRRTM3 |
| ENSG00000198754 | OXCT2 |
| ENSG00000198758 | EPS8L3 |
| ENSG00000198759 | EGFL6 |
| ENSG00000198765 | SYCP1 |
| ENSG00000198768 | APCDD1L |
| ENSG00000198774 | RASSF9 |
| ENSG00000198788 | MUC2 |
| ENSG00000198796 | ALPK2 |
| ENSG00000198797 | BRINP2 |
| ENSG00000198798 | MAGEB3 |
| ENSG00000198807 | PAX9 |
| ENSG00000198812 | LRRC10 |
| ENSG00000198822 | GRM3 |
| ENSG00000198842 | DUSP27 |
| ENSG00000198854 | C1orf68 |
| ENSG00000198870 | STKLD1 |
| ENSG00000198881 | ASB12 |
| ENSG00000198883 | PNMA5 |
| ENSG00000198889 | DCAF12L1 |
| ENSG00000198930 | CSAG1 |
| ENSG00000198939 | ZFP2 |
| ENSG00000198963 | RORB |
| ENSG00000198965 | OR10R2 |
| ENSG00000198967 | OR10Z1 |
| ENSG00000203661 | OR2T5 |
| ENSG00000203663 | OR2L2 |
| ENSG00000203685 | C1orf95 |
| ENSG00000203690 | TCP10 |
| ENSG00000203697 | CAPN8 |
| ENSG00000203722 | RAET1G |
| ENSG00000203724 | C1orf53 |
| ENSG00000203727 | SAMD5 |
| ENSG00000203730 | TEDDM1 |
| ENSG00000203733 | GJE1 |
| ENSG00000203734 | ECT2L |
| ENSG00000203737 | GPR52 |
| ENSG00000203740 | METTL11B |
| ENSG00000203756 | TMEM244 |
| ENSG00000203757 | OR6K3 |
| ENSG00000203780 | FANK1 |
| ENSG00000203782 | LOR |
| ENSG00000203783 | PRR9 |
| ENSG00000203784 | LELP1 |
| ENSG00000203785 | SPRR2E |
| ENSG00000203786 | KPRP |
| ENSG00000203795 | FAM24A |
| ENSG00000203805 | PLPP4 |
| ENSG00000203818 | HIST2H3PS2 |
| ENSG00000203837 | PNLIPRP3 |
| ENSG00000203857 | HSD3B1 |
| ENSG00000203859 | HSD3B2 |
| ENSG00000203867 | RBM20 |
| ENSG00000203870 | SMIM9 |
| ENSG00000203877 | RIPPLY2 |
| ENSG00000203907 | OOEP |
| ENSG00000203908 | KHDC3L |
| ENSG00000203909 | DPPA5 |
| ENSG00000203910 | C1orf146 |
| ENSG00000203923 | SPANXN1 |
| ENSG00000203926 | SPANXA2 |
| ENSG00000203933 | CXorf66 |
| ENSG00000203942 | C10orf62 |
| ENSG00000203943 | SAMD13 |
| ENSG00000203952 | CCDC160 |
| ENSG00000203963 | C1orf141 |
| ENSG00000203970 | DEFB110 |
| ENSG00000203972 | GLYATL3 |
| ENSG00000203985 | LDLRAD1 |
| ENSG00000203989 | RHOXF2B |
| ENSG00000203995 | ZYG11A |
| ENSG00000204003 | RP11-216L13.16 |
| ENSG00000204006 | C1orf185 |
| ENSG00000204007 | GLT6D1 |
| ENSG00000204019 | CT83 |
| ENSG00000204021 | LIPK |
| ENSG00000204022 | LIPJ |
| ENSG00000204033 | LRIT2 |
| ENSG00000204052 | LRRC73 |
| ENSG00000204060 | FOXO6 |
| ENSG00000204065 | TCEAL5 |
| ENSG00000204071 | TCEAL6 |
| ENSG00000204086 | RPA4 |
| ENSG00000204099 | NEU4 |
| ENSG00000204128 | C2orf72 |
| ENSG00000204140 | CLPSL1 |
| ENSG00000204147 | ASAH2B |
| ENSG00000204174 | NPY4R |
| ENSG00000204175 | GPRIN2 |
| ENSG00000204183 | GDF5OS |
| ENSG00000204193 | TXNDC8 |
| ENSG00000204195 | AWAT1 |
| ENSG00000204246 | OR13C3 |
| ENSG00000204278 | TMEM235 |
| ENSG00000204279 | PAGE3 |
| ENSG00000204290 | BTNL2 |
| ENSG00000204293 | OR8B2 |
| ENSG00000204296 | C6orf10 |
| ENSG00000204300 | TMEM225 |
| ENSG00000204311 | DFNB59 |
| ENSG00000204334 | ERICH2 |
| ENSG00000204335 | SP5 |
| ENSG00000204347 | BTBD17 |
| ENSG00000204352 | C9orf129 |
| ENSG00000204361 | NXPE2 |
| ENSG00000204363 | SPANXN5 |
| ENSG00000204382 | XAGE1B |
| ENSG00000204385 | SLC44A4 |
| ENSG00000204414 | CSHL1 |
| ENSG00000204422 | XXbac-BPG32J3.20 |
| ENSG00000204442 | FAM155A |
| ENSG00000204449 | TRIM49C |
| ENSG00000204450 | TRIM64 |
| ENSG00000204478 | PRAMEF20 |
| ENSG00000204479 | PRAMEF17 |
| ENSG00000204480 | PRAMEF19 |
| ENSG00000204481 | PRAMEF14 |
| ENSG00000204501 | PRAMEF9 |
| ENSG00000204510 | PRAMEF7 |
| ENSG00000204511 | MCCD1 |
| ENSG00000204518 | AADACL4 |
| ENSG00000204532 | ZSCAN5CP |
| ENSG00000204538 | PSORS1C2 |
| ENSG00000204539 | CDSN |
| ENSG00000204540 | PSORS1C1 |
| ENSG00000204542 | C6orf15 |
| ENSG00000204544 | MUC21 |
| ENSG00000204548 | DEFB121 |
| ENSG00000204571 | KRTAP5-11 |
| ENSG00000204572 | KRTAP5-10 |
| ENSG00000204583 | LRCOL1 |
| ENSG00000204595 | DPRX |
| ENSG00000204610 | TRIM15 |
| ENSG00000204612 | FOXB2 |
| ENSG00000204616 | TRIM31 |
| ENSG00000204624 | PTCHD2 |
| ENSG00000204640 | NMS |
| ENSG00000204653 | ASPDH |
| ENSG00000204655 | MOG |
| ENSG00000204657 | OR2H2 |
| ENSG00000204659 | CBY3 |
| ENSG00000204669 | C9orf57 |
| ENSG00000204671 | IL31 |
| ENSG00000204677 | FAM153C |
| ENSG00000204682 | CASC10 |
| ENSG00000204683 | C10orf113 |
| ENSG00000204687 | MAS1L |
| ENSG00000204688 | OR2H1 |
| ENSG00000204694 | OR11A1 |
| ENSG00000204695 | OR14J1 |
| ENSG00000204700 | OR2J2 |
| ENSG00000204701 | OR2J3 |
| ENSG00000204703 | OR2B3 |
| ENSG00000204704 | OR2W1 |
| ENSG00000204711 | C9orf135 |
| ENSG00000204740 | MALRD1 |
| ENSG00000204779 | FOXD4L5 |
| ENSG00000204790 | CBWD6 |
| ENSG00000204849 | SPATA31A1 |
| ENSG00000204866 | IGFL2 |
| ENSG00000204869 | IGFL4 |
| ENSG00000204873 | KRTAP9-3 |
| ENSG00000204880 | KRTAP4-8 |
| ENSG00000204882 | GPR20 |
| ENSG00000204887 | KRTAP1-4 |
| ENSG00000204889 | KRT40 |
| ENSG00000204897 | KRT25 |
| ENSG00000204909 | SPINK9 |
| ENSG00000204913 | LRRC3C |
| ENSG00000204918 | PRR20B |
| ENSG00000204919 | PRR20A |
| ENSG00000204928 | GRXCR2 |
| ENSG00000204930 | FAM221B |
| ENSG00000204941 | PSG5 |
| ENSG00000204950 | LRRC10B |
| ENSG00000204952 | FBXO47 |
| ENSG00000204956 | PCDHGA1 |
| ENSG00000204961 | PCDHA9 |
| ENSG00000204962 | PCDHA8 |
| ENSG00000204963 | PCDHA7 |
| ENSG00000204965 | PCDHA5 |
| ENSG00000204967 | PCDHA4 |
| ENSG00000204969 | PCDHA2 |
| ENSG00000204970 | PCDHA1 |
| ENSG00000204978 | ERICH4 |
| ENSG00000204979 | MS4A13 |
| ENSG00000205002 | AARD |
| ENSG00000205022 | PABPN1L |
| ENSG00000205029 | OR5D16 |
| ENSG00000205030 | OR5L2 |
| ENSG00000205057 | CLLU1OS |
| ENSG00000205076 | LGALS7 |
| ENSG00000205086 | C2orf91 |
| ENSG00000205097 | FRG2 |
| ENSG00000205108 | FAM205A |
| ENSG00000205111 | CDKL4 |
| ENSG00000205116 | TMEM88B |
| ENSG00000205126 | ACCSL |
| ENSG00000205129 | C4orf47 |
| ENSG00000205143 | ARID3C |
| ENSG00000205177 | C11orf91 |
| ENSG00000205186 | FABP9 |
| ENSG00000205209 | SCGB2B2 |
| ENSG00000205212 | CCDC144NL |
| ENSG00000205277 | MUC12 |
| ENSG00000205279 | CTXN3 |
| ENSG00000205301 | MGAT4D |
| ENSG00000205327 | OR6C68 |
| ENSG00000205328 | OR6C65 |
| ENSG00000205329 | OR6C3 |
| ENSG00000205330 | OR6C1 |
| ENSG00000205358 | MT1H |
| ENSG00000205359 | SLCO6A1 |
| ENSG00000205362 | MT1A |
| ENSG00000205363 | C15orf59 |
| ENSG00000205403 | CFI |
| ENSG00000205409 | OR52E6 |
| ENSG00000205426 | KRT81 |
| ENSG00000205436 | EXOC3L4 |
| ENSG00000205439 | KRTAP12-3 |
| ENSG00000205442 | IZUMO3 |
| ENSG00000205445 | KRTAP10-2 |
| ENSG00000205456 | TP53TG3D |
| ENSG00000205457 | TP53TG3C |
| ENSG00000205495 | OR52J3 |
| ENSG00000205496 | OR51A2 |
| ENSG00000205497 | OR51A4 |
| ENSG00000205549 | C9orf92 |
| ENSG00000205642 | VCX3B |
| ENSG00000205649 | HTN3 |
| ENSG00000205667 | ARSH |
| ENSG00000205669 | ACOT6 |
| ENSG00000205678 | TECRL |
| ENSG00000205693 | MANSC4 |
| ENSG00000205718 | MBD3L4 |
| ENSG00000205754 | SLCO1B7 |
| ENSG00000205755 | CRLF2 |
| ENSG00000205777 | GAGE1 |
| ENSG00000205795 | CYS1 |
| ENSG00000205832 | C16orf96 |
| ENSG00000205835 | GMNC |
| ENSG00000205838 | TTC23L |
| ENSG00000205856 | C22orf42 |
| ENSG00000205857 | NANOGNB |
| ENSG00000205858 | LRRC72 |
| ENSG00000205863 | C1QTNF9B |
| ENSG00000205864 | KRTAP5-6 |
| ENSG00000205867 | KRTAP5-2 |
| ENSG00000205869 | KRTAP5-1 |
| ENSG00000205882 | DEFB134 |
| ENSG00000205883 | DEFB135 |
| ENSG00000205884 | DEFB136 |
| ENSG00000205899 | BHLHA9 |
| ENSG00000205916 | DAZ4 |
| ENSG00000205929 | C21orf62 |
| ENSG00000205944 | DAZ2 |
| ENSG00000206013 | IFITM5 |
| ENSG00000206026 | SMIM21 |
| ENSG00000206043 | C18orf63 |
| ENSG00000206069 | TMEM211 |
| ENSG00000206072 | SERPINB11 |
| ENSG00000206073 | SERPINB4 |
| ENSG00000206075 | SERPINB5 |
| ENSG00000206102 | KRTAP19-8 |
| ENSG00000206104 | KRTAP20-3 |
| ENSG00000206105 | KRTAP20-4 |
| ENSG00000206106 | KRTAP22-2 |
| ENSG00000206107 | KRTAP27-1 |
| ENSG00000206113 | CFAP99 |
| ENSG00000206127 | GOLGA8O |
| ENSG00000206150 | RNASE13 |
| ENSG00000206181 | TCEB3B |
| ENSG00000206199 | ANKUB1 |
| ENSG00000206203 | TSSK2 |
| ENSG00000206260 | PRR23A |
| ENSG00000206262 | FOXL2NB |
| ENSG00000206384 | COL6A6 |
| ENSG00000206422 | LRRC30 |
| ENSG00000206432 | TMEM200C |
| ENSG00000206483 | TXNRD3NB |
| ENSG00000206531 | CD200R1L |
| ENSG00000206535 | LNP1 |
| ENSG00000206536 | OR5K3 |
| ENSG00000206538 | VGLL3 |
| ENSG00000206557 | TRIM71 |
| ENSG00000206559 | ZCWPW2 |
| ENSG00000206579 | XKR4 |
| ENSG00000211448 | DIO2 |
| ENSG00000211452 | DIO1 |
| ENSG00000212122 | TSSK1B |
| ENSG00000212124 | TAS2R19 |
| ENSG00000212126 | TAS2R50 |
| ENSG00000212128 | TAS2R13 |
| ENSG00000212643 | ZRSR1 |
| ENSG00000212657 | KRTAP16-1 |
| ENSG00000212658 | KRTAP29-1 |
| ENSG00000212659 | KRTAP9-6 |
| ENSG00000212710 | CTAGE1 |
| ENSG00000212721 | KRTAP4-11 |
| ENSG00000212722 | KRTAP4-9 |
| ENSG00000212724 | KRTAP2-3 |
| ENSG00000212725 | KRTAP2-1 |
| ENSG00000212807 | OR2A42 |
| ENSG00000212899 | KRTAP3-3 |
| ENSG00000212900 | KRTAP3-2 |
| ENSG00000212901 | KRTAP3-1 |
| ENSG00000212933 | KRTAP12-4 |
| ENSG00000212935 | KRTAP10-3 |
| ENSG00000212938 | KRTAP6-3 |
| ENSG00000212993 | POU5F1B |
| ENSG00000213022 | KLK9 |
| ENSG00000213023 | SYT3 |
| ENSG00000213029 | SPHAR |
| ENSG00000213030 | CGB8 |
| ENSG00000213171 | LINGO4 |
| ENSG00000213204 | RP3-382I10.7 |
| ENSG00000213218 | CSH2 |
| ENSG00000213265 | TSGA13 |
| ENSG00000213401 | MAGEA12 |
| ENSG00000213416 | KRTAP4-12 |
| ENSG00000213417 | KRTAP2-4 |
| ENSG00000213424 | KRT222 |
| ENSG00000213512 | GBP7 |
| ENSG00000213578 | CPLX3 |
| ENSG00000213759 | UGT2B11 |
| ENSG00000213822 | CEACAM18 |
| ENSG00000213886 | UBD |
| ENSG00000213892 | CEACAM16 |
| ENSG00000213921 | LEUTX |
| ENSG00000213927 | CCL27 |
| ENSG00000213973 | ZNF99 |
| ENSG00000213996 | TM6SF2 |
| ENSG00000214042 | IFNA7 |
| ENSG00000214050 | FBXO16 |
| ENSG00000214097 | SMCO1 |
| ENSG00000214102 | WEE2 |
| ENSG00000214107 | MAGEB1 |
| ENSG00000214128 | TMEM213 |
| ENSG00000214215 | C12orf74 |
| ENSG00000214216 | IQCJ |
| ENSG00000214265 | RP11-701H24.9 |
| ENSG00000214285 | NPS |
| ENSG00000214290 | COLCA2 |
| ENSG00000214336 | FOXI3 |
| ENSG00000214338 | SOGA3 |
| ENSG00000214360 | EFCAB9 |
| ENSG00000214376 | VSTM5 |
| ENSG00000214414 | TRIM77 |
| ENSG00000214415 | GNAT3 |
| ENSG00000214491 | SEC14L6 |
| ENSG00000214510 | SPINK13 |
| ENSG00000214511 | HIGD1C |
| ENSG00000214513 | NOTO |
| ENSG00000214518 | KRTAP2-2 |
| ENSG00000214575 | CPEB1 |
| ENSG00000214642 | DEFB113 |
| ENSG00000214643 | DEFB133 |
| ENSG00000214652 | ZNF727 |
| ENSG00000214681 | IQCF5 |
| ENSG00000214686 | IQCF6 |
| ENSG00000214694 | ARHGEF33 |
| ENSG00000214700 | C12orf71 |
| ENSG00000214711 | CAPN14 |
| ENSG00000214732 | RP1-139D8.6 |
| ENSG00000214782 | MS4A18 |
| ENSG00000214814 | FER1L6 |
| ENSG00000214819 | CDRT15L2 |
| ENSG00000214842 | RAD51AP2 |
| ENSG00000214860 | EVPLL |
| ENSG00000214866 | DCDC2C |
| ENSG00000214891 | TRIM64C |
| ENSG00000214897 | U82695.5 |
| ENSG00000214929 | SPATA31D1 |
| ENSG00000214943 | GPR33 |
| ENSG00000214946 | TBC1D26 |
| ENSG00000214954 | LRRC69 |
| ENSG00000214978 | GSG1L2 |
| ENSG00000215009 | ACSM4 |
| ENSG00000215018 | COL28A1 |
| ENSG00000215029 | TCP11X2 |
| ENSG00000215045 | GRID2IP |
| ENSG00000215113 | CXorf49B |
| ENSG00000215115 | CXorf49 |
| ENSG00000215131 | C16orf90 |
| ENSG00000215174 | NLRP2P |
| ENSG00000215182 | MUC5AC |
| ENSG00000215183 | MSMP |
| ENSG00000215186 | GOLGA6B |
| ENSG00000215187 | FAM166B |
| ENSG00000215203 | GRXCR1 |
| ENSG00000215217 | C5orf49 |
| ENSG00000215218 | UBE2QL1 |
| ENSG00000215262 | KCNU1 |
| ENSG00000215269 | GAGE12G |
| ENSG00000215274 | GAGE10 |
| ENSG00000215277 | RNF212B |
| ENSG00000215343 | ZNF705D |
| ENSG00000215356 | ZNF705B |
| ENSG00000215372 | ZNF705G |
| ENSG00000215397 | SCRT2 |
| ENSG00000215454 | KRTAP10-4 |
| ENSG00000215455 | KRTAP10-1 |
| ENSG00000215474 | SKOR2 |
| ENSG00000215475 | SIAH3 |
| ENSG00000215545 | DEFB116 |
| ENSG00000215547 | DEFB115 |
| ENSG00000215568 | GAB4 |
| ENSG00000215595 | C20orf202 |
| ENSG00000215612 | HMX1 |
| ENSG00000215644 | GCGR |
| ENSG00000215704 | CELA2B |
| ENSG00000215853 | RPTN |
| ENSG00000215906 | LACTBL1 |
| ENSG00000215910 | C1orf167 |
| ENSG00000216588 | IGSF23 |
| ENSG00000216649 | GAGE12E |
| ENSG00000217236 | SP9 |
| ENSG00000217442 | SYCE3 |
| ENSG00000218336 | TENM3 |
| ENSG00000218819 | TDRD15 |
| ENSG00000218823 | PAPOLB |
| ENSG00000219435 | TEX40 |
| ENSG00000219438 | FAM19A5 |
| ENSG00000221813 | OR6B1 |
| ENSG00000221818 | EBF2 |
| ENSG00000221826 | PSG3 |
| ENSG00000221836 | OR2A5 |
| ENSG00000221837 | KRTAP10-9 |
| ENSG00000221840 | OR4A5 |
| ENSG00000221843 | C2orf16 |
| ENSG00000221852 | KRTAP1-5 |
| ENSG00000221855 | TAS2R41 |
| ENSG00000221858 | OR2A12 |
| ENSG00000221859 | KRTAP10-10 |
| ENSG00000221864 | KRTAP12-2 |
| ENSG00000221867 | MAGEA3 |
| ENSG00000221870 | TMEM257 |
| ENSG00000221874 | ZNF816-ZNF321P |
| ENSG00000221878 | PSG7 |
| ENSG00000221880 | KRTAP1-3 |
| ENSG00000221882 | OR3A2 |
| ENSG00000221887 | HMSD |
| ENSG00000221888 | OR1C1 |
| ENSG00000221900 | POM121L12 |
| ENSG00000221910 | OR2F2 |
| ENSG00000221916 | C19orf73 |
| ENSG00000221931 | OR6X1 |
| ENSG00000221932 | HEPN1 |
| ENSG00000221933 | OR2A25 |
| ENSG00000221938 | OR2A14 |
| ENSG00000221947 | XKR9 |
| ENSG00000221954 | OR4C12 |
| ENSG00000221961 | PRR21 |
| ENSG00000221962 | TMEM14EP |
| ENSG00000221970 | OR2A1 |
| ENSG00000221972 | C3orf36 |
| ENSG00000221977 | OR4E2 |
| ENSG00000221986 | MYBPHL |
| ENSG00000221989 | OR2A2 |
| ENSG00000222014 | RAB6C |
| ENSG00000222018 | C21orf140 |
| ENSG00000222028 | PSMB11 |
| ENSG00000222036 | POTEG |
| ENSG00000222038 | POTEJ |
| ENSG00000222046 | DCDC2B |
| ENSG00000223417 | TRIM49D1 |
| ENSG00000223443 | USP17L2 |
| ENSG00000223510 | CDRT15 |
| ENSG00000223569 | USP17L15 |
| ENSG00000223572 | CKMT1A |
| ENSG00000223591 | CENPVP1 |
| ENSG00000223601 | EBLN1 |
| ENSG00000223614 | ZNF735 |
| ENSG00000223638 | RFPL4A |
| ENSG00000223658 | C1GALT1C1L |
| ENSG00000223802 | CERS1 |
| ENSG00000223953 | C1QTNF5 |
| ENSG00000224089 | CT47A10 |
| ENSG00000224109 | CENPVP3 |
| ENSG00000224586 | GPX5 |
| ENSG00000224659 | GAGE12J |
| ENSG00000224689 | ZNF812 |
| ENSG00000224902 | GAGE12H |
| ENSG00000224982 | TMEM233 |
| ENSG00000225110 | LL0XNC01-16G2.1 |
| ENSG00000225327 | USP17L3 |
| ENSG00000225362 | CT62 |
| ENSG00000225526 | MKRN2OS |
| ENSG00000225556 | C2CD4D |
| ENSG00000225781 | OR6V1 |
| ENSG00000225805 | CTD-2313N18.7 |
| ENSG00000225899 | FRG2B |
| ENSG00000225932 | CTAGE4 |
| ENSG00000225950 | NTF4 |
| ENSG00000225968 | ELFN1 |
| ENSG00000226023 | CT47A6 |
| ENSG00000226288 | OR52I2 |
| ENSG00000226321 | CROCC2 |
| ENSG00000226372 | DCAF8L1 |
| ENSG00000226430 | USP17L7 |
| ENSG00000226600 | CT47A9 |
| ENSG00000226650 | KIF4B |
| ENSG00000226685 | CT47A12 |
| ENSG00000226761 | TAS2R46 |
| ENSG00000226784 | PGAM4 |
| ENSG00000226887 | ERVMER34-1 |
| ENSG00000226929 | CT47A11 |
| ENSG00000226941 | RBMY1J |
| ENSG00000227059 | ANHX |
| ENSG00000227140 | USP17L5 |
| ENSG00000227151 | PRR20D |
| ENSG00000227234 | SPANXB1 |
| ENSG00000227471 | AKR1B15 |
| ENSG00000227488 | GAGE12D |
| ENSG00000227551 | USP17L12 |
| ENSG00000227729 | RD3L |
| ENSG00000227868 | C1orf234 |
| ENSG00000227877 | MRLN |
| ENSG00000228075 | BOD1L2 |
| ENSG00000228083 | IFNA14 |
| ENSG00000228144 | RP11-745O10.4 |
| ENSG00000228198 | OR2M3 |
| ENSG00000228517 | CT47A7 |
| ENSG00000228567 | VN1R4 |
| ENSG00000228607 | CLDN25 |
| ENSG00000228836 | CT45A4 |
| ENSG00000228856 | USP17L30 |
| ENSG00000228927 | TSPY3 |
| ENSG00000229292 | RFPL4AL1 |
| ENSG00000229415 | SFTA3 |
| ENSG00000229453 | SPINK8 |
| ENSG00000229544 | NKX1-2 |
| ENSG00000229549 | TSPY8 |
| ENSG00000229571 | PRAMEF26 |
| ENSG00000229579 | USP17L26 |
| ENSG00000229637 | PRAC2 |
| ENSG00000229665 | PRR20C |
| ENSG00000229676 | ZNF492 |
| ENSG00000229924 | FAM90A26 |
| ENSG00000229937 | PRPS1L1 |
| ENSG00000229972 | IQCF3 |
| ENSG00000230031 | POTEB2 |
| ENSG00000230062 | ANKRD66 |
| ENSG00000230178 | OR4F3 |
| ENSG00000230347 | CT47A8 |
| ENSG00000230430 | USP17L25 |
| ENSG00000230453 | ANKRD18B |
| ENSG00000230510 | PPP5D1 |
| ENSG00000230522 | MBD3L2 |
| ENSG00000230549 | USP17L1 |
| ENSG00000230594 | CT47A4 |
| ENSG00000230657 | PRB4 |
| ENSG00000230667 | SETSIP |
| ENSG00000230778 | ANKRD63 |
| ENSG00000230797 | YY2 |
| ENSG00000230873 | STMND1 |
| ENSG00000231051 | USP17L28 |
| ENSG00000231068 | KRTAP21-3 |
| ENSG00000231192 | OR5H1 |
| ENSG00000231213 | PLSCR5 |
| ENSG00000231256 | C17orf105 |
| ENSG00000231274 | SBK3 |
| ENSG00000231396 | USP17L10 |
| ENSG00000231637 | USP17L29 |
| ENSG00000231672 | DIRC3 |
| ENSG00000231738 | TSPAN19 |
| ENSG00000231824 | C18orf42 |
| ENSG00000231861 | OR5K2 |
| ENSG00000231924 | PSG1 |
| ENSG00000232030 | MAGEB6P1 |
| ENSG00000232040 | ZBED9 |
| ENSG00000232196 | MTRNR2L4 |
| ENSG00000232237 | ASCL5 |
| ENSG00000232258 | TMEM114 |
| ENSG00000232263 | KRTAP25-1 |
| ENSG00000232264 | USP17L24 |
| ENSG00000232268 | OR52I1 |
| ENSG00000232382 | OR5K1 |
| ENSG00000232399 | USP17L13 |
| ENSG00000232423 | PRAMEF6 |
| ENSG00000232948 | DEFB130 |
| ENSG00000233041 | PHGR1 |
| ENSG00000233050 | RP11-1236K1.1 |
| ENSG00000233136 | USP17L11 |
| ENSG00000233198 | RNF224 |
| ENSG00000233232 | NPIPB7 |
| ENSG00000233412 | OR5H15 |
| ENSG00000233436 | BTBD18 |
| ENSG00000233438 | AC109829.1 |
| ENSG00000233670 | PIRT |
| ENSG00000233701 | PRR23C |
| ENSG00000233757 | AC092835.2 |
| ENSG00000233802 | TRIM49D2 |
| ENSG00000233803 | TSPY4 |
| ENSG00000233816 | IFNA13 |
| ENSG00000233917 | POTEB |
| ENSG00000233932 | CTXN2 |
| ENSG00000234068 | PAGE2 |
| ENSG00000234224 | TMEM229A |
| ENSG00000234278 | PRR20E |
| ENSG00000234409 | CCDC188 |
| ENSG00000234414 | RBMY1A1 |
| ENSG00000234438 | KBTBD13 |
| ENSG00000234469 | CLDN34 |
| ENSG00000234560 | OR10G8 |
| ENSG00000234602 | MCIDAS |
| ENSG00000234776 | C11orf94 |
| ENSG00000234829 | IFNA17 |
| ENSG00000234906 | APOC2 |
| ENSG00000235034 | C19orf81 |
| ENSG00000235098 | ANKRD65 |
| ENSG00000235109 | ZSCAN31 |
| ENSG00000235268 | KDM4E |
| ENSG00000235376 | RPEL1 |
| ENSG00000235608 | NKX1-1 |
| ENSG00000235631 | RNF148 |
| ENSG00000235711 | ANKRD34C |
| ENSG00000235718 | MFRP |
| ENSG00000235780 | USP17L27 |
| ENSG00000235942 | LCE6A |
| ENSG00000235961 | PNMA6A |
| ENSG00000236027 | PATE3 |
| ENSG00000236032 | OR5H14 |
| ENSG00000236125 | USP17L4 |
| ENSG00000236126 | CT47A3 |
| ENSG00000236334 | PPIAL4G |
| ENSG00000236362 | GAGE12F |
| ENSG00000236371 | CT47A1 |
| ENSG00000236398 | TAS2R39 |
| ENSG00000236424 | TSPY10 |
| ENSG00000236446 | CT47B1 |
| ENSG00000236637 | IFNA4 |
| ENSG00000236699 | ARHGEF38 |
| ENSG00000236737 | GAGE12B |
| ENSG00000236761 | CTAGE9 |
| ENSG00000236782 | RP11-96L14.7 |
| ENSG00000236980 | C3orf84 |
| ENSG00000236981 | OR10G9 |
| ENSG00000237038 | USP17L8 |
| ENSG00000237110 | TAAR9 |
| ENSG00000237136 | C4orf51 |
| ENSG00000237247 | MBD3L5 |
| ENSG00000237289 | CKMT1B |
| ENSG00000237330 | RNF223 |
| ENSG00000237353 | PATE4 |
| ENSG00000237388 | OR4A47 |
| ENSG00000237412 | PRSS56 |
| ENSG00000237452 | BHMG1 |
| ENSG00000237515 | SHISA9 |
| ENSG00000237521 | OR7E24 |
| ENSG00000237671 | GAGE12C |
| ENSG00000237693 | IRGM |
| ENSG00000237763 | AMY1A |
| ENSG00000237847 | FAM231A |
| ENSG00000239388 | ASB14 |
| ENSG00000239389 | PCDHA13 |
| ENSG00000239590 | OR1J4 |
| ENSG00000239605 | C2orf61 |
| ENSG00000239642 | MEIKIN |
| ENSG00000239810 | WI2-3308P17.2 |
| ENSG00000239886 | KRTAP9-2 |
| ENSG00000240021 | TEX35 |
| ENSG00000240224 | UGT1A5 |
| ENSG00000240386 | LCE1F |
| ENSG00000240432 | KRTAP13-3 |
| ENSG00000240542 | KRTAP9-1 |
| ENSG00000240563 | L1TD1 |
| ENSG00000240654 | C1QTNF9 |
| ENSG00000240694 | PNMA2 |
| ENSG00000240720 | LRRD1 |
| ENSG00000240747 | KRBOX1 |
| ENSG00000240764 | PCDHGC5 |
| ENSG00000240871 | KRTAP4-7 |
| ENSG00000241119 | UGT1A9 |
| ENSG00000241123 | KRTAP10-5 |
| ENSG00000241128 | OR14A2 |
| ENSG00000241186 | TDGF1 |
| ENSG00000241233 | KRTAP5-8 |
| ENSG00000241241 | KRTAP4-16P |
| ENSG00000241322 | CDRT1 |
| ENSG00000241476 | SSX2 |
| ENSG00000241563 | CORT |
| ENSG00000241595 | KRTAP9-4 |
| ENSG00000241598 | KRTAP5-4 |
| ENSG00000241635 | UGT1A1 |
| ENSG00000241690 | RP5-860F19.8 |
| ENSG00000241697 | TMEFF1 |
| ENSG00000241794 | SPRR2A |
| ENSG00000241935 | HOGA1 |
| ENSG00000241962 | RP11-111H13.1 |
| ENSG00000242019 | KIR3DL3 |
| ENSG00000242173 | ARHGDIG |
| ENSG00000242180 | OR51B5 |
| ENSG00000242220 | TCP10L |
| ENSG00000242221 | PSG2 |
| ENSG00000242362 | CT47A2 |
| ENSG00000242366 | UGT1A8 |
| ENSG00000242389 | RBMY1E |
| ENSG00000242419 | PCDHGC4 |
| ENSG00000242515 | UGT1A10 |
| ENSG00000242689 | CNTF |
| ENSG00000242715 | CCDC169 |
| ENSG00000242866 | STRC |
| ENSG00000242875 | RBMY1B |
| ENSG00000242950 | ERVW-1 |
| ENSG00000243073 | PRAMEF4 |
| ENSG00000243130 | PSG11 |
| ENSG00000243135 | UGT1A3 |
| ENSG00000243137 | PSG4 |
| ENSG00000243232 | PCDHAC2 |
| ENSG00000243440 | AF165138.7 |
| ENSG00000243444 | PALM2 |
| ENSG00000243489 | KRTAP10-11 |
| ENSG00000243501 | RP11-257K9.8 |
| ENSG00000243509 | TNFRSF6B |
| ENSG00000243543 | WFDC6 |
| ENSG00000243627 | AP000322.53 |
| ENSG00000243710 | CFAP57 |
| ENSG00000243729 | OR5V1 |
| ENSG00000243896 | OR2A7 |
| ENSG00000243910 | TUBA4B |
| ENSG00000243978 | RGAG1 |
| ENSG00000244020 | MT1HL1 |
| ENSG00000244025 | KRTAP19-3 |
| ENSG00000244057 | LCE3C |
| ENSG00000244094 | SPRR2F |
| ENSG00000244122 | UGT1A7 |
| ENSG00000244255 | XXbac-BPG116M5.17 |
| ENSG00000244355 | LY6G6D |
| ENSG00000244362 | KRTAP19-7 |
| ENSG00000244395 | RBMY1D |
| ENSG00000244411 | KRTAP5-7 |
| ENSG00000244414 | CFHR1 |
| ENSG00000244474 | UGT1A4 |
| ENSG00000244476 | ERVFRD-1 |
| ENSG00000244537 | KRTAP4-2 |
| ENSG00000244588 | RAD21L1 |
| ENSG00000244623 | OR2AE1 |
| ENSG00000244624 | KRTAP20-1 |
| ENSG00000244693 | CTAGE8 |
| ENSG00000244694 | PTCHD4 |
| ENSG00000244752 | CRYBB2 |
| ENSG00000247595 | SPTY2D1-AS1 |
| ENSG00000248167 | TRIM39-RPP21 |
| ENSG00000248235 | AC037459.4 |
| ENSG00000248329 | APELA |
| ENSG00000248383 | PCDHAC1 |
| ENSG00000248405 | PRR5-ARHGAP8 |
| ENSG00000248485 | PCP4L1 |
| ENSG00000248710 | RP11-432B6.3 |
| ENSG00000248713 | RP11-766F14.2 |
| ENSG00000248920 | USP17L19 |
| ENSG00000248933 | USP17L22 |
| ENSG00000248993 | XXbac-BPG181M17.5 |
| ENSG00000249104 | USP17L17 |
| ENSG00000249139 | EPPIN-WFDC6 |
| ENSG00000249141 | RP11-514O12.4 |
| ENSG00000249158 | PCDHA11 |
| ENSG00000249209 | AP000304.12 |
| ENSG00000249222 | ATP5L2 |
| ENSG00000249242 | TMEM150C |
| ENSG00000249481 | SPATS1 |
| ENSG00000249581 | CLRN2 |
| ENSG00000249590 | RP4-539M6.19 |
| ENSG00000249693 | THEGL |
| ENSG00000249715 | FER1L5 |
| ENSG00000249773 | RP11-15K19.2 |
| ENSG00000249811 | USP17L21 |
| ENSG00000249853 | HS3ST5 |
| ENSG00000249860 | MTRNR2L5 |
| ENSG00000249861 | LGALS16 |
| ENSG00000249884 | RNF103-CHMP3 |
| ENSG00000249931 | GOLGA8K |
| ENSG00000249961 | CCDC79 |
| ENSG00000249967 | RP11-548K23.11 |
| ENSG00000250120 | PCDHA10 |
| ENSG00000250254 | PTTG2 |
| ENSG00000250305 | KIAA1456 |
| ENSG00000250349 | RP5-972B16.2 |
| ENSG00000250423 | KIAA1210 |
| ENSG00000250424 | RP5-877J2.1 |
| ENSG00000250486 | FAM218A |
| ENSG00000250588 | IQCJ-SCHIP1 |
| ENSG00000250641 | XXbac-BPG32J3.19 |
| ENSG00000250709 | CCDC169-SOHLH2 |
| ENSG00000250719 | RP11-322N21.2 |
| ENSG00000250741 | NT5C1B-RDH14 |
| ENSG00000250745 | USP17L20 |
| ENSG00000250799 | PRODH2 |
| ENSG00000250844 | USP17L18 |
| ENSG00000250913 | USP17L23 |
| ENSG00000251012 | RP11-484M3.5 |
| ENSG00000251184 | RP11-101E3.5 |
| ENSG00000251246 | RP11-540D14.8 |
| ENSG00000251258 | RFPL4B |
| ENSG00000251287 | ALG1L2 |
| ENSG00000251357 | AP000350.10 |
| ENSG00000251380 | DCANP1 |
| ENSG00000251493 | FOXD1 |
| ENSG00000251537 | RP11-385D13.1 |
| ENSG00000251569 | RP11-724O16.1 |
| ENSG00000251655 | PRB1 |
| ENSG00000251664 | PCDHA12 |
| ENSG00000251692 | PTX4 |
| ENSG00000253117 | OC90 |
| ENSG00000253148 | RGS21 |
| ENSG00000253159 | PCDHGA12 |
| ENSG00000253304 | TMEM200B |
| ENSG00000253309 | SERPINE3 |
| ENSG00000253313 | C1orf210 |
| ENSG00000253457 | SMIM18 |
| ENSG00000253485 | PCDHGA5 |
| ENSG00000253506 | NACA2 |
| ENSG00000253537 | PCDHGA7 |
| ENSG00000253548 | PYDC2 |
| ENSG00000253598 | SLC10A5 |
| ENSG00000253731 | PCDHGA6 |
| ENSG00000253767 | PCDHGA8 |
| ENSG00000253831 | ETV3L |
| ENSG00000253846 | PCDHGA10 |
| ENSG00000253873 | PCDHGA11 |
| ENSG00000253910 | PCDHGB2 |
| ENSG00000253953 | PCDHGB4 |
| ENSG00000253958 | CLDN23 |
| ENSG00000254122 | PCDHGB7 |
| ENSG00000254221 | PCDHGB1 |
| ENSG00000254245 | PCDHGA3 |
| ENSG00000254440 | PBOV1 |
| ENSG00000254445 | HSPB2-C11orf52 |
| ENSG00000254462 | TMX2-CTNND1 |
| ENSG00000254466 | OR4D10 |
| ENSG00000254535 | PABPC4L |
| ENSG00000254536 | RP11-108K14.8 |
| ENSG00000254550 | OMP |
| ENSG00000254553 | RP1-27O5.3 |
| ENSG00000254585 | MAGEL2 |
| ENSG00000254598 | CSNK2A3 |
| ENSG00000254636 | ARMS2 |
| ENSG00000254647 | INS |
| ENSG00000254656 | RTL1 |
| ENSG00000254673 | RP11-598P20.5 |
| ENSG00000254706 | RP11-565P22.6 |
| ENSG00000254726 | MEX3A |
| ENSG00000254732 | RP11-691N7.6 |
| ENSG00000254737 | OR10G4 |
| ENSG00000254806 | SYS1-DBNDD2 |
| ENSG00000254834 | OR5M10 |
| ENSG00000254852 | NPIPA2 |
| ENSG00000254959 | INMT-FAM188B |
| ENSG00000254997 | KRTAP5-9 |
| ENSG00000255009 | UBTFL1 |
| ENSG00000255012 | OR5M1 |
| ENSG00000255054 | RP1-317E23.6 |
| ENSG00000255071 | SAA2-SAA4 |
| ENSG00000255072 | PIGY |
| ENSG00000255104 | TBC1D26 |
| ENSG00000255151 | GLYATL1P3 |
| ENSG00000255181 | CCDC166 |
| ENSG00000255192 | NANOGP8 |
| ENSG00000255221 | CARD17 |
| ENSG00000255223 | OR5M11 |
| ENSG00000255245 | FXYD6-FXYD2 |
| ENSG00000255251 | PRR23D1 |
| ENSG00000255275 | RP13-279N23.2 |
| ENSG00000255292 | AP002884.2 |
| ENSG00000255298 | OR8G5 |
| ENSG00000255307 | OR52B2 |
| ENSG00000255330 | RP3-403A15.5 |
| ENSG00000255346 | NOX5 |
| ENSG00000255359 | CCDC179 |
| ENSG00000255374 | TAS2R43 |
| ENSG00000255378 | PRR23D2 |
| ENSG00000255408 | PCDHA3 |
| ENSG00000255432 | RP11-831H9.11 |
| ENSG00000255501 | CARD18 |
| ENSG00000255524 | NPIPB8 |
| ENSG00000255582 | OR10G2 |
| ENSG00000255663 | RP11-212D19.4 |
| ENSG00000255690 | TRIL |
| ENSG00000255713 | OR4D2 |
| ENSG00000255767 | RP13-512J5.1 |
| ENSG00000255804 | OR6J1 |
| ENSG00000255835 | RP4-559A3.7 |
| ENSG00000255837 | TAS2R20 |
| ENSG00000255863 | AC073610.5 |
| ENSG00000255872 | RP11-613M10.9 |
| ENSG00000255974 | CYP2A6 |
| ENSG00000256029 | RP11-190A12.7 |
| ENSG00000256045 | MTRNR2L10 |
| ENSG00000256061 | DYX1C1 |
| ENSG00000256100 | AP000721.4 |
| ENSG00000256162 | SMLR1 |
| ENSG00000256188 | TAS2R30 |
| ENSG00000256206 | RP11-140L24.4 |
| ENSG00000256222 | MTRNR2L3 |
| ENSG00000256349 | CTD-3074O7.11 |
| ENSG00000256374 | PPIAL4D |
| ENSG00000256394 | ASIC5 |
| ENSG00000256407 | RP11-446E24.4 |
| ENSG00000256436 | TAS2R31 |
| ENSG00000256566 | RP4-734P14.4 |
| ENSG00000256632 | RP13-672B3.2 |
| ENSG00000256762 | STH |
| ENSG00000256797 | KLRF2 |
| ENSG00000256812 | CAPNS2 |
| ENSG00000256825 | CTD-2140B24.4 |
| ENSG00000256861 | RP11-512M8.5 |
| ENSG00000256870 | SLC5A8 |
| ENSG00000256892 | MTRNR2L7 |
| ENSG00000256966 | RP11-613M10.8 |
| ENSG00000256977 | LIMS3 |
| ENSG00000256980 | KHDC1L |
| ENSG00000257008 | GPR142 |
| ENSG00000257046 | RP11-545J16.1 |
| ENSG00000257062 | RP11-125O5.2 |
| ENSG00000257065 | RP3-468K18.5 |
| ENSG00000257115 | OR11H12 |
| ENSG00000257127 | CLLU1 |
| ENSG00000257138 | TAS2R38 |
| ENSG00000257184 | HOXA10-HOXA9 |
| ENSG00000257207 | AC108938.5 |
| ENSG00000257341 | AL928654.7 |
| ENSG00000257355 | CTD-2006C1.10 |
| ENSG00000257411 | RP11-603J24.9 |
| ENSG00000257446 | ZNF878 |
| ENSG00000257921 | RP11-571M6.15 |
| ENSG00000258052 | AC025263.3 |
| ENSG00000258064 | RP11-293I14.2 |
| ENSG00000258083 | OR9A4 |
| ENSG00000258150 | RP11-345J4.3 |
| ENSG00000258223 | PRSS58 |
| ENSG00000258405 | ZNF578 |
| ENSG00000258417 | RP11-240B13.2 |
| ENSG00000258436 | RNASE12 |
| ENSG00000258453 | OR11H2 |
| ENSG00000258466 | RP11-1012A1.4 |
| ENSG00000258484 | SPESP1 |
| ENSG00000258529 | RP11-108O10.8 |
| ENSG00000258588 | TRIM6-TRIM34 |
| ENSG00000258653 | RP5-1021I20.4 |
| ENSG00000258677 | RP11-463D19.2 |
| ENSG00000258691 | RP11-404P21.8 |
| ENSG00000258713 | C20orf141 |
| ENSG00000258724 | CTD-2135J3.4 |
| ENSG00000258728 | RP11-195F19.29 |
| ENSG00000258817 | OR4C13 |
| ENSG00000258864 | CTC-554D6.1 |
| ENSG00000258873 | DUXA |
| ENSG00000258881 | AC007040.11 |
| ENSG00000258941 | RP11-407N17.3 |
| ENSG00000258986 | TMEM179 |
| ENSG00000258989 | RP11-47I22.4 |
| ENSG00000258992 | TSPY1 |
| ENSG00000259060 | RP11-14J7.7 |
| ENSG00000259066 | RP11-371E8.4 |
| ENSG00000259112 | NDUFC2-KCTD14 |
| ENSG00000259120 | SMIM6 |
| ENSG00000259164 | RP11-463C8.4 |
| ENSG00000259224 | SLC35G6 |
| ENSG00000259371 | RP11-468E2.6 |
| ENSG00000259511 | UBE2Q2L |
| ENSG00000259571 | BLID |
| ENSG00000259680 | RP11-812E19.9 |
| ENSG00000259784 | RP11-20I23.3 |
| ENSG00000259900 | RP11-343C2.7 |
| ENSG00000260001 | TGFBR3L |
| ENSG00000260007 | RP11-315D16.2 |
| ENSG00000260092 | RP11-77K12.7 |
| ENSG00000260170 | RP11-96O20.4 |
| ENSG00000260230 | FRRS1L |
| ENSG00000260234 | RP11-166N6.3 |
| ENSG00000260272 | RP11-20I23.1 |
| ENSG00000260371 | RP11-343C2.9 |
| ENSG00000260428 | SCX |
| ENSG00000260548 | RP6-24A23.6 |
| ENSG00000260836 | RP11-152F13.10 |
| ENSG00000260851 | RP11-403P17.5 |
| ENSG00000260869 | AC002310.13 |
| ENSG00000261115 | TMEM178B |
| ENSG00000261130 | RP11-1021N1.1 |
| ENSG00000261147 | RP11-697E2.6 |
| ENSG00000261210 | CLEC19A |
| ENSG00000261247 | GOLGA8T |
| ENSG00000261272 | MUC22 |
| ENSG00000261427 | CTD-2349B8.1 |
| ENSG00000261456 | TUBB8 |
| ENSG00000261509 | TP53TG3B |
| ENSG00000261582 | RP4-614O4.11 |
| ENSG00000261587 | TMEM249 |
| ENSG00000261594 | TPBGL |
| ENSG00000261603 | PRSS46 |
| ENSG00000261611 | AC010547.9 |
| ENSG00000261652 | C15orf65 |
| ENSG00000261667 | RP11-520P18.5 |
| ENSG00000261678 | SCRT1 |
| ENSG00000261701 | HPR |
| ENSG00000261717 | RP11-77K12.1 |
| ENSG00000261732 | LA16c-431H6.6 |
| ENSG00000261739 | GOLGA8S |
| ENSG00000261787 | TCF24 |
| ENSG00000261793 | RP11-350O14.18 |
| ENSG00000261794 | GOLGA8H |
| ENSG00000261832 | RP11-435I10.4 |
| ENSG00000261833 | RP11-58C22.1 |
| ENSG00000261915 | RP11-542C16.2 |
| ENSG00000261934 | PCDHGA9 |
| ENSG00000261949 | GFY |
| ENSG00000262180 | OCLM |
| ENSG00000262209 | PCDHGB3 |
| ENSG00000262302 | RP1-4G17.5 |
| ENSG00000262406 | MMP12 |
| ENSG00000262484 | CCER2 |
| ENSG00000262526 | CTD-2545G14.7 |
| ENSG00000262560 | RP11-296A16.1 |
| ENSG00000262576 | PCDHGA4 |
| ENSG00000262628 | OR1D5 |
| ENSG00000262633 | RP11-156P1.2 |
| ENSG00000262660 | RP13-1032I1.10 |
| ENSG00000262730 | RP11-1099M24.7 |
| ENSG00000263201 | CTC-479C5.6 |
| ENSG00000263620 | RP11-599B13.6 |
| ENSG00000263639 | MSMB |
| ENSG00000263715 | CRHR1 |
| ENSG00000263761 | GDF2 |
| ENSG00000263809 | RP11-849F2.7 |
| ENSG00000264058 | RP5-1028K7.3 |
| ENSG00000264187 | RP11-45M22.4 |
| ENSG00000264324 | RP11-287D1.3 |
| ENSG00000264424 | MYH4 |
| ENSG00000264545 | RP11-145E5.5 |
| ENSG00000264668 | RP13-582O9.6 |
| ENSG00000264813 | CTD-2501B8.1 |
| ENSG00000265107 | GJA5 |
| ENSG00000265203 | RBP3 |
| ENSG00000265264 | TIMM10B |
| ENSG00000265303 | CTD-2510F5.6 |
| ENSG00000265590 | AP000275.65 |
| ENSG00000265763 | ZNF488 |
| ENSG00000265817 | FSBP |
| ENSG00000265818 | EEF1E1-BLOC1S5 |
| ENSG00000266076 | CTD-2535L24.2 |
| ENSG00000266202 | RP1-66C13.4 |
| ENSG00000266302 | RP11-219A15.1 |
| ENSG00000266524 | GDF10 |
| ENSG00000266728 | AC015688.3 |
| ENSG00000266733 | TBC1D29 |
| ENSG00000266953 | RP11-618P17.4 |
| ENSG00000266997 | RP11-886H22.1 |
| ENSG00000267001 | AC006538.4 |
| ENSG00000267022 | AC084219.2 |
| ENSG00000267110 | CTD-2587H24.4 |
| ENSG00000267127 | RP11-795F19.5 |
| ENSG00000267140 | RP11-322E11.6 |
| ENSG00000267157 | CTB-54O9.9 |
| ENSG00000267168 | RP11-318A15.7 |
| ENSG00000267173 | CTC-512J12.6 |
| ENSG00000267179 | CTD-2006C1.13 |
| ENSG00000267206 | LCN6 |
| ENSG00000267261 | CTD-2132N18.3 |
| ENSG00000267314 | AC104532.2 |
| ENSG00000267318 | RP11-178C3.1 |
| ENSG00000267335 | CTB-60B18.6 |
| ENSG00000267360 | CTC-454I21.3 |
| ENSG00000267467 | APOC4 |
| ENSG00000267477 | CTC-398G3.6 |
| ENSG00000267545 | AC005779.2 |
| ENSG00000267552 | CTD-2528L19.4 |
| ENSG00000267561 | RP5-1052I5.2 |
| ENSG00000267618 | RAD51L3-RFFL |
| ENSG00000267631 | CGB1 |
| ENSG00000267699 | RP11-729L2.2 |
| ENSG00000267706 | CTD-2105E13.6 |
| ENSG00000267748 | CTB-102L5.4 |
| ENSG00000267795 | SMIM22 |
| ENSG00000267881 | AC011513.3 |
| ENSG00000267909 | CCDC177 |
| ENSG00000267952 | CTD-2207O23.12 |
| ENSG00000267978 | MAGEA9B |
| ENSG00000268009 | SSX4 |
| ENSG00000268083 | AC104534.3 |
| ENSG00000268089 | GABRQ |
| ENSG00000268104 | SLC6A14 |
| ENSG00000268107 | AC003005.4 |
| ENSG00000268133 | AC003002.4 |
| ENSG00000268163 | AC004076.9 |
| ENSG00000268173 | AC007192.4 |
| ENSG00000268182 | SMIM17 |
| ENSG00000268193 | AC002985.3 |
| ENSG00000268221 | OPN1MW |
| ENSG00000268223 | ARL14EPL |
| ENSG00000268279 | RP11-434D12.1 |
| ENSG00000268320 | SCGB1C2 |
| ENSG00000268361 | L34079.2 |
| ENSG00000268400 | CTD-3214H19.4 |
| ENSG00000268434 | AC011530.4 |
| ENSG00000268447 | SSX2B |
| ENSG00000268465 | CTC-273B12.7 |
| ENSG00000268533 | AC004076.7 |
| ENSG00000268606 | MAGEA2 |
| ENSG00000268614 | CTD-2207O23.10 |
| ENSG00000268629 | TEX13A |
| ENSG00000268643 | AC006486.9 |
| ENSG00000268651 | CTAG1A |
| ENSG00000268714 | CTD-2287O16.3 |
| ENSG00000268738 | HSFX2 |
| ENSG00000268750 | CTD-2583A14.10 |
| ENSG00000268797 | CTC-490E21.12 |
| ENSG00000268870 | CTD-3105H18.16 |
| ENSG00000268964 | ERVV-2 |
| ENSG00000268975 | MIA-RAB4B |
| ENSG00000268988 | SPANXN2 |
| ENSG00000268991 | AL021920.2 |
| ENSG00000269026 | AC003006.7 |
| ENSG00000269028 | MTRNR2L12 |
| ENSG00000269035 | CTD-2521M24.10 |
| ENSG00000269058 | CALR3 |
| ENSG00000269067 | ZNF728 |
| ENSG00000269095 | AC010646.3 |
| ENSG00000269113 | TRABD2B |
| ENSG00000269179 | CTC-326K19.6 |
| ENSG00000269190 | FBXO17 |
| ENSG00000269237 | CTD-2561J22.3 |
| ENSG00000269307 | CTD-2278I10.6 |
| ENSG00000269403 | CTD-2616J11.11 |
| ENSG00000269405 | NXF2 |
| ENSG00000269433 | OPN1MW3 |
| ENSG00000269437 | NXF2B |
| ENSG00000269469 | CTD-3148I10.9 |
| ENSG00000269476 | CTD-2583A14.9 |
| ENSG00000269502 | DMRTC1 |
| ENSG00000269526 | ERVV-1 |
| ENSG00000269533 | AC003002.6 |
| ENSG00000269545 | CTD-3138B18.4 |
| ENSG00000269547 | CTC-360G5.8 |
| ENSG00000269586 | CT45A10 |
| ENSG00000269590 | CTD-2192J16.24 |
| ENSG00000269699 | ZIM2 |
| ENSG00000269741 | CTC-518B2.8 |
| ENSG00000269755 | CTD-3105H18.18 |
| ENSG00000269791 | SSX4B |
| ENSG00000269855 | RNF225 |
| ENSG00000269881 | AC004754.3 |
| ENSG00000269964 | MEI4 |
| ENSG00000270011 | ZNF559-ZNF177 |
| ENSG00000270024 | C8orf44-SGK3 |
| ENSG00000270099 | RP11-248J23.7 |
| ENSG00000270106 | TSNAX-DISC1 |
| ENSG00000270168 | LA16c-380H5.3 |
| ENSG00000270188 | MTRNR2L11 |
| ENSG00000270249 | RP11-514P8.7 |
| ENSG00000270299 | RP5-850E9.3 |
| ENSG00000270316 | BORCS7-ASMT |
| ENSG00000270394 | MTRNR2L13 |
| ENSG00000270617 | URGCP-MRPS24 |
| ENSG00000270672 | MTRNR2L6 |
| ENSG00000270765 | GAS2L2 |
| ENSG00000270806 | C17orf50 |
| ENSG00000270885 | RASL10B |
| ENSG00000270898 | GPR75-ASB3 |
| ENSG00000270946 | CT45A9 |
| ENSG00000271079 | CTAGE15 |
| ENSG00000271271 | UGT2A2 |
| ENSG00000271321 | CTAGE6 |
| ENSG00000271567 | PPIAL4E |
| ENSG00000271698 | GS1-393G12.13 |
| ENSG00000271723 | MROH7-TTC4 |
| ENSG00000271793 | RP11-321N4.5 |
| ENSG00000271810 | RP11-426L16.10 |
| ENSG00000271949 | RP11-302M6.4 |
| ENSG00000272058 | FAM231D |
| ENSG00000272104 | XXcos-LUCA11.5 |
| ENSG00000272162 | RP11-637O19.3 |
| ENSG00000272297 | RP11-215A19.2 |
| ENSG00000272305 | RP11-894J14.5 |
| ENSG00000272442 | RP11-444E17.6 |
| ENSG00000272514 | CFAP206 |
| ENSG00000272647 | GS1-259H13.13 |
| ENSG00000272674 | PCDHB16 |
| ENSG00000272741 | RP11-447L10.1 |
| ENSG00000272772 | CTD-2410N18.5 |
| ENSG00000272804 | KRTAP10-7 |
| ENSG00000272896 | RP11-216L13.17 |
| ENSG00000272897 | RP1-309K20.6 |
| ENSG00000272968 | RBAK-RBAKDN |
| ENSG00000273003 | ARL2-SNX15 |
| ENSG00000273025 | RP11-106M3.5 |
| ENSG00000273045 | C2orf15 |
| ENSG00000273049 | RP11-834C11.12 |
| ENSG00000273079 | GRIN2B |
| ENSG00000273088 | RP11-201K10.3 |
| ENSG00000273111 | LYPD4 |
| ENSG00000273155 | RP11-38C17.1 |
| ENSG00000273167 | RP11-307N16.6 |
| ENSG00000273171 | CTB-96E2.2 |
| ENSG00000273217 | CTC-432M15.3 |
| ENSG00000273269 | RP11-761B3.1 |
| ENSG00000273274 | ZBTB8B |
| ENSG00000273291 | RP11-136C24.3 |
| ENSG00000273294 | C1QTNF3-AMACR |
| ENSG00000273331 | TM4SF19-TCTEX1D2 |
| ENSG00000273398 | RP11-474G23.1 |
| ENSG00000273513 | TBC1D3K |
| ENSG00000273547 | OR4F16 |
| ENSG00000273696 | CT45A7 |
| ENSG00000273706 | LHX1 |
| ENSG00000273734 | LLfos-48D6.2 |
| ENSG00000273777 | CEACAM20 |
| ENSG00000274068 | RP11-475E11.9 |
| ENSG00000274102 | OR4M2 |
| ENSG00000274183 | H2AFB1 |
| ENSG00000274209 | ANTXRL |
| ENSG00000274252 | GGTLC3 |
| ENSG00000274286 | ADRA2B |
| ENSG00000274322 | RP11-314N13.10 |
| ENSG00000274443 | C8orf89 |
| ENSG00000274529 | SEBOX |
| ENSG00000274600 | RIMBP3B |
| ENSG00000274736 | CCL23 |
| ENSG00000274744 | TCEB3CL2 |
| ENSG00000274749 | KRTAP7-1 |
| ENSG00000274750 | HIST1H3E |
| ENSG00000274791 | F8A2 |
| ENSG00000274874 | RP11-214K3.25 |
| ENSG00000274933 | TBC1D3I |
| ENSG00000275034 | TP53TG3E |
| ENSG00000275038 | RP11-546B8.6 |
| ENSG00000275152 | CCL16 |
| ENSG00000275163 | RP11-385J1.3 |
| ENSG00000275356 | C7orf77 |
| ENSG00000275410 | HNF1B |
| ENSG00000275489 | C17orf98 |
| ENSG00000275572 | GRIFIN |
| ENSG00000275591 | XKR5 |
| ENSG00000275663 | HIST1H4G |
| ENSG00000275674 | RP11-697E2.12 |
| ENSG00000275688 | CCL15-CCL14 |
| ENSG00000275718 | CCL15 |
| ENSG00000275722 | LYZL6 |
| ENSG00000275774 | HNRNPCL2 |
| ENSG00000276076 | CH507-152C13.3 |
| ENSG00000276087 | RP11-507M3.1 |
| ENSG00000276119 | OR13C2 |
| ENSG00000276302 | RP5-874C20.8 |
| ENSG00000276368 | HIST1H2AJ |
| ENSG00000276410 | HIST1H2BB |
| ENSG00000276418 | RP11-26J3.4 |
| ENSG00000276430 | FAM25C |
| ENSG00000276490 | RP11-400G3.5 |
| ENSG00000276547 | PCDHGB5 |
| ENSG00000276612 | CH507-9B2.4 |
| ENSG00000276747 | PADI6 |
| ENSG00000276911 | RP4-608O15.3 |
| ENSG00000277288 | C10orf142 |
| ENSG00000277322 | GOLGA6L6 |
| ENSG00000277399 | GPR179 |
| ENSG00000277481 | PKD1L3 |
| ENSG00000277494 | GPIHBP1 |
| ENSG00000277535 | RP13-347D8.7 |
| ENSG00000277556 | OR13C5 |
| ENSG00000277611 | RP1-138B7.6 |
| ENSG00000277669 | AC009133.22 |
| ENSG00000277726 | RP4-635E18.9 |
| ENSG00000277758 | ABC7-42404400C24.1 |
| ENSG00000277858 | H2AFB2 |
| ENSG00000277865 | GOLGA6L22 |
| ENSG00000277877 | RP11-11N7.5 |
| ENSG00000277893 | SRD5A2 |
| ENSG00000277932 | OR52E5 |
| ENSG00000277971 | XXbac-B562F10.12 |
| ENSG00000278023 | RDM1 |
| ENSG00000278057 | TEX28 |
| ENSG00000278085 | CT45A8 |
| ENSG00000278139 | PIK3R3 |
| ENSG00000278318 | ZNF229 |
| ENSG00000278463 | HIST1H2AB |
| ENSG00000278499 | RP11-457D20.2 |
| ENSG00000278500 | AC009336.19 |
| ENSG00000278505 | C17orf78 |
| ENSG00000278522 | POTEB3 |
| ENSG00000278570 | NR2E3 |
| ENSG00000278646 | RP1-321E8.5 |
| ENSG00000278674 | RP11-49K24.9 |
| ENSG00000278685 | IQCA1L |
| ENSG00000278848 | TP53TG3F |
| ENSG00000279068 | CH17-140K24.5 |
| ENSG00000279132 | FAM231C |
| ENSG00000279408 | RP11-294C11.3 |
| ENSG00000279493 | CH507-9B2.2 |
| ENSG00000279624 | RP5-1042K10.14 |
| ENSG00000279956 | RP11-310N16.1 |
| ENSG00000279968 | GVQW2 |
| ENSG00000279983 | CH17-140K24.8 |
| ENSG00000280148 | RP3-468K18.7 |
| ENSG00000280165 | PCDH20 |
| ENSG00000280571 | RP4-613B23.5 |
| ENSG00000280778 | CH17-270A2.2 |
| ENSG00000280893 | AC009133.23 |
| ENSG00000280969 | RPS4Y2 |
| ENSG00000281028 | RP11-717K11.2 |
| ENSG00000281039 | RP4-777O23.3 |
| ENSG00000281593 | GS1-114I9.3 |
| ENSG00000281613 | RP11-506B6.7 |
| ENSG00000281883 | RP11-65B7.2 |
| ENSG00000281938 | CTB-127M13.1 |
| ENSG00000282218 | RP1-179P9.3 |
| ENSG00000282246 | RP11-295P9.13 |
| ENSG00000282278 | RP11-231C18.3 |
| ENSG00000282301 | CYP3A7-CYP3A51P |
| ENSG00000282419 | RP5-1052M9.5 |
| ENSG00000282804 | RP3-369A17.6 |
| ENSG00000282815 | RP13-147D17.3 |
| ENSGR0000002586 | CD99 |
| ENSGR0000124333 | VAMP7 |
| ENSGR0000124334 | IL9R |
| ENSGR0000167393 | PPP2R3B |
| ENSGR0000168939 | SPRY3 |
| ENSGR0000169084 | DHRSX |
| ENSGR0000169093 | ASMTL |
| ENSGR0000169100 | SLC25A6 |
| ENSGR0000178605 | GTPBP6 |
| ENSGR0000182162 | P2RY8 |
| ENSGR0000182378 | PLCXD1 |
| ENSGR0000185291 | IL3RA |
| ENSGR0000185960 | SHOX |
| ENSGR0000196433 | ASMT |
| ENSGR0000197976 | AKAP17A |
| ENSGR0000198223 | CSF2RA |
| ENSGR0000205755 | CRLF2 |
| ENSGR0000214717 | ZBED1 |

1.-66. (canceled)

67. A method comprising:
(a) obtaining a sample comprising a plurality of cell-free nucleic acid molecules, the plurality of cell-free nucleic acid molecules comprising a plurality of cell-free RNA molecules from a biological sample of an individual or a plurality of DNA molecules complementary to the plurality of cell-free RNA molecules;
(b) obtaining a panel of nucleic acid molecules that enriches for nucleic acids from a subset of a whole exome, wherein the subset comprises at least 5% of target genes identified in Table 3, wherein the target genes are rarely abundant as cell-free RNA transcripts within a plurality of control liquid biopsies; and
(c) enriching from the sample for the nucleic acids by contacting the sample with the panel of nucleic acid molecules.

68. The method of claim 67, wherein the panel of nucleic acid molecules enriches for at least 30% of the target genes identified in Table 3.

69. The method of claim 67, wherein the panel of nucleic acid molecules enriches for genes associated with a plurality of tissue types.

70. The method of claim 69, wherein the plurality of tissue types comprises two or more members selected from bladder, kidney, liver, lung, pancreas, prostate, and breast.

71. The method of claim 67, wherein the panel of nucleic acid molecules enriches for genes associated with a plurality of cell types.

72. The method of claim 67, wherein the panel of nucleic acid molecules enriches for genes associated with a plurality of cancer types.

73. The method of claim 72, wherein the plurality of cancer types comprises two or more members selected from lung adenocarcinomas (LUAD), pancreatic adenocarcinoma (PAAD), prostate adenocarcinoma (PRAD), and hepatocellular carcinoma (LIHC).

74. The method of claim 67, wherein the panel of nucleic acid molecules enriches for lung cancer-associated genes.

75. The method of claim 67, wherein the panel of nucleic acid molecules enriches for bladder cancer-associated genes.

76. The method of claim 67, wherein the panel of nucleic acid molecules is a set of nucleic acid probes for targeted capture hybridization or a set of nucleic acid primers for targeted amplification.

77. The method of claim 67, wherein the panel of nucleic acid molecules lacks platelet-associated genes.

78. The method of claim 67, further comprising:
generating sequencing read data of the nucleic acids; and
aligning the sequencing read data to a reference database to identify at least one member of the target genes.

79. The method of claim 78, wherein the at least one member comprises a plurality of members of the target genes.

80. The method of claim 78, further comprising determining a gene expression profile of the at least one member of the target genes based on the aligning.

81. The method of claim 80, wherein the individual is known to have a biological condition, and wherein the method further comprises (i) comparing the gene expression profile to a control gene expression profile of cell-free RNA libraries from a cohort of control subjects and (ii) identifying at least one gene that is differentially expressed in the individual as compared to the cohort of control subjects.

82. The method of claim 80, further comprising determining a presence of a gene expression signature within the gene expression profile, wherein the gene expression signature is indicative of the individual's likelihood of having a biological condition.

83. The method of claim 82, wherein the determining the presence of the gene expression signature comprises generating an enrichment score of at least one predetermined gene within the gene expression profile.

84. The method of claim 83, wherein the at least one predetermined gene comprises a plurality of predetermined genes.

85. The method of claim 81, further comprising excluding, in silico, a gene expression contribution of platelets from the gene expression profile.

86. The method of claim 78, further comprising identifying, based at least in part on the at least one member of the target genes, a somatic alteration selected from a single nucleotide variant (SNV), an insertion or deletion (indel), a gene fusion, and a splice variant.

87. The method of claim 78, further comprising identifying, based at least in part on the at least one member of the target genes, a mechanism of resistance to a treatment for the individual.

88. The method of claim 78, further comprising determining, based at least in part on the at least one member of the target genes, a biological condition of the individual.

89. The method of claim 88, further comprising determining, based at least in part on the at least one member of the target genes, the individual's response to a treatment for the biological condition.

90. The method of claim 88, further comprising determining, based at least in part on the at least one member of the target genes, a treatment strategy for the biological condition of the individual.

91. The method of claim 90, further comprising subjecting the individual to the treatment strategy.

92. The method of claim 88, wherein the biological condition is cancer.

93. The method of claim 67, wherein the biological sample is blood, plasma, or urine.

94. The method of claim 78, further comprising analyzing, via a machine learning model, a gene expression profile of the at least one member of the target genes, a somatic alteration derived from the at least one member of the target genes, or both to determine the individual's likelihood of having a biological condition.

95. The method of claim 94, wherein the machine learning model is based on elastic net, random forest, XGBoost, or neural network.

96. A method comprising:
(a) obtaining, by a computer system, sequencing read data of a plurality of cell-free RNA molecules from a biological sample of an individual;
(b) aligning, by the computer system, the sequencing read data to a reference database to identify a first gene expression profile of a plurality of genes from which the plurality of cell-free RNA molecules is transcribed;
(c) excluding, by the computer system, a gene expression contribution of platelets from the first gene expression profile, thereby generating a second gene expression profile of the plurality of genes that lacks the gene expression contribution from the platelets; and
(d) determining, by the computer system, a gene expression signature of at least one gene within the second gene expression profile, wherein the gene expression signature is associated with a biological condition of the individual.
